(19) United States
(12) Patent Application Publication
(10) Pub. No.: US 2026/0202745 A1
(43) Pub. Date: Jul. 16, 2026

(54) RESIST COMPOSITION, LAMINATE, AND PATTERNING PROCESS

(71) Applicant: SHIN-ETSU CHEMICAL CO., LTD., Tokyo (JP)

(72) Inventors: Shun KIKUCHI, Joetsu-shi (JP); Kousuke OHYAMA, Joetsu-shi (JP); Ryo NISHIKAWA, Joetsu-shi (JP); Satoshi KUSAMA, Joetsu-shi (JP); Masaki OHASHI, Joetsu-shi (JP)

(73) Assignee: SHIN-ETSU CHEMICAL CO., LTD., Tokyo (JP)

(21) Appl. No.: 19/447,086

(22) Filed: Jan. 13, 2026

(30) Foreign Application Priority Data
Jan. 16, 2025 (JP) 2025-006320

(51) Int. Cl. G03F 7/004; C07D 347/00; C08F 20/04; C08G 63/00; G03F 7/00; G03F 7/038; G03F 7/039; G03F 7/20

(52) U.S. Cl. CPC G03F 7/0045; C07D 347/00; C08F 20/04; C08G 63/00; G03F 7/038; G03F 7/039; G03F 7/2004; G03F 7/70033

(57) ABSTRACT

The present invention is a resist composition including at least one kind of hypervalent iodine compounds selected from the following formulae (1), (2), and (3), a carboxy group-containing compound, and a solvent. In the formula, n1 represents 0 to 2; n2 represents 0 to 2; n3 represents 0 to 3; R¹¹ to R¹³ represent a halogen atom or a hydrocarbyl group having 1 to 10 carbon atoms; X¹ to X³ represent *—O—X^{A1}— or *—X^{A2}(—R¹⁴)—C(═O)—; X^{A1} represents a carbonyl group or a hydrocarbylene group having 1 to 10 carbon atoms; X^{A2} represents nitrogen or sulfur; and R¹⁴ represents a hydrogen atom, a halogen atom, or a hydrocarbyl group having 1 to 20 carbon atoms. This can provide a non-chemically amplified resist composition that is excellent in sensitivity and a resolution limit in photolithography with a high-energy beam, in particular in electron beam (EB) lithography and EUV lithography.

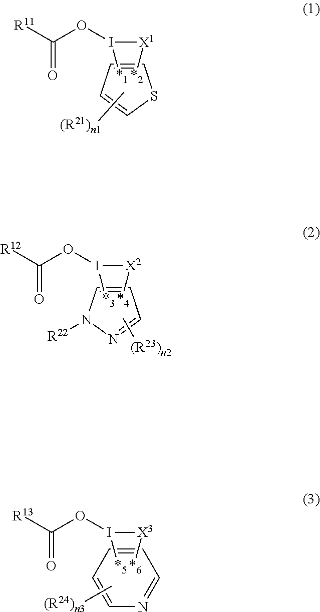

### TECHNICAL FIELD

The present invention relates to a resist composition, a laminate, and a patterning process using the resist composition.

### BACKGROUND ART

Along with the expansion of the IoT market, LSIs are further required to have a higher degree of integration, higher speed, and lower power consumption, and miniaturization of pattern rules is in rapid progress. In particular, logic devices lead the miniaturization. State-of-the-art miniaturization techniques implemented include volume manufacturing of 10-nm node devices by double patterning, triple patterning, and quadruple patterning with ArF immersion lithography, and furthermore studies about 7-nm node devices with next-generation extreme-ultraviolet (EUV) lithography at a wavelength of 13.5 nm are in progress.

With the progress of miniaturization, image blur due to acid diffusion is problematic (Non Patent Document 1). In order to ensure resolution in fine patterns with a critical dimension of 45 nm or less, it is proposed that not only an enhancement in dissolution contrast, as conventionally proposed, but also control of acid diffusion are important (Non Patent Document 2) However, in chemically amplified resist compositions, sensitivity and contrast are enhanced by acid diffusion; therefore, when acid diffusion is attempted to be inhibited as much as possible by lowering post-exposure bake (PEB) temperature or shortening PEB time, the sensitivity and contrast deteriorate remarkably.

It is effective to inhibit acid diffusion by the addition of acid generators that generate a bulky acid. There is then a proposal for copolymerizing acid generators of onium salts having polymerizable olefins, with polymers. However, in patterning on resist films with a critical dimension of 16 nm or less, it is considered impossible to form a pattern with the chemically amplified resist compositions from the viewpoint of acid diffusion, and it is desired to develop non-chemically amplified resist compositions.

Examples of materials for the non-chemically amplified resist compositions include polymethyl methacrylate (PMMA). PMMA is a positive-type resist material whose solubility in an organic solvent developer increases by decreasing molecular weight due to scission of the main chain by EUV irradiation.

Hydrogen silsesquioxane (HSQ) is a negative-type resist material that is made insoluble in an alkali developer due to crosslinking by a condensation reaction of silanol generated by EUV irradiation. Chlorine-substituted calixarene also serves as a negative-type resist material. These negative-type resist materials have a small molecular size before crosslinking, do not cause any blur due to acid diffusion, have small edge roughness and very high resolvability, and therefore, are used as a pattern-transfer material to exhibit a resolution limit of an exposure apparatus. However, these materials have insufficient sensitivity and require further improvement.

Examples of factors making material development for EUV lithography difficult include a small number of photons in EUV exposure. The energy of EUV is much higher than that of ArF excimer laser beam, and the number of photons in the EUV exposure is one-fourteenth of that in the ArF exposure. Furthermore, the dimensions of a pattern formed by the EUV exposure are less than half of those formed by the ArF exposure. Therefore, the EUV exposure is readily affected by variations in the number of photons. The variation in the number of photons in the region of radiation light at extremely short wavelengths is shot noise as a physical phenomenon, and the influence of this shot noise cannot be eliminated. Therefore, so-called probability theory (stochastics) attracts attention. Although the influence of the shot noise cannot be eliminated, how to reduce this influence is under discussion. Phenomena are observed, in which the influence of the shot noise leads to not only increases in critical dimension uniformity (CDU) and line width roughness (LWR), but also blocking of a hole at a probability of one in several million. Such blocking a hole causes a failure of electrical conduction, stopping transistor operation and therefore adversely affecting the performance of the entire device. In light of practical sensitivity, resist compositions mainly containing PMMA or HSQ are highly affected by stochastics, and have not yet achieved the desired resolving performance.

As a method from the resist side to reduce the influence of the shot noise, introduction of elements with high absorption of EUV light has attracted attention. Patent Document 1 proposes a chemically amplified resist composition having an iodine atom with high absorption of the EUV light. However, as described above, the chemically amplified resist compositions cannot achieve excellent resolving performance in EUV lithography, where a process dimension will continue to decrease. In particular, for line-and-space patterns, as the process dimension decreases, collapse of a pattern and disconnection increase significantly.

Reducing these leads to improvements in a resolution limit.

Patent Document 2 proposes a negative-type resist composition in which a tin compound is used. This composition mainly contains a tin element with high absorption of EUV light, and therefore, it is improved in stochastics, and high sensitivity/high resolvability can be achieved. However, so-called such metal resists have many problems, such as insufficient solubility in solvents for resists, storage stability, and defects due to residues after etching. Furthermore, since the metal resist is primarily a negative-type material in which exposed area becomes a metal oxide and thus becomes insoluble in developers, applying it to contact hole patterning requires an additional inversion process step, raising cost concerns.

### CITATION LIST

### Patent Literature

Patent Document 1: JP2018-5224A
Patent Document 2: JP2021-503482A

### Non Patent Literature

Non Patent Document 1: SPIE Vol. 5039 p1 (2003)
Non Patent Document 2: SPIE Vol. 6520 p65203L-1 (2007)

### SUMMARY OF INVENTION

### Technical Problem

The present invention has been made in view of the above circumstances, and an object thereof is to provide a non-chemically amplified resist composition that is excellent in sensitivity and a resolution limit in photolithography with a high-energy beam, in particular, in EUV lithography; a laminate using the resist composition; and a patterning process using the resist composition.

### Solution to Problem

To solve the above problem, the present invention provides a resist composition comprising at least one kind of hypervalent iodine compounds selected from the following formulae (1), (2), and (3), a carboxy group-containing compound, and a solvent,

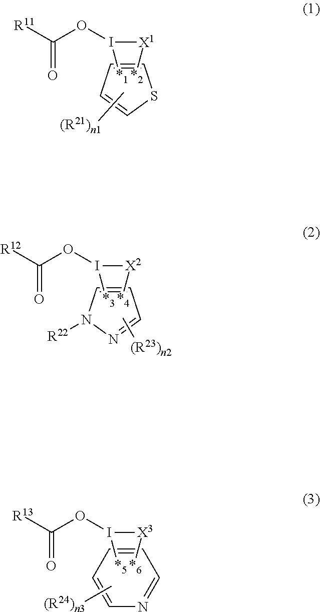

wherein n1 represents 0, 1, or 2; n2 represents 0, 1, or 2; n3 represents 0, 1, 2, or 3; R¹¹ to R¹³ each independently represent a halogen atom or a hydrocarbyl group having 1 to 10 carbon atoms and optionally having a heteroatom; X¹ to X³ each independently represent *—O—X^{A1}— or *—X^{A2} (—R¹⁴)—C(═O)—; X³ may be *—S—X^{A1}—; X^{A1} represents a carbonyl group or a hydrocarbylene group having 1 to 10 carbon atoms and optionally having a heteroatom; X^{A2} represents nitrogen or sulfur, and when X^{A2} is nitrogen, R¹⁴ may be included; R¹⁴ represents a hydrogen atom, a halogen atom, or a hydrocarbyl group or ester having 1 to 20 carbon atoms and optionally having a heteroatom; “*” represents a bond to an iodine atom in the formula; *1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle; R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN; R²² may be a hydrogen atom; When n1 is 2 or more, R²¹s each may be identical to or different from each other, and a plurality of R²¹s may be bonded to each other to form a ring together with the carbon atoms of the heteroaromatic ring to which they are bonded; and when n3 is 2 or more, R²⁴s each may be identical to or different from each other, and a plurality of R²⁴s may be bonded to each other to form a ring together with the carbon atoms of the heteroaromatic ring to which they are bonded, provided that R²² is X² when X² is bonded to a nitrogen atom to which R²² is bonded.

Such a resist composition is a non-chemically amplified resist composition, which is excellent in sensitivity and a resolution limit in photolithography with a high-energy beam, in particular in electron beam (EB) lithography and EUV lithography.

Further, the carboxy group-containing compound is preferably either or both of a polymer having a repeating unit represented by the following formula (4) and a compound represented by the following formula (5),

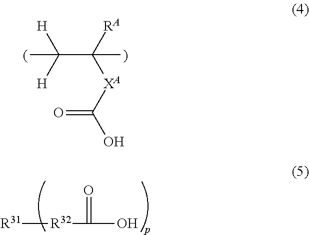

wherein R^{A} represents a hydrogen atom, a halogen atom, a methyl group, or a trifluoromethyl group; X^{A} represents a single bond, a phenylene group, a naphthylene group, or *—C(═O)—O—X^{A3}—; X^{A3} represents a saturated hydrocarbylene group having 1 to 20 carbon atoms, a phenylene group, or a naphthylene group, and the saturated hydrocarbylene group may have a hydroxy group, an ether bond, an ester bond, or a lactone ring; “*” represents a bond to a carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, and when “p” is 2, R³¹ may be an ether bond, a carbonyl group, an azo group, a thioether bond, a carbonate bond, a carbamate bond, a sulfinyl group, or a sulfonyl group; In addition, part or all of the hydrogen atoms of the p-valent hydrocarbon group or the p-valent heterocyclic group may be substituted with a group having a heteroatom, and part of the —CH₂— groups of the p-valent hydrocarbon group may be substituted with a group having a heteroatom; R³² represents a single bond or a hydrocarbylene group having 1 to 20 carbon atoms, part or all of the hydrogen atoms of the hydrocarbylene group may be substituted with a group having a heteroatom, and part of the —CH₂— groups of the hydrocarbylene group may be substituted with a group having a heteroatom; and when “p” is 2, 3, or 4, R³²s each may be identical to or different from each other.

Such a compound is particularly suitable for use as the carboxy group-containing compound.

Further, the present invention provides a laminate comprising a substrate and a resist film, which is a film of the above resist composition, on the substrate.

Such a laminate is a laminate using a non-chemically amplified resist composition, which is excellent in sensitivity and a resolution limit in photolithography with a high-energy beam, in particular in electron beam (EB) lithography and EUV lithography.

Further, the inventive laminate further comprises a resist underlayer film between the substrate and the resist film.

The inventive resist composition may also be formed in this manner as needed.

Further, the resist film preferably contains a product of a ligand-exchange reaction between the hypervalent iodine compound and the carboxy group-containing compound.

Such a resist film is excellent in sensitivity and a resolution limit, and can form a pattern of both a positive-type and a negative-type.

Further, the present invention provides a patterning process comprising the steps of: forming a resist film on a substrate or on a resist underlayer film of a substrate having the resist underlayer film laminated thereon, using the above resist composition;
exposing the resist film by a high-energy beam; and developing the exposed resist film by using a developer.

Such a patterning process is excellent in sensitivity and a resolution limit in photolithography with a high-energy beam, in particular in electron beam (EB) lithography and EUV lithography.

Further, an i-line, a KrF excimer laser beam, an ArF excimer laser beam, an electron beam, or an extreme ultraviolet ray is preferably used as the high-energy beam.

In the inventive patterning process, such high-energy beams may be particularly suitably used.

In the inventive patterning process, the developer used may be one that dissolves exposed areas and does not dissolve unexposed areas.

Or, the developer used may be one that dissolves unexposed areas and does not dissolve exposed areas.

The inventive resist film can form a pattern of both a positive-type and a negative-type.

### Advantageous Effects of Invention

The inventive resist composition can achieve both high sensitivity and high resolvability, particularly in photolithography using an i-line, a KrF excimer laser beam, an ArF excimer laser beam, an EB, or an EUV, and is extremely useful for forming a fine pattern.

### DESCRIPTION OF EMBODIMENTS

As described above, it has been desired to develop a non-chemically amplified resist composition that is excellent in sensitivity and a resolution limit in photolithography with a high-energy beam, in particular, in EUV lithography; a laminate using the resist composition; and a patterning process using the resist composition.

To achieve the object, the present inventors have studied earnestly and found out that a resist composition mainly containing a predetermined hypervalent iodine compound and a carboxy group-containing compound (a polymer or a low-molecular compound) could give a resist film exhibiting excellent resolution and was extremely useful for precise fine processing, and have completed the present invention.

That is, the present invention is a resist composition comprising at least one kind of hypervalent iodine compounds selected from the following formulae (1), (2), and (3), a carboxy group-containing compound, and a solvent,

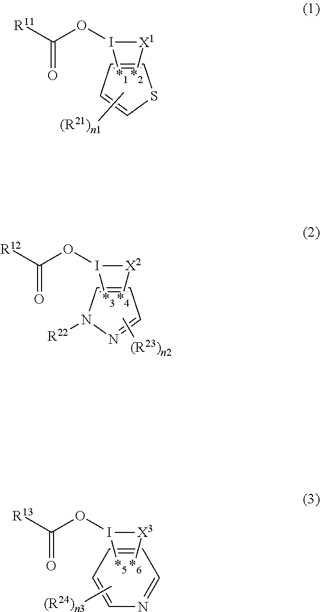

wherein n1 represents 0, 1, or 2; n2 represents 0, 1, or 2; n3 represents 0, 1, 2, or 3; R¹¹ to R¹³ each independently represent a halogen atom or a hydrocarbyl group having 1 to 10 carbon atoms and optionally having a heteroatom; X¹ to X³ each independently represent *—O—X^{A1}— or *—X^{A2}(—R¹⁴)—C(═O)—; X³ may be *—S—X^{A1}—; X^{A1} represents a carbonyl group or a hydrocarbylene group having 1 to 10 carbon atoms and optionally having a heteroatom; X^{A2} represents nitrogen or sulfur, and when X^{A2} is nitrogen, R¹⁴ may be included; R¹⁴ represents a hydrogen atom, a halogen atom, or a hydrocarbyl group or ester having 1 to 20 carbon atoms and optionally having a heteroatom; “*” represents a bond to an iodine atom in the formula; *1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle; R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN; R²² may be a hydrogen atom; When n1 is 2 or more, R²¹s each may be identical to or different from each other, and a plurality of R²¹s may be bonded to each other to form a ring together with the carbon atoms of the heteroaromatic ring to which they are bonded; and when n3 is 2 or more, R²⁴s each may be identical to or different from each other, and a plurality of R²⁴s may be bonded to each other to form a ring together with the carbon atoms of the heteroaromatic ring to which they are bonded, provided that R²² is X² when X² is bonded to a nitrogen atom to which R²² is bonded.

Hereinafter, the present invention will be described in detail, but the present invention is not limited thereto. It should be understood that, in the present specification, the recitations of numerical ranges by endpoints include all numbers subsumed within that range (e.g., “0 to 3” includes 0, 1, 2, and 3).

### [Resist Composition]

The inventive resist composition mainly contains a predetermined hypervalent iodine compound, a carboxy group-containing compound, and a solvent.

### [Hypervalent Iodine Compound]

“Hypervalent iodine compound” is a general term for iodine compounds having valence electrons formally exceeding the octet rule, and examples include tricoordinate iodine compounds (iodine (III) compounds) having an oxidation number of +3, and pentacoordinate iodine compounds (iodine (V) compounds) having an oxidation number of +5.

The hypervalent iodine compound, which is a main component of the inventive resist composition, is at least one kind of tricoordinate hypervalent iodine compounds selected from the following formulae (1), (2), and (3).

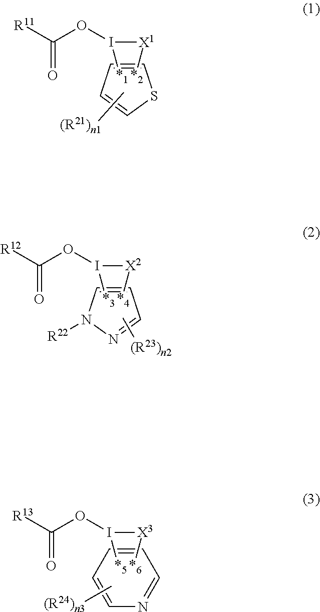

In the formulae, n1 represents 0, 1, or 2; n2 represents 0, 1, or 2; n3 represents 0, 1, 2, or 3; R¹¹ to R¹³ each independently represent a halogen atom or a hydrocarbyl group having 1 to 10 carbon atoms and optionally having a heteroatom; X¹ to X³ each independently represent *—O—X^{A1}— or *—X^{A2}(—R¹⁴)—C(═O)—; X³ may be *—S-X^{A}1—; X^{A1} represents a carbonyl group or a hydrocarbylene group having 1 to 10 carbon atoms and optionally having a heteroatom; X^{A2} represents nitrogen or sulfur, and when X^{A2} is nitrogen, R¹⁴ may be included; R¹⁴ represents a hydrogen atom, a halogen atom, or a hydrocarbyl group or ester having 1 to 20 carbon atoms and optionally having a heteroatom; “*” represents a bond to an iodine atom in the formula; *1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle; R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN; R²² may be a hydrogen atom; When n1 is 2 or more, R²¹s each may be identical to or different from each other, and a plurality of R²¹s may be bonded to each other to form a ring together with the carbon atoms of the heteroaromatic ring to which they are bonded; and when n3 is 2 or more, R²⁴s each may be identical to or different from each other, and a plurality of R²⁴s may be bonded to each other to form a ring together with the carbon atoms of the heteroaromatic ring to which they are bonded, provided that R²² is X² when X² is bonded to a nitrogen atom to which R²² is bonded.

In the formula, n1 represents 0, 1, or 2, n2 represents 0, 1, or 2, and n3 represents 0, 1, 2, or 3.

R¹¹ to R¹³ each independently represent a halogen atom or a hydrocarbyl group having 1 to 10 carbon atoms and optionally having a heteroatom.

Examples of the halogen atom represented by R¹¹ to R¹³ include a fluorine atom, a chlorine atom, a bromine atom, and an iodine atom. The hydrocarbyl group having 1 to 10 carbon atoms, represented by R¹¹ to R¹³, may be saturated or unsaturated, and may be any of linear, branched, or cyclic. Specific examples thereof include: alkyl groups having 1 to 10 carbon atoms, such as a methyl group, an ethyl group, a propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group, and a n-decyl group; saturated cyclic hydrocarbyl groups having 3 to 10 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, tricyclo[5.2.1.0^{2,6}]decanyl group, and an adamantyl group; alkenyl groups, such as a vinyl group and an allyl group; aryl groups having 6 to 10 carbon atoms, such as a phenyl group and a naphthyl group; and any group obtained by combination thereof. Part or all of the hydrogen atoms of the hydrocarbyl group may be substituted with a group having a heteroatom, such as an oxygen atom, a sulfur atom, a nitrogen atom, or a halogen atom, part of the —CH₂— groups of the hydrocarbyl group may be substituted with a group having a heteroatom, such as an oxygen atom, a sulfur atom, or a nitrogen atom, and as a result, a hydroxy group, a cyano group, a halogen atom, a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), or the like may be contained. R¹¹ to R¹³ are each preferably a hydrocarbyl group having 1 to 4 carbon atoms.

X¹ to X³ each independently represent *—O—X^{A}1— or *—X^{A}2(—R¹⁴)—C(═O)—. X³ may be *—S—X^{A1}—. X^{A1} represents a carbonyl group or a hydrocarbylene group having 1 to 10 carbon atoms and optionally having a heteroatom. X^{A2} represents nitrogen or sulfur, and when X^{A2} is nitrogen, R¹⁴ may be included. R¹⁴ represents a hydrogen atom, a halogen atom, or a hydrocarbyl group or ester having 1 to 20 carbon atoms and optionally having a heteroatom. “*” represents a bond to an iodine atom in the formula.

*1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle.

X^{A1} represents a carbonyl group or a hydrocarbylene group having 1 to 10 carbon atoms and optionally having a heteroatom. The hydrocarbylene group having 1 to 10 carbon atoms may be saturated or unsaturated, and may be linear, branched, or cyclic. Specific examples include: alkylene groups having 1 to 10 carbon atoms, such as a methanediyl group, an ethane-1,1-diyl group, an ethane-1,2-diyl group, a propane-1,1-diyl group, a propane-1,2-diyl group, a propane-1,3-diyl group, a propane-2,2-diyl group, a butane-2,3-diyl group, a butane-1,4-diyl group, a 2-methylpropane-1,2-diyl group, a pentane-1,5-diyl group, a hexane-1,6-diyl group, a heptane-1,7-diyl group, an octane-1,8-diyl group, a nonane-1,9-diyl group, and a decane-1,10-diyl group; saturated cyclic hydrocarbylene groups having 3 to 10 carbon atoms, such as a cyclopentane diyl group, a cyclohexane diyl group, a norbornane diyl group, an adamantane diyl group, and a tricyclo[5.2.1.0^{2,6}]decanediyl group; alkenylene groups having 2 to 10 carbon atoms, such as a vinylene group and a propynylene group; arylene groups having 6 to 10 carbon atoms, such as a phenylene group, a methylphenylene group, an ethylphenylene group, a n-propylphenylene group, an isopropylphenylene group, a n-butylphenylene group, and a naphthylene group; and groups obtained by combining these. Furthermore, part or all of the hydrogen atoms of the hydrocarbylene group may be substituted with a group having a heteroatom, such as an oxygen atom, a sulfur atom, a nitrogen atom, or a halogen atom, part of the —CH₂— groups of the hydrocarbylene group may be substituted with a group having a heteroatom, such as an oxygen atom, a sulfur atom, or a nitrogen atom, and as a result, a hydroxy group, a cyano group, a halogenated alkyl group, a halogen atom, a carbonyl group, an ether bond, a thioether bond, an ester bond, a sulfonate ester bond, a carbonate bond, a carbamate bond, a lactone ring, a sultone ring, a carboxylic anhydride (—C(═O)—O—C(═O)—), or the like may be contained. X^{A1} is preferably a carbonyl group, a hydrocarbylene group having 1 to 4 carbon atoms, or a fluorinated hydrocarbylene group having 1 to 4 carbon atoms.

X^{A2} represents nitrogen or sulfur, and when X^{A2} is nitrogen, R¹⁴ may be included. R¹⁴ represents a hydrogen atom, a halogen atom, or a hydrocarbyl group or ester having 1 to 20 carbon atoms and optionally having a heteroatom. Specific examples of the halogen atom and the hydrocarbyl group represented by R¹⁴ include the same as those exemplified as the halogen atom and the hydrocarbyl group each represented by R¹¹ to R¹³.

R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN. R²² may be a hydrogen atom. When n1 is 2 or more, R²¹s each may be identical to or different from each other, and a plurality of R²¹S may be bonded to each other to form a ring together with the carbon atoms of the heteroaromatic ring to which they are bonded. When n3 is 2 or more, R²⁴s each may be identical to or different from each other, and a plurality of R²⁴s may be bonded to each other to form a ring together with the carbon atoms of the heteroaromatic ring to which they are bonded.

Examples of the halogen atom represented by R²¹ to R²⁴ include a fluorine atom, a chlorine atom, a bromine atom, and an iodine atom. The hydrocarbyl group having 1 to 40 carbon atoms, represented by R²¹ to R²⁴, may be saturated or unsaturated, and may be any of linear, branched, or cyclic. Specific examples thereof include: alkyl groups having 1 to 40 carbon atoms, such as a methyl group, an ethyl group, a propyl group, an isopropyl group, a n-butyl group, an isobutyl group, a sec-butyl group, a tert-butyl group, a n-pentyl group, a tert-pentyl group, a n-hexyl group, a n-octyl group, a 2-ethylhexyl group, a n-nonyl group and a n-decyl group; saturated cyclic hydrocarbyl groups having 3 to 40 carbon atoms, such as a cyclopentyl group, a cyclohexyl group, a cyclopentylmethyl group, a cyclopentylethyl group, a cyclopentylbutyl group, a cyclohexylmethyl group, a cyclohexylethyl group, a cyclohexylbutyl group, a norbornyl group, a tricyclo[5.2.1.0^{2,6}]decanyl group, an adamantyl group, and an adamantylmethyl group; and aryl groups having 6 to 40 carbon atoms, such as a phenyl group, a naphthyl group, and an anthracenyl group. Part or all of the hydrogen atoms of the hydrocarbyl group may be substituted with a group having a heteroatom, such as an oxygen atom, a sulfur atom, a nitrogen atom, or a halogen atom, part of the —CH₂—groups of the hydrocarbyl group may be substituted with a group having a heteroatom, such as an oxygen atom, a sulfur atom, or a nitrogen atom, and as a result, a hydroxy group, a cyano group, a halogen atom, a carbonyl group, an ether bond, a thioether bond, an ester bond, a sulfonic acid ester bond, a carbonate bond, a carbamate bond, an amide bond, a lactone ring, a sultone ring, a carboxylic anhydride (—C(═O)—O—C(═O)—), or the like may be contained. Furthermore, R²¹ to R²⁴ can substitute at any position in the aromatic ring of the formula, provided that R²² is X² when X² is bonded to the nitrogen atom to which R²² is bonded.

Specific examples of the hypervalent iodine compound represented by the formula (1) include the following, but are not limited thereto. In the following formulae, *1 and *2 represent the same as defined above.

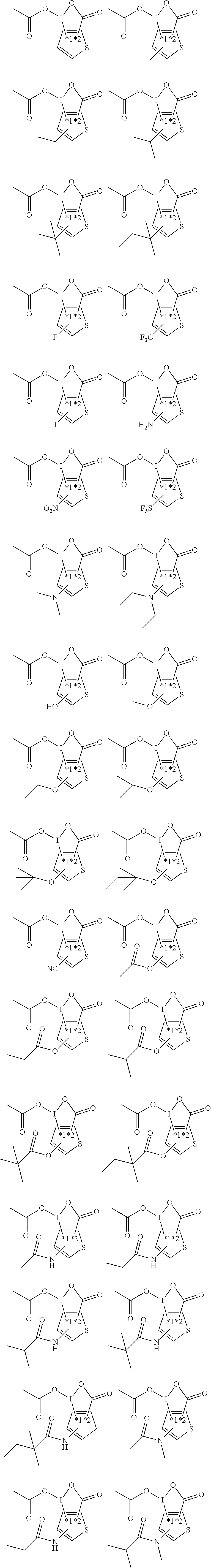

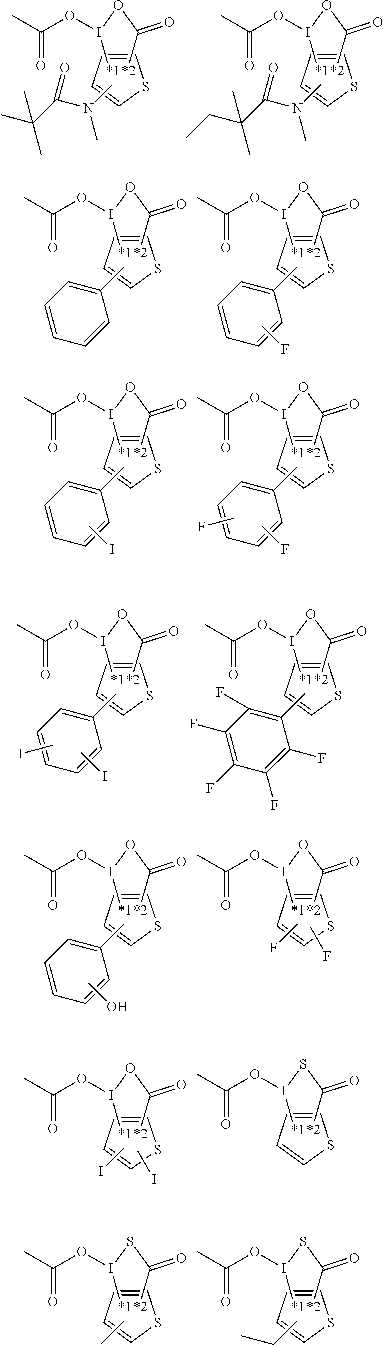

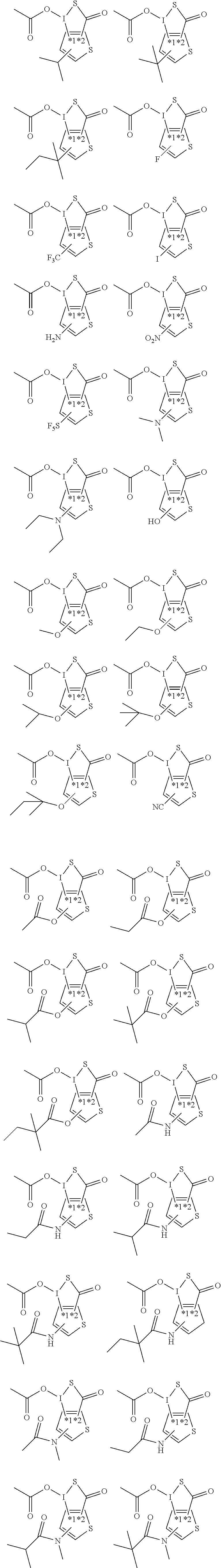

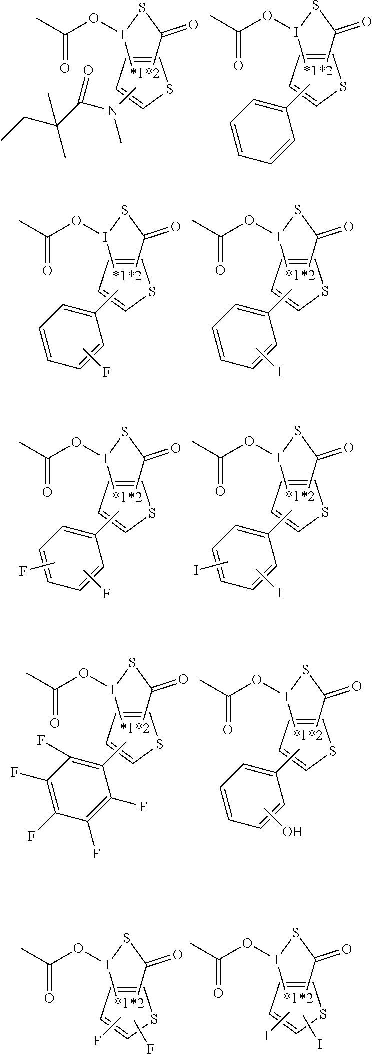

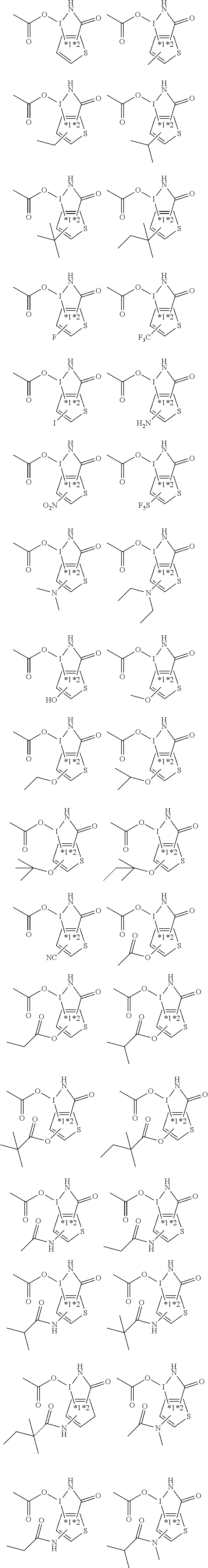

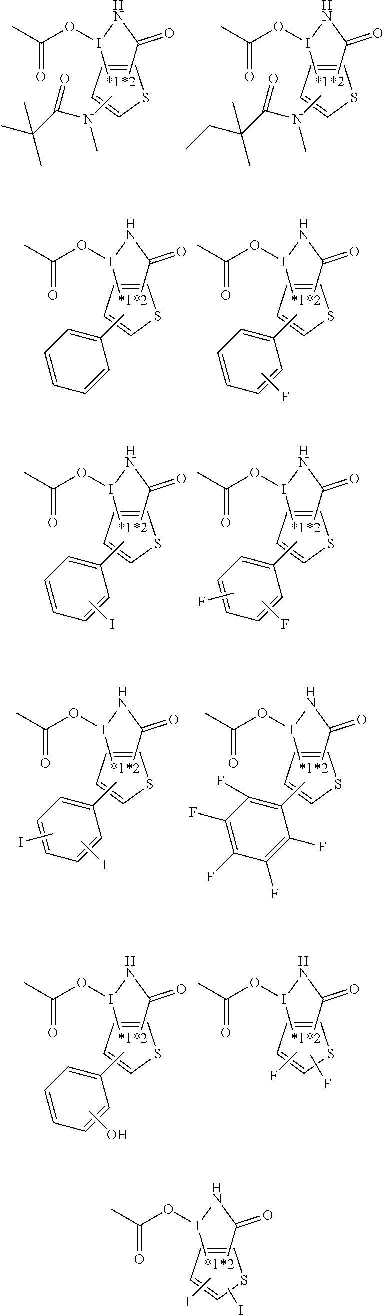

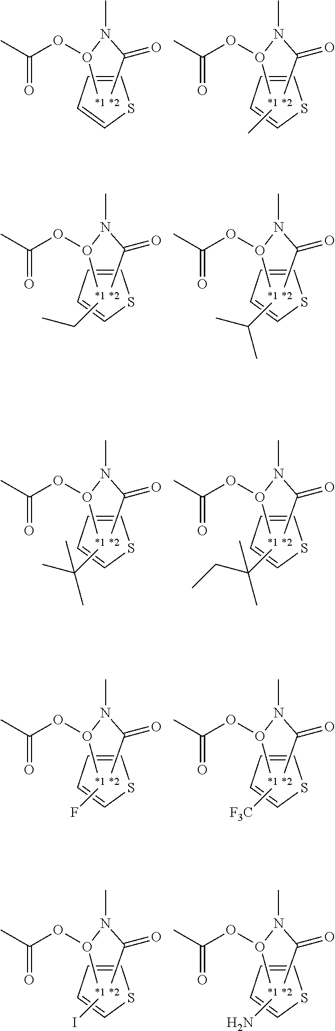

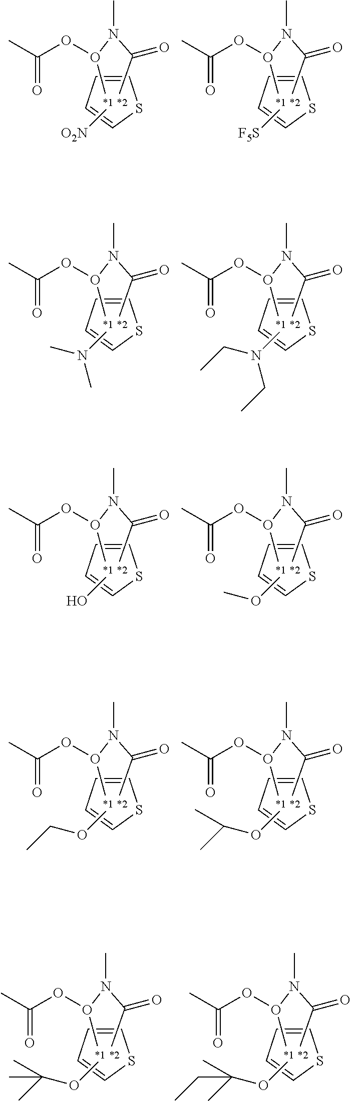

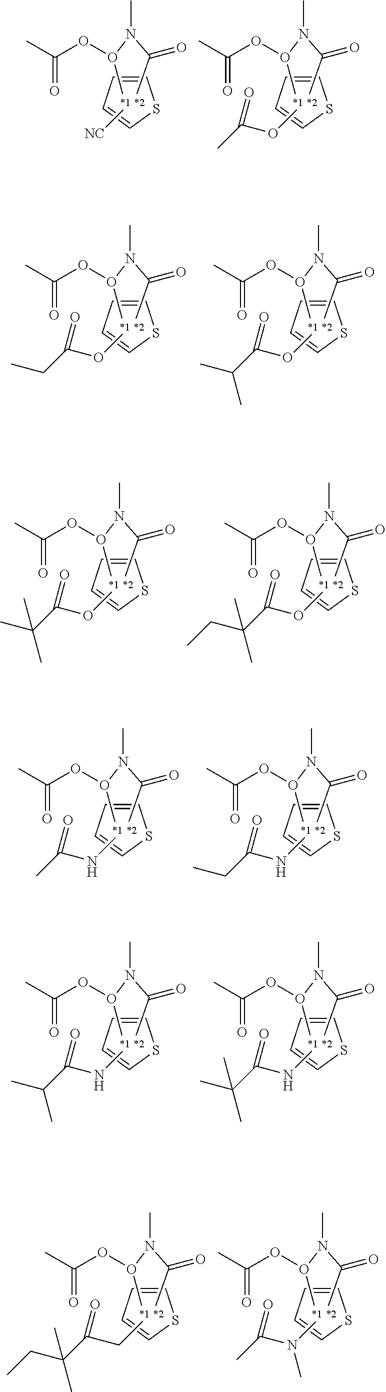

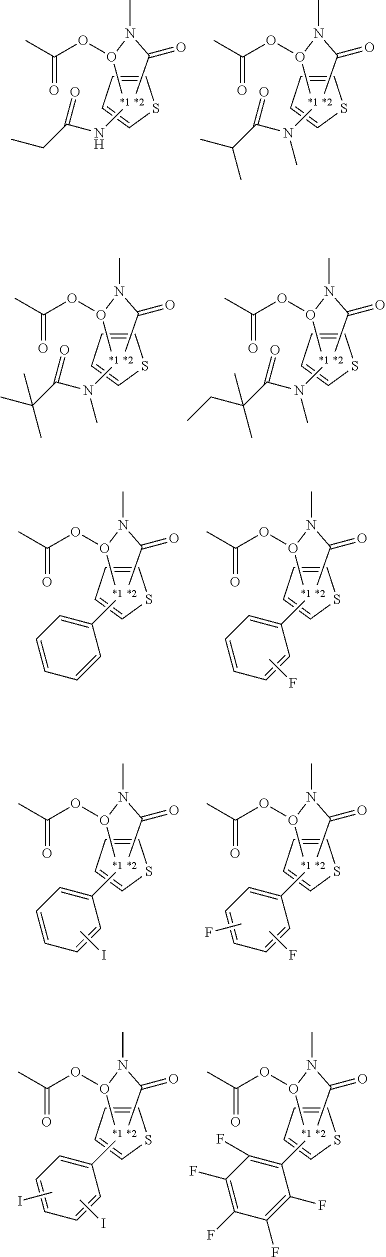

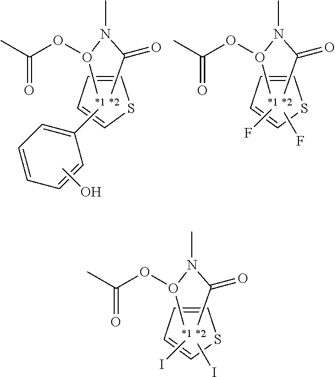

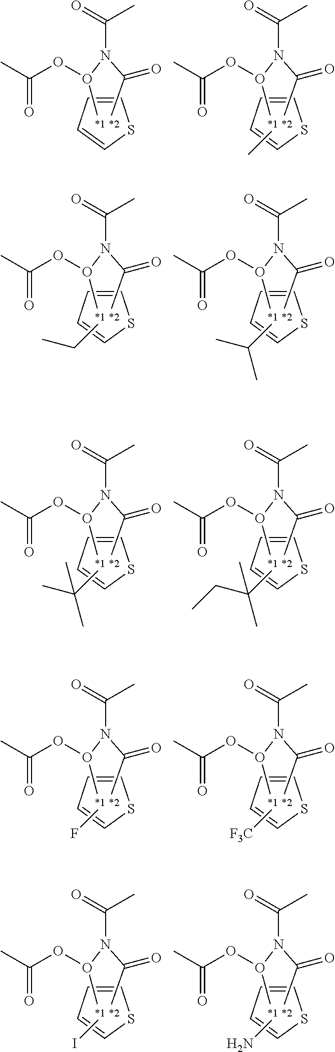

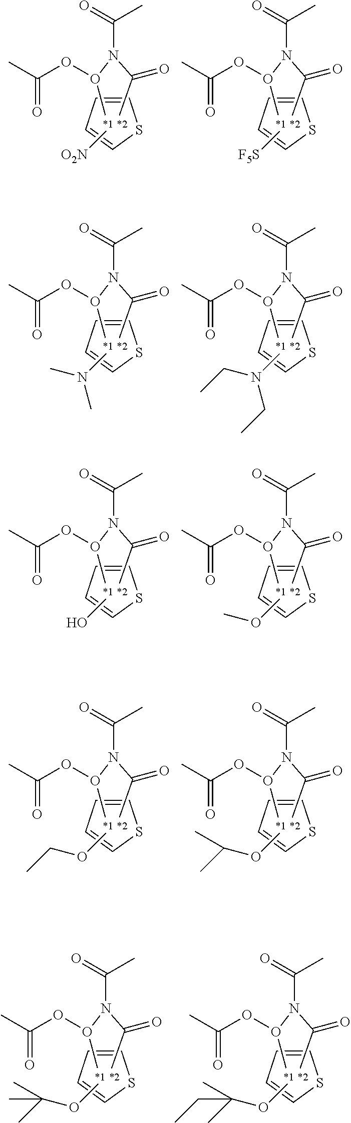

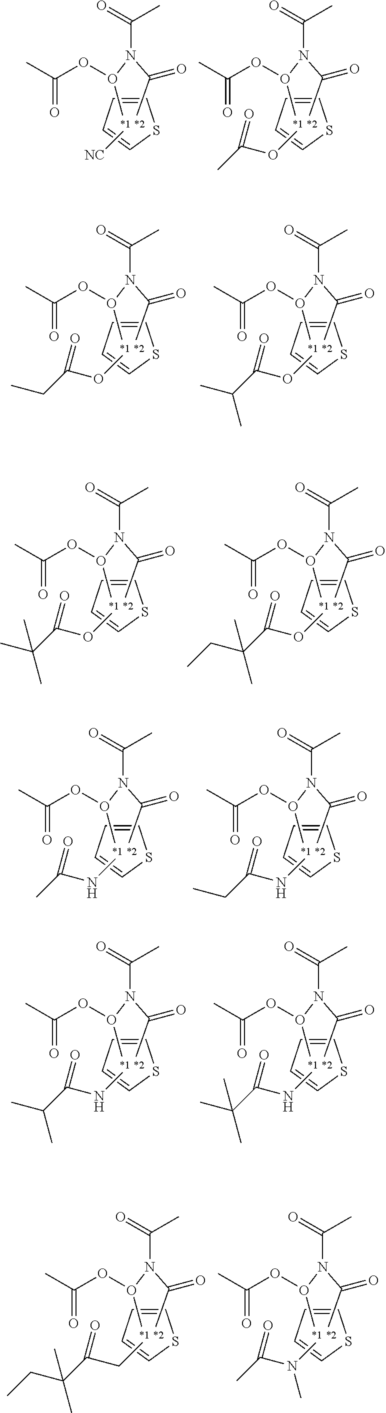

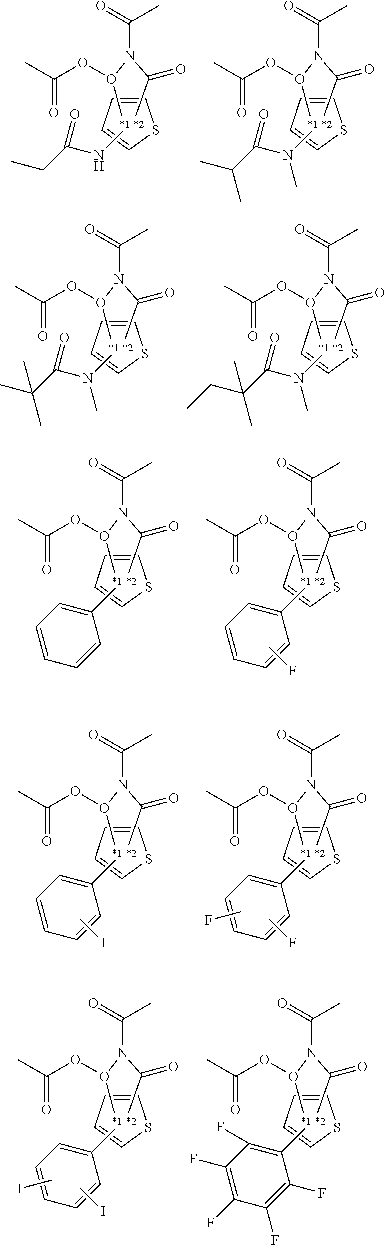

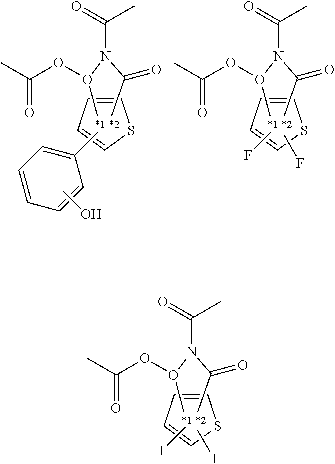

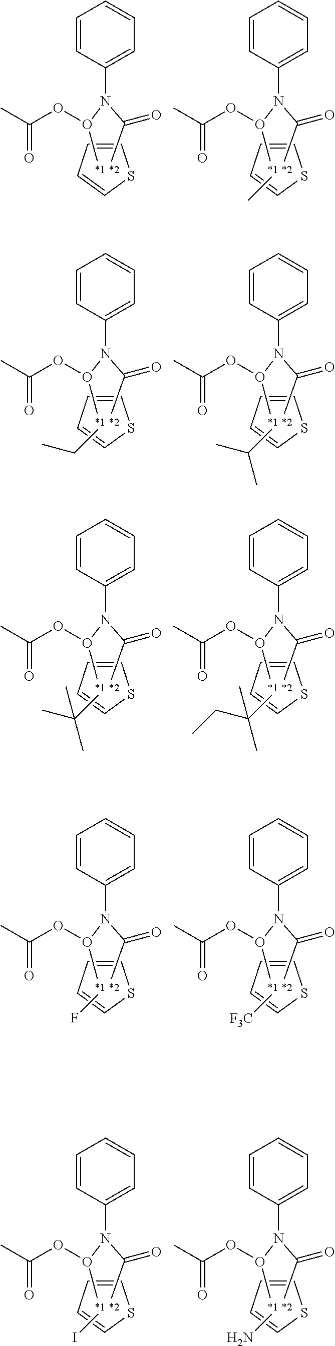

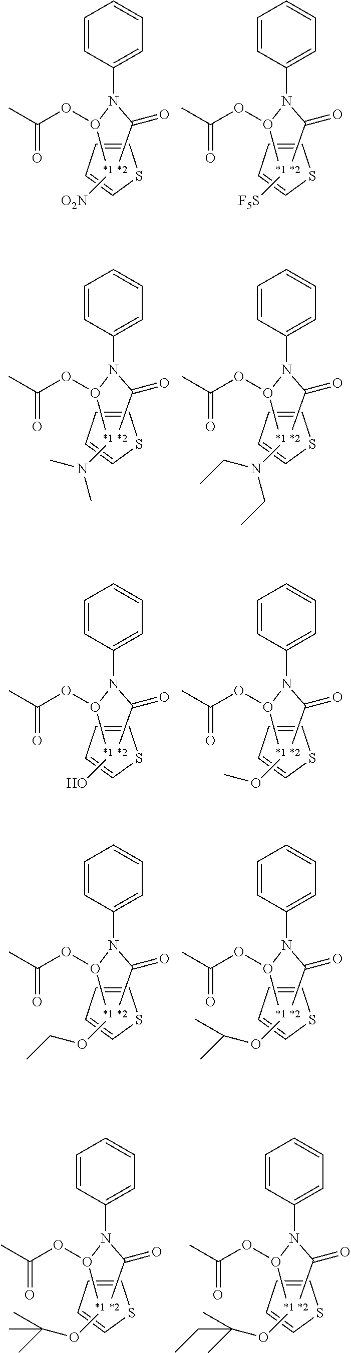

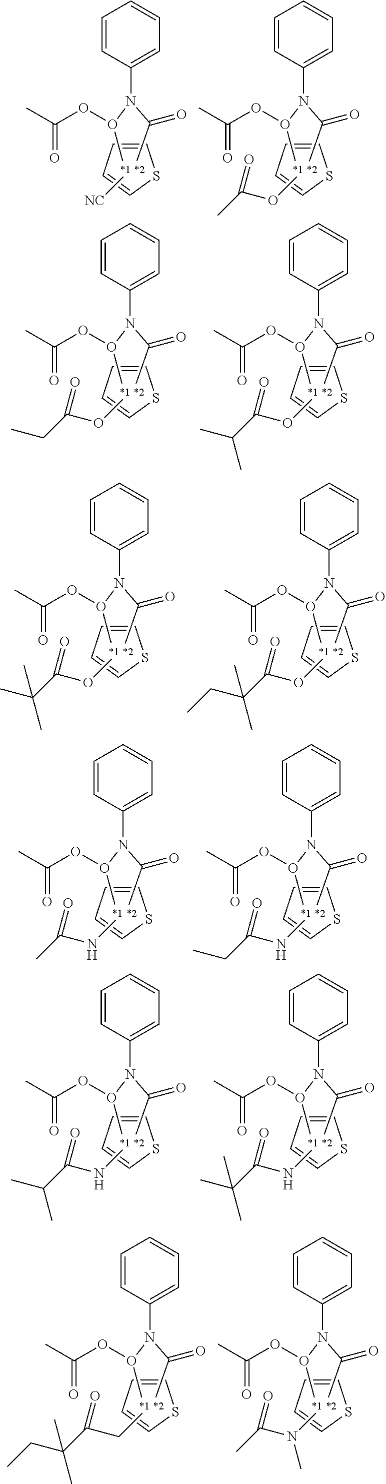

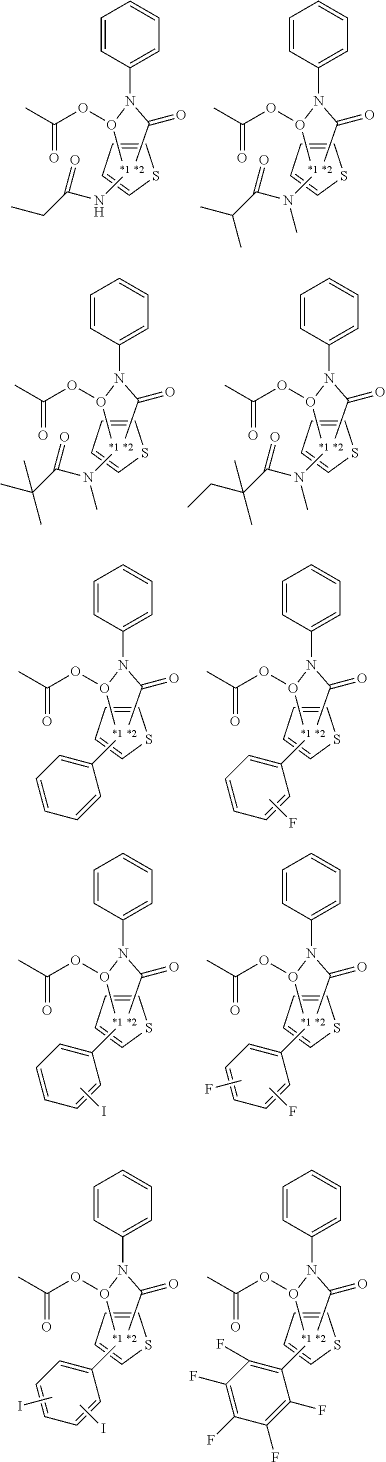

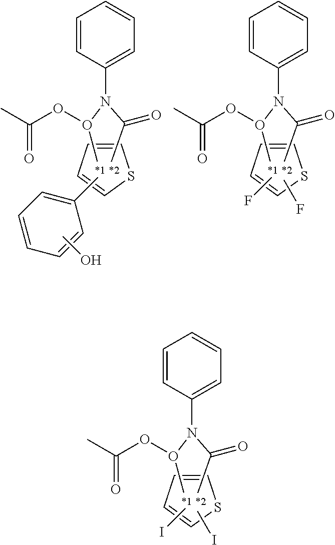

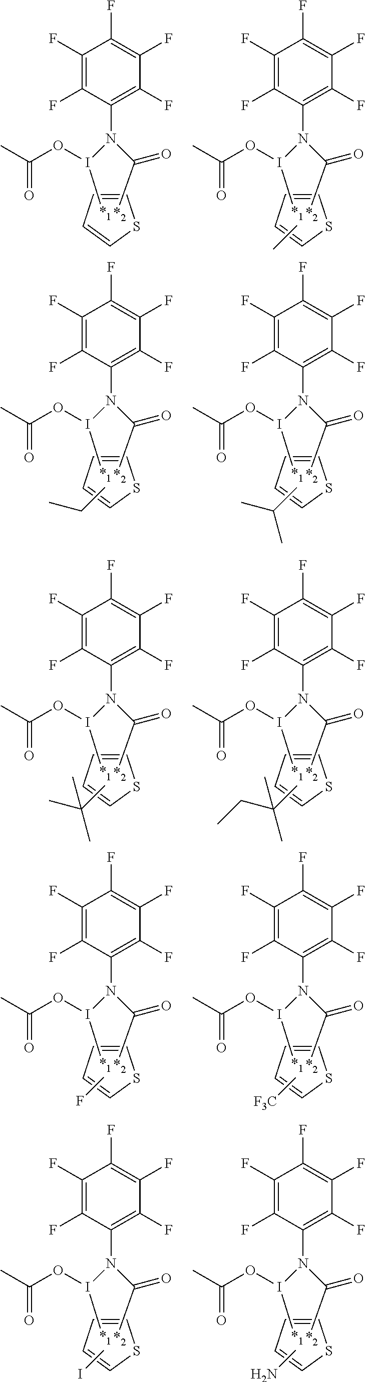

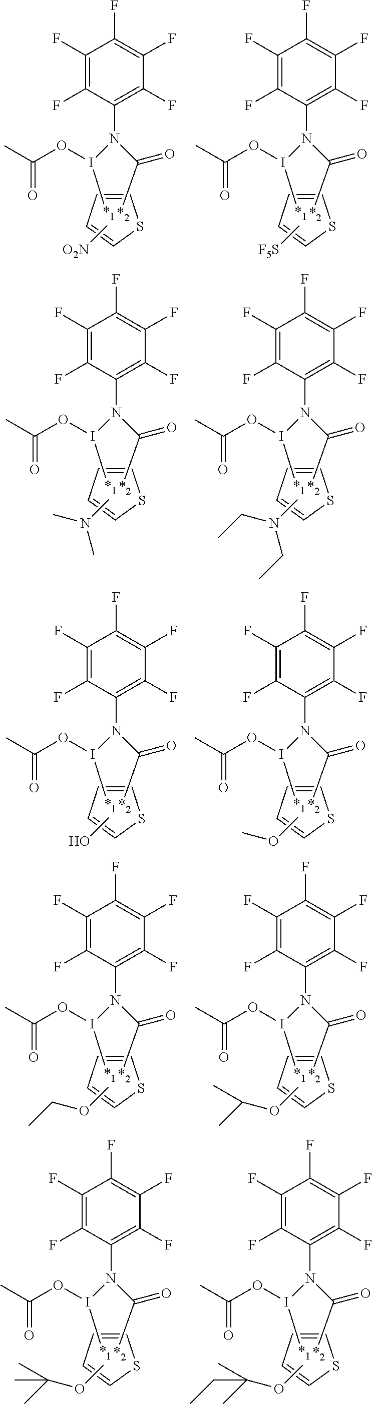

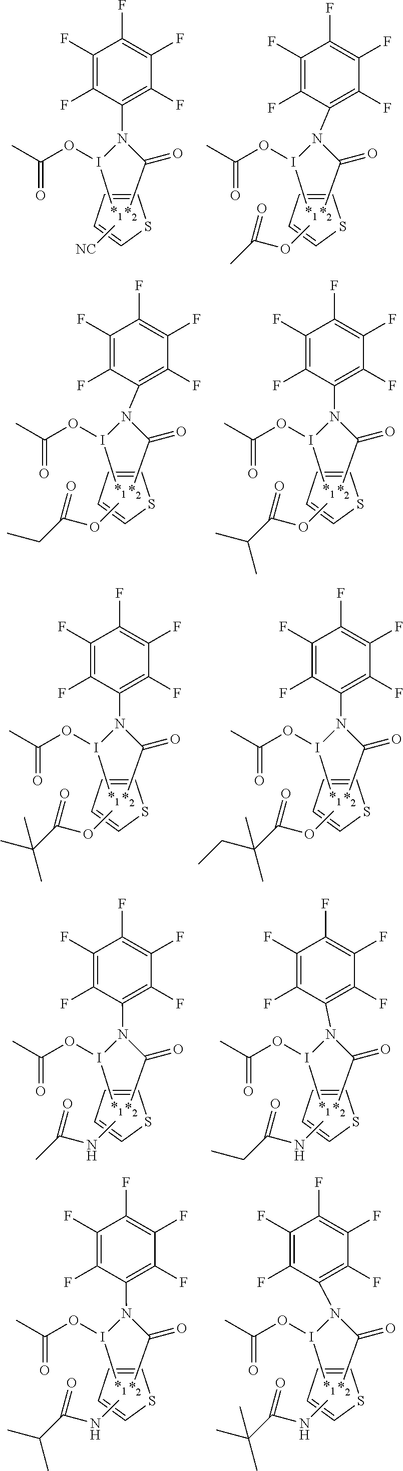

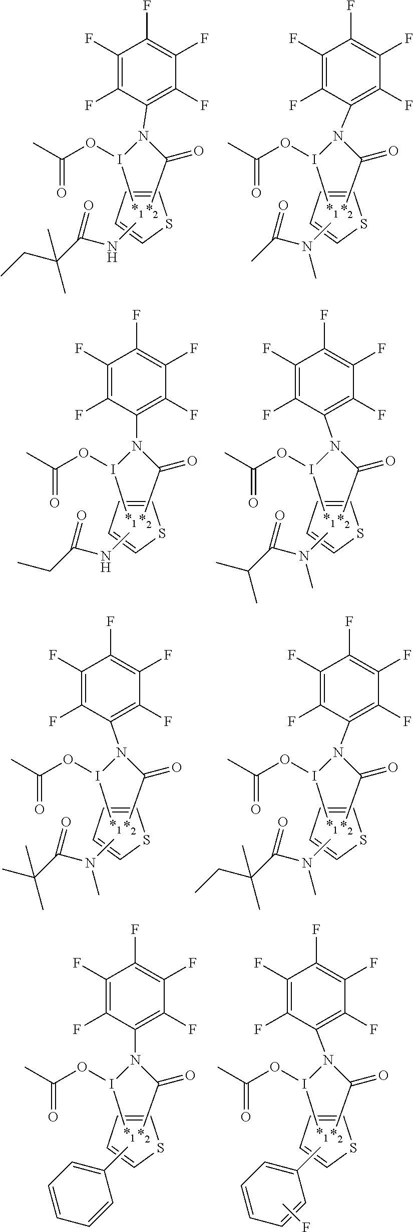

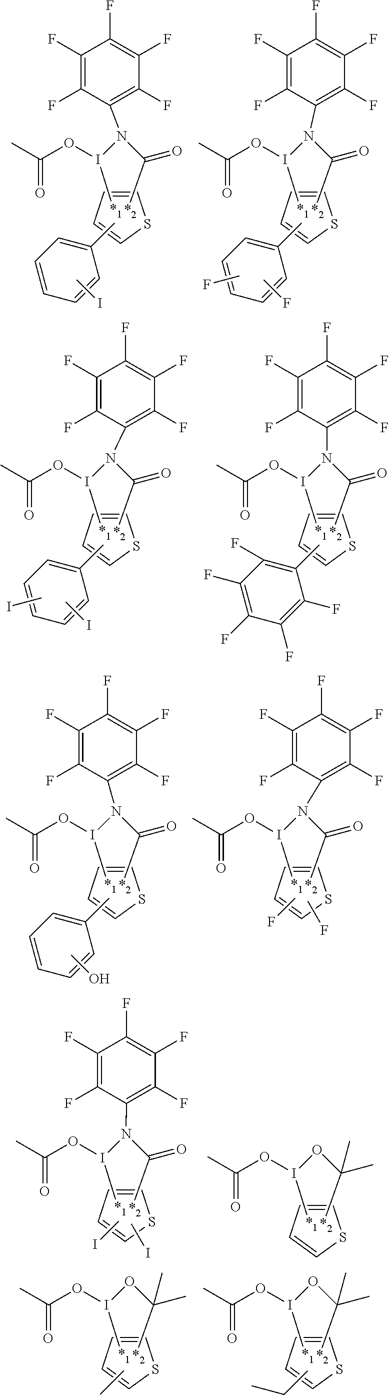

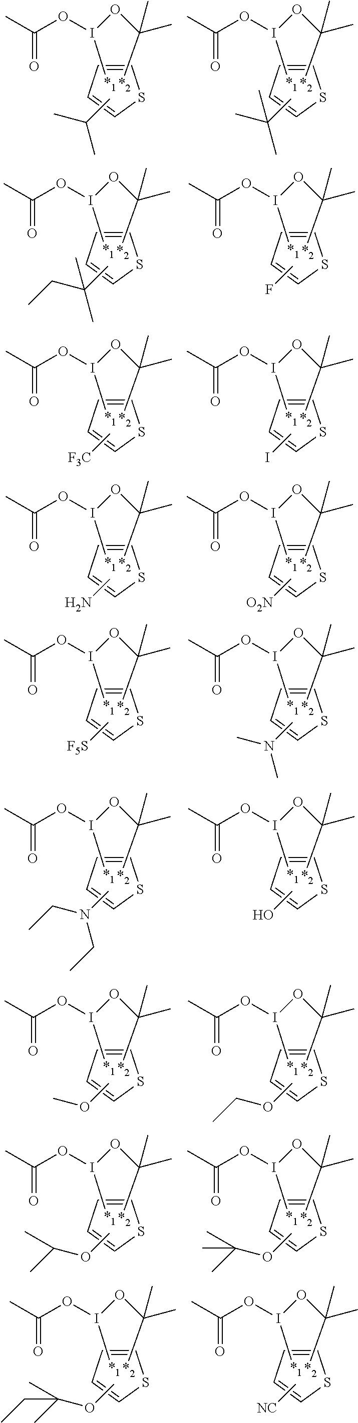

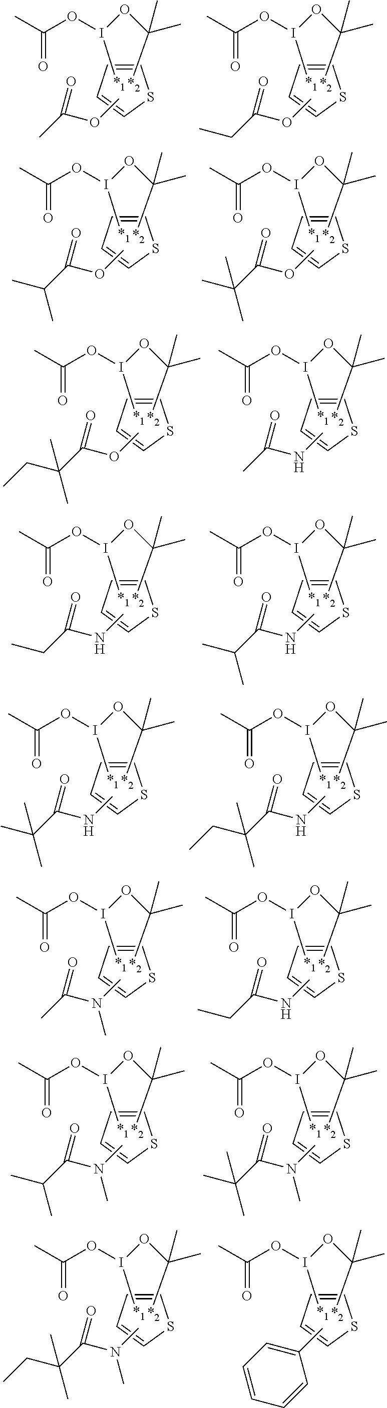

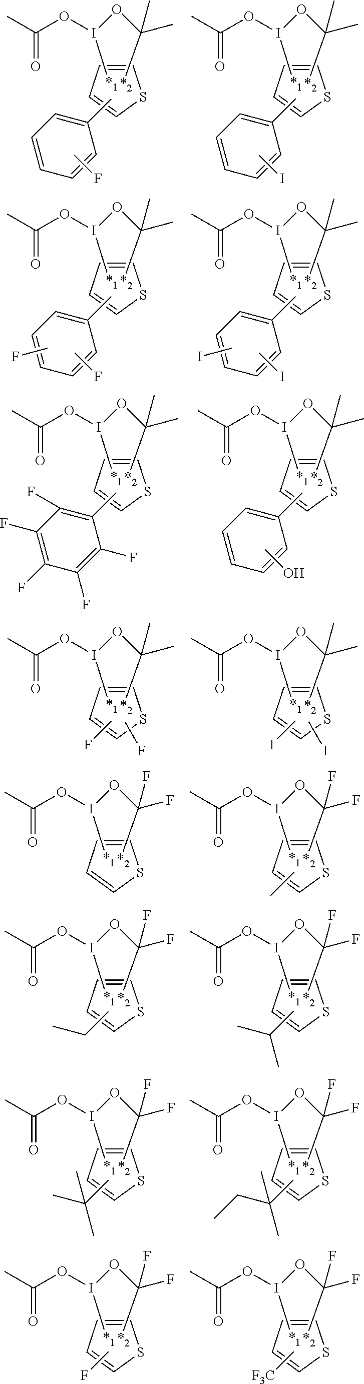

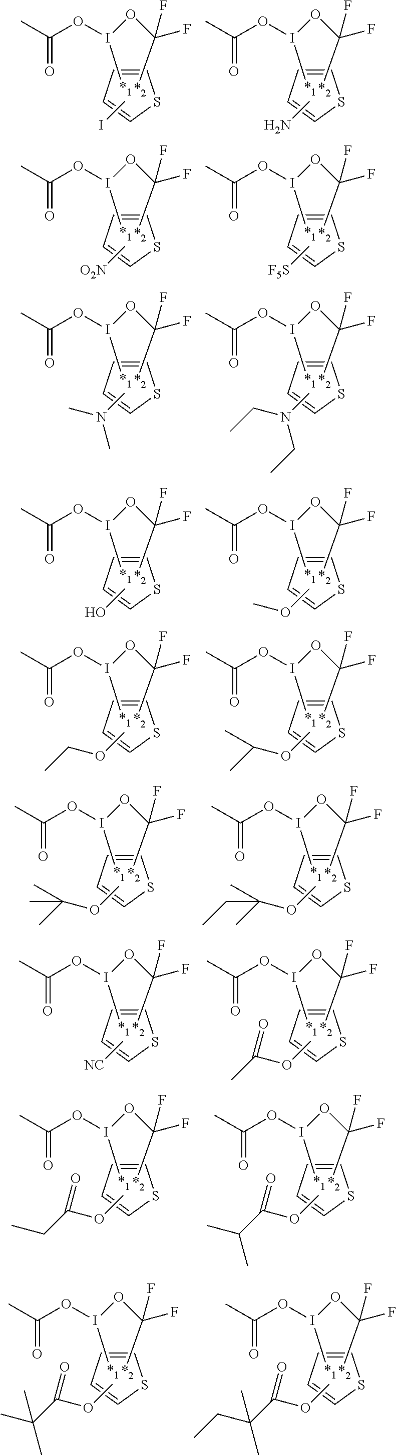

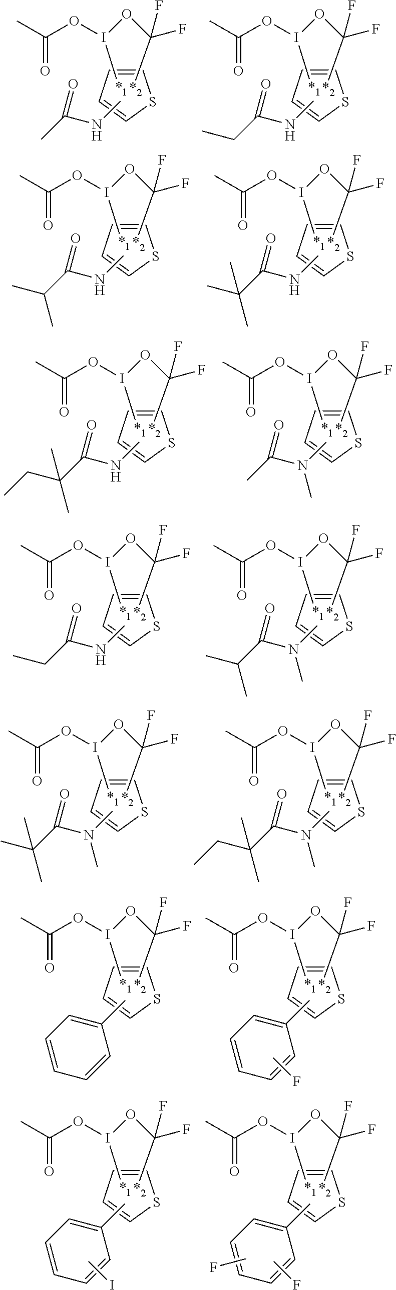

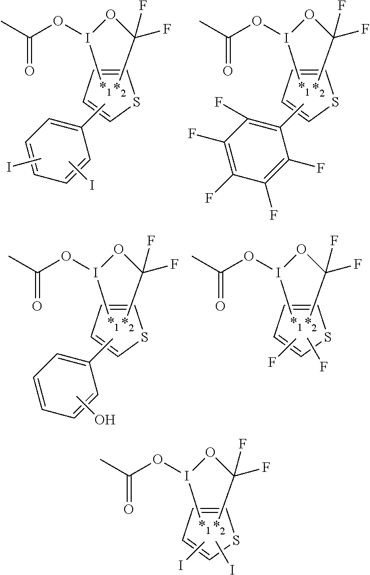

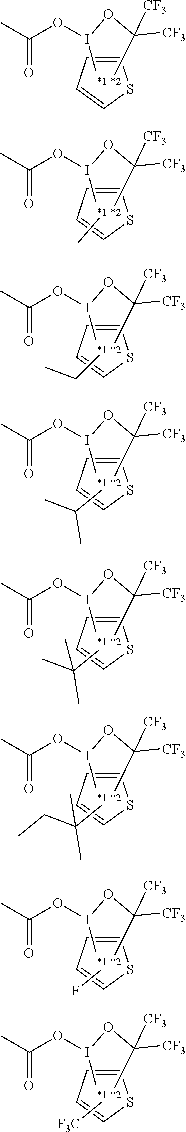

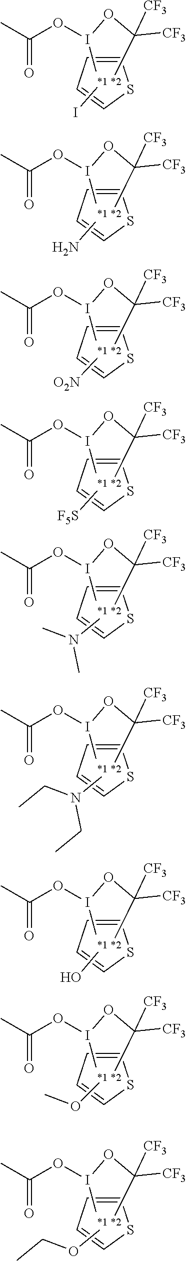

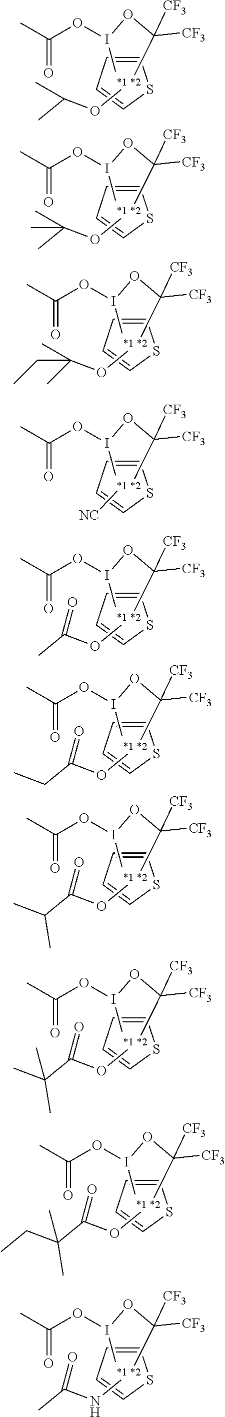

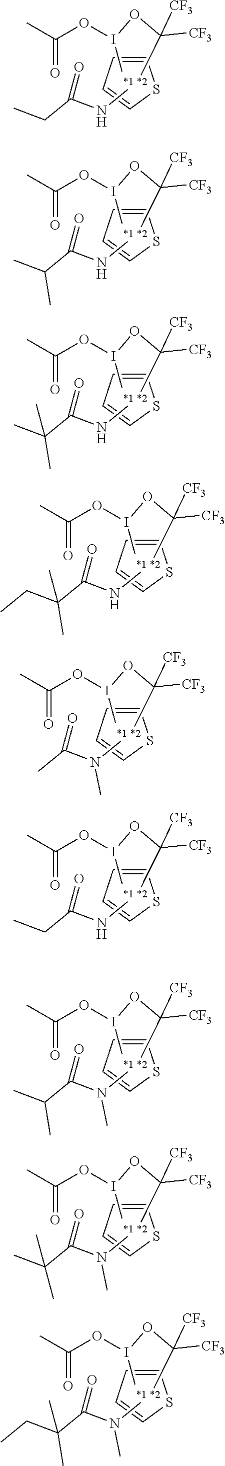

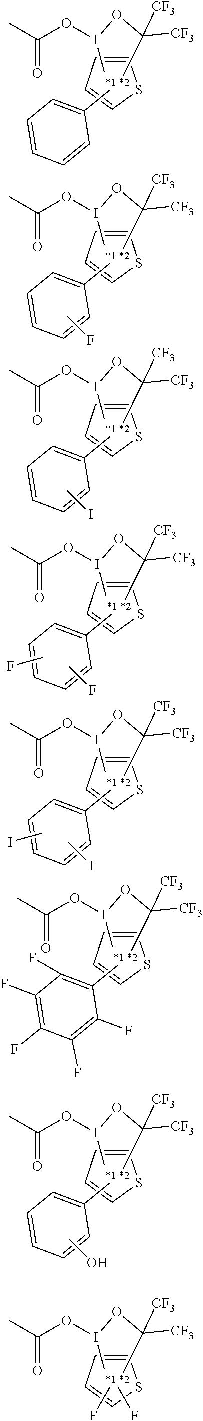

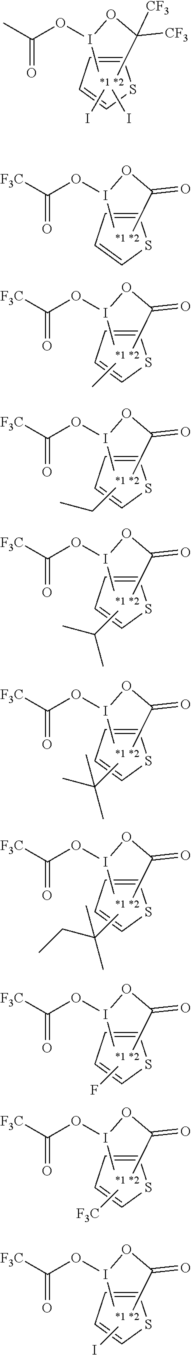

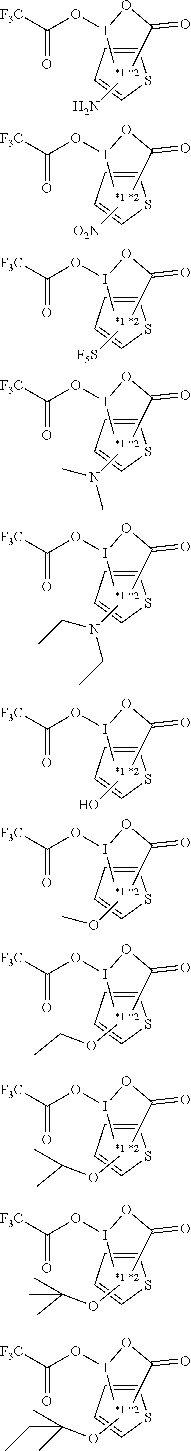

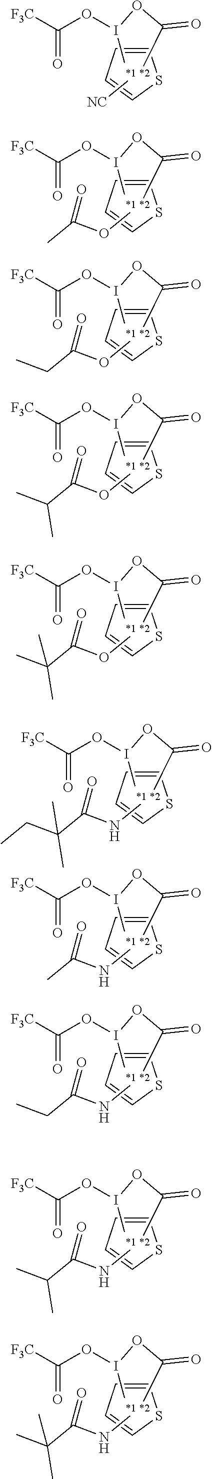

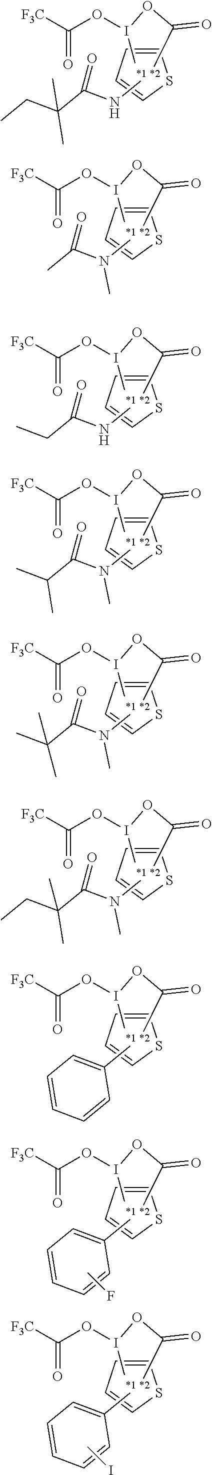

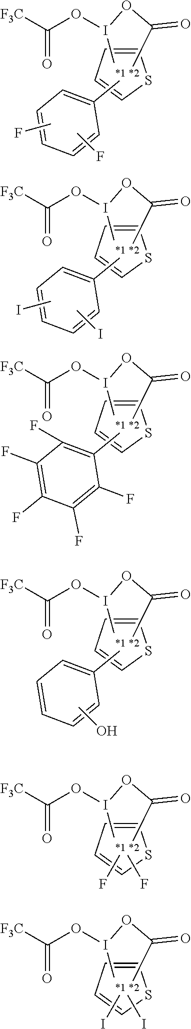

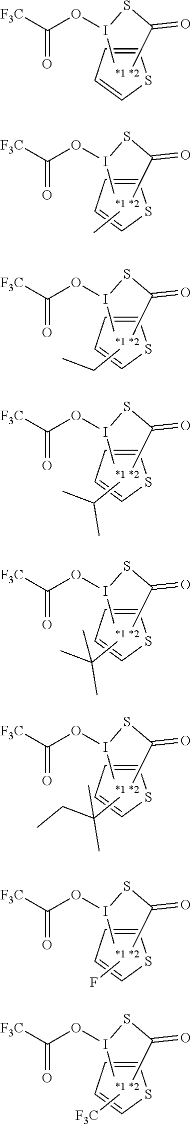

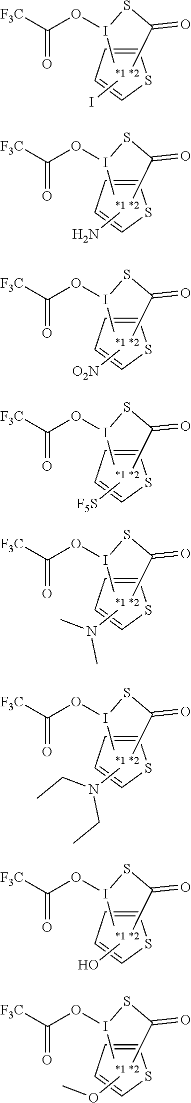

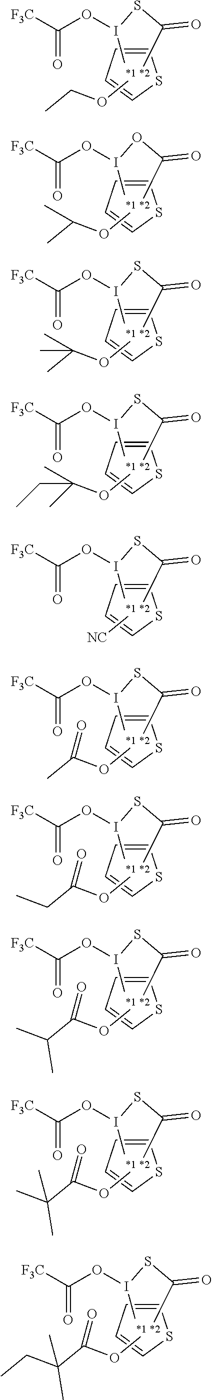

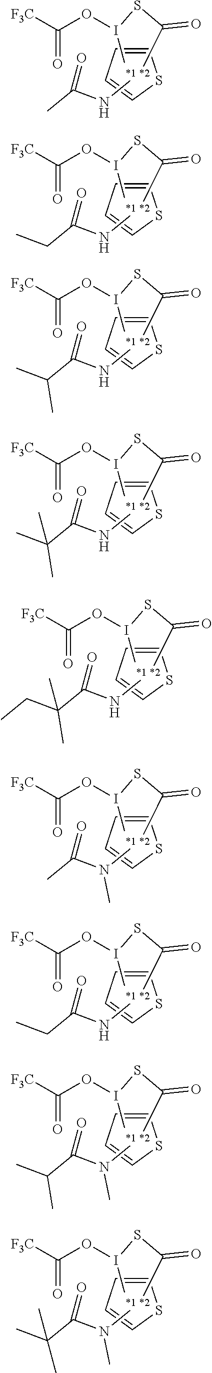

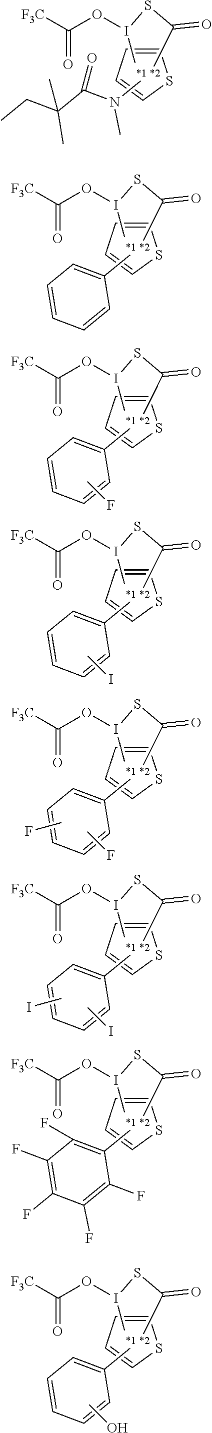

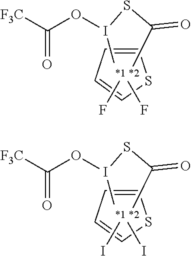

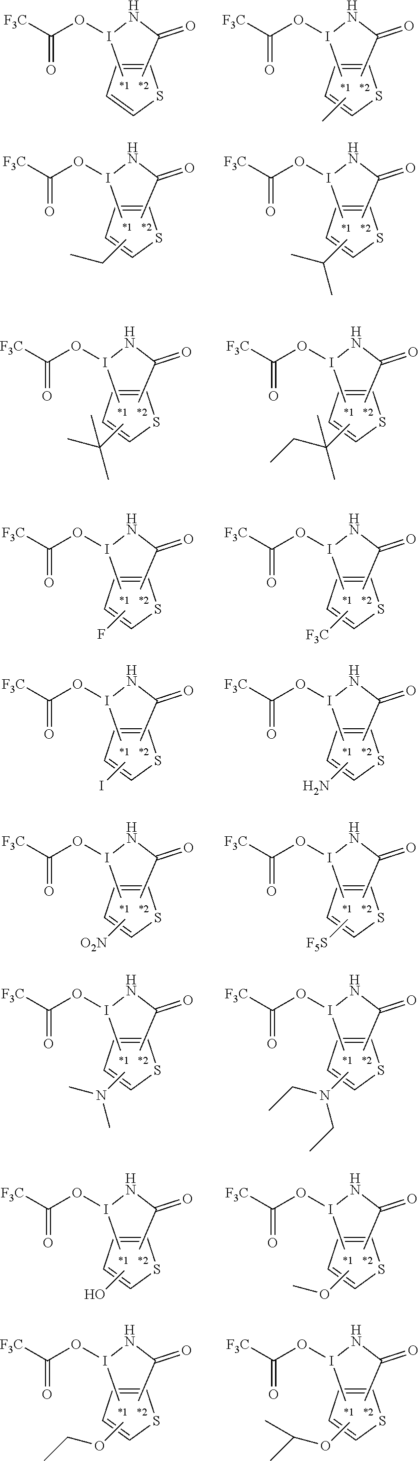

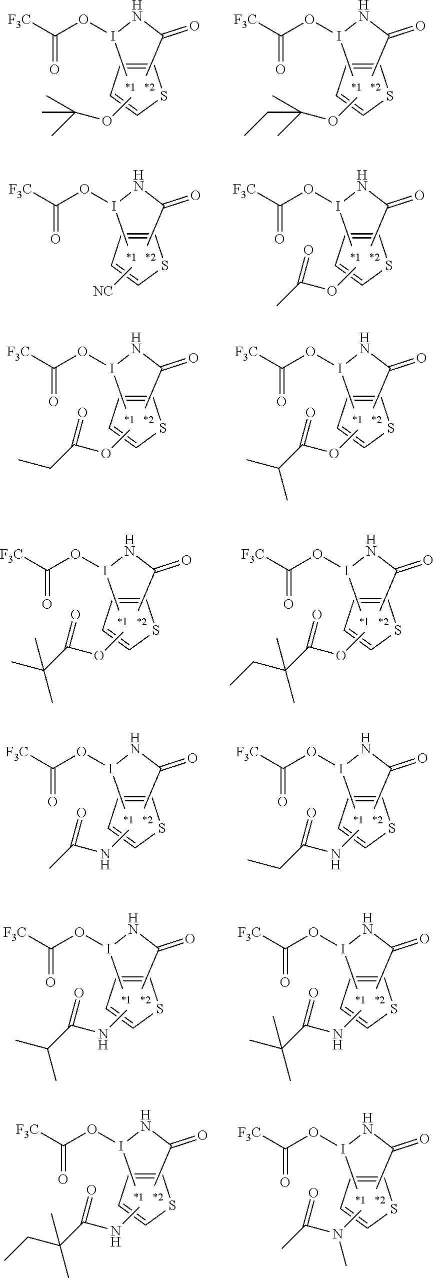

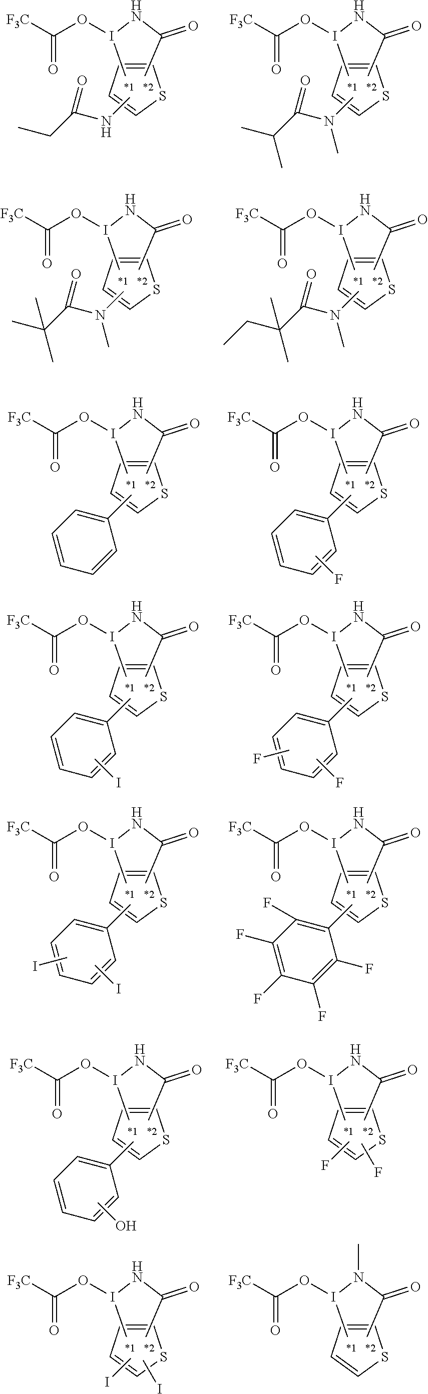

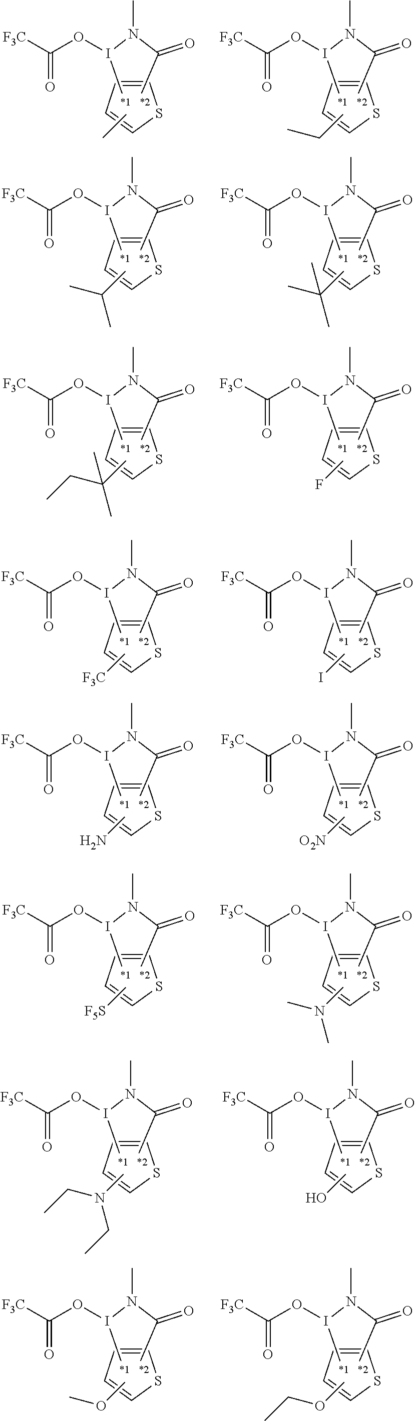

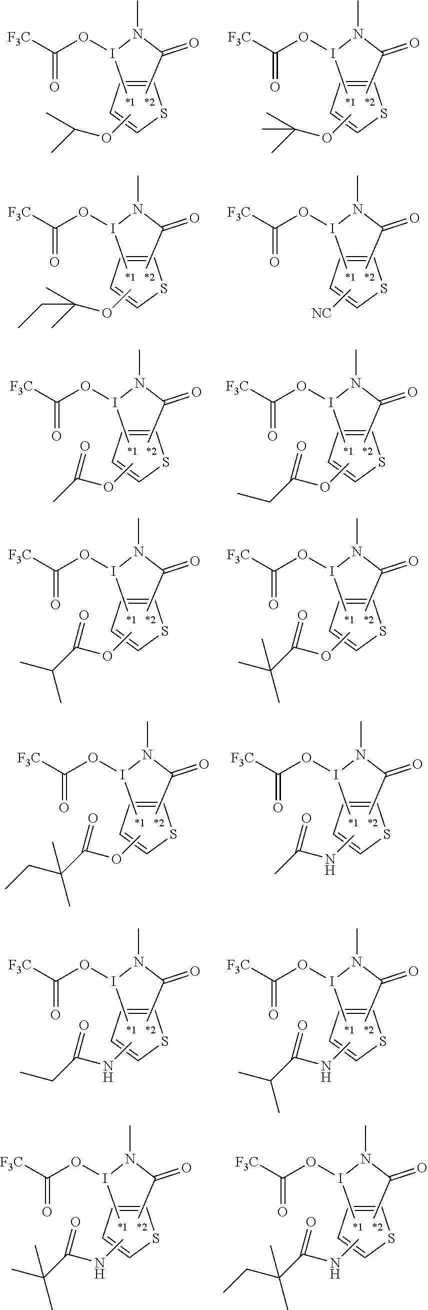

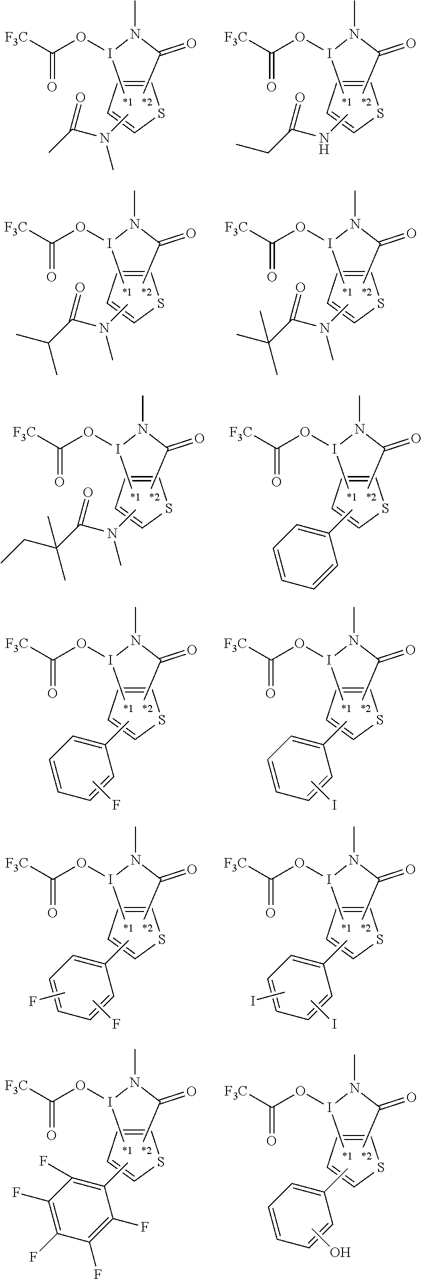

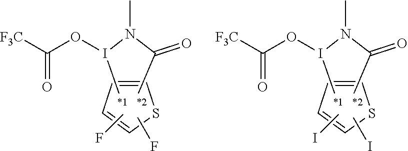

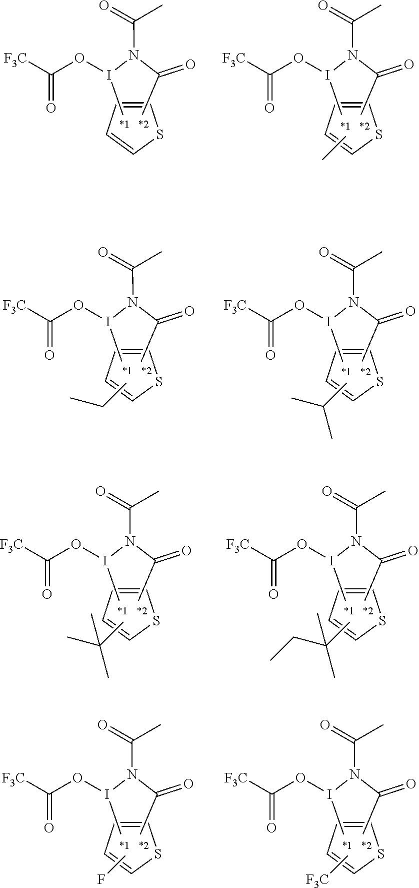

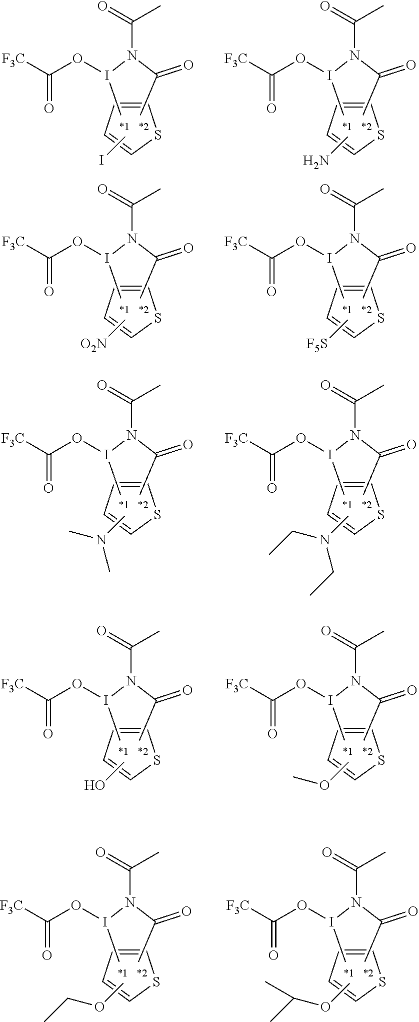

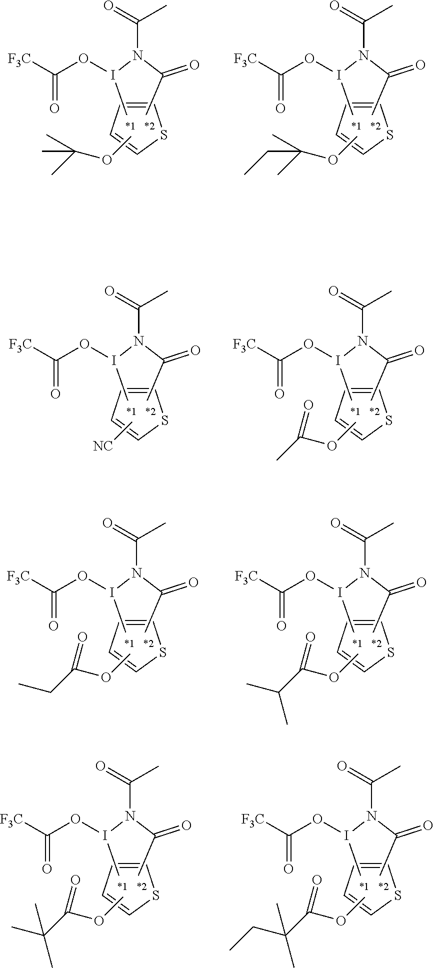

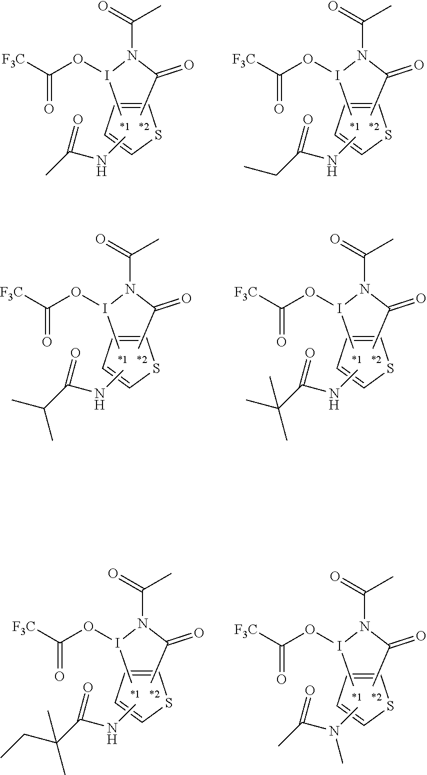

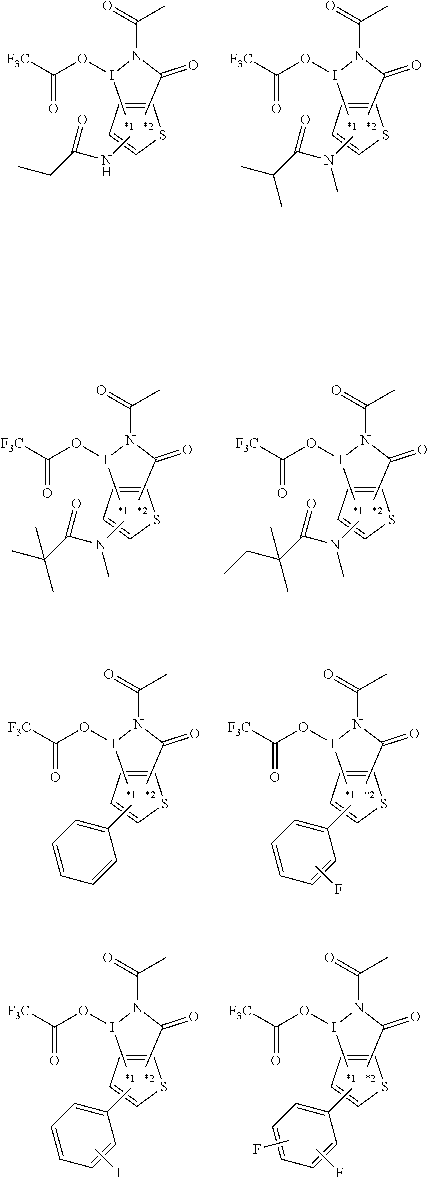

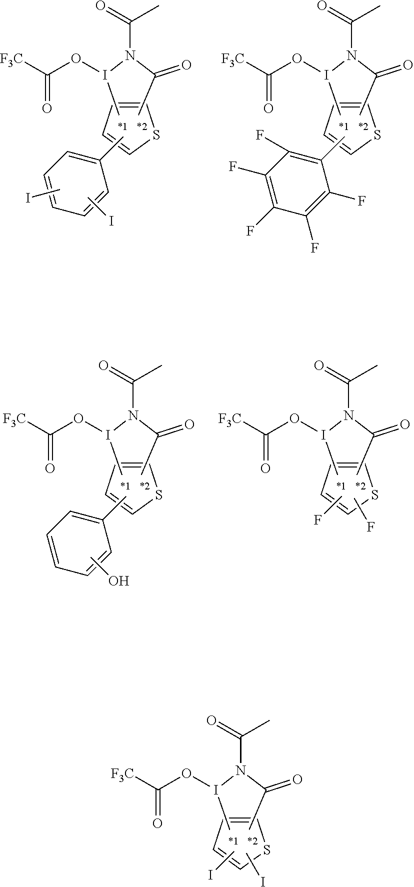

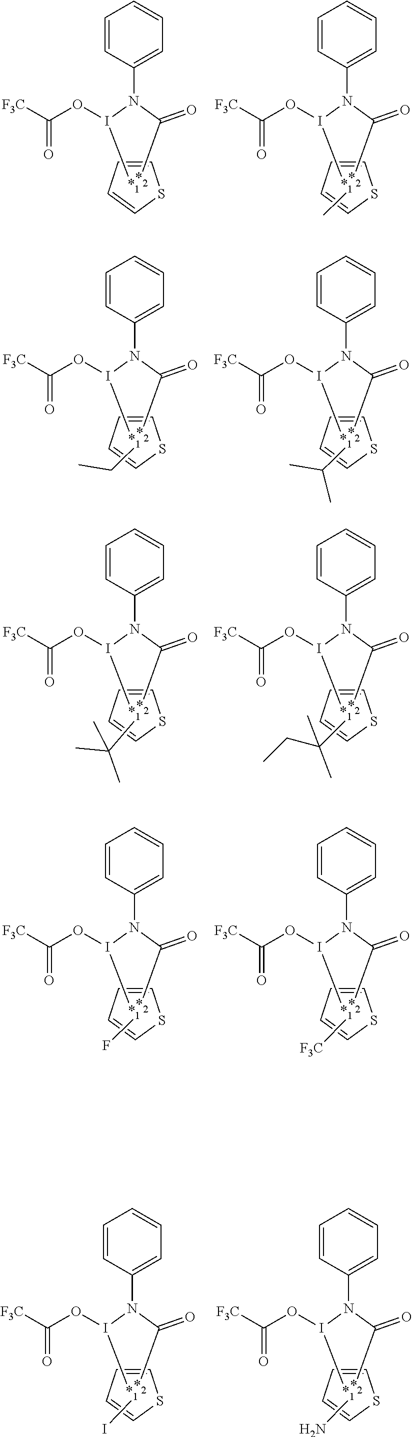

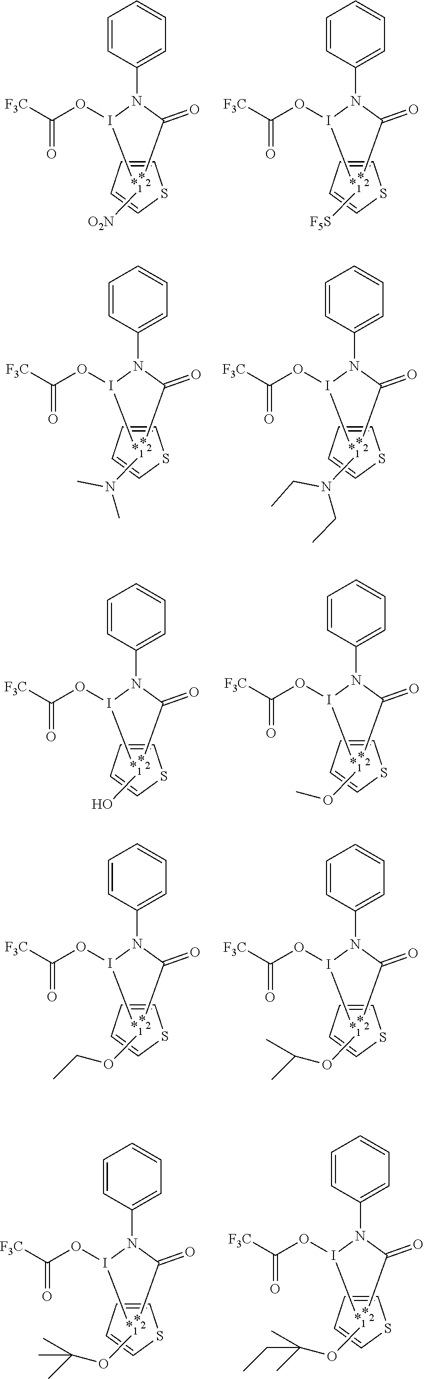

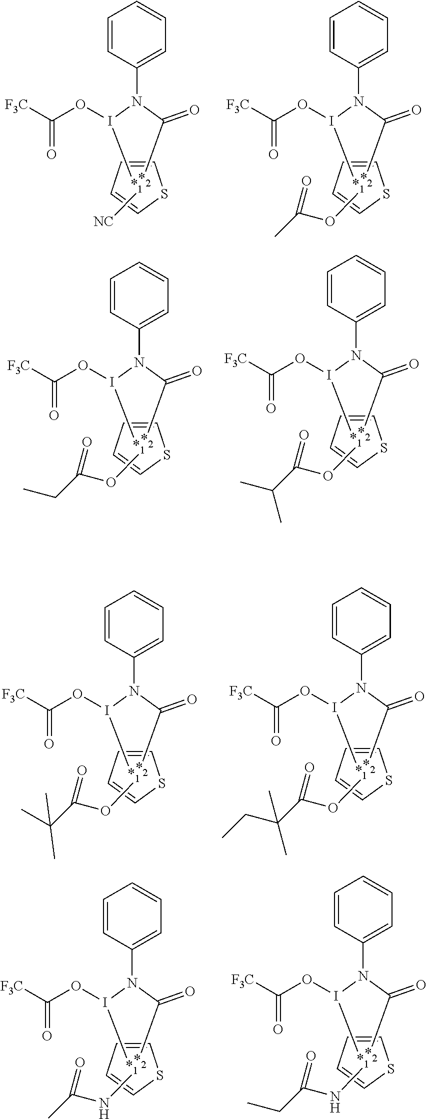

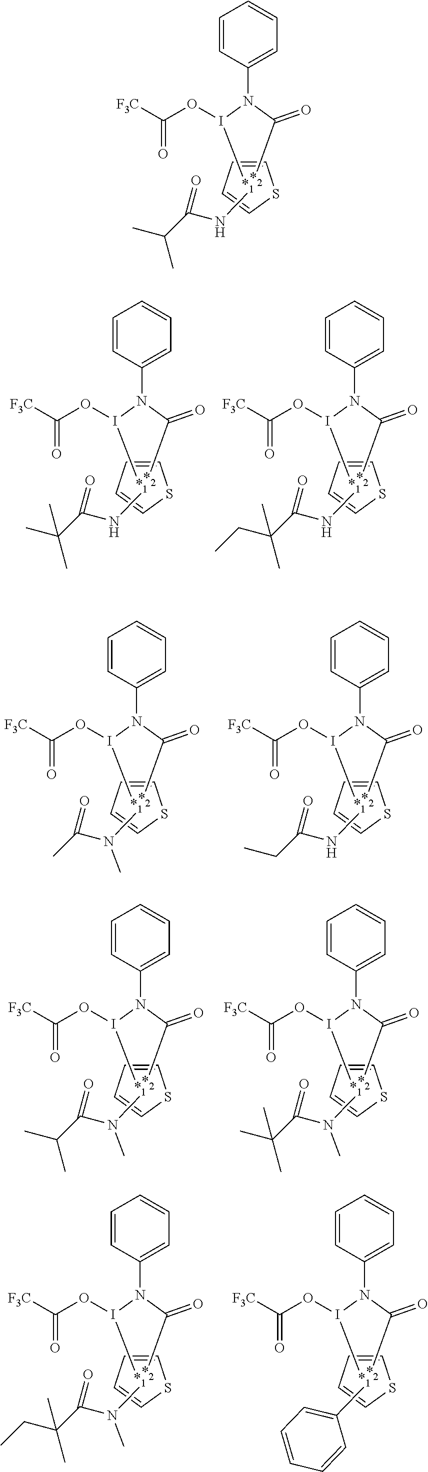

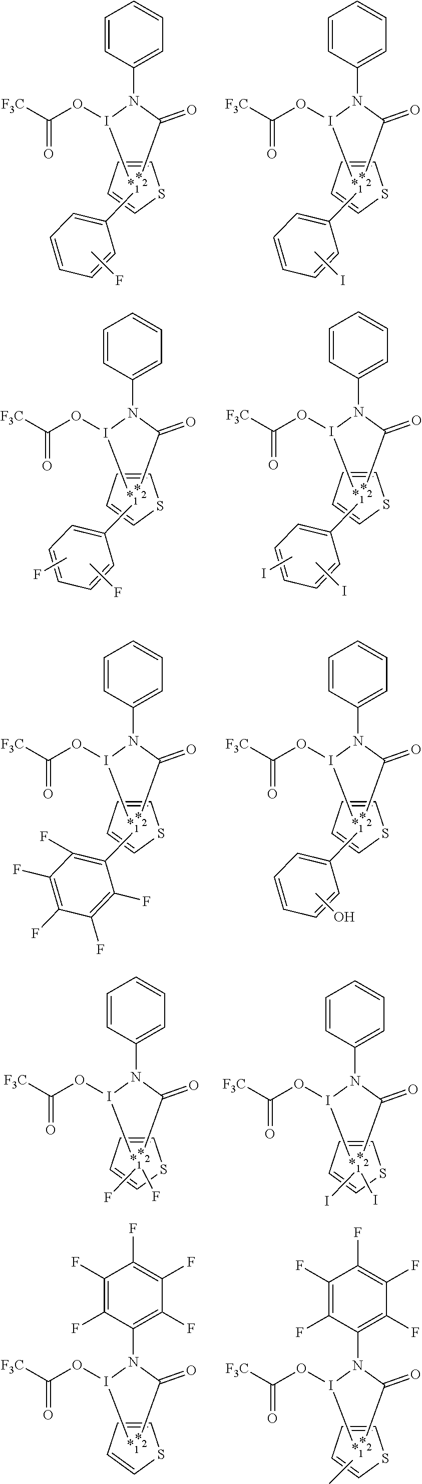

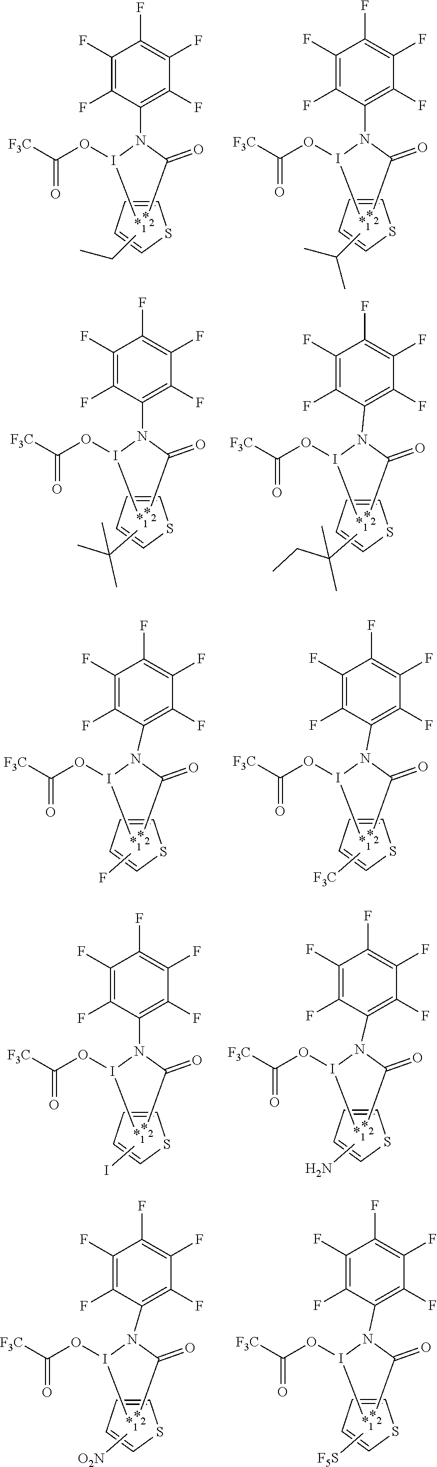

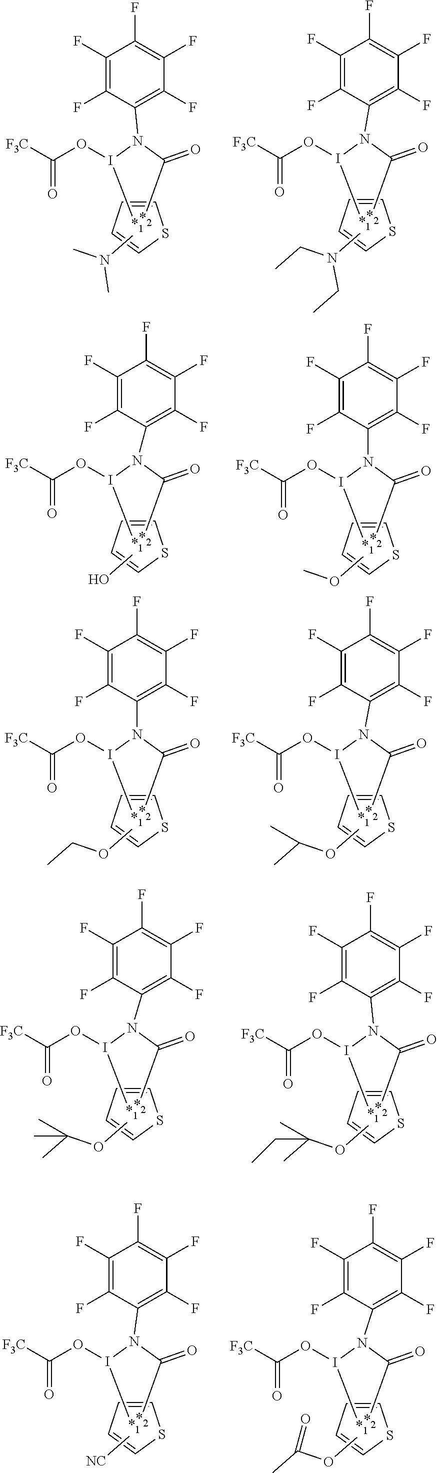

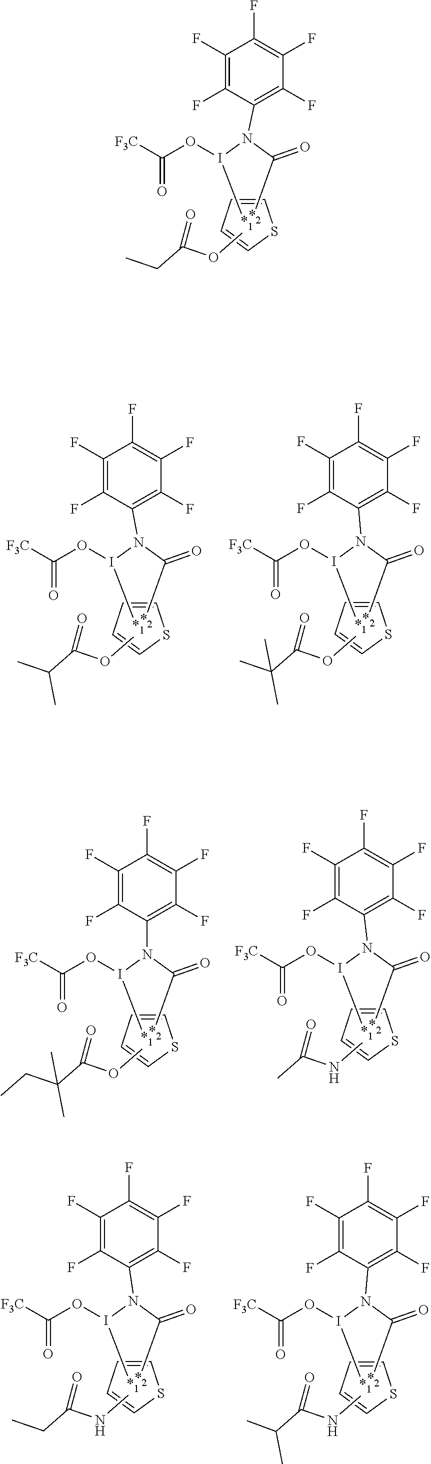

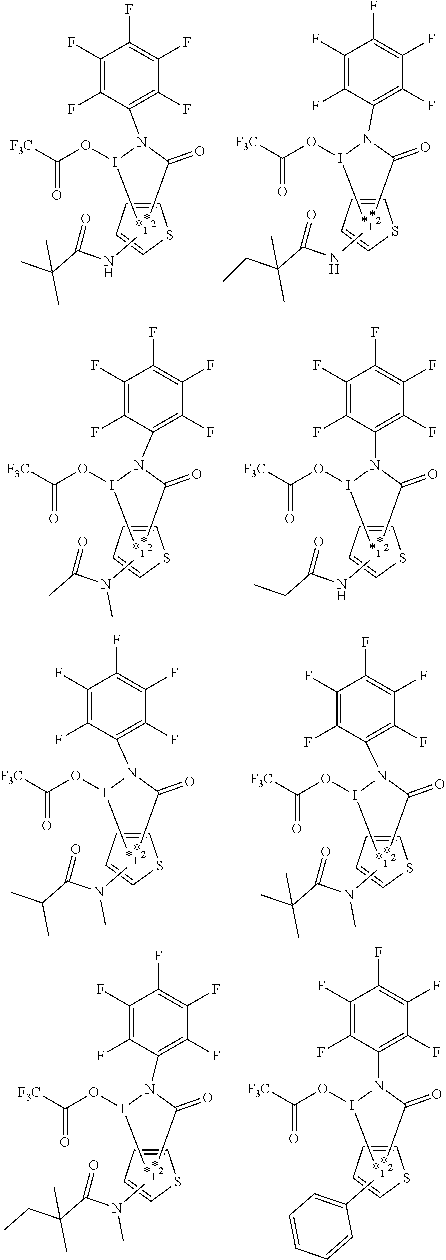

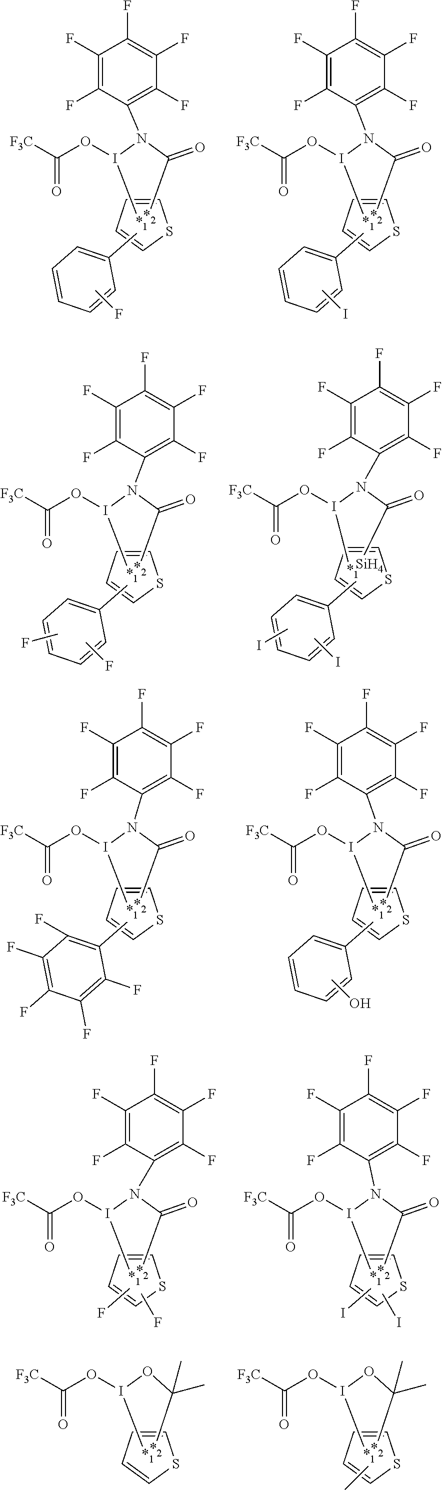

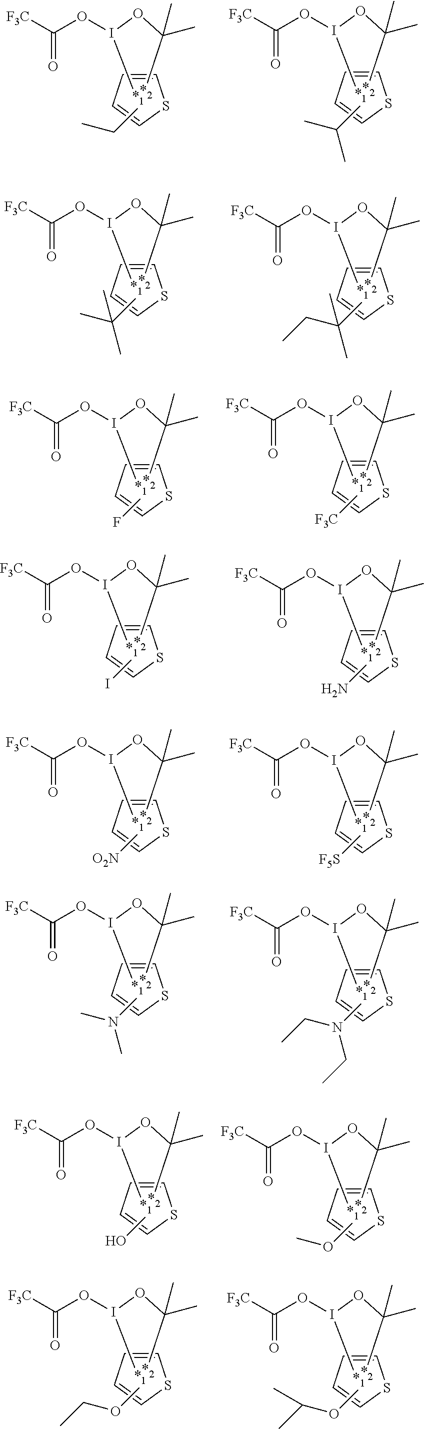

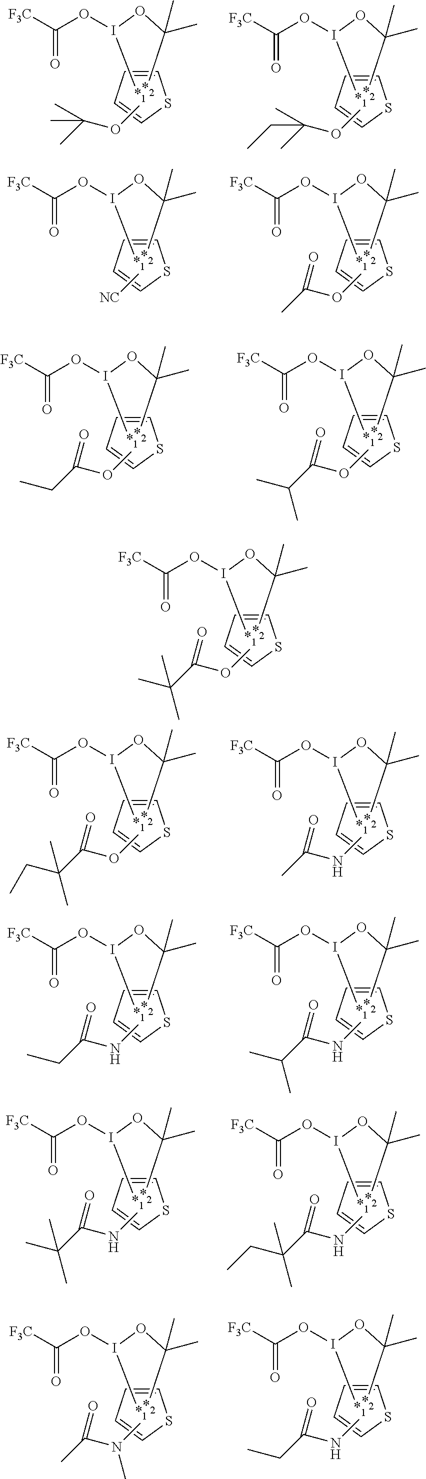

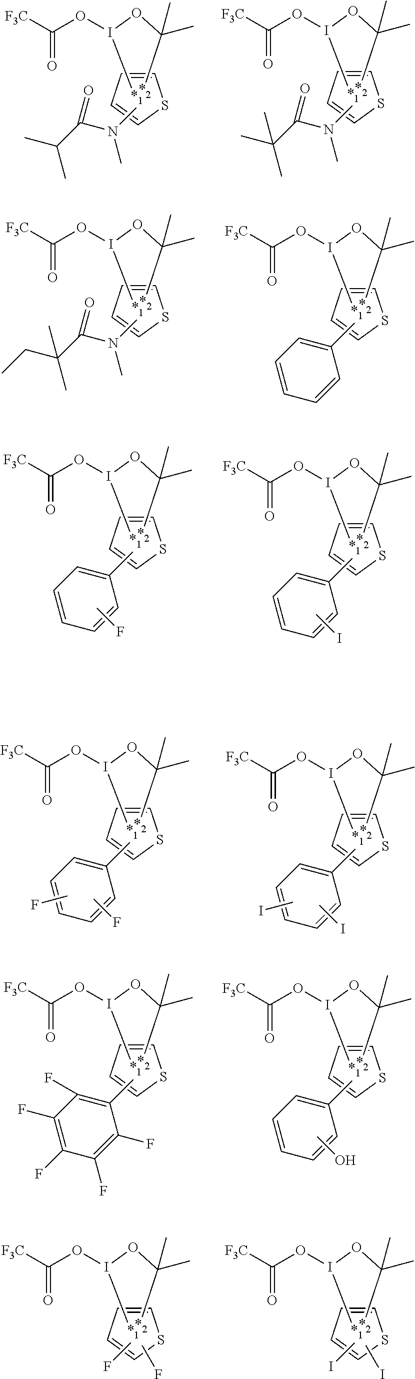

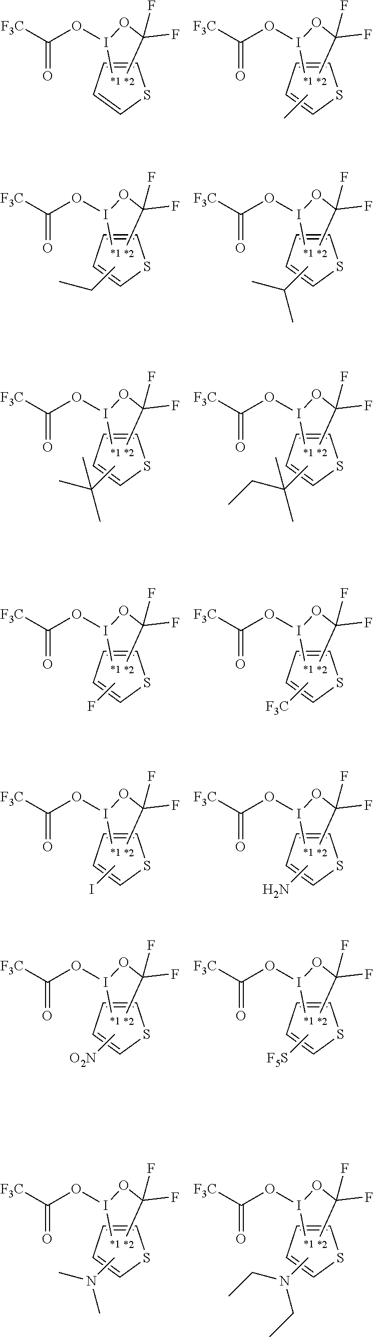

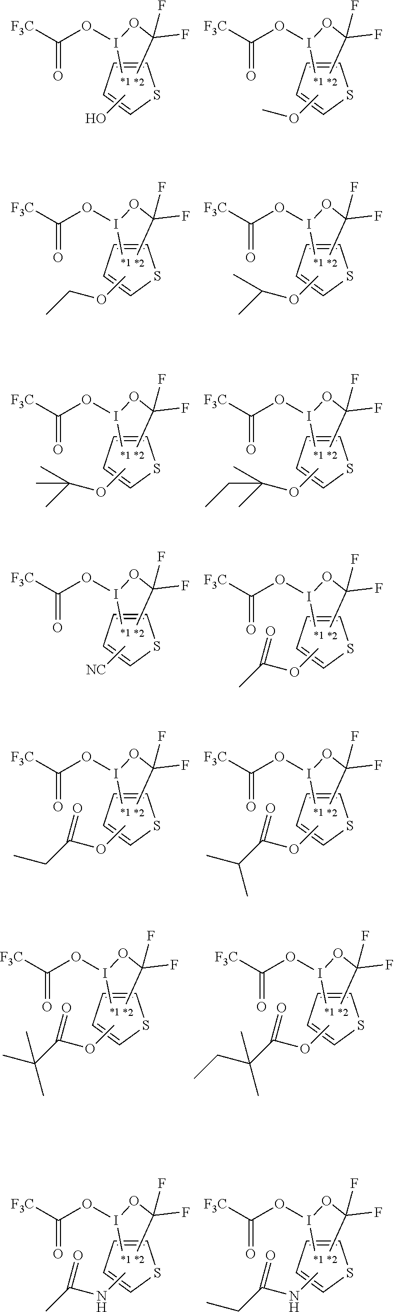

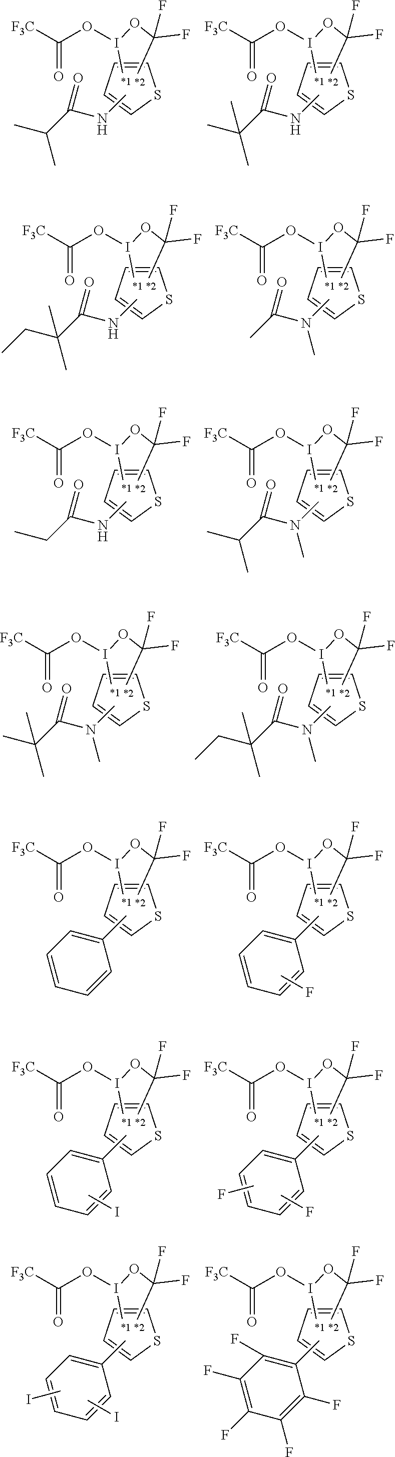

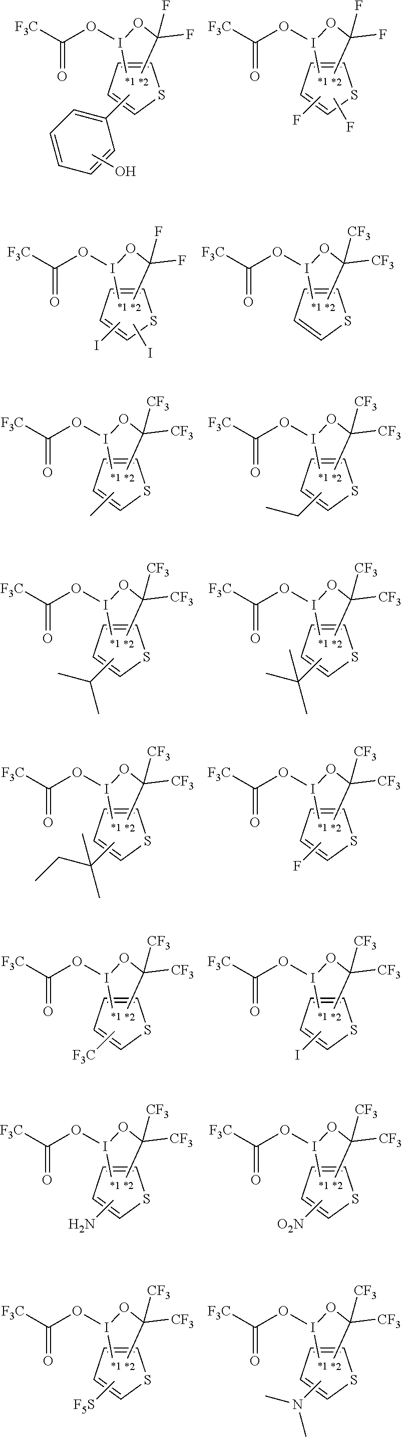

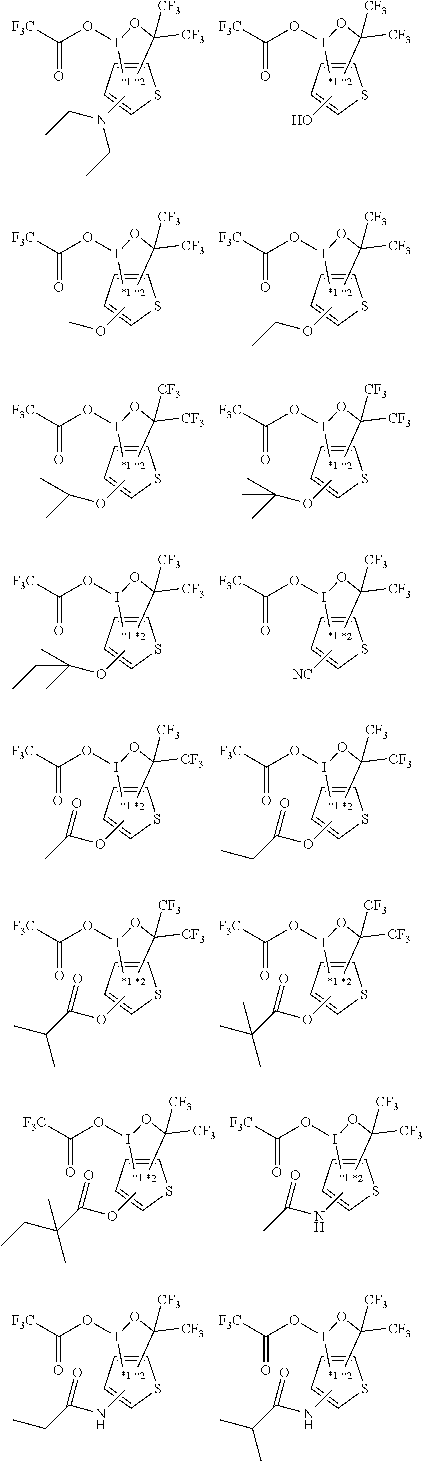

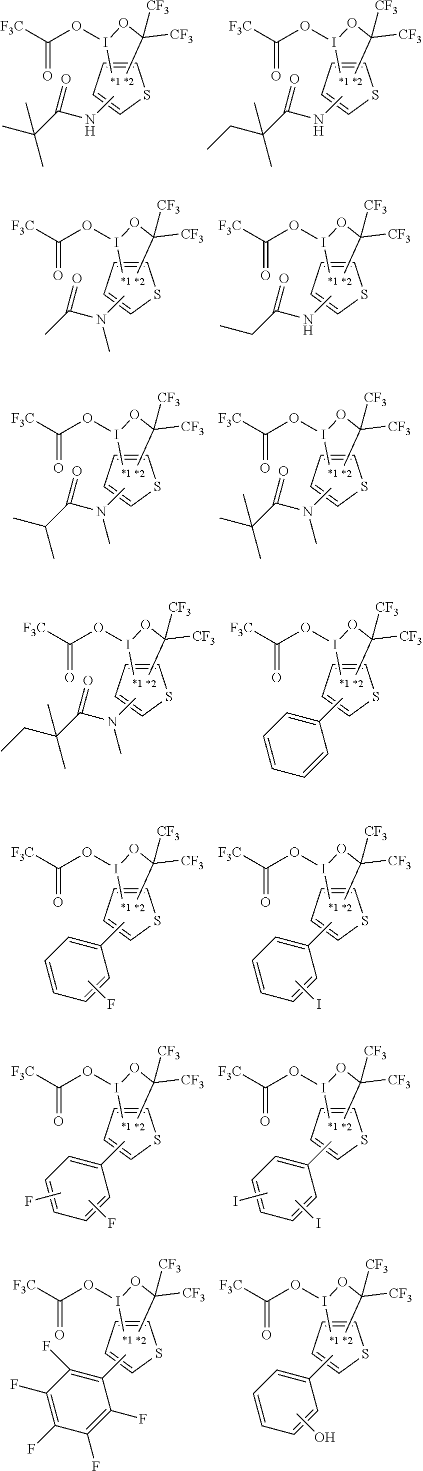

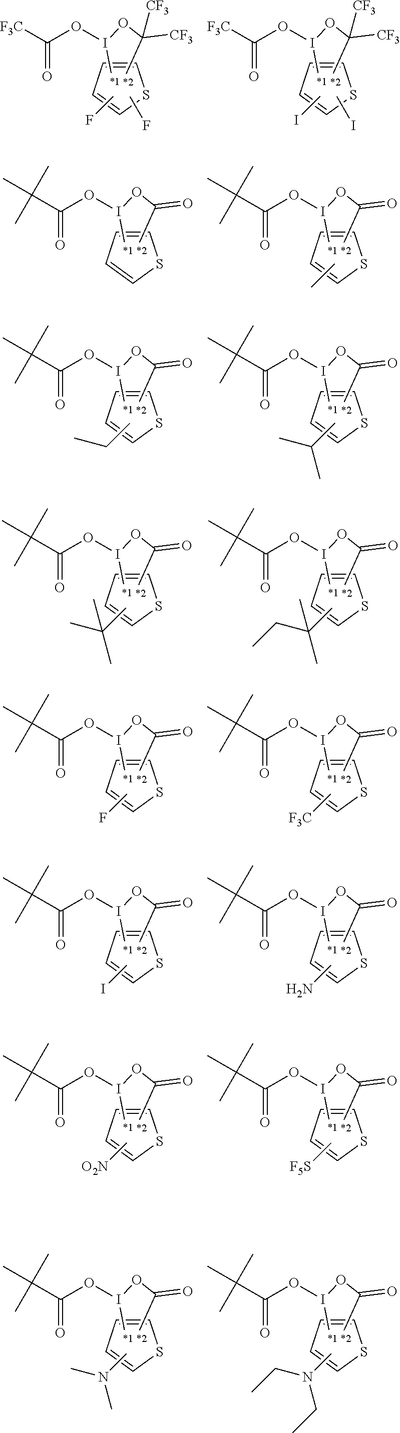

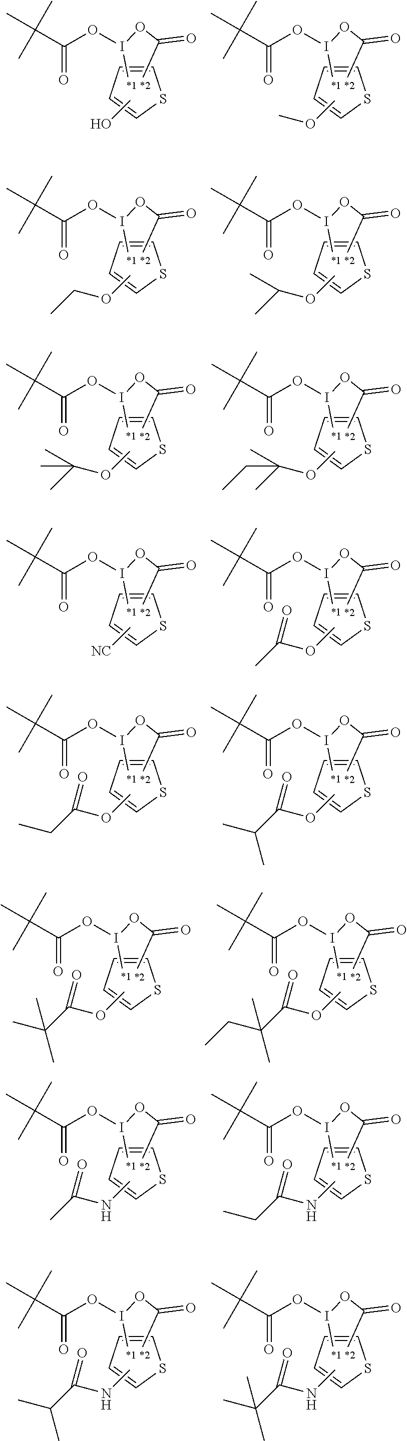

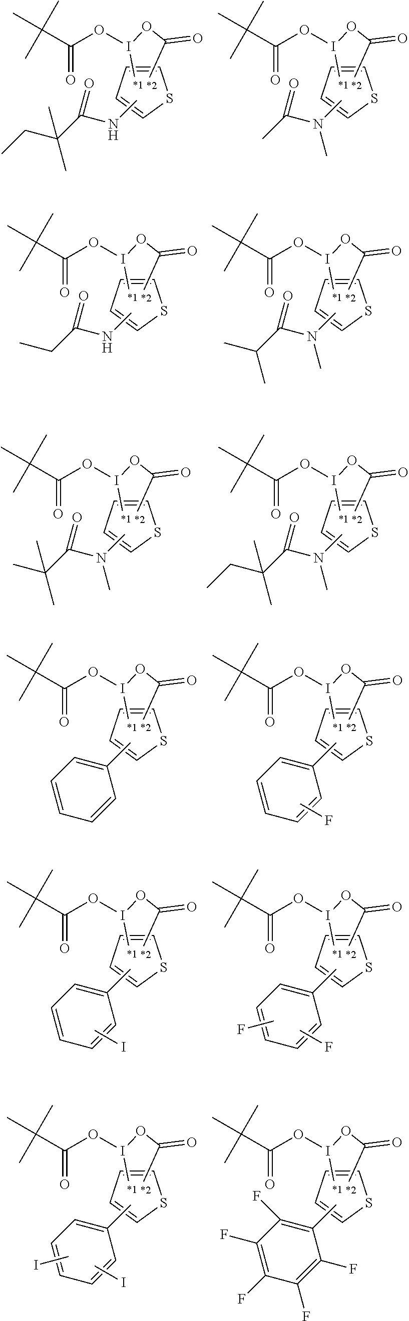

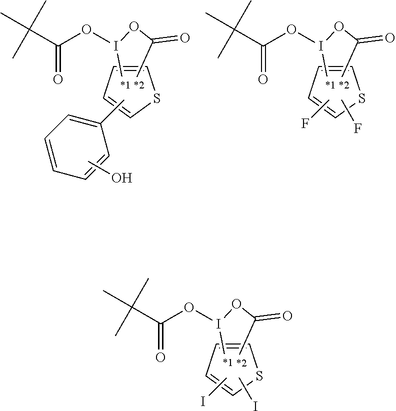

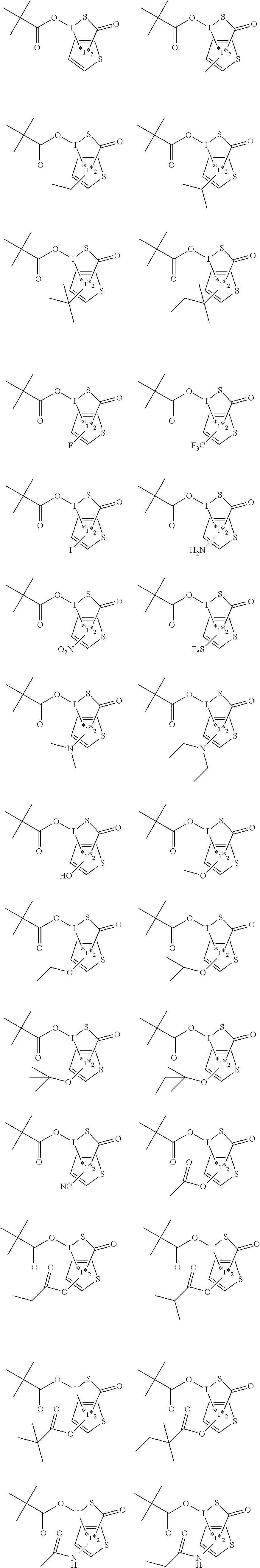

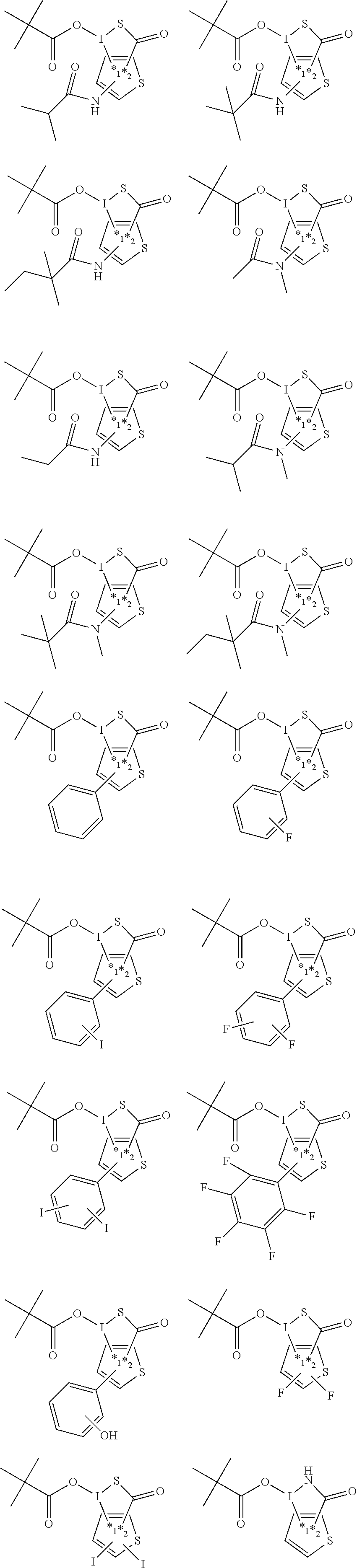

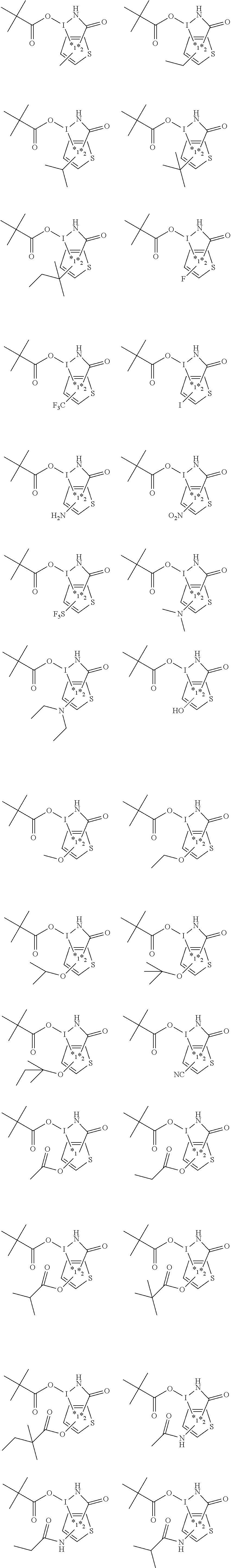

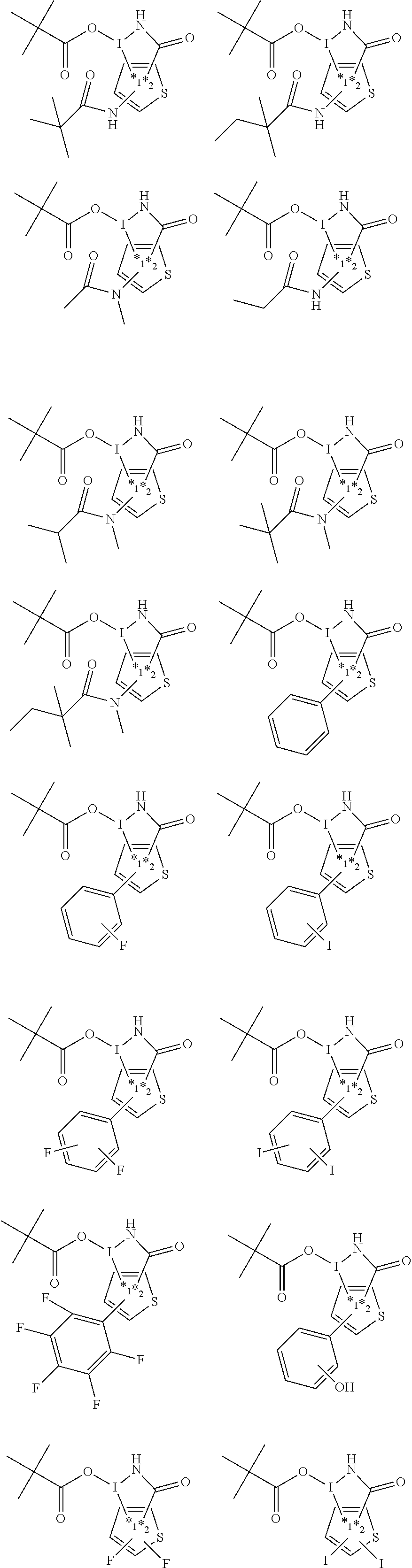

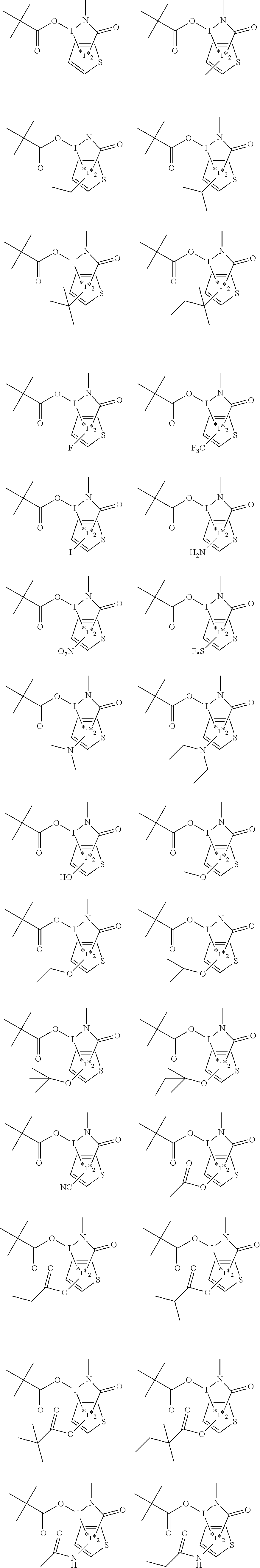

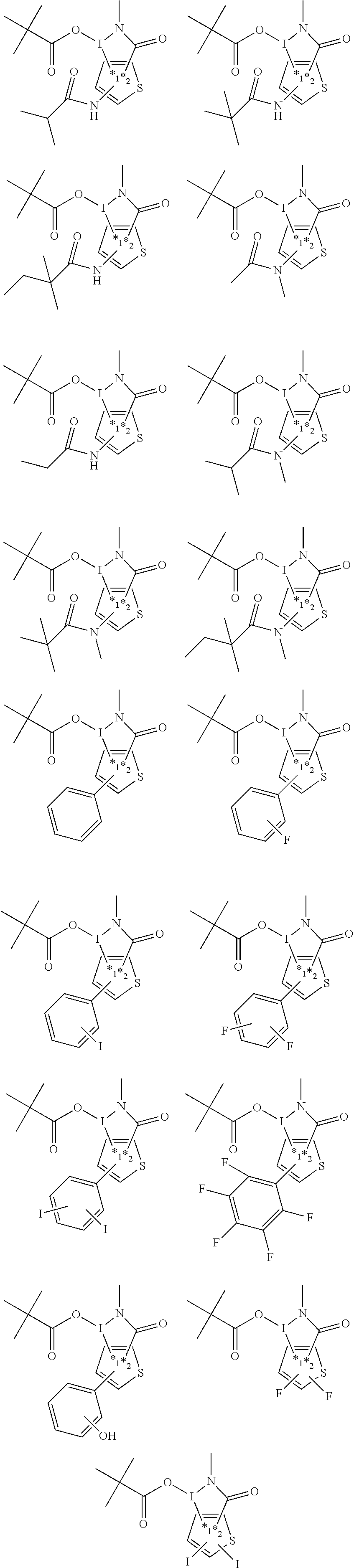

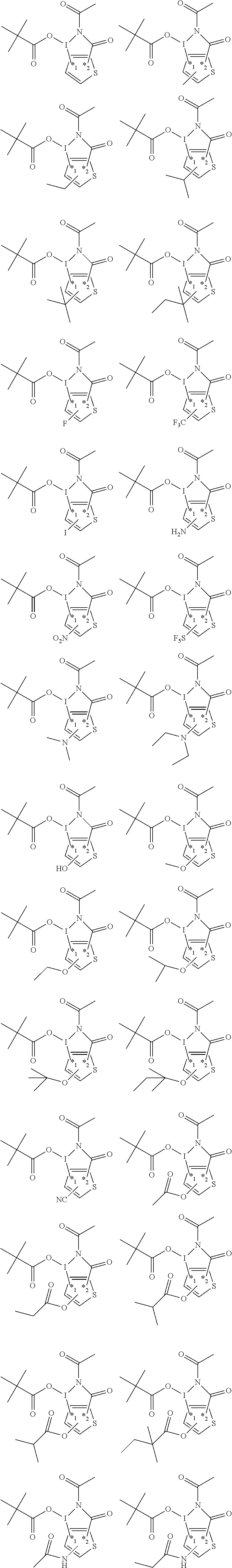

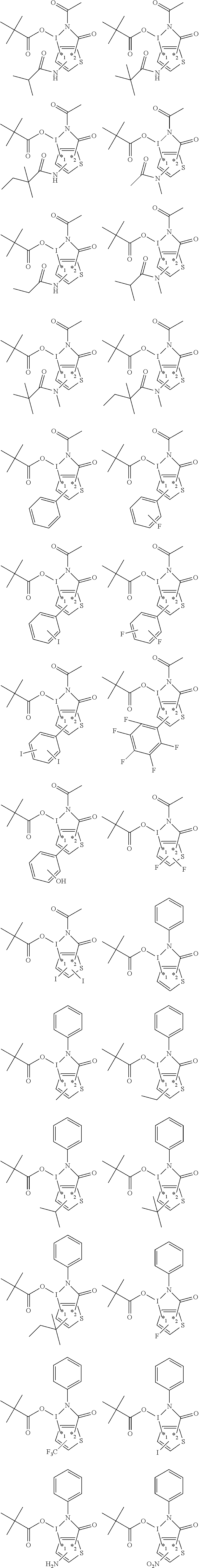

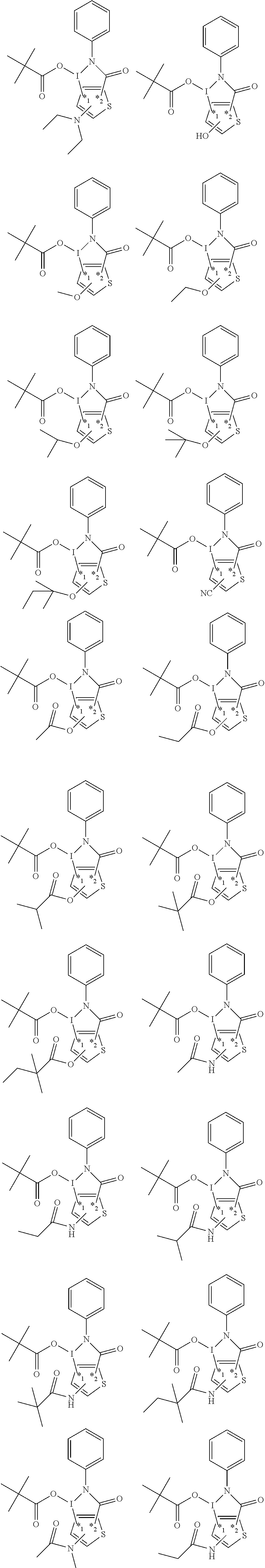

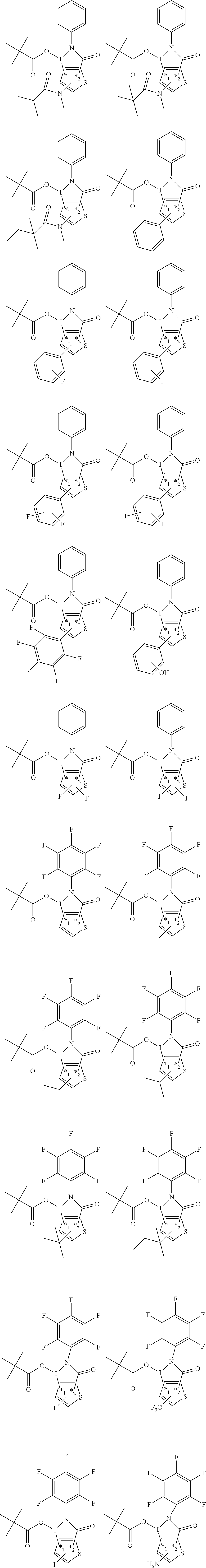

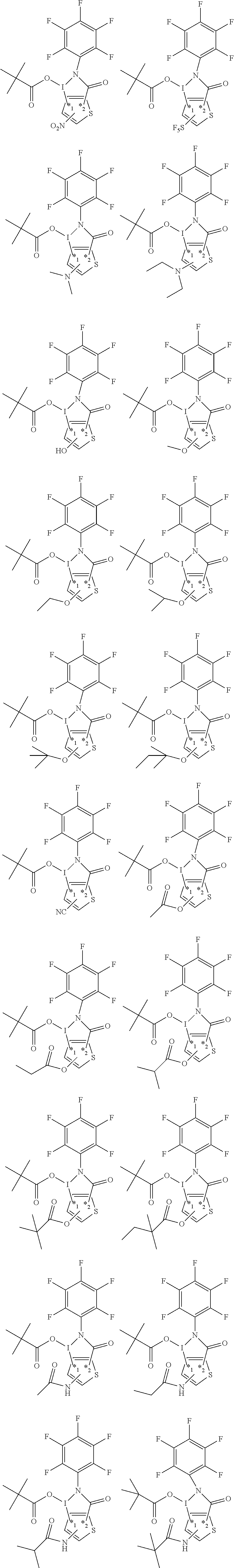

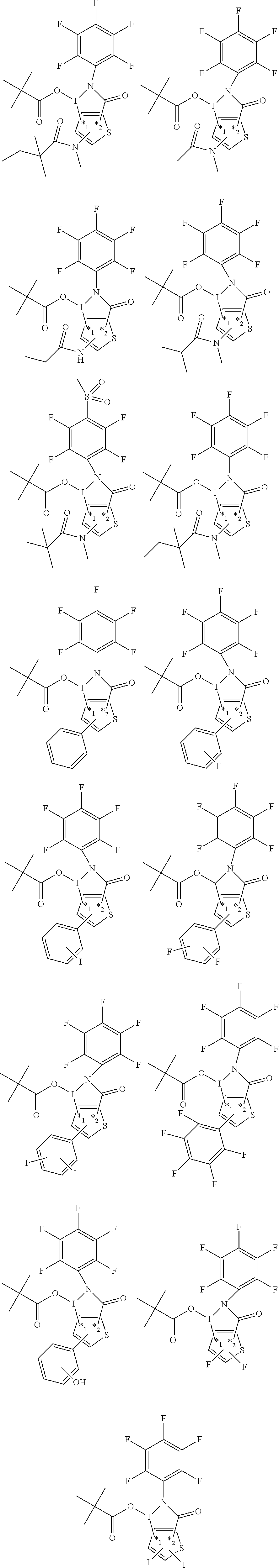

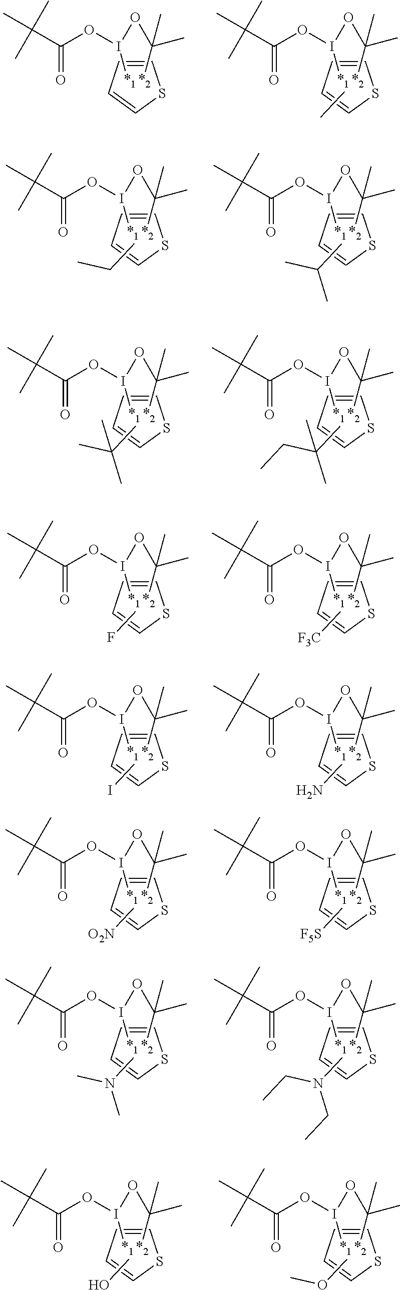

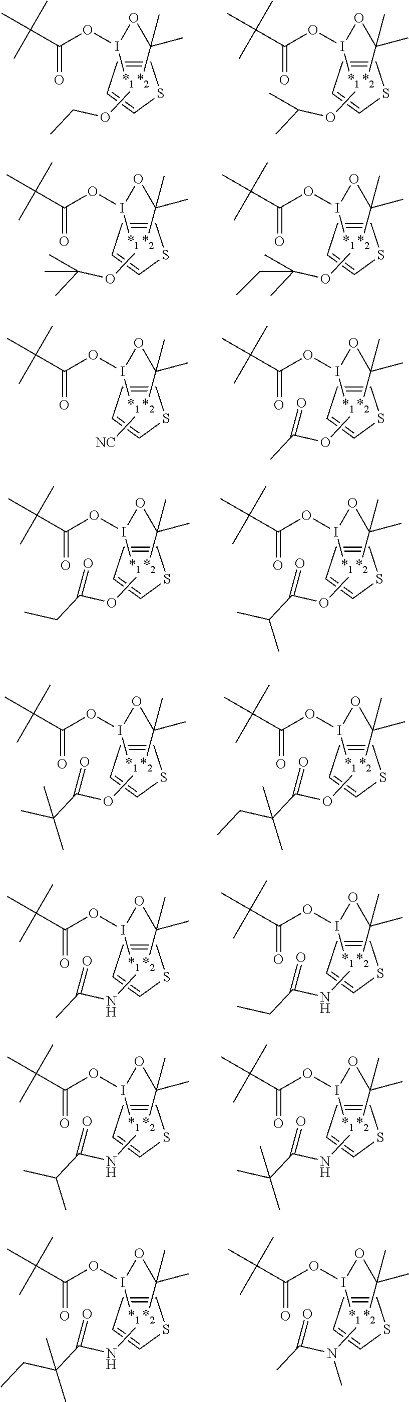

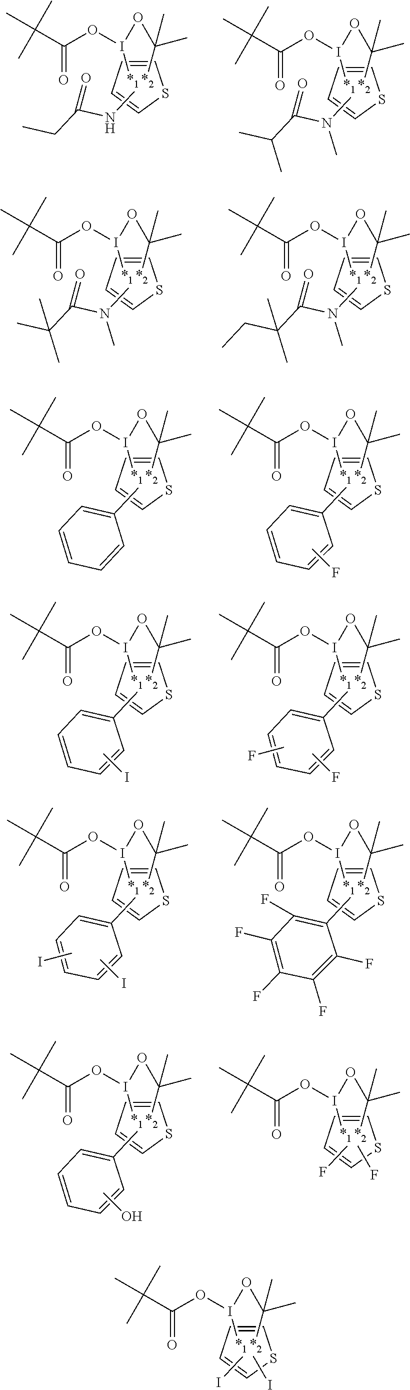

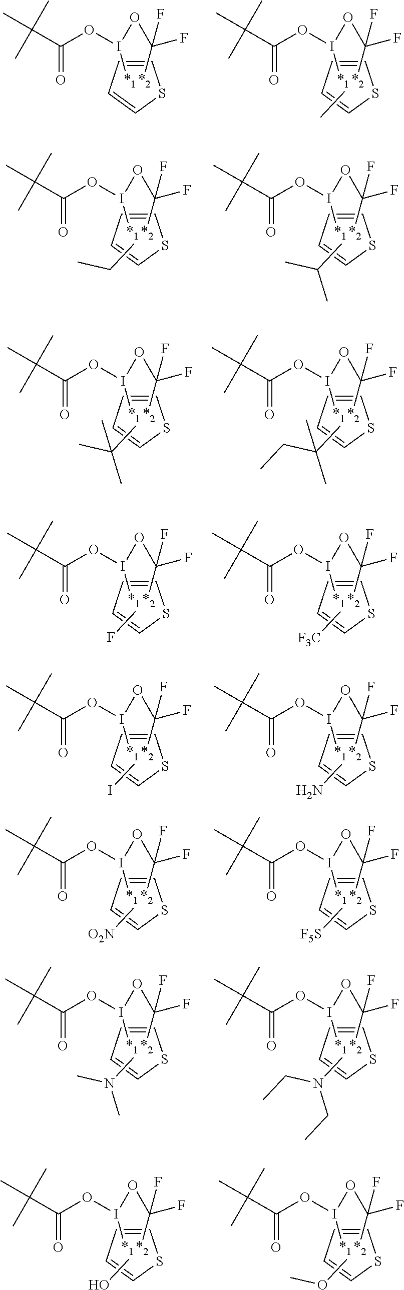

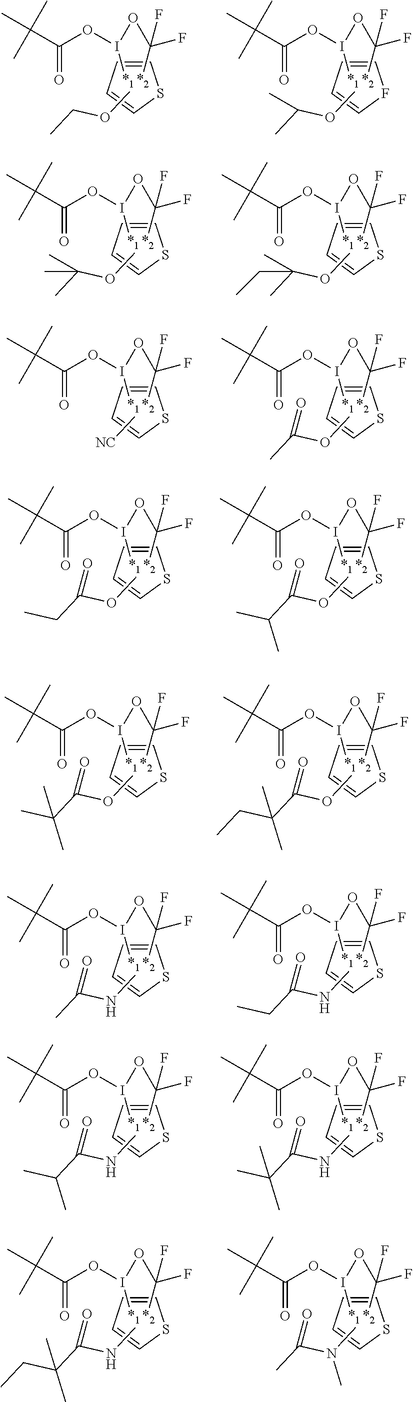

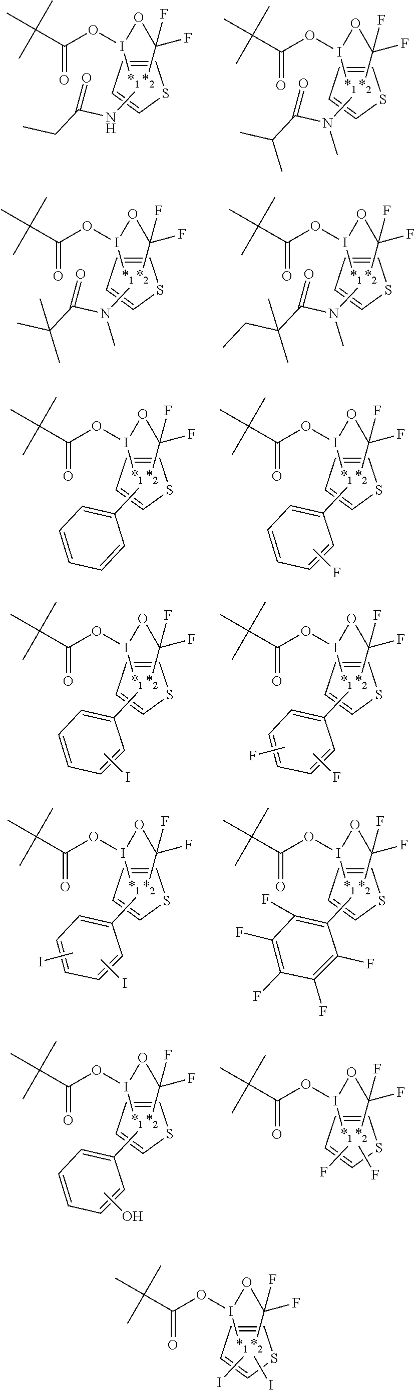

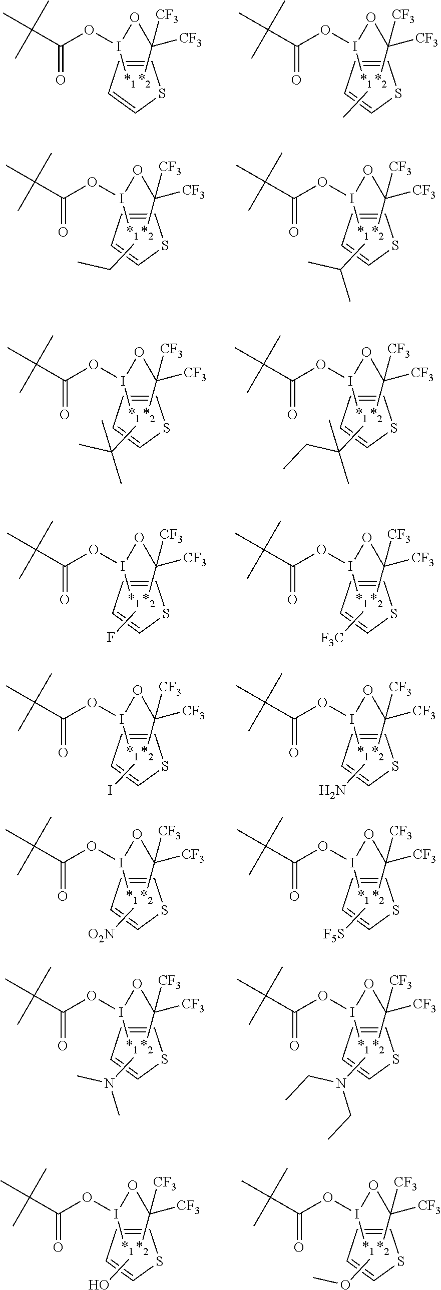

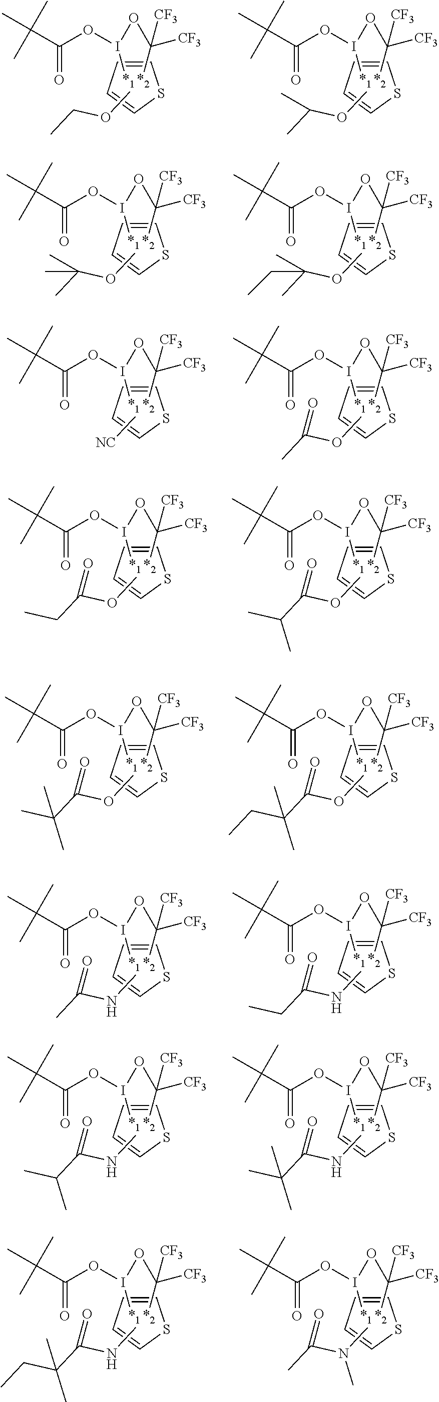

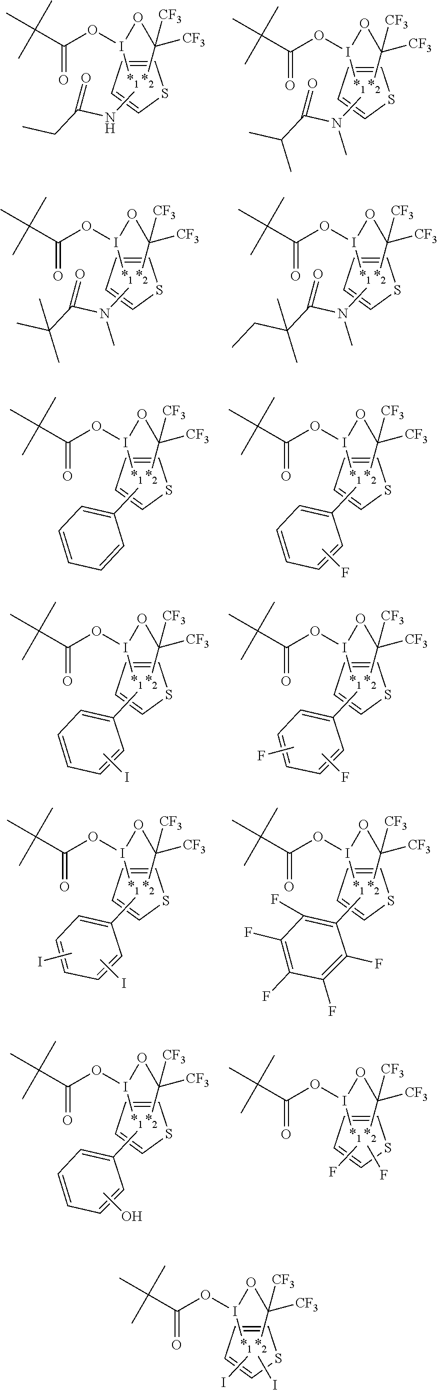

Specific examples of the hypervalent iodine compound represented by the formula (2) include the following, but are not limited thereto. In the following formulae, *3 and *4 represent the same as defined above.

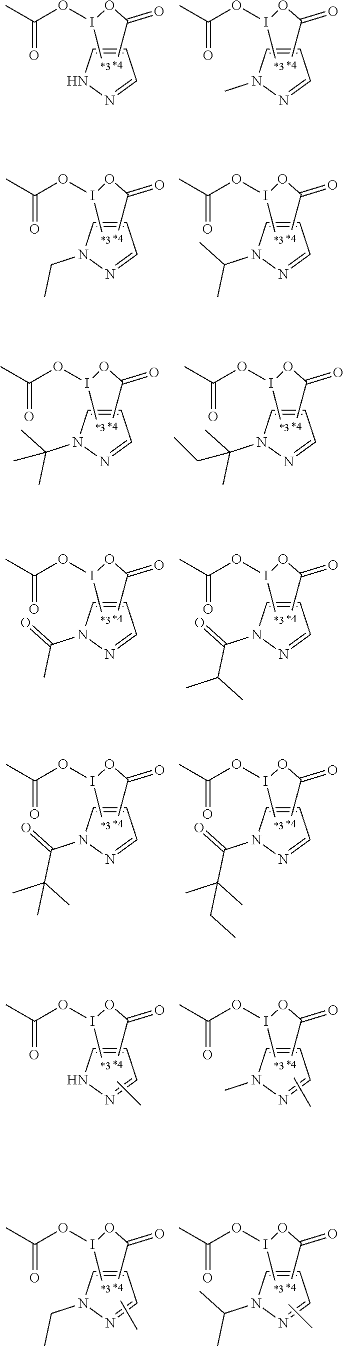

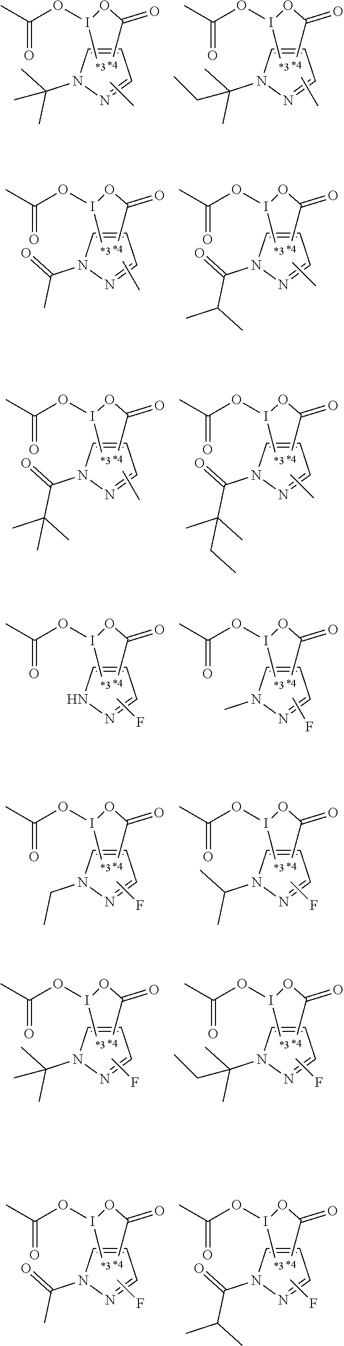

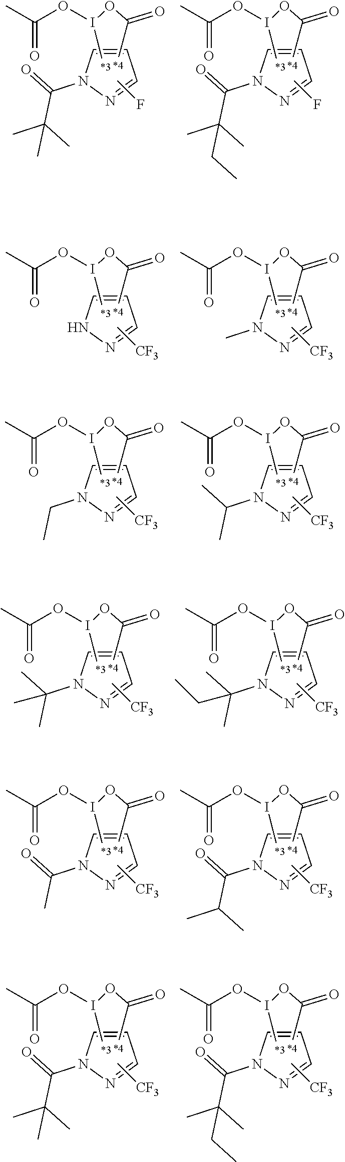

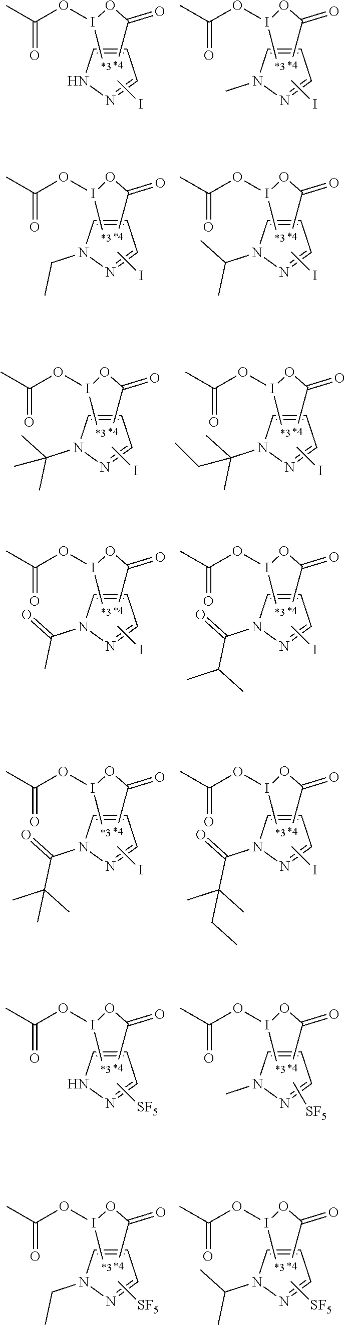

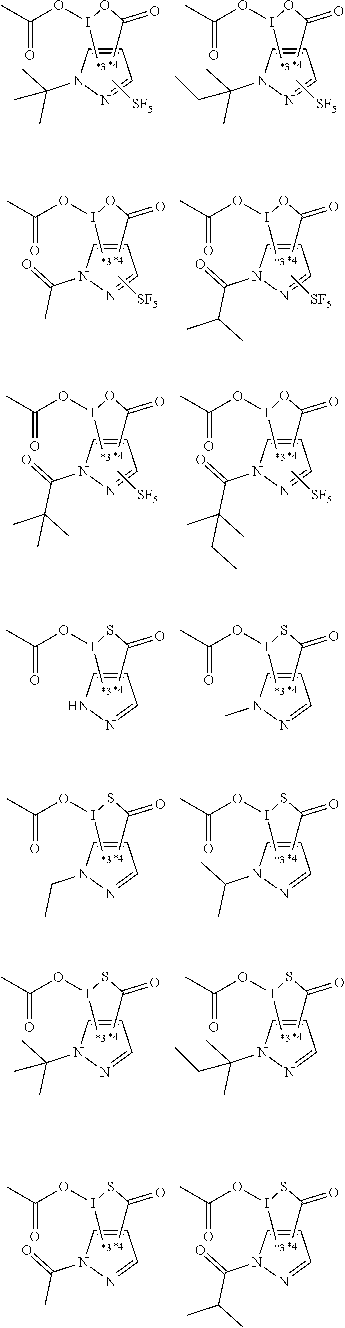

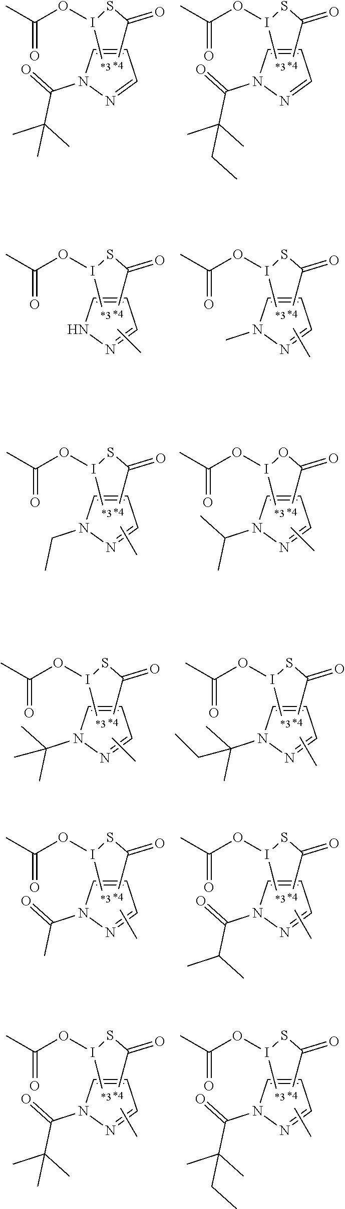

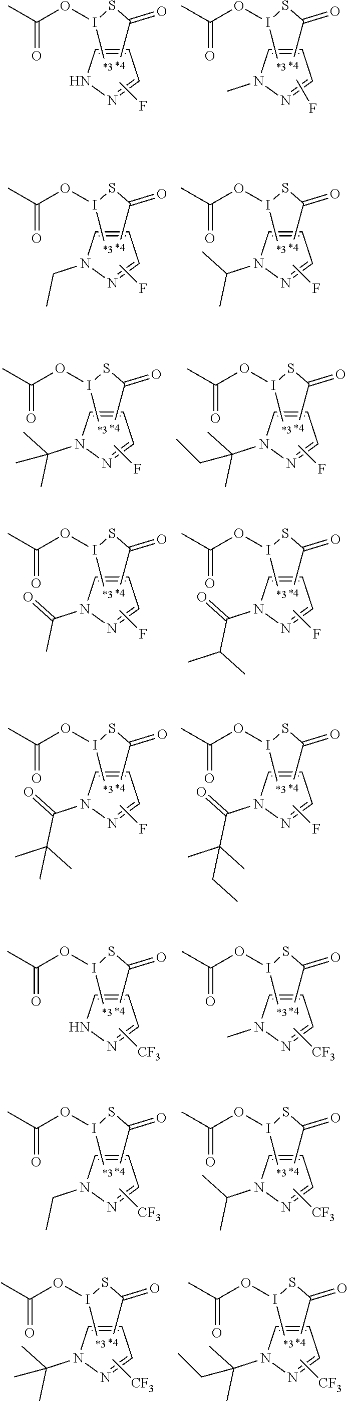

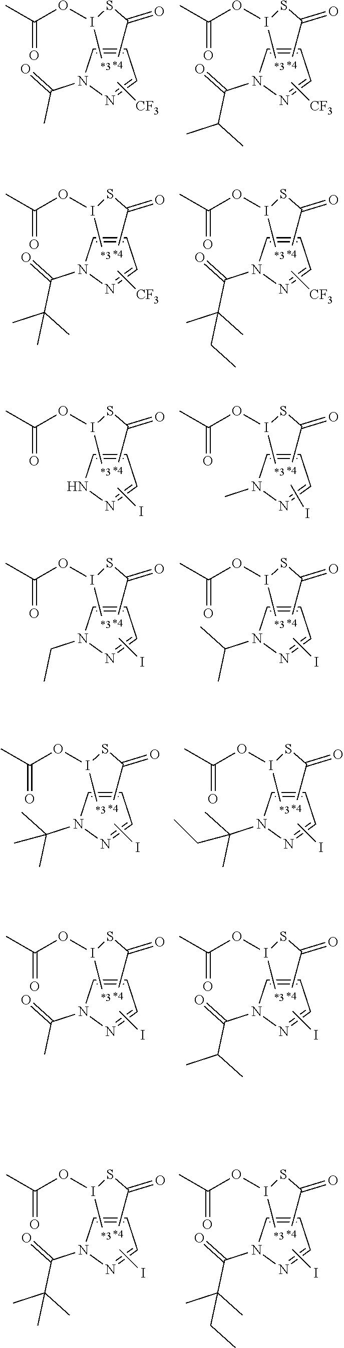

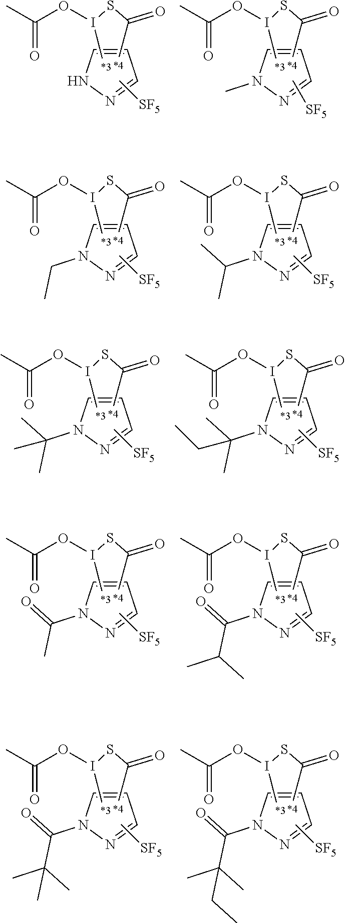

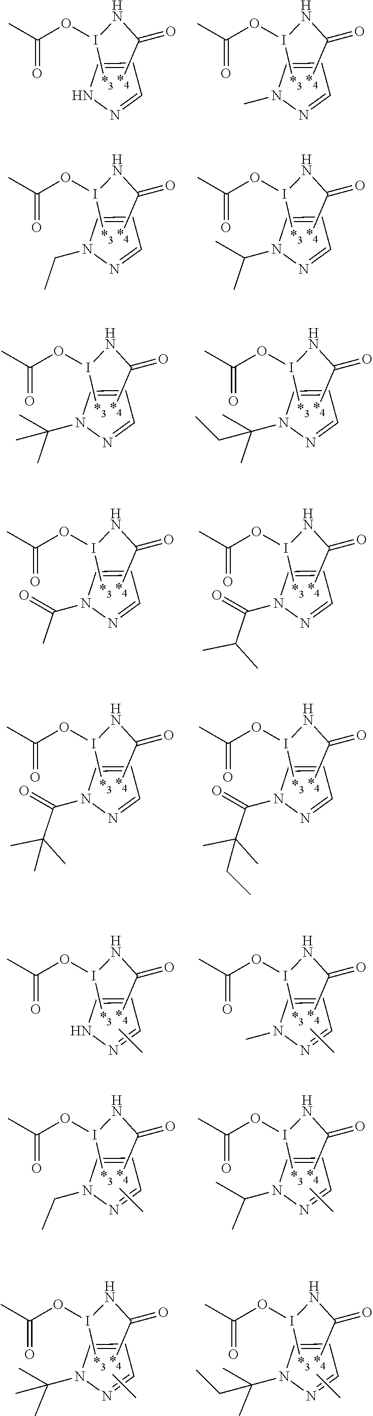

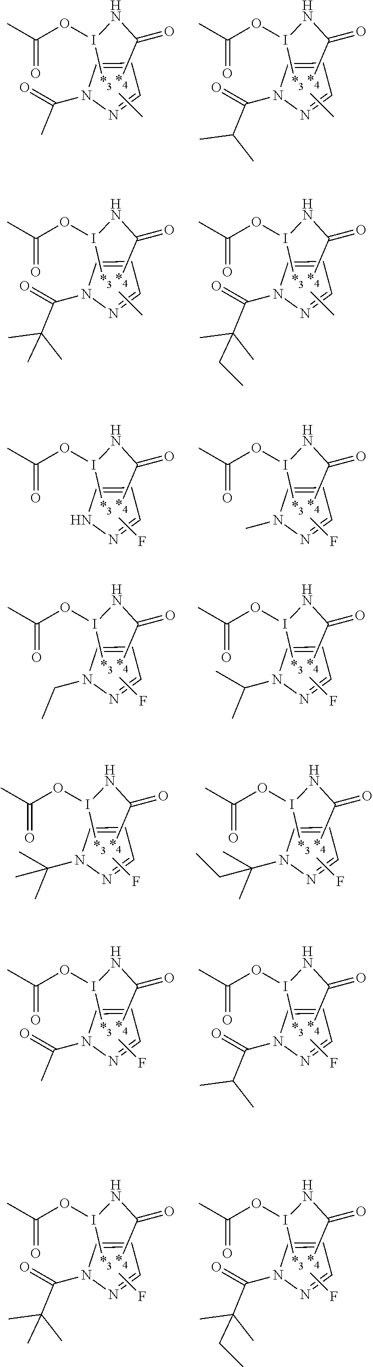

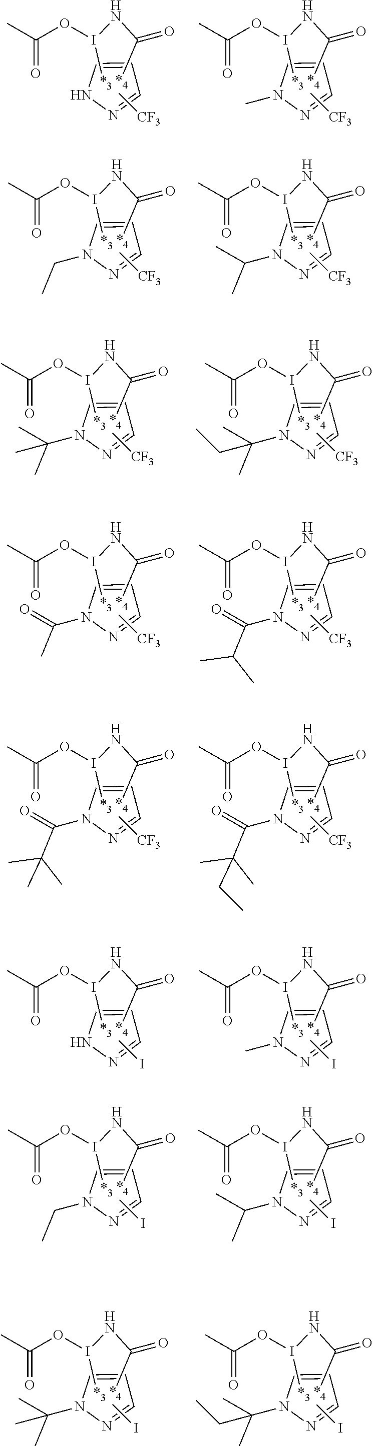

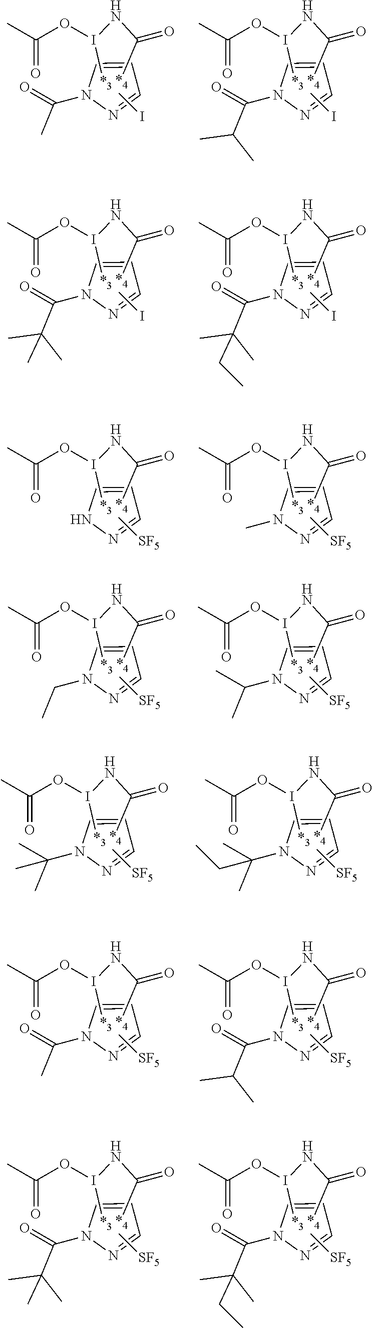

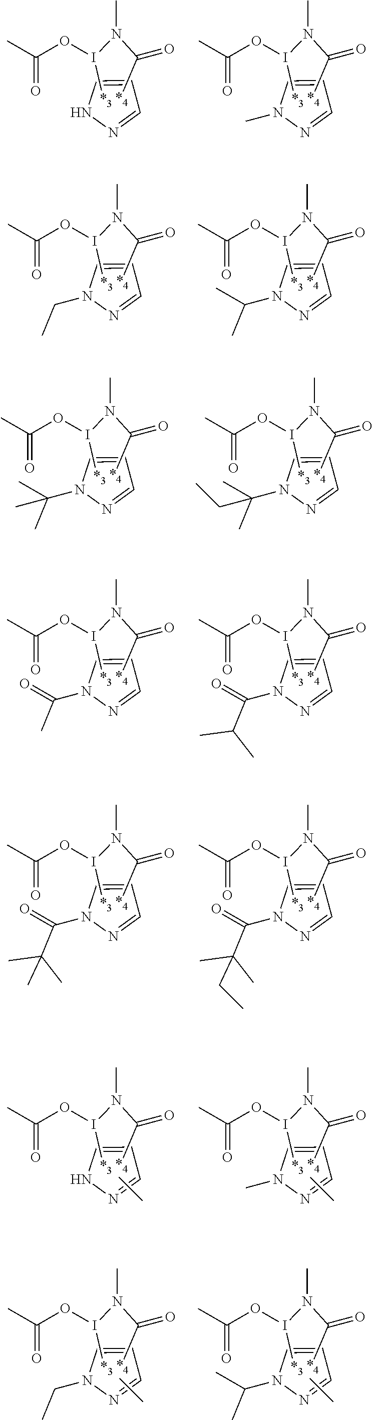

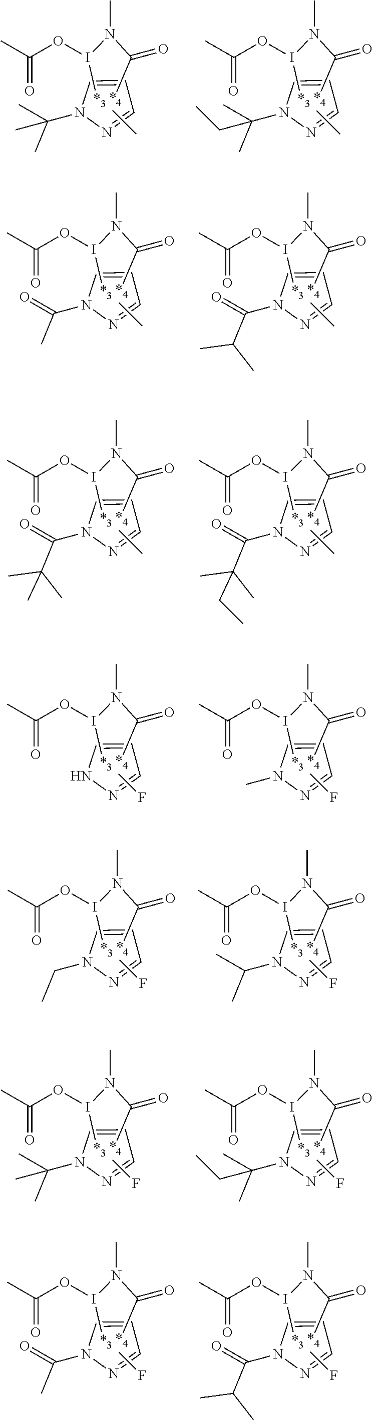

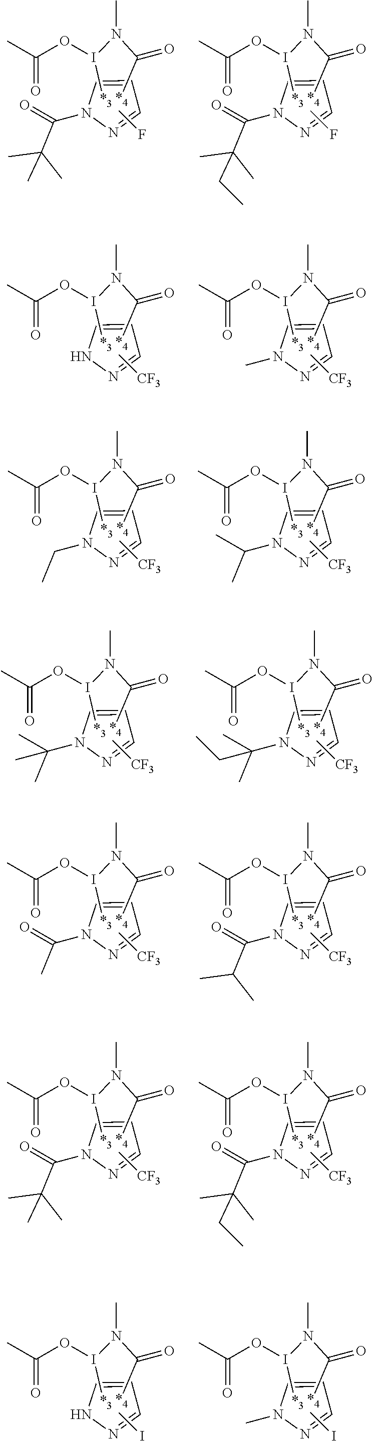

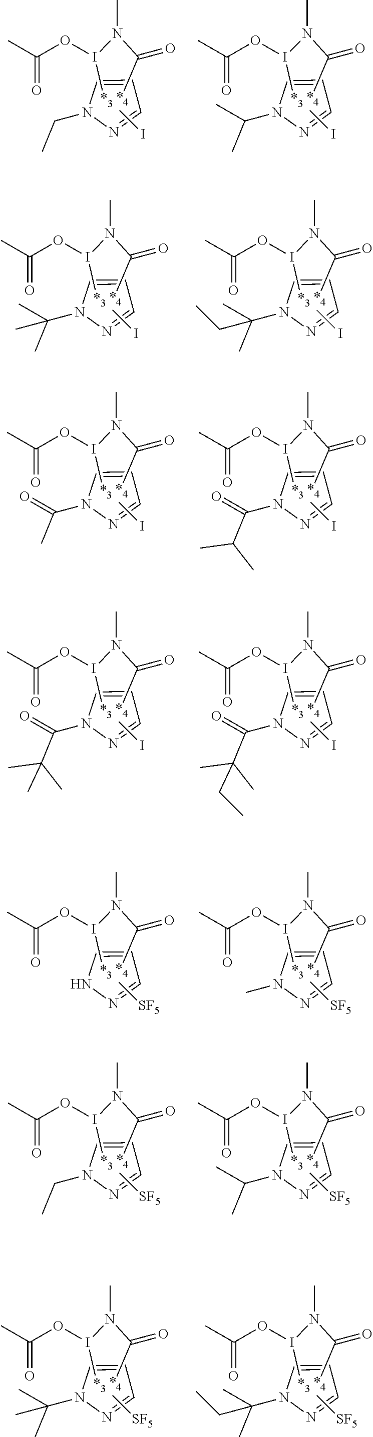

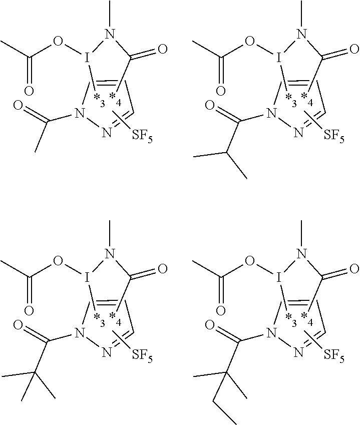

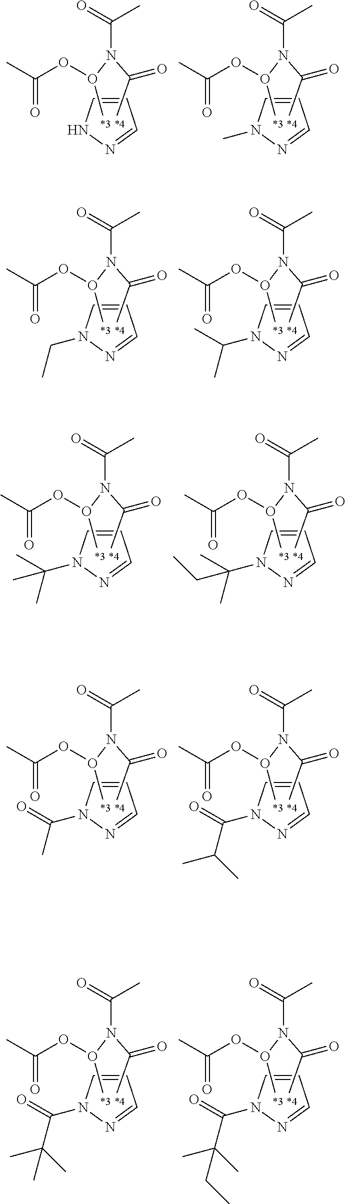

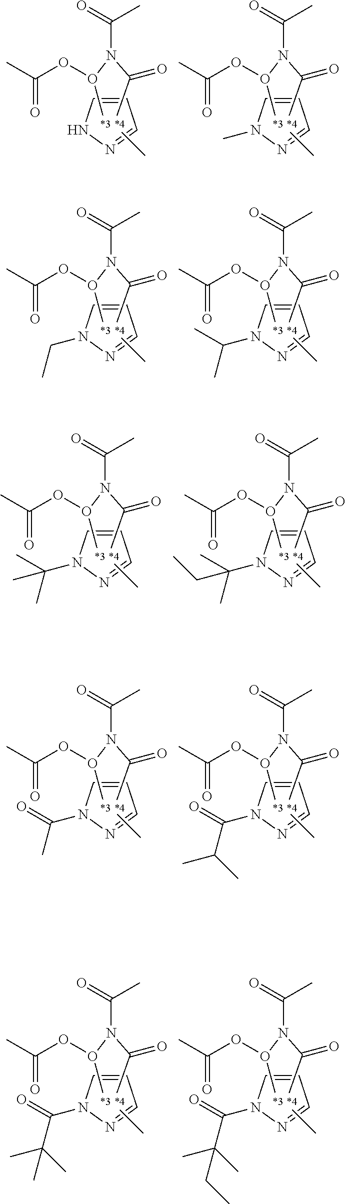

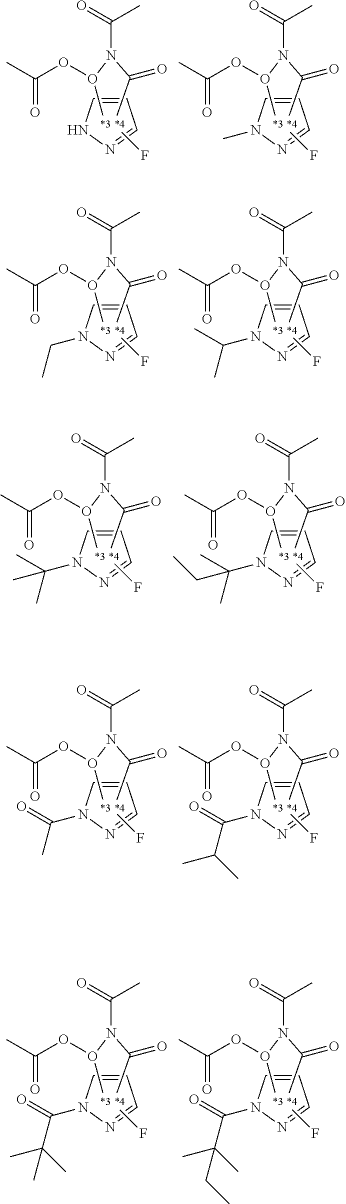

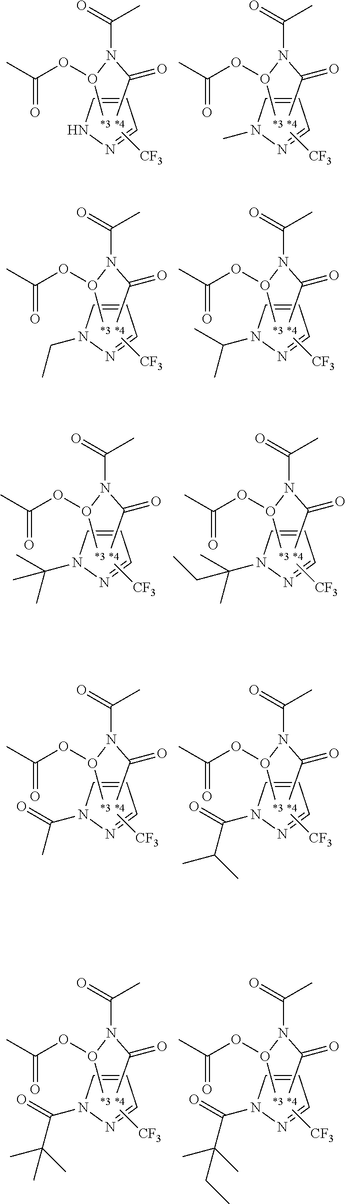

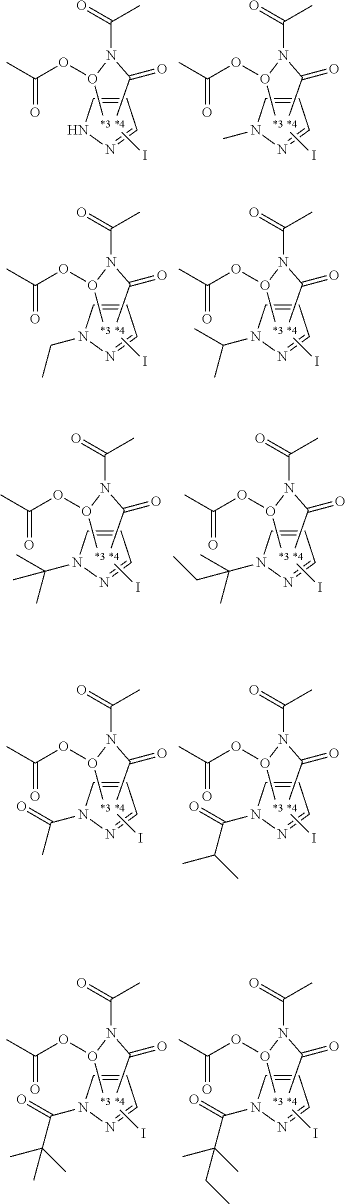

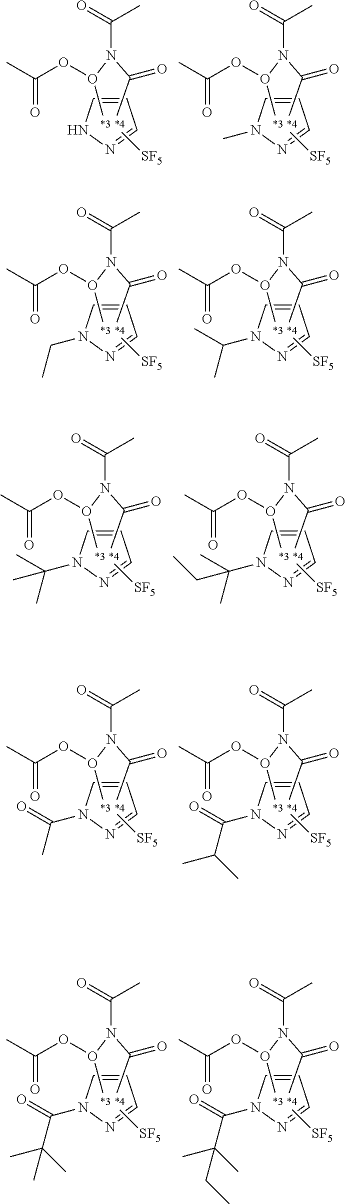

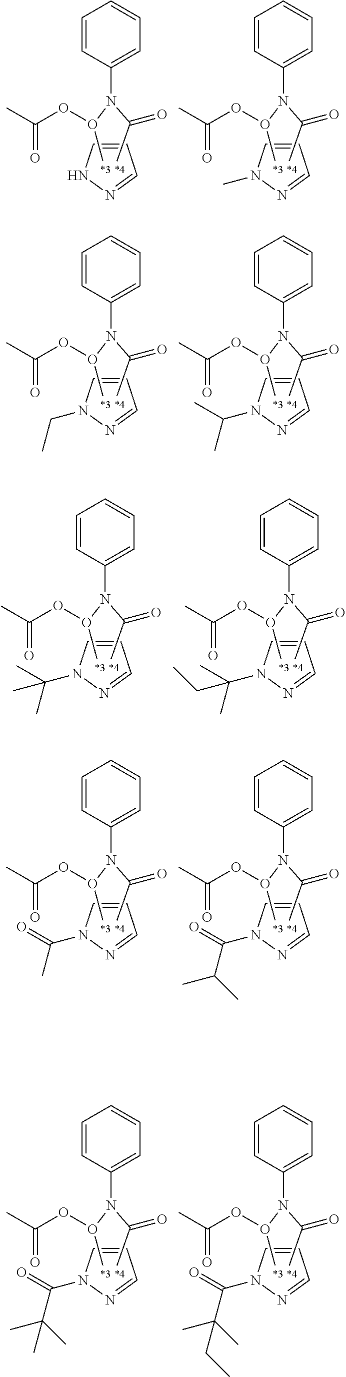

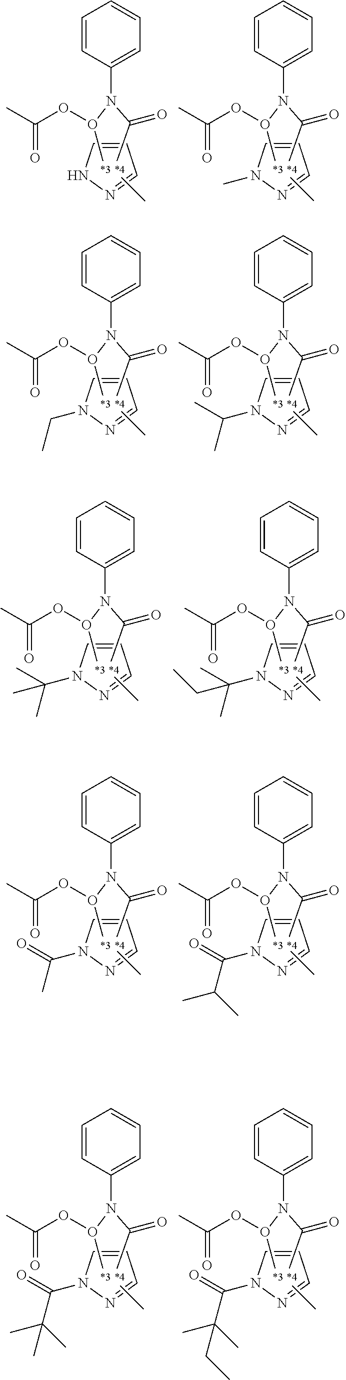

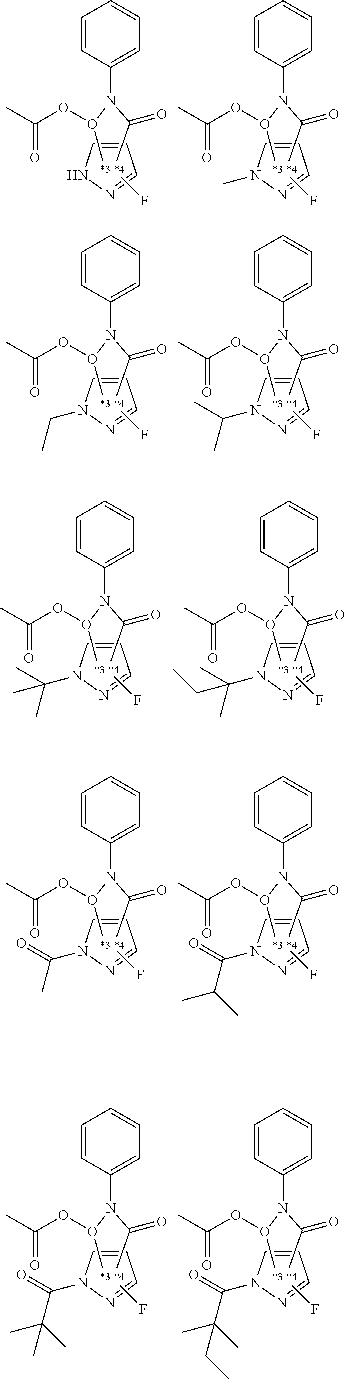

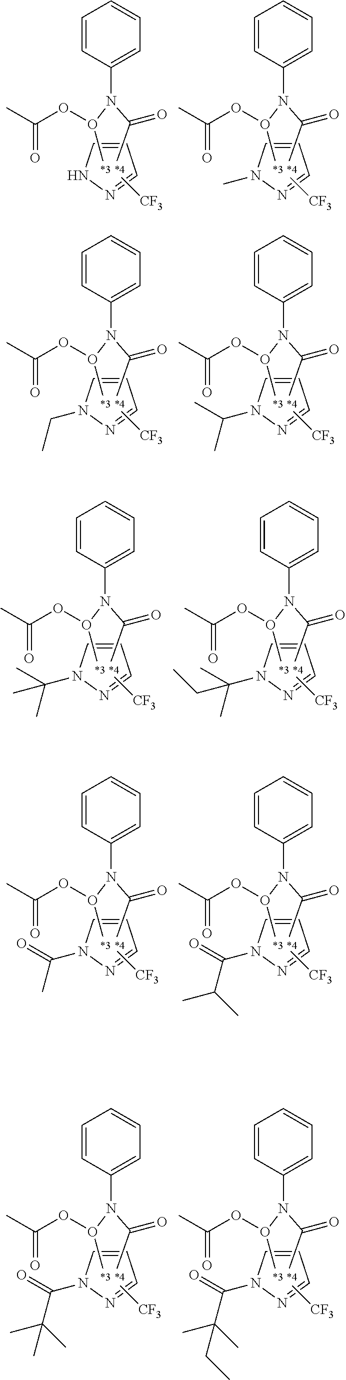

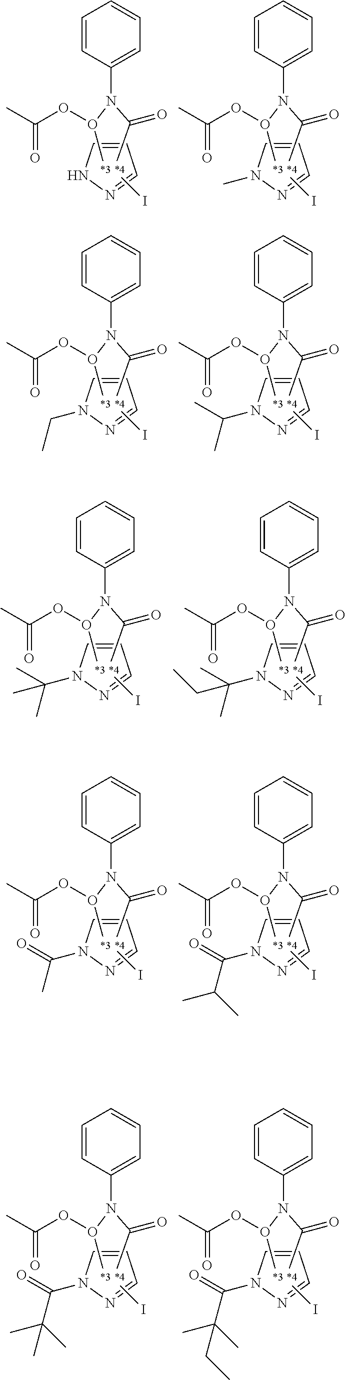

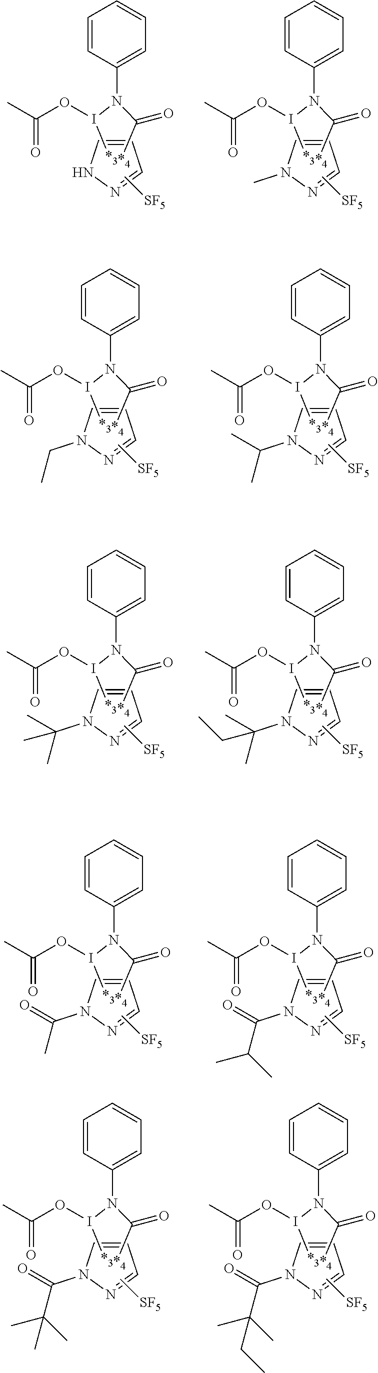

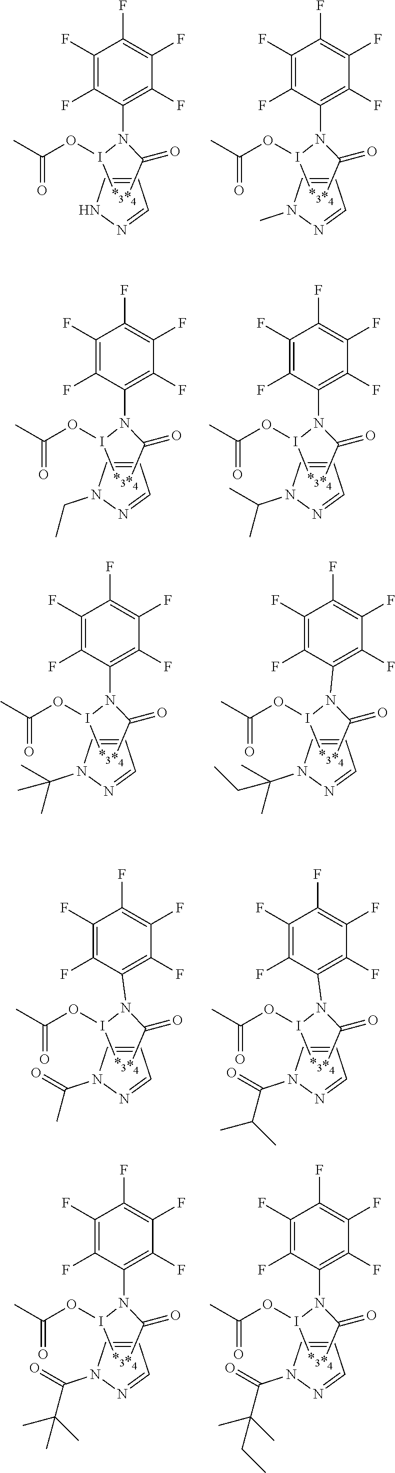

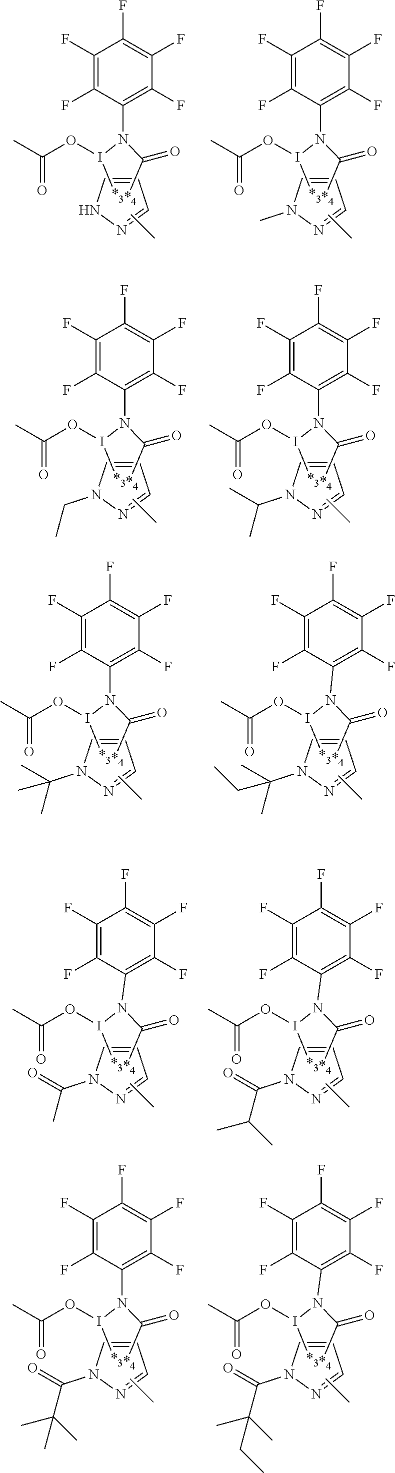

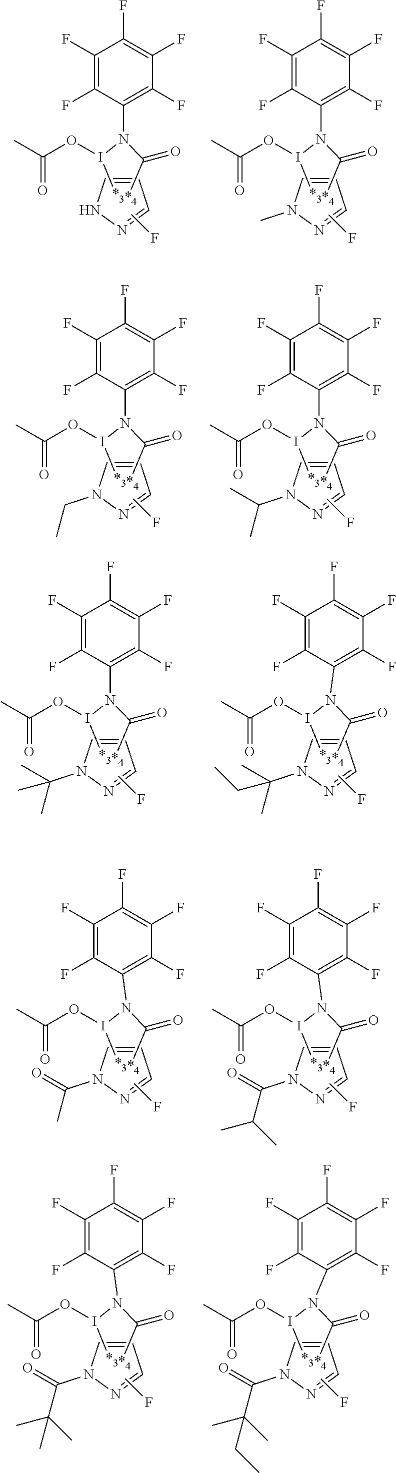

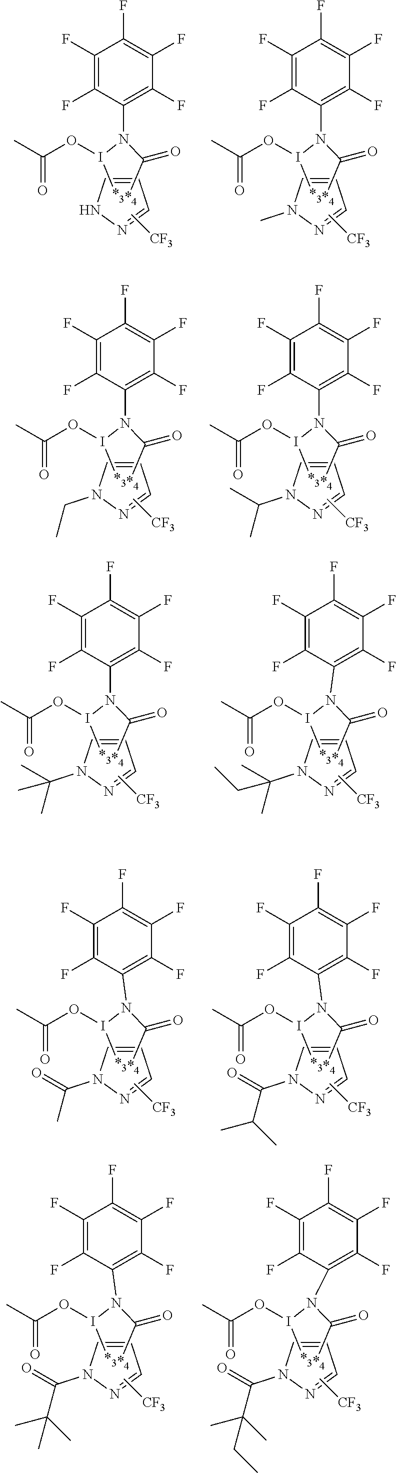

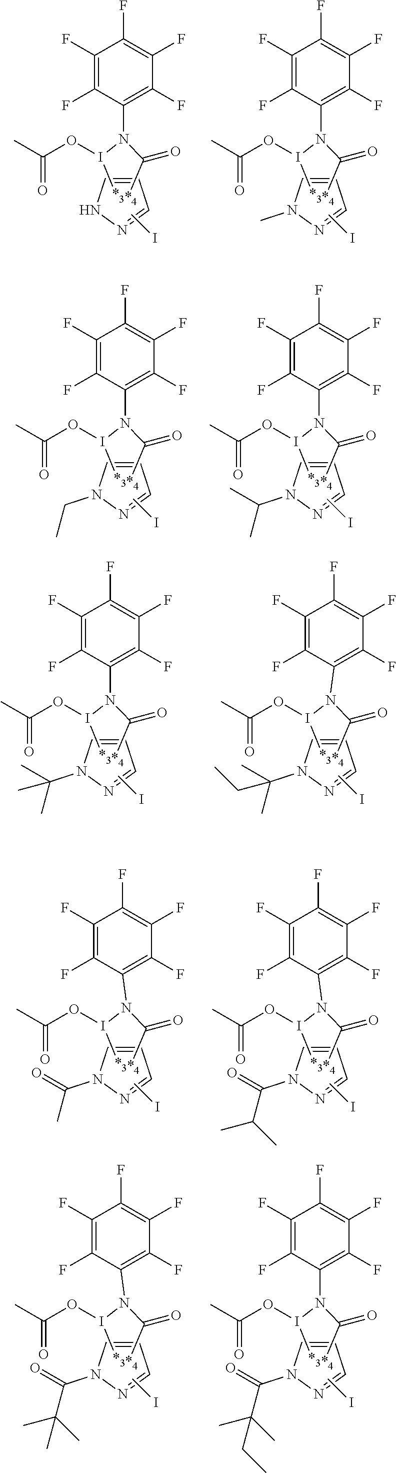

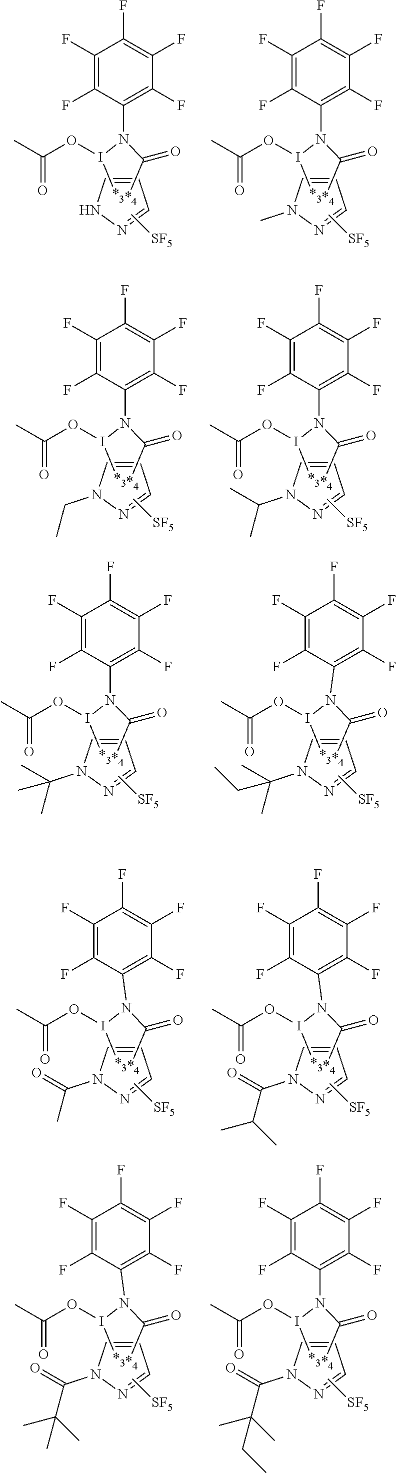

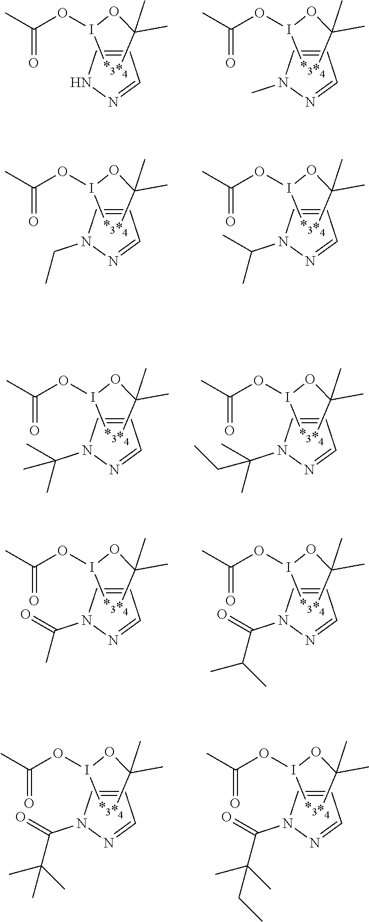

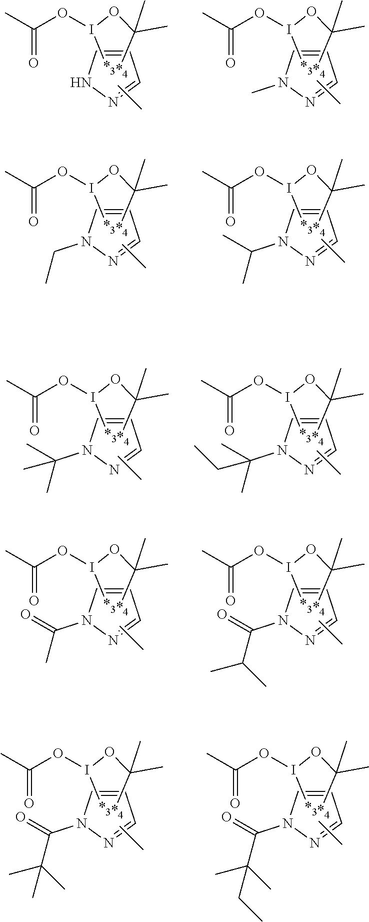

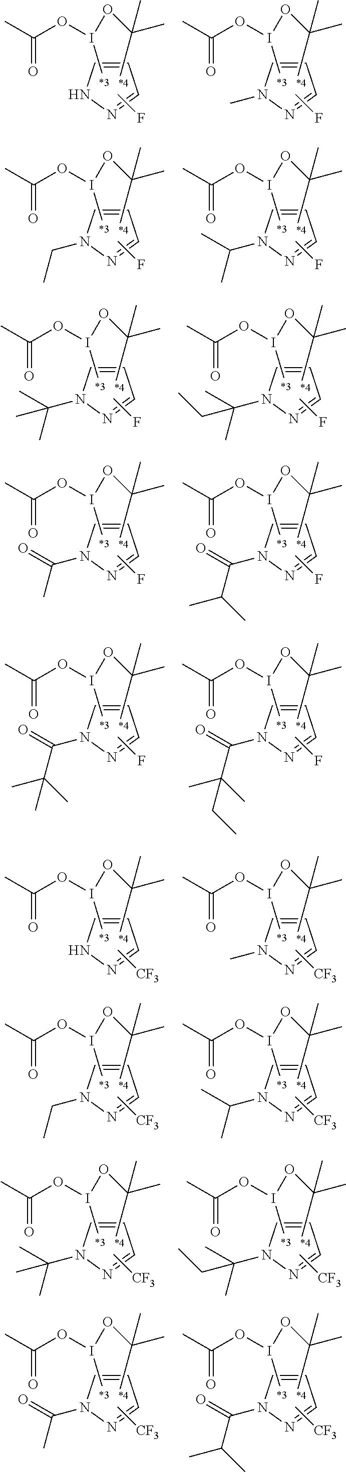

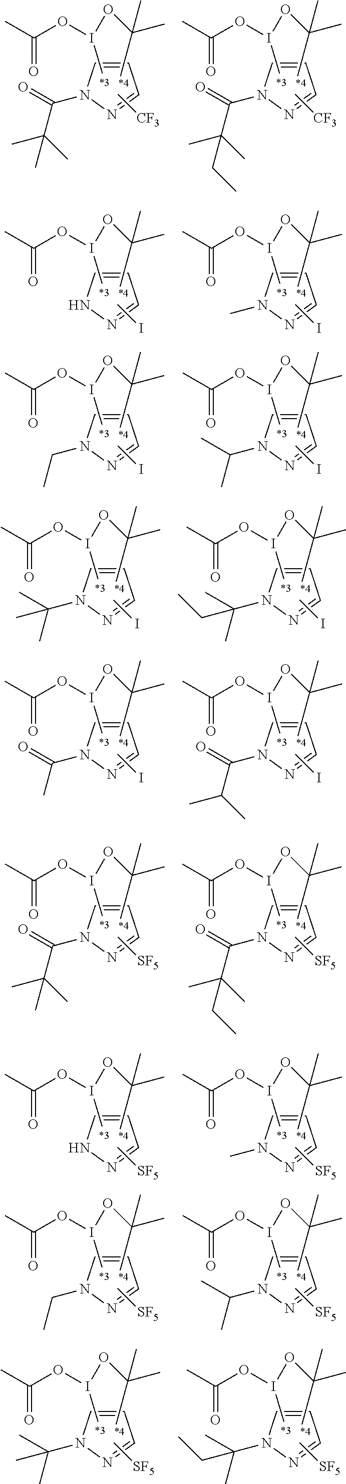

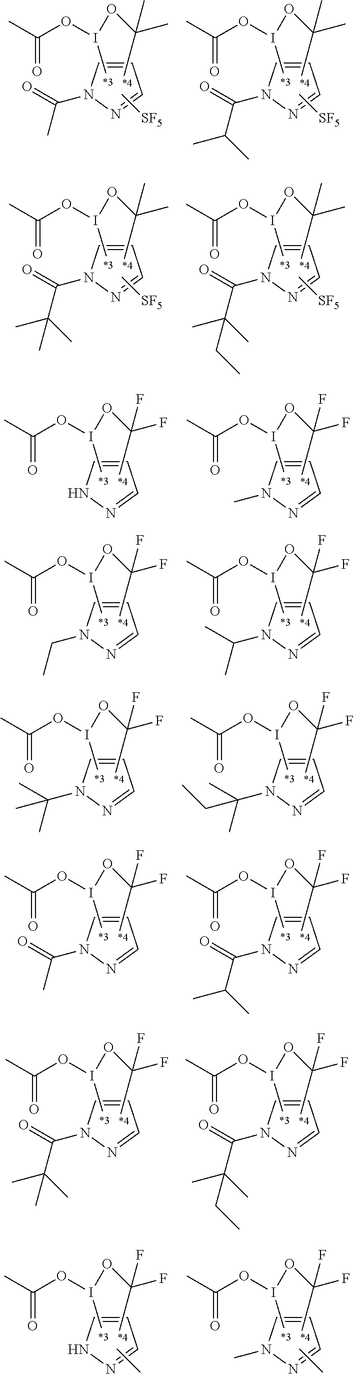

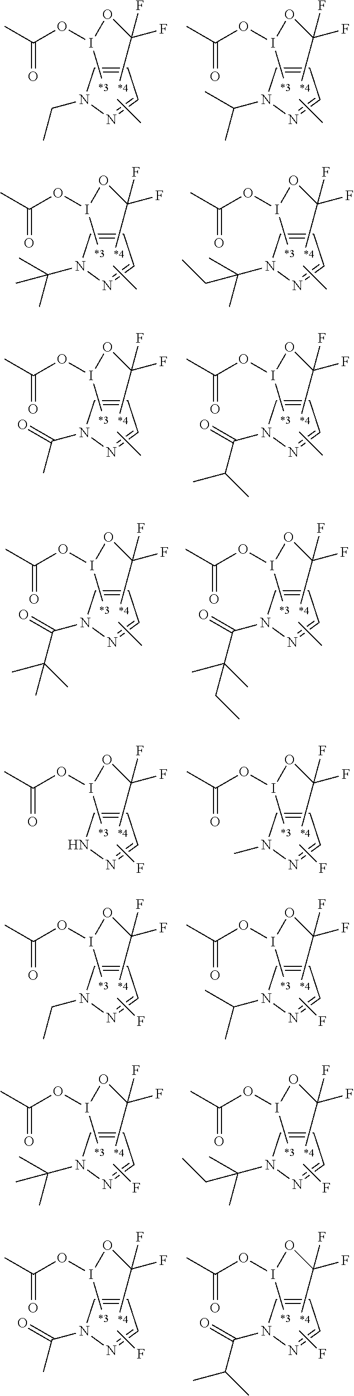

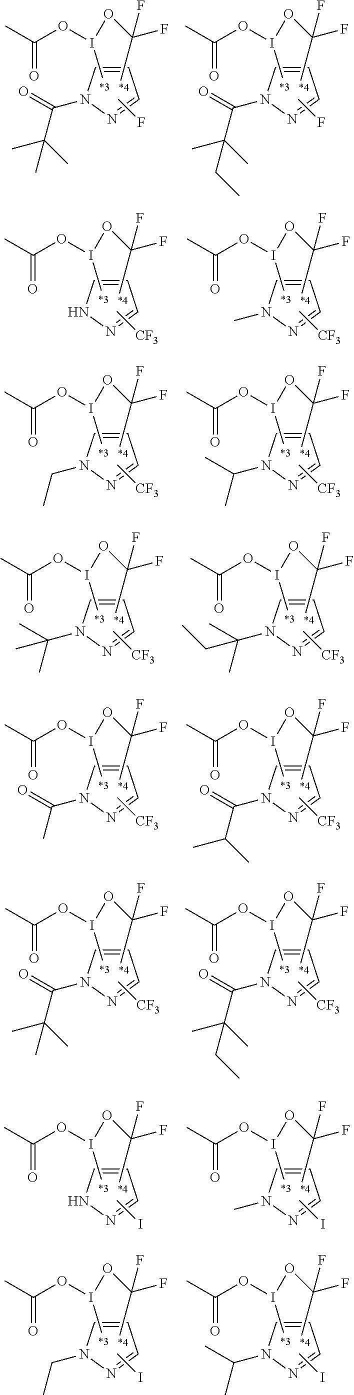

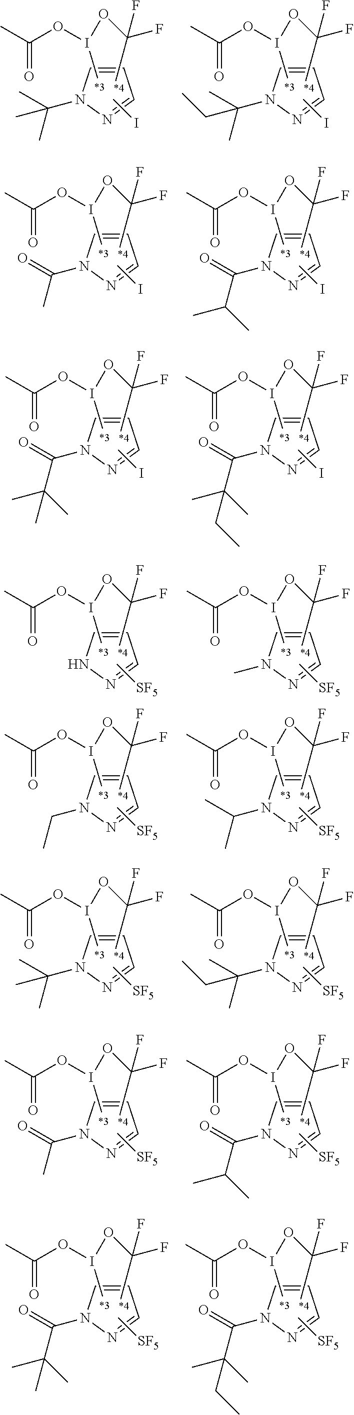

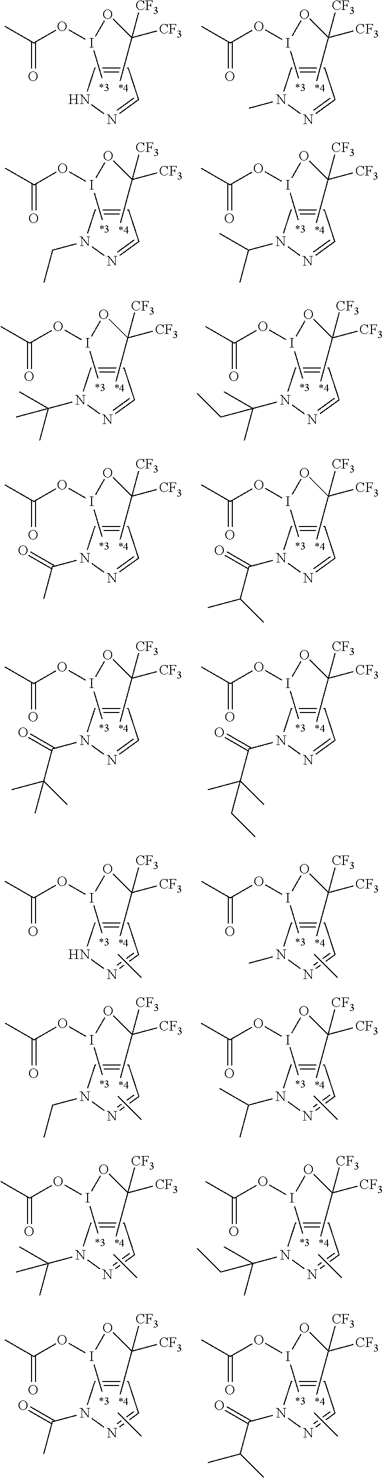

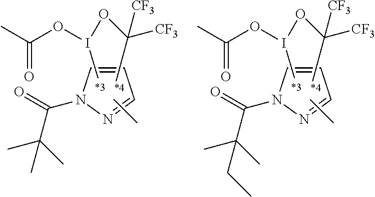

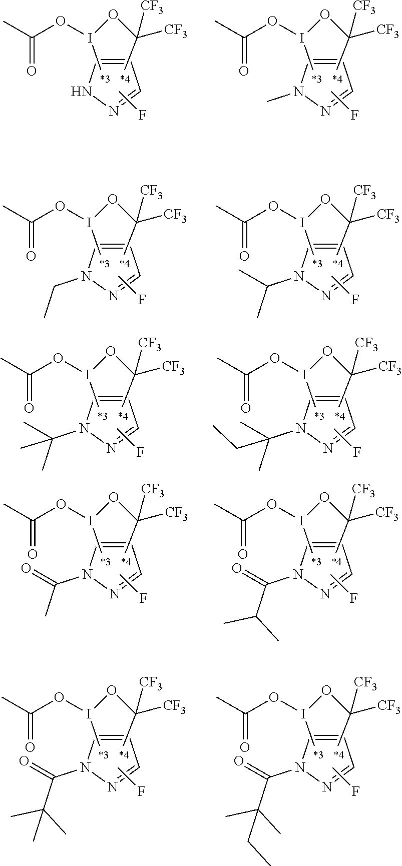

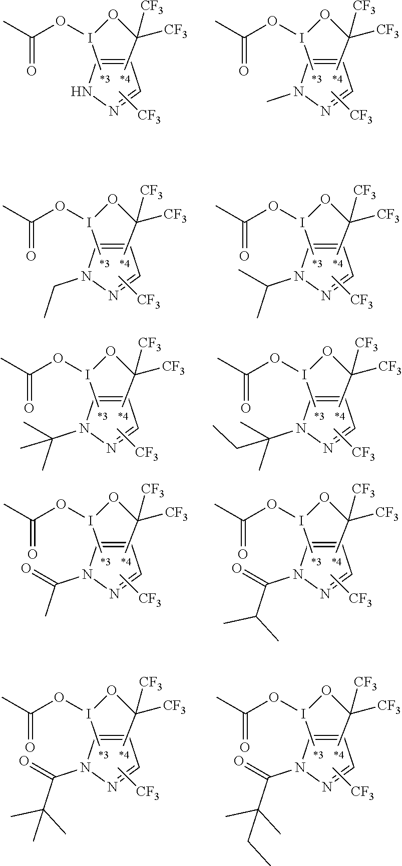

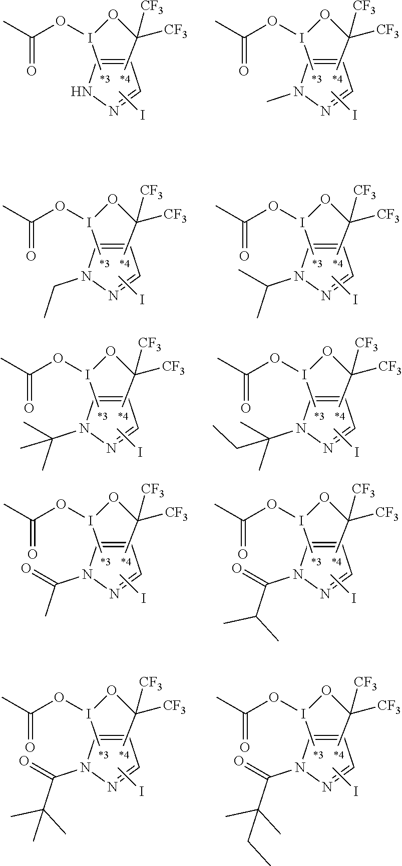

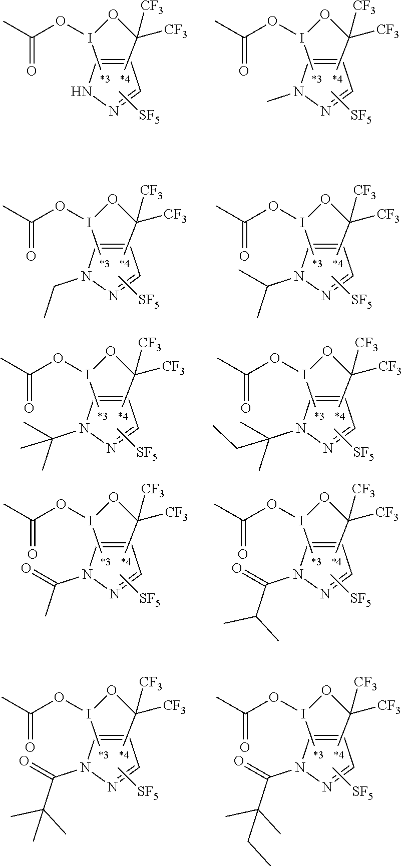

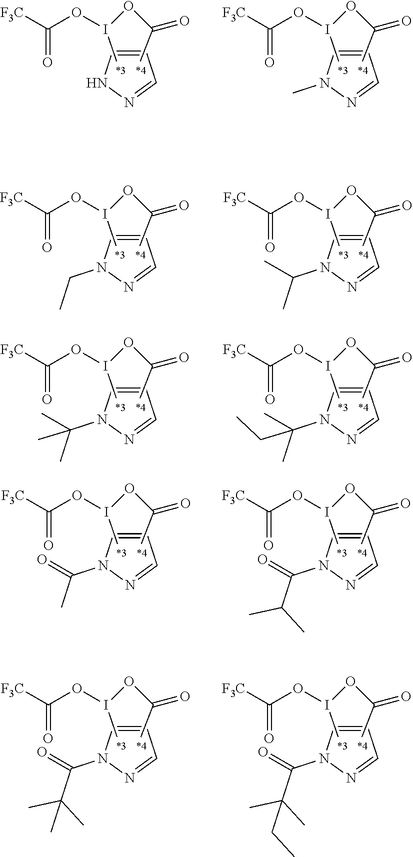

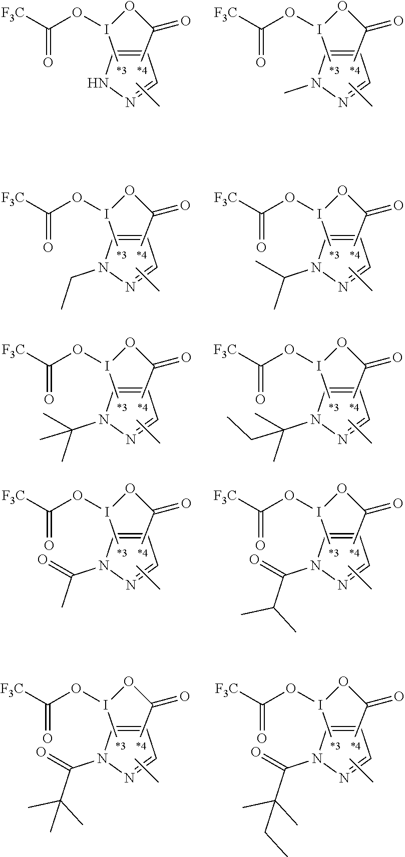

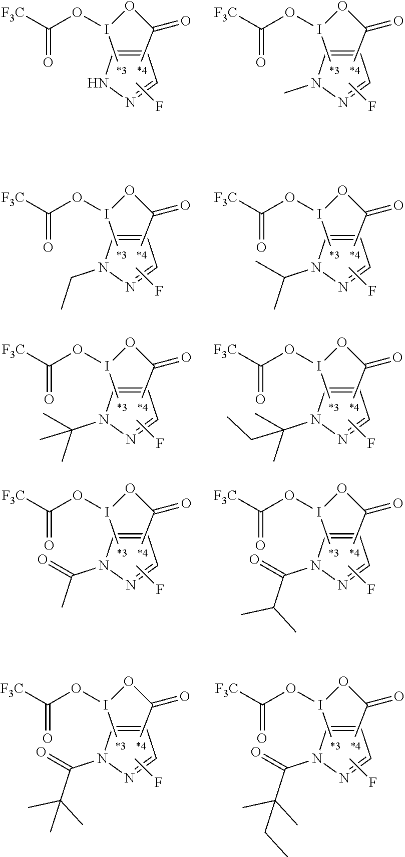

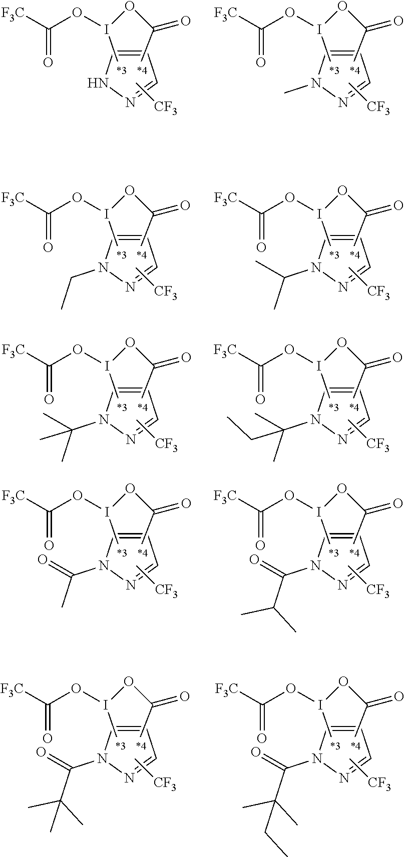

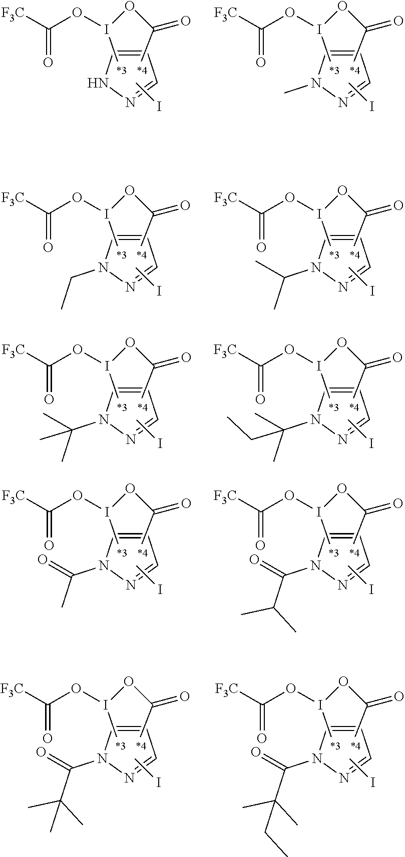

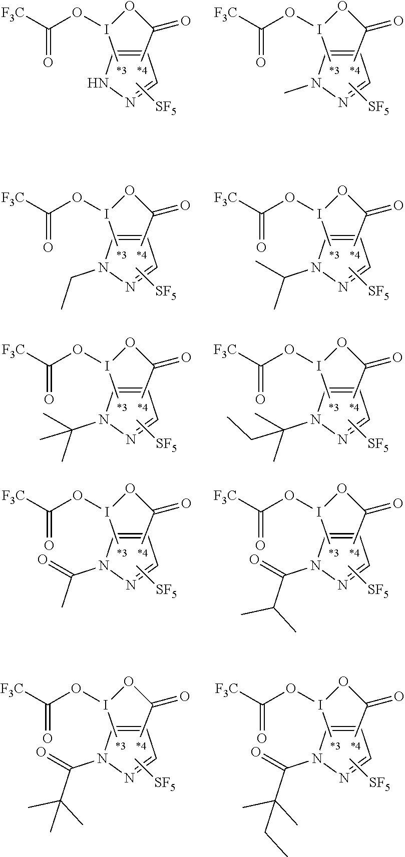

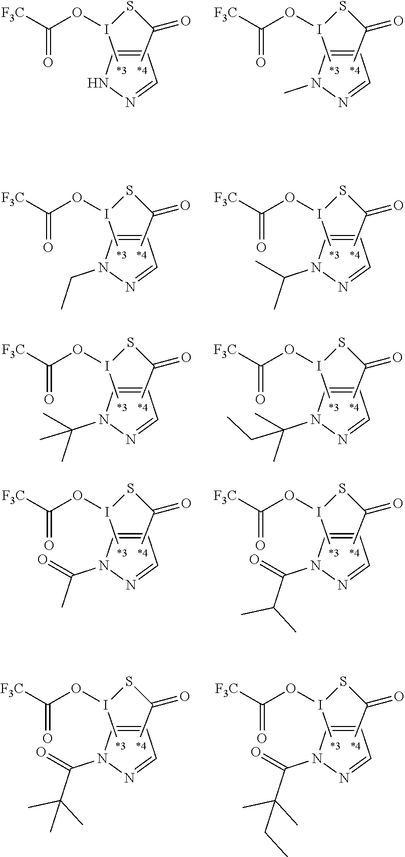

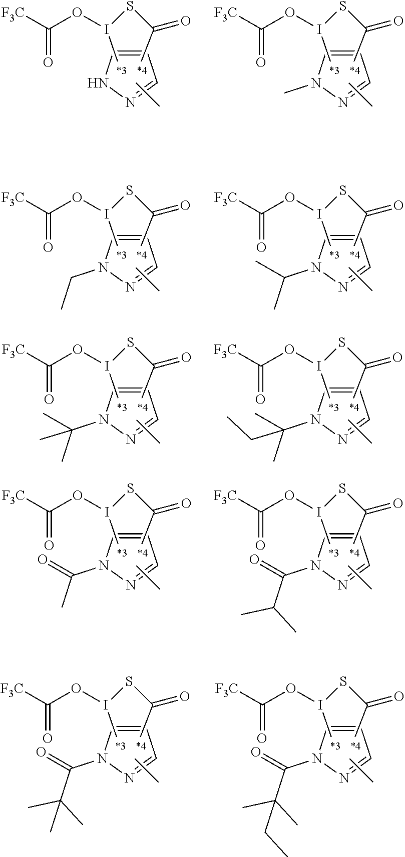

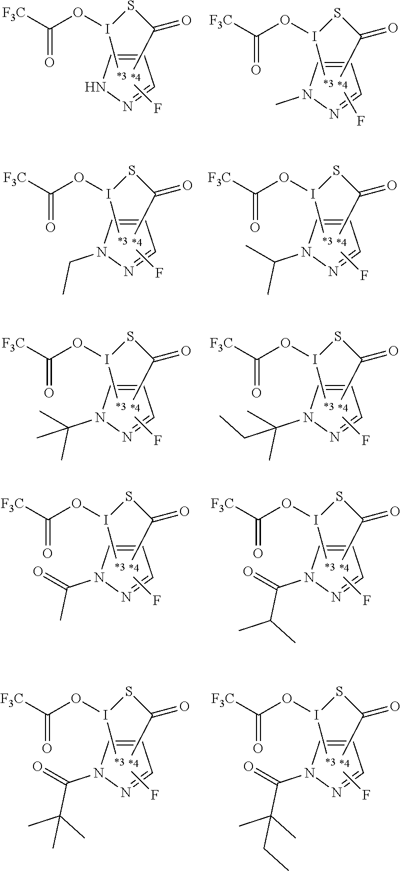

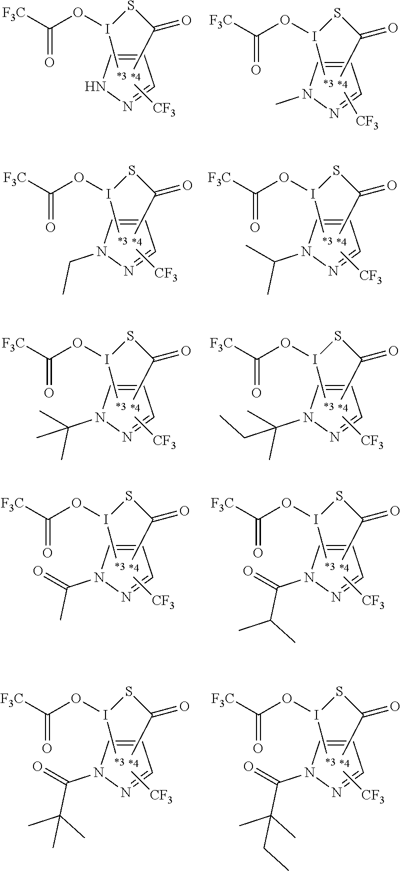

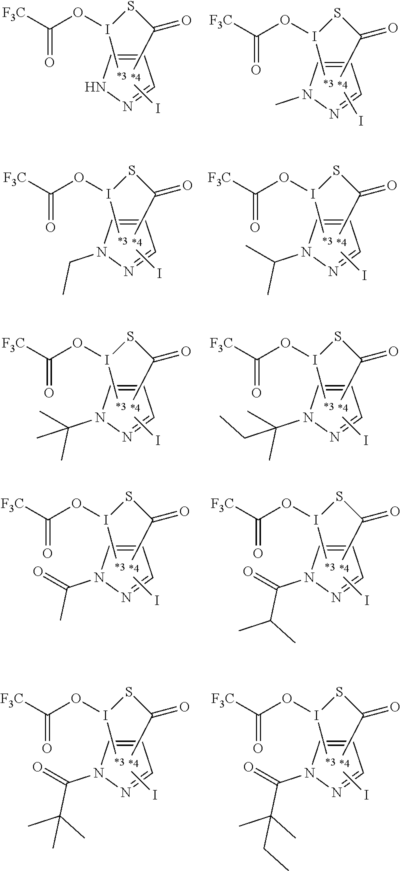

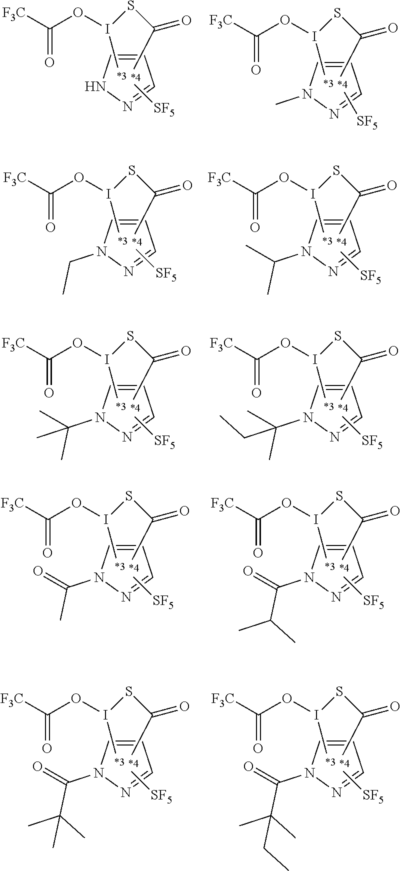

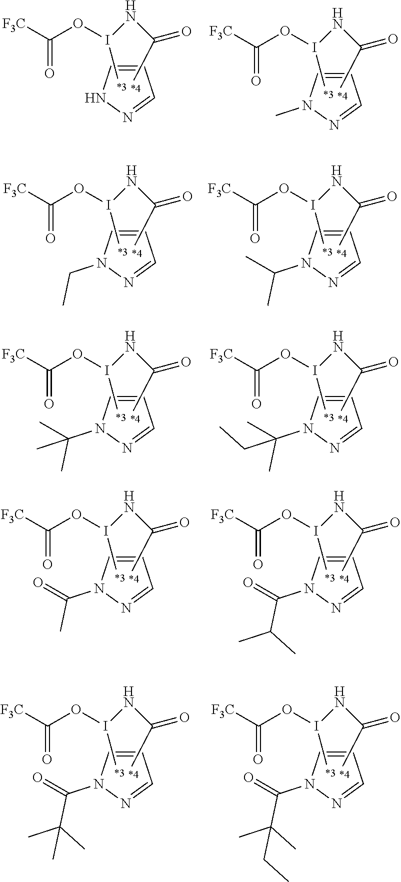

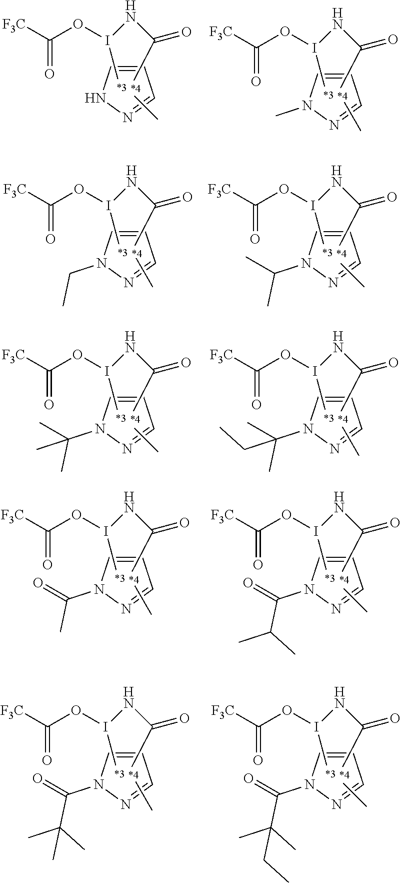

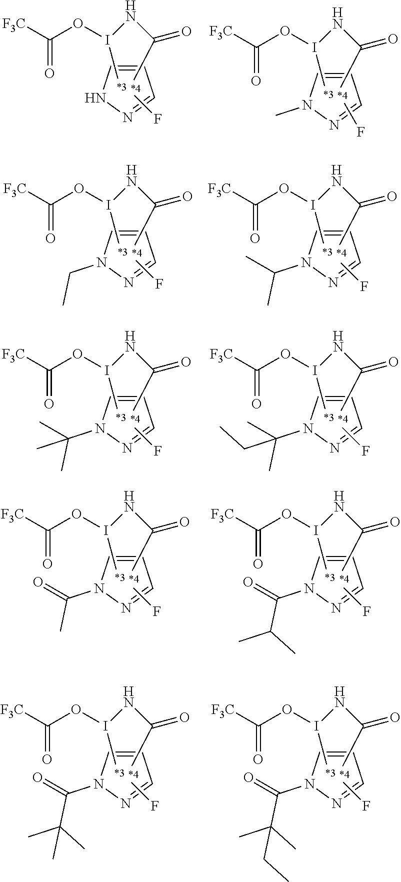

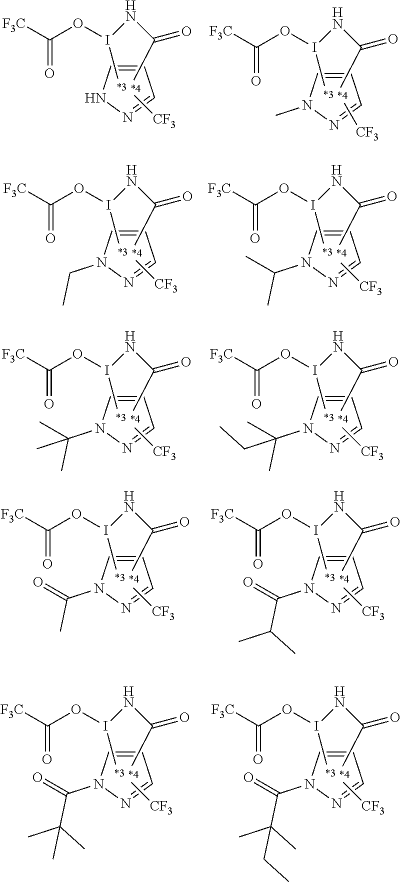

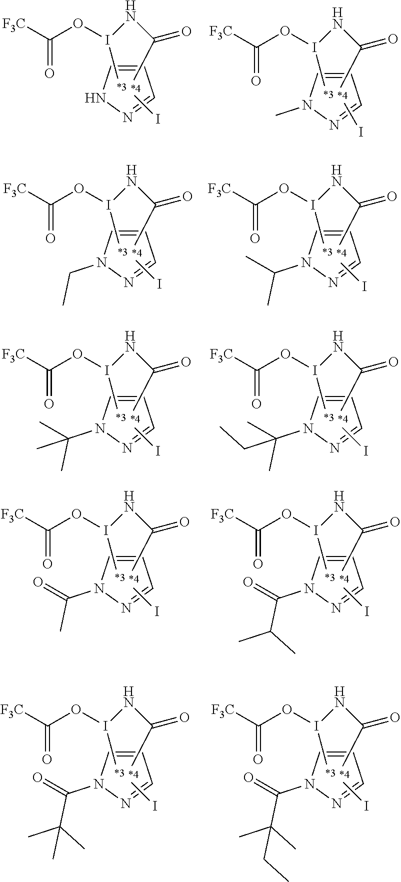

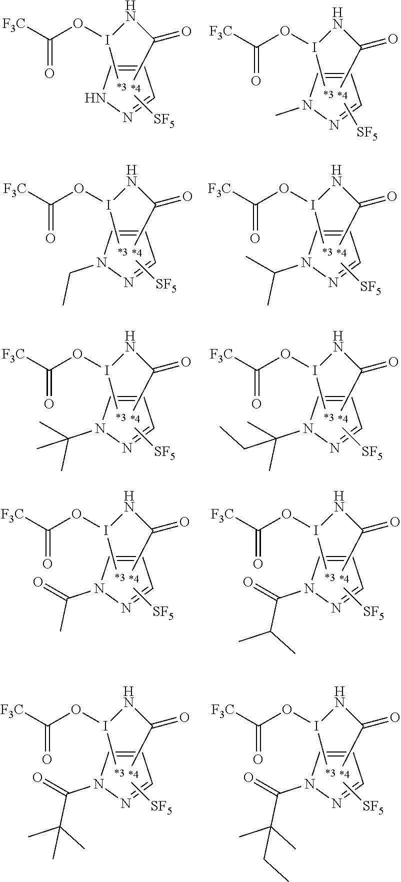

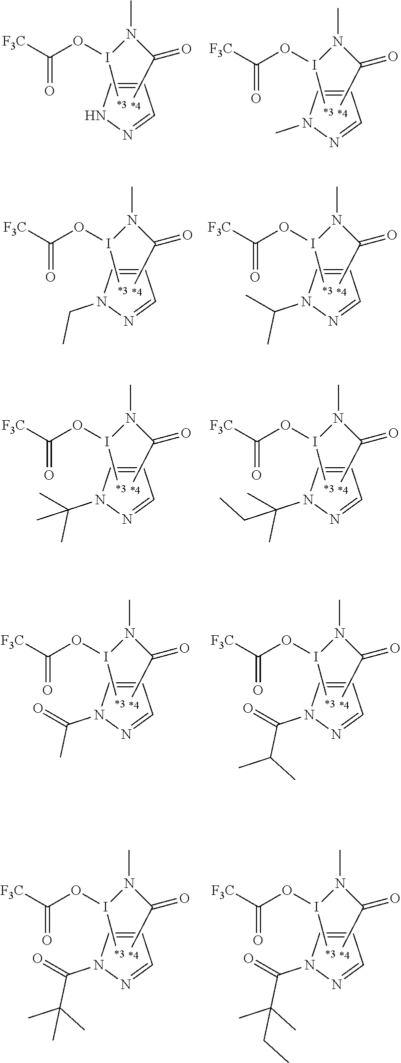

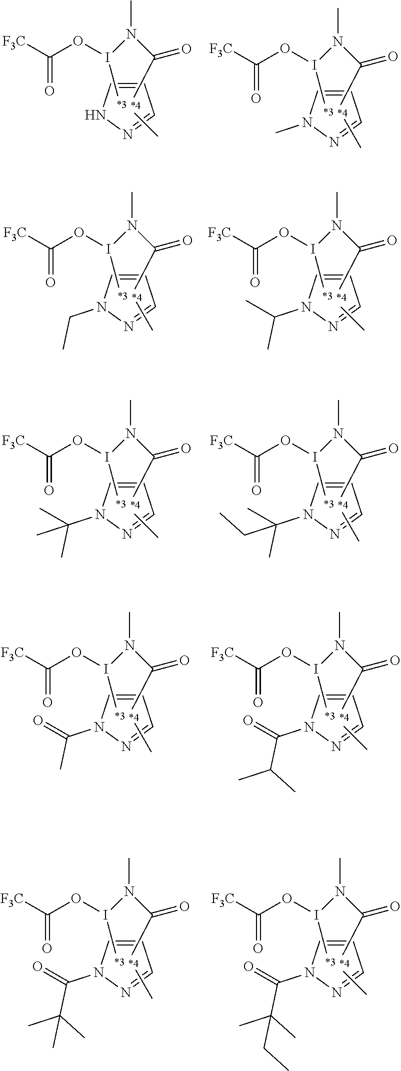

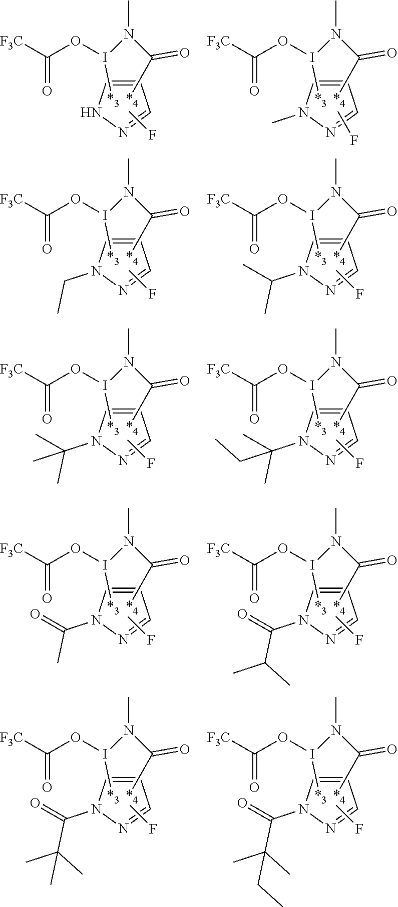

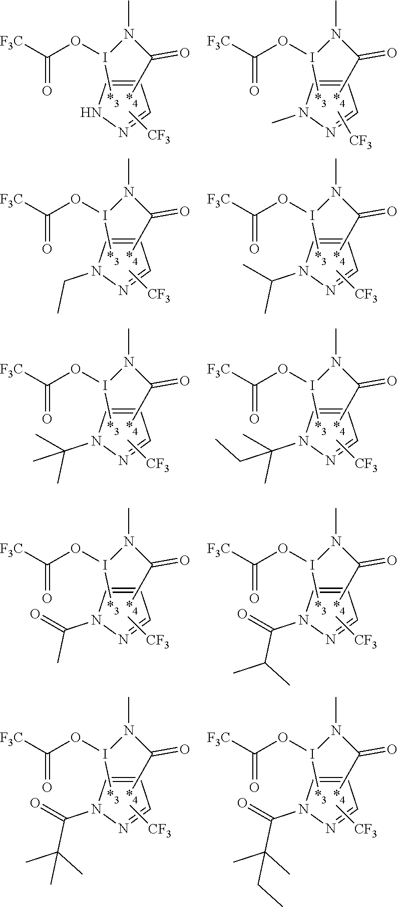

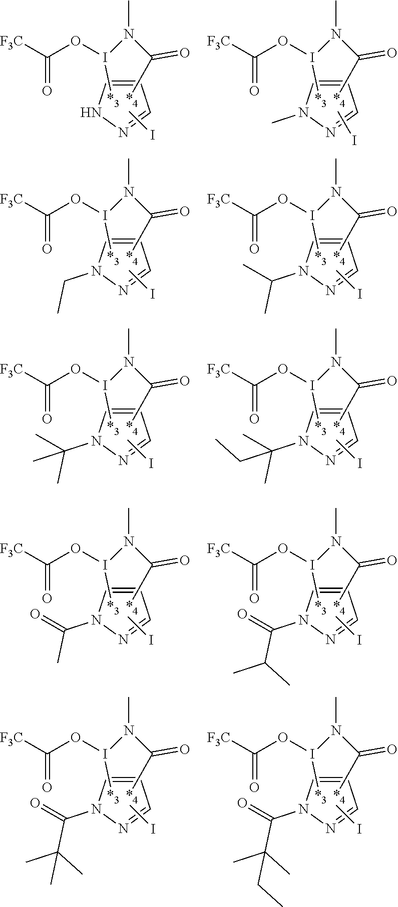

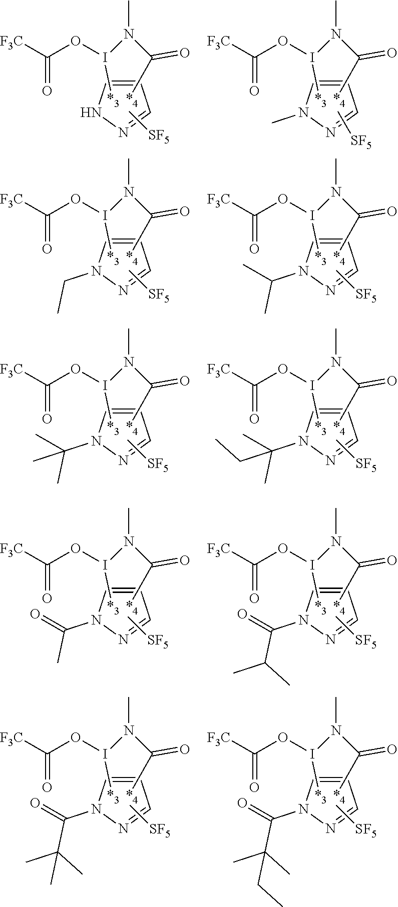

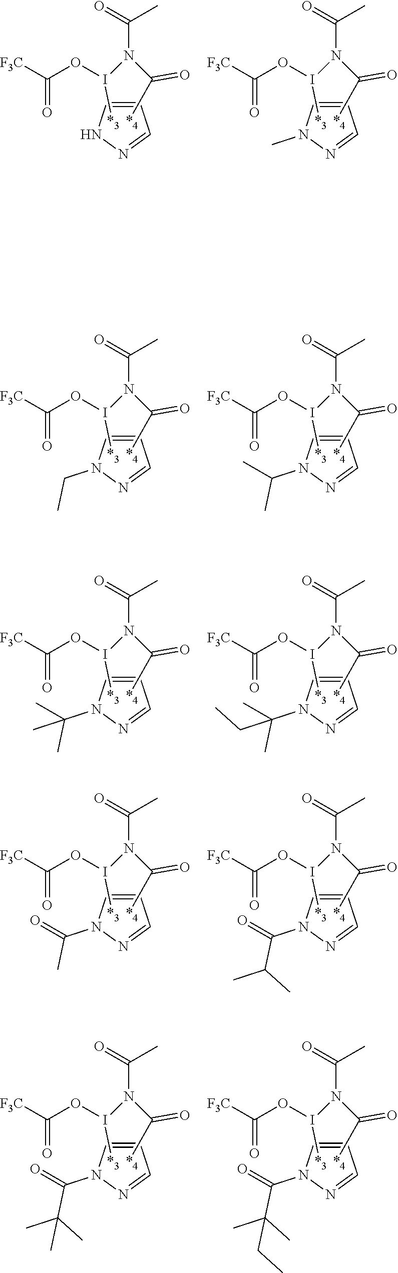

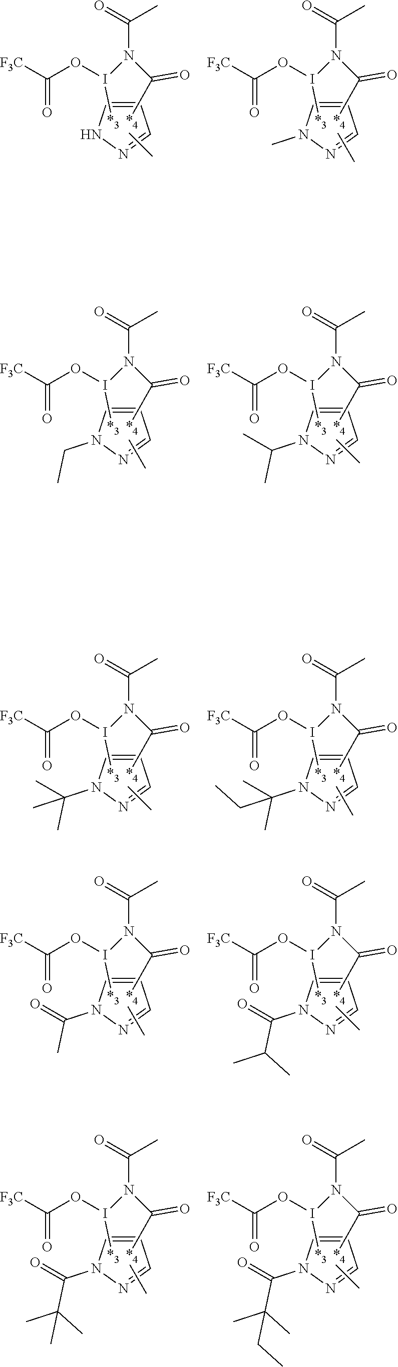

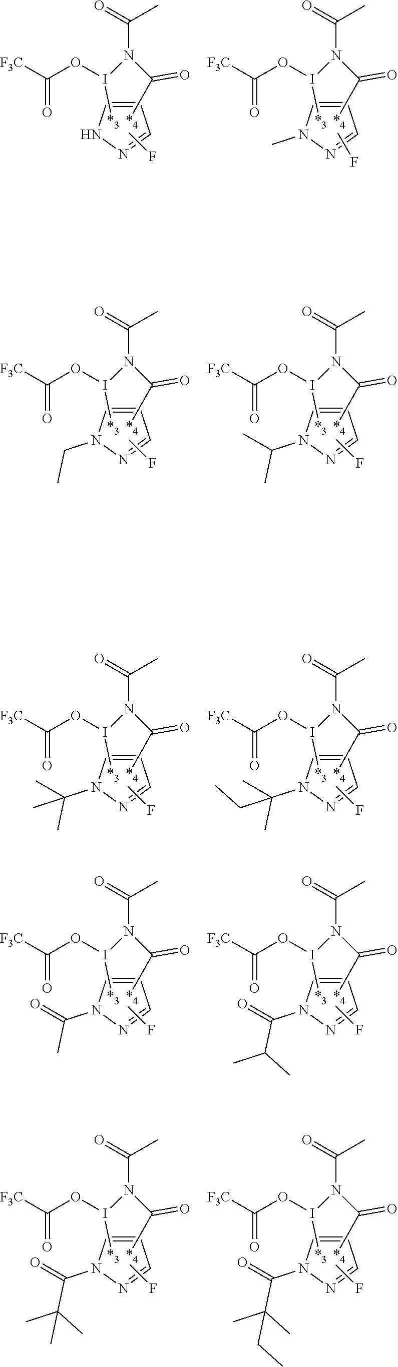

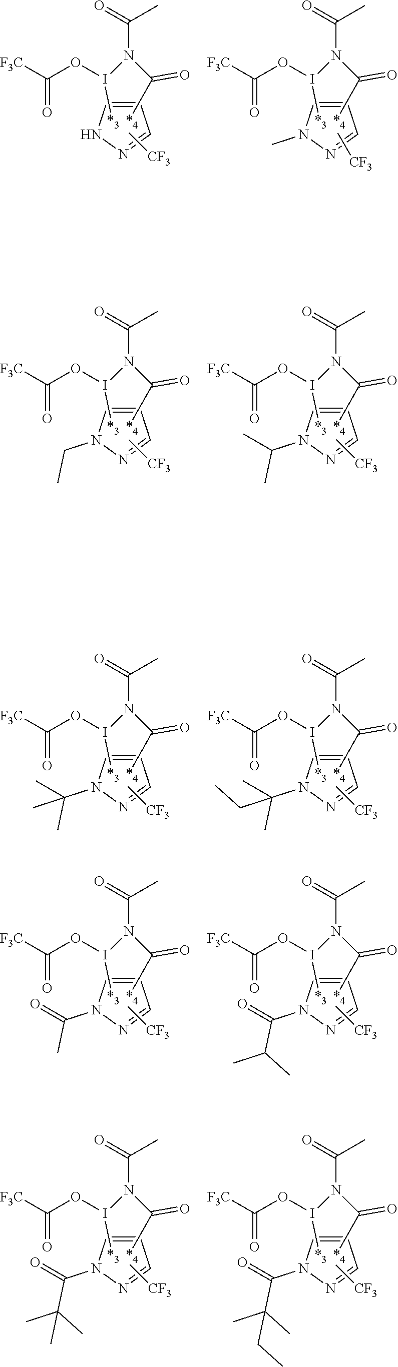

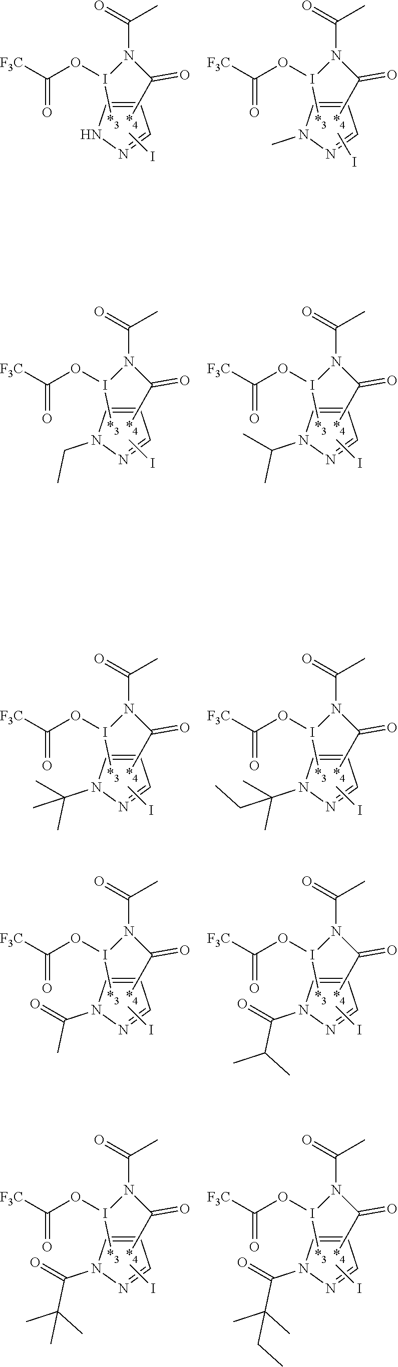

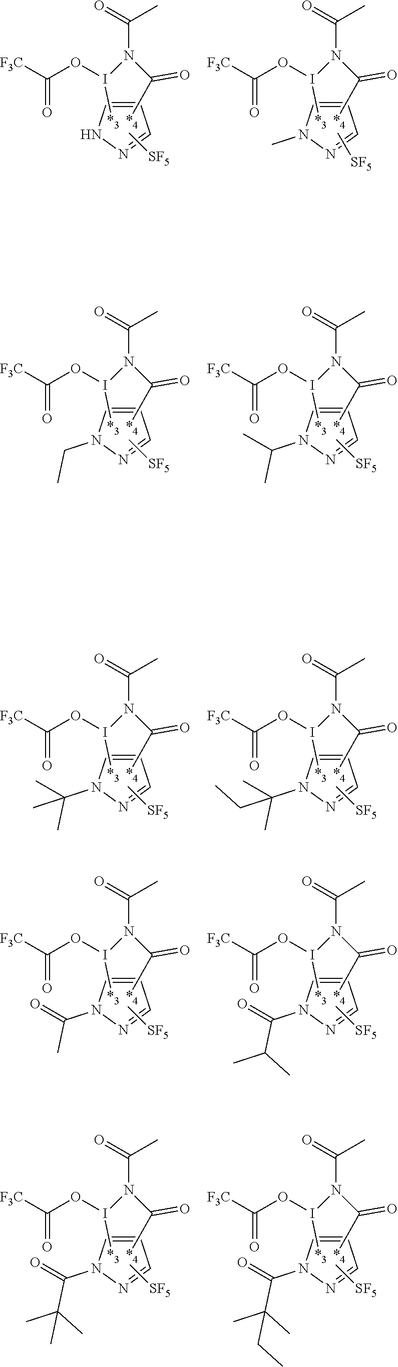

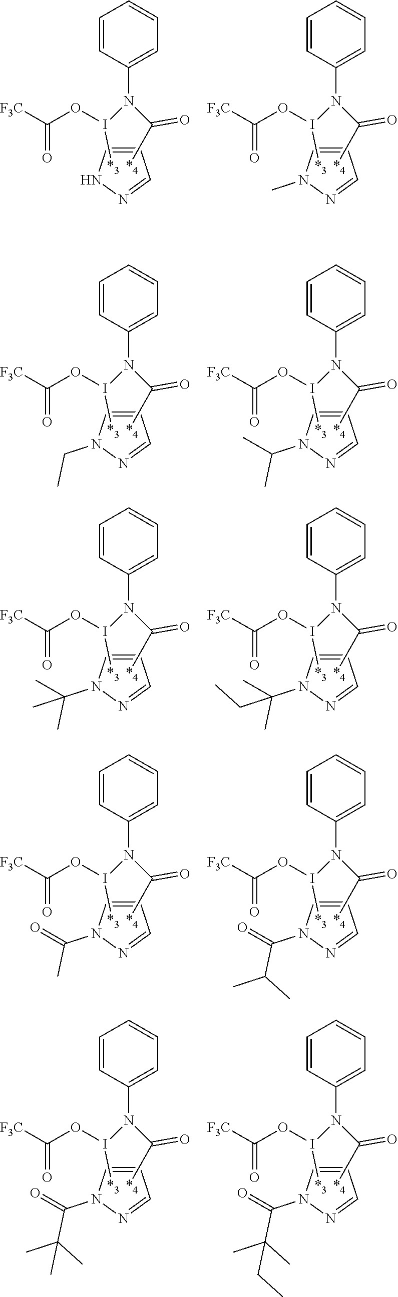

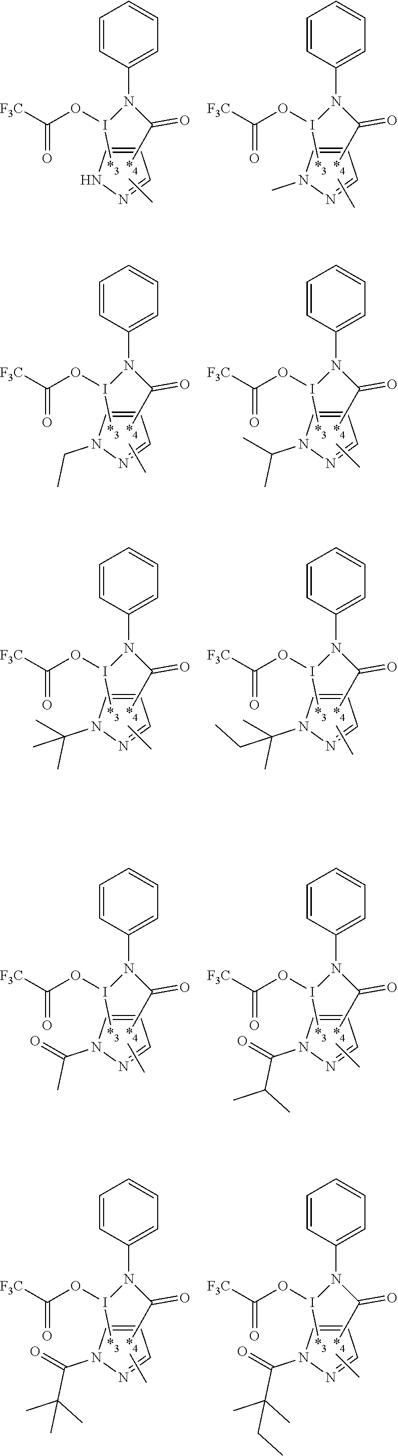

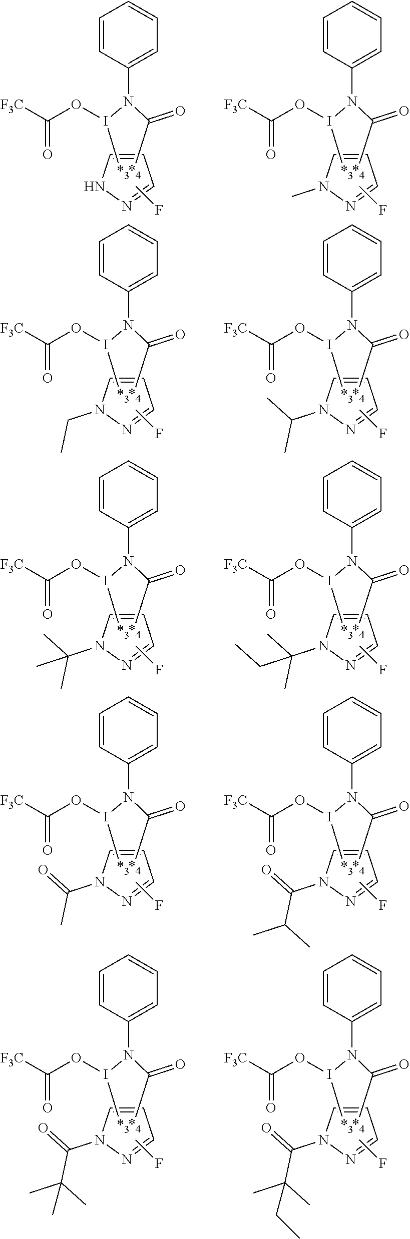

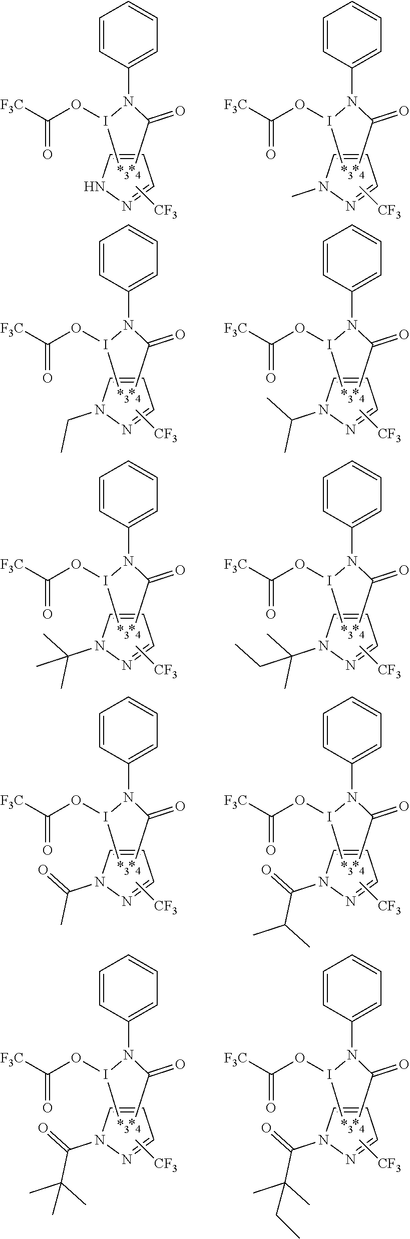

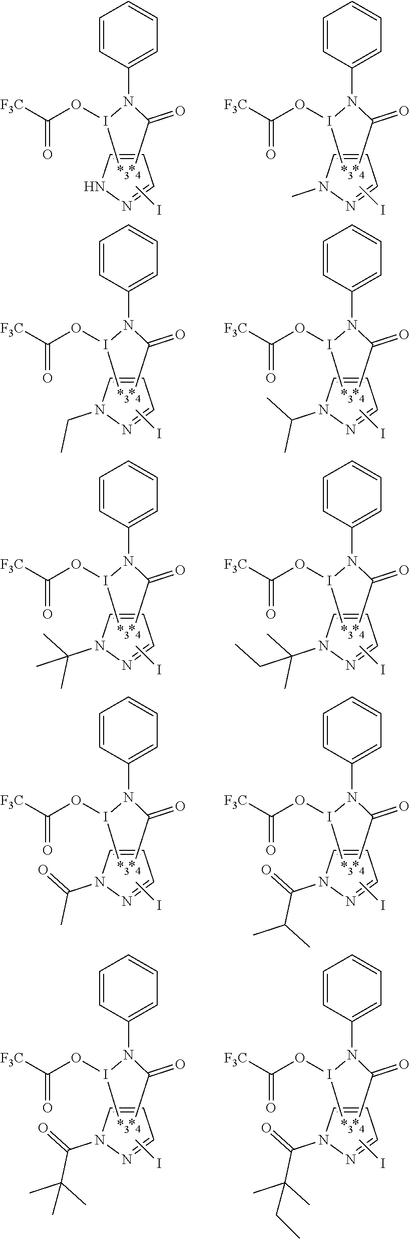

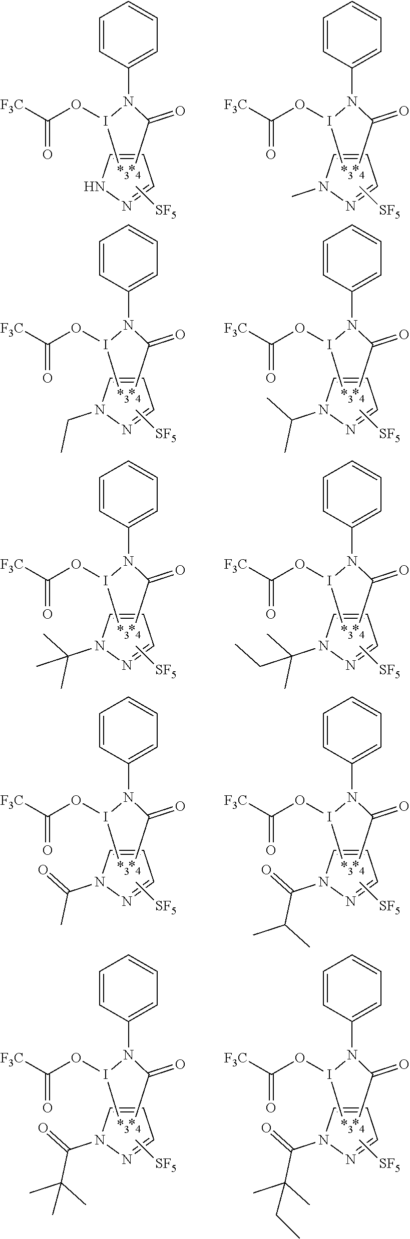

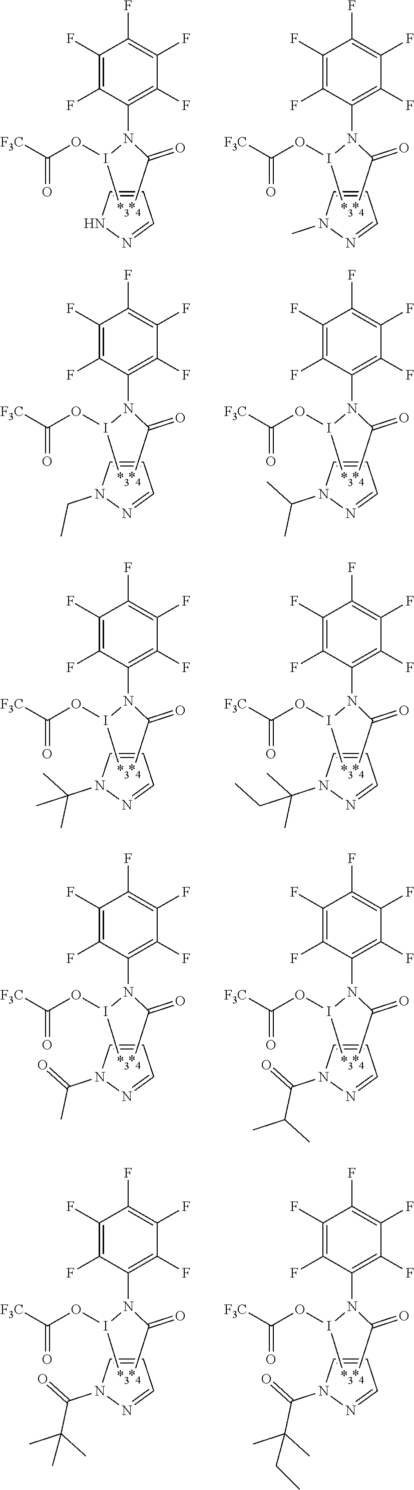

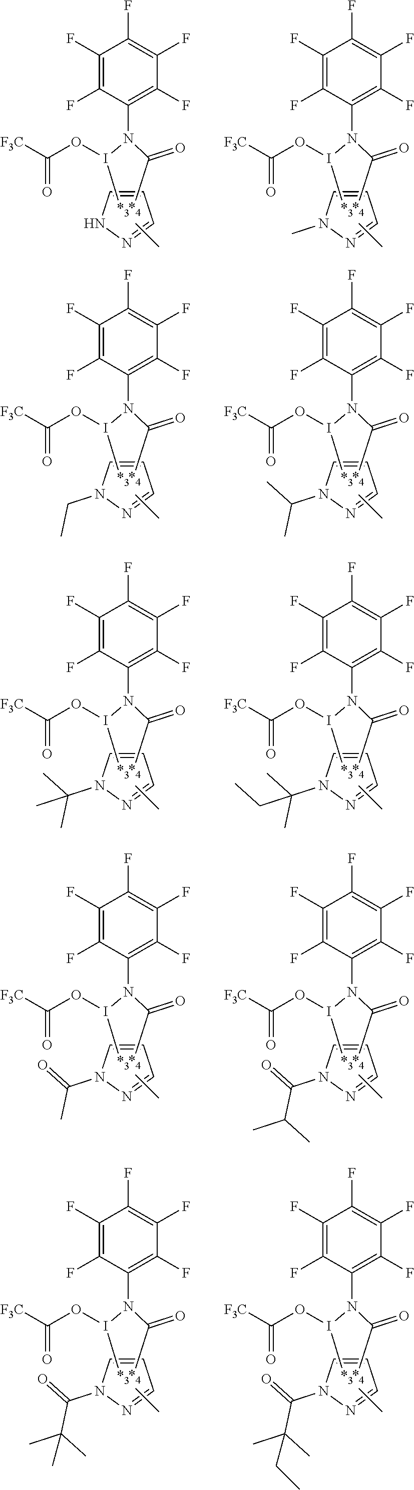

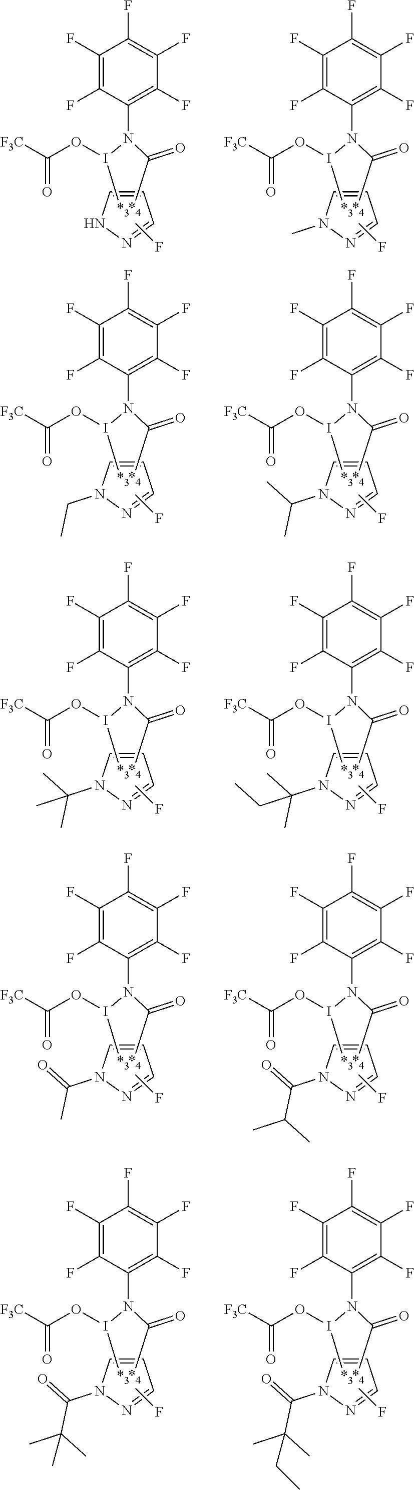

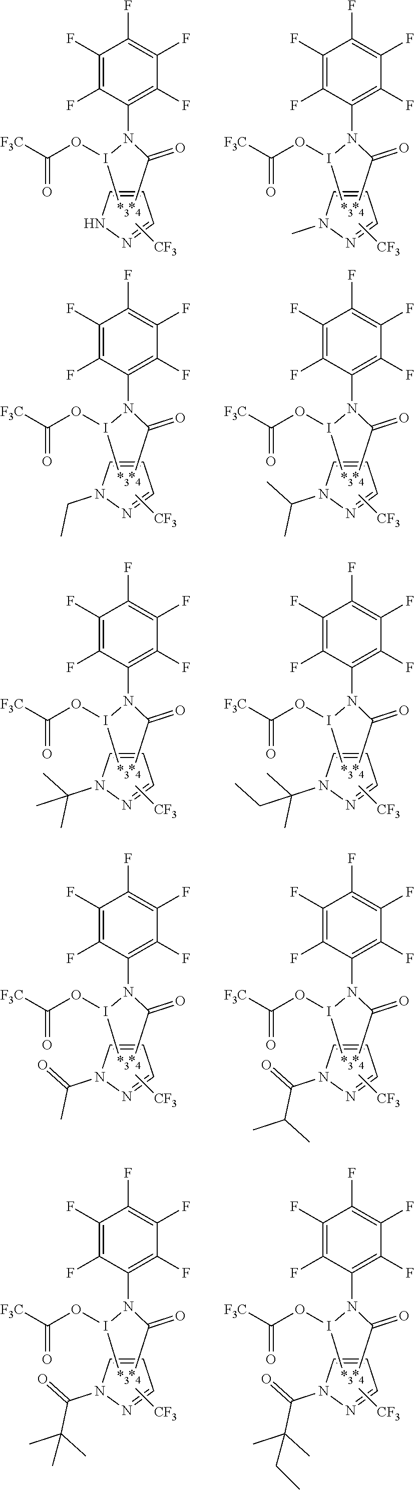

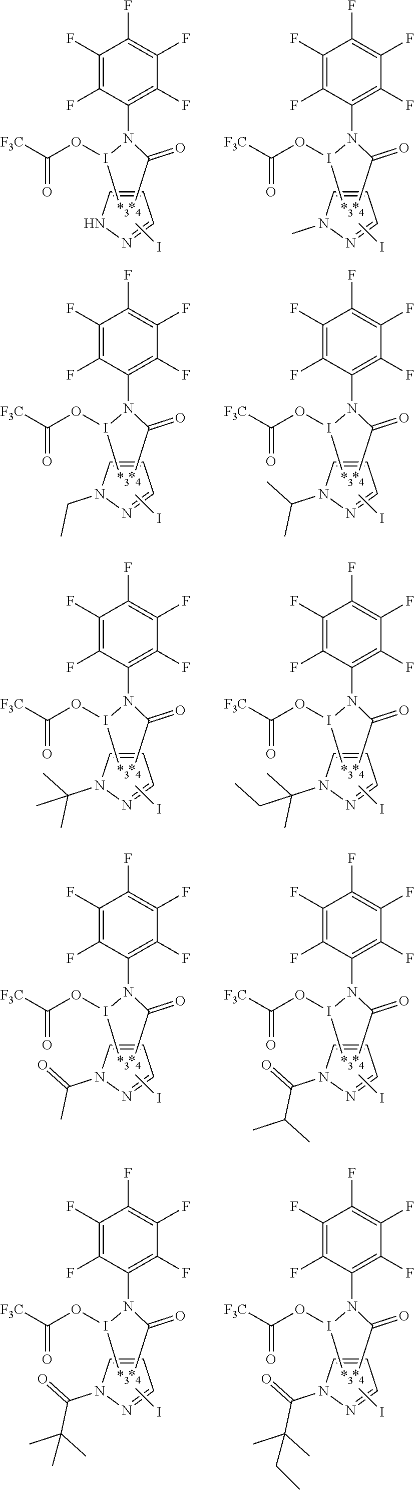

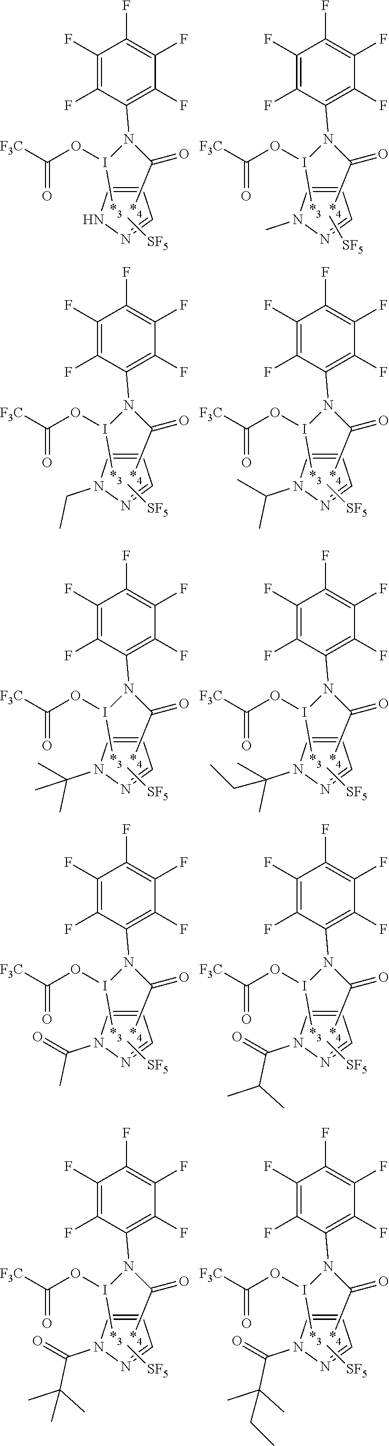

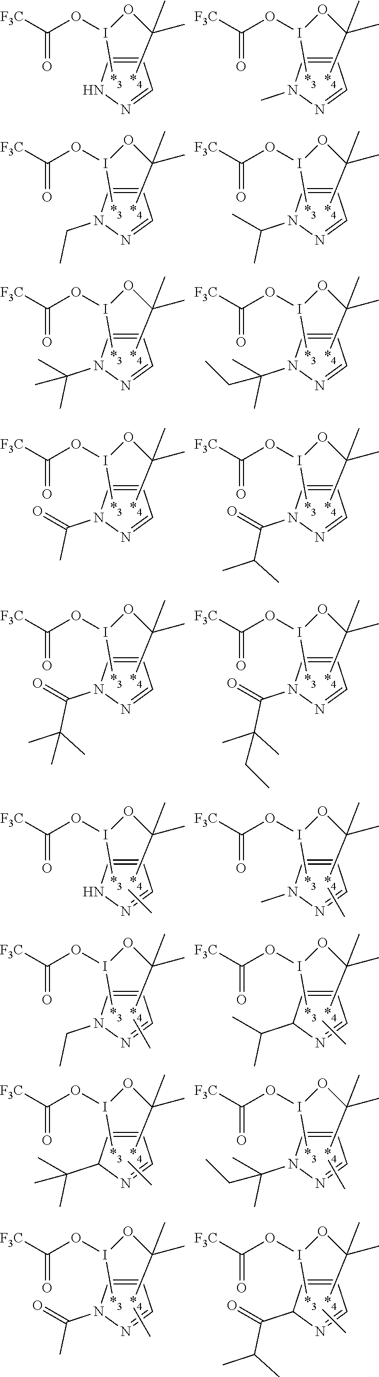

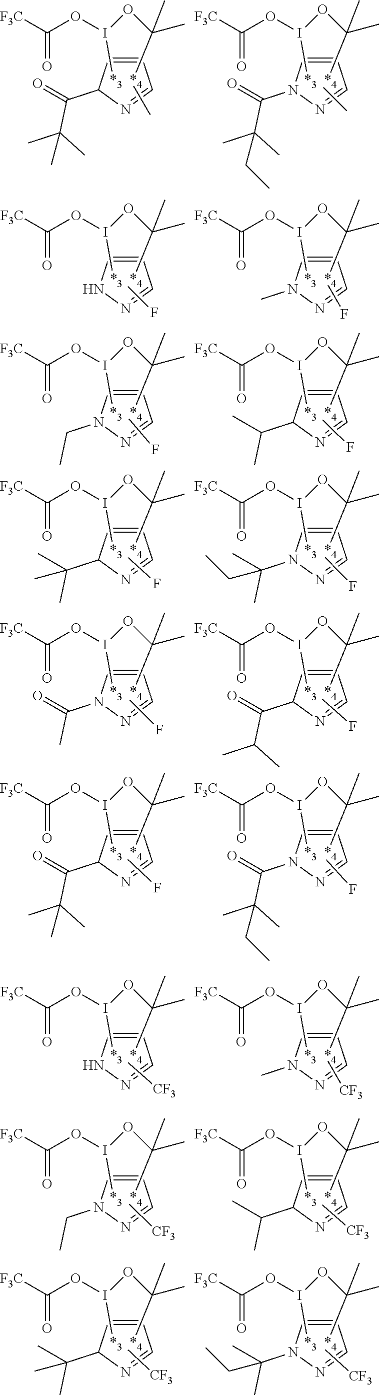

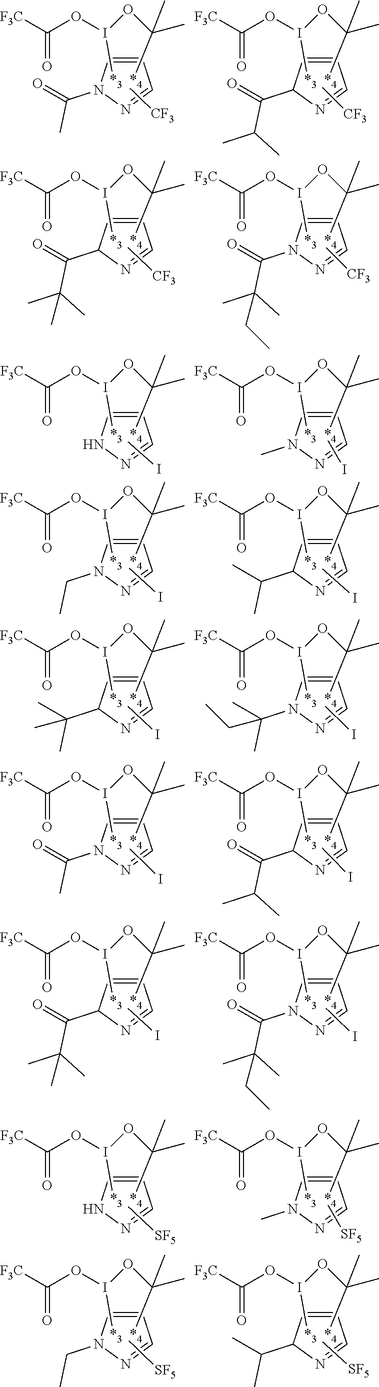

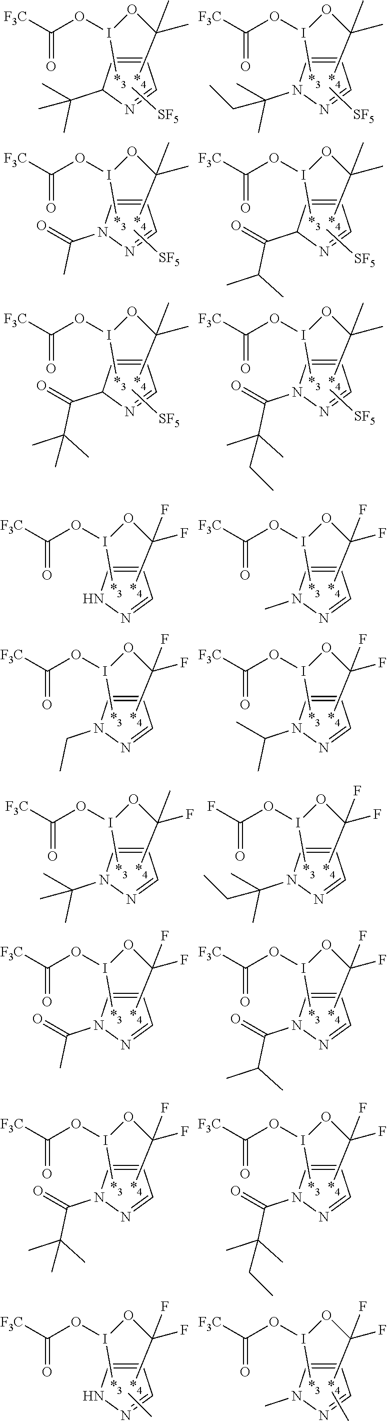

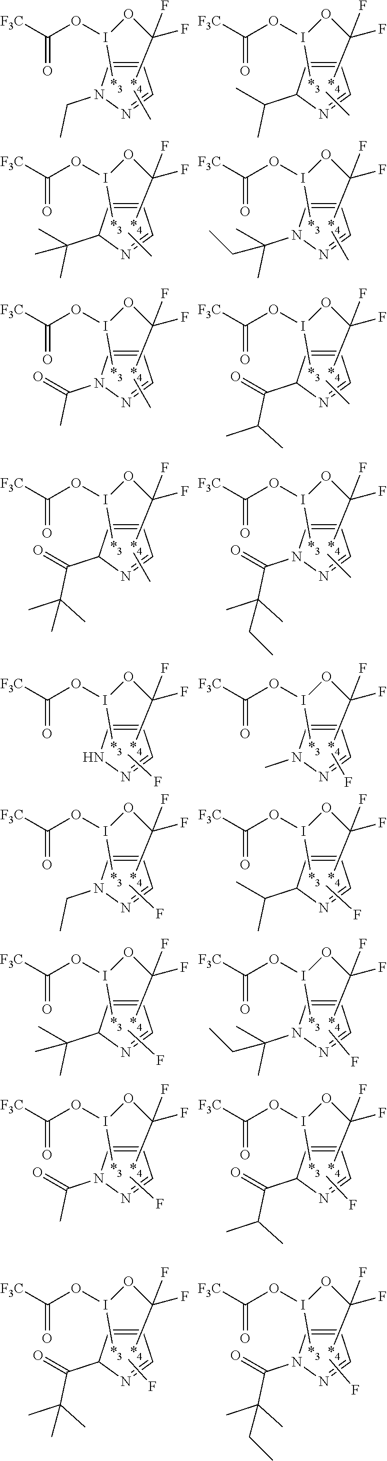

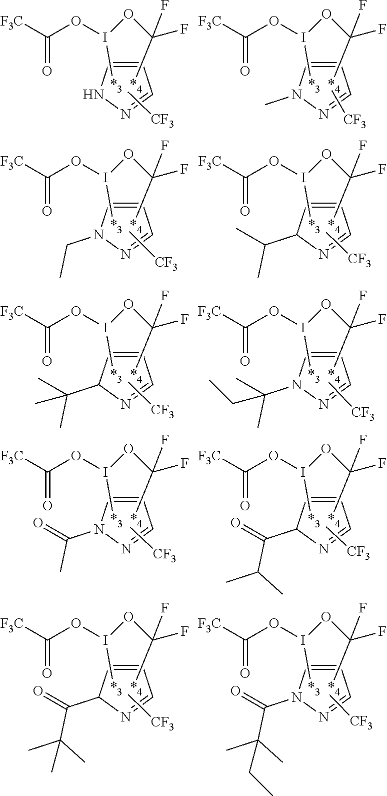

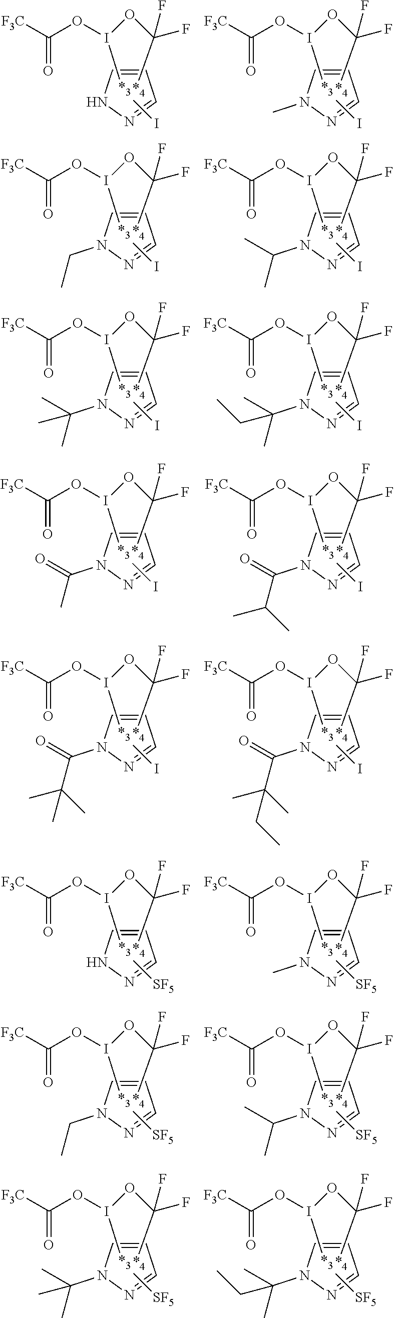

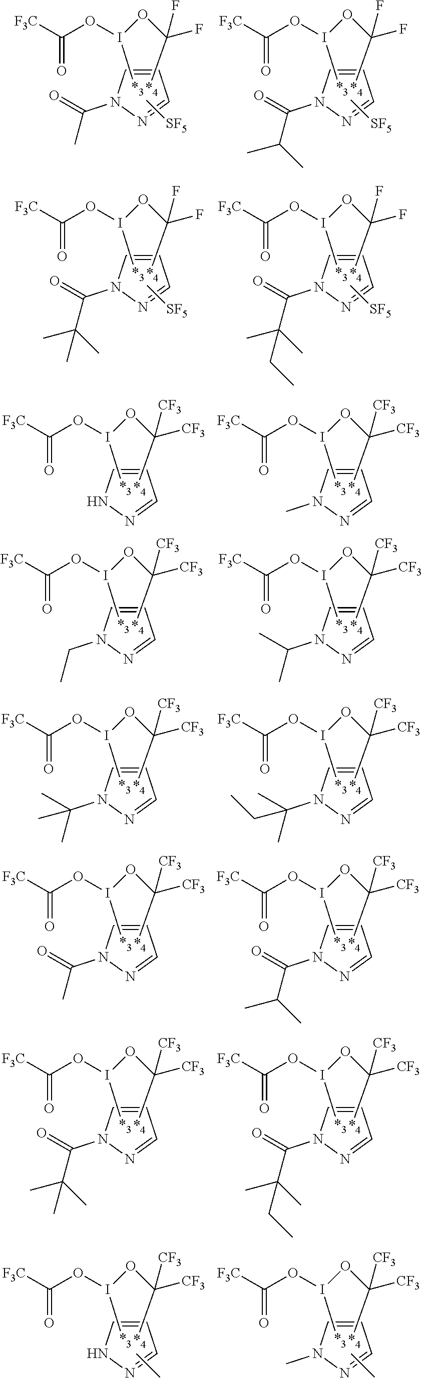

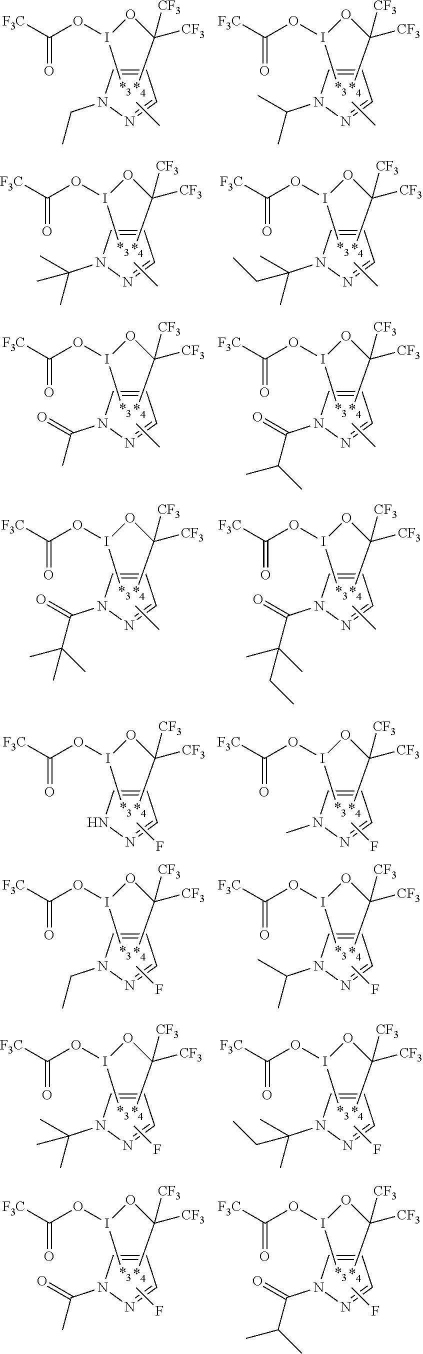

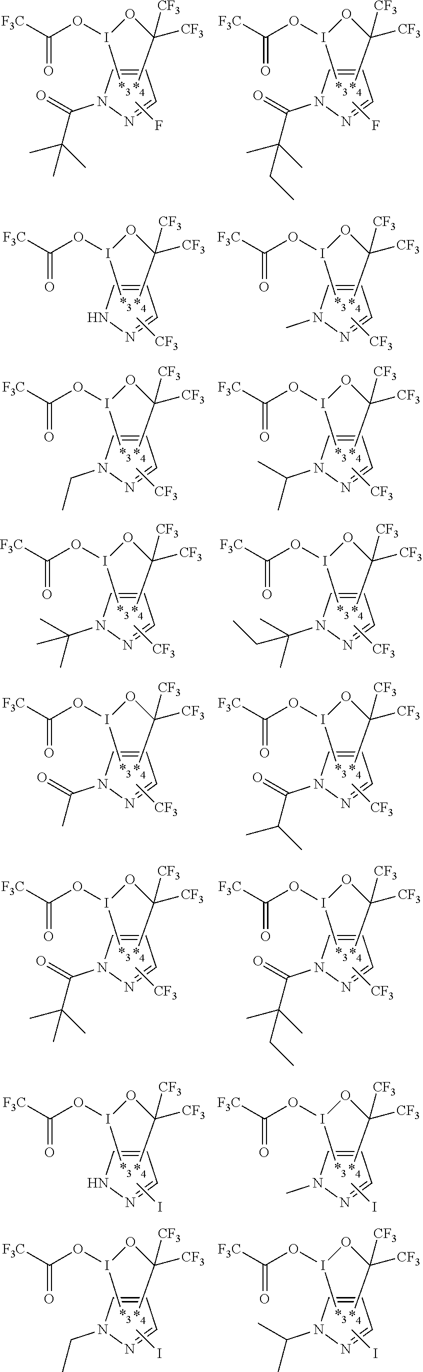

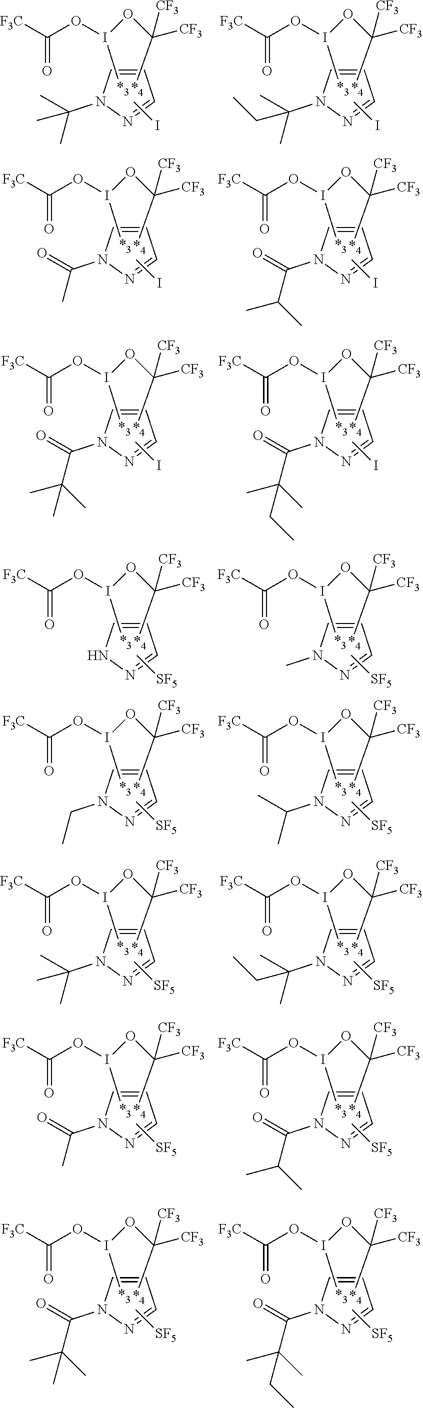

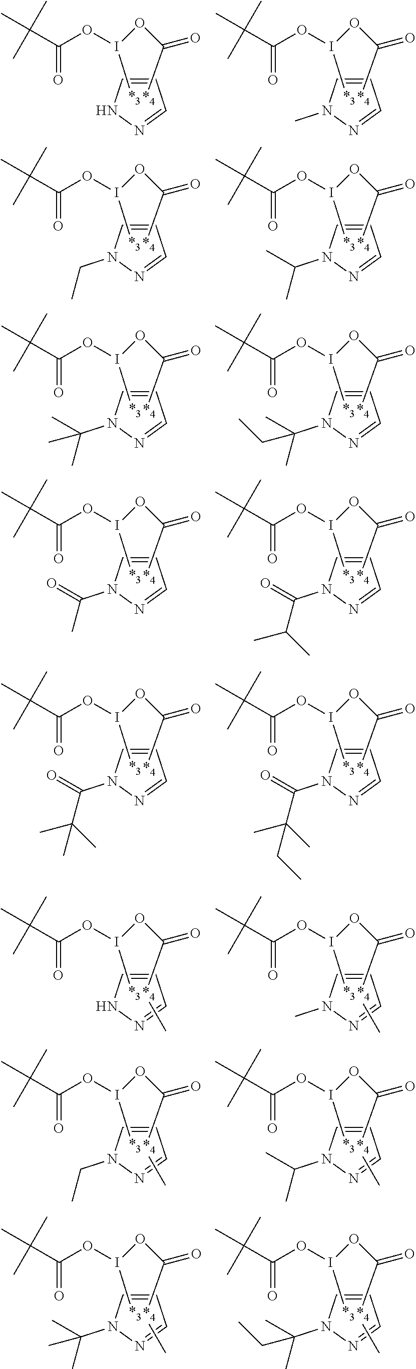

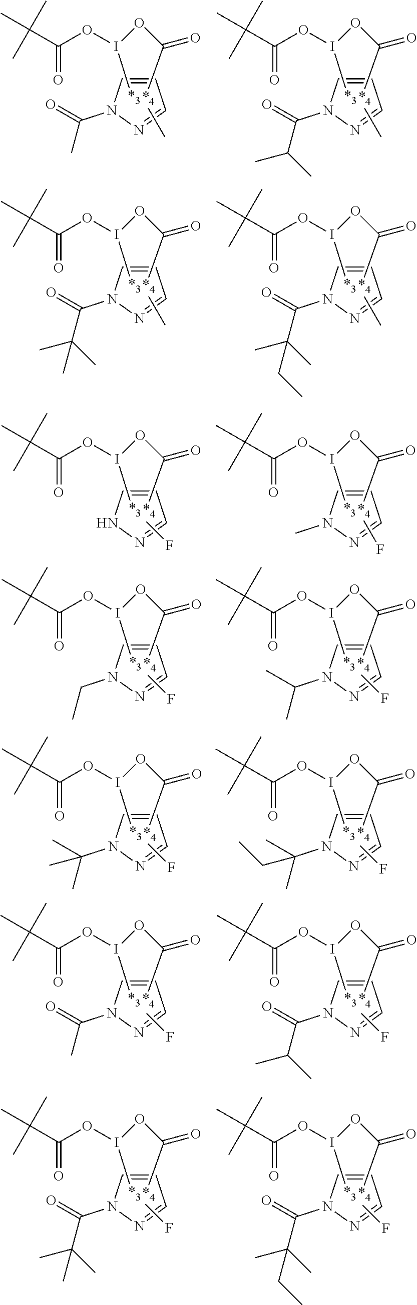

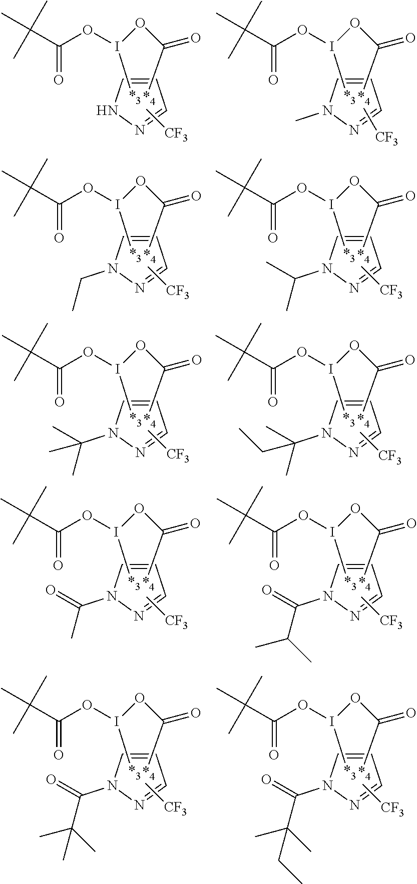

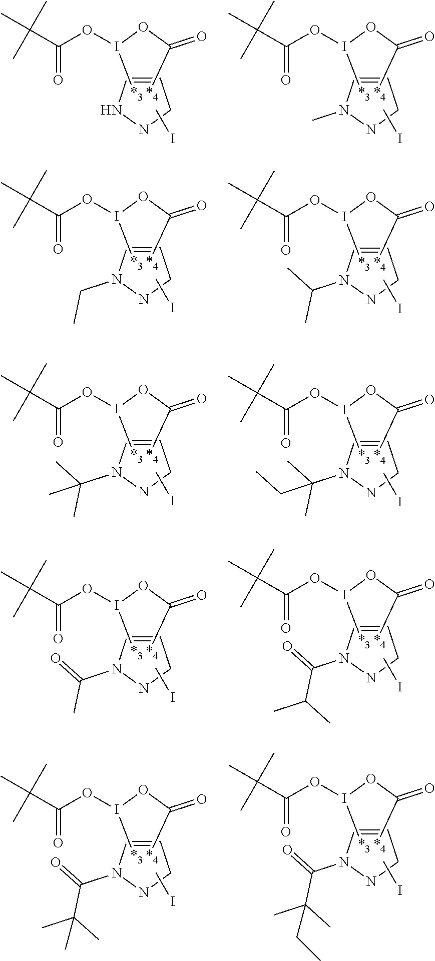

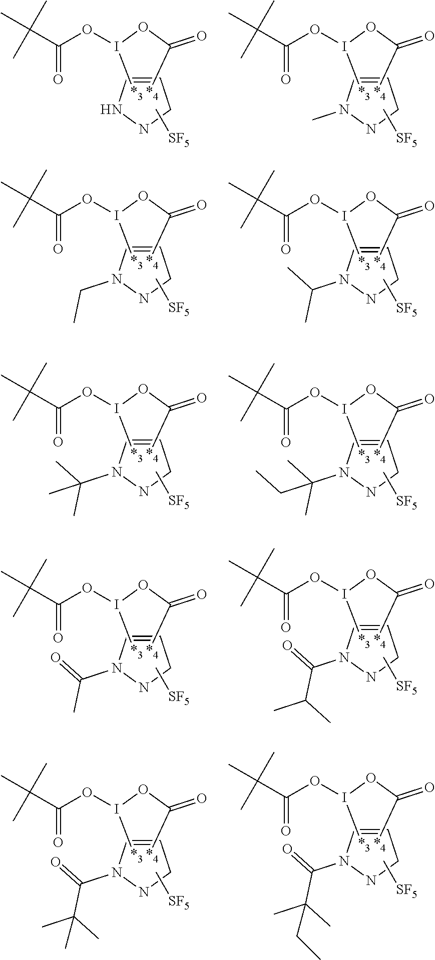

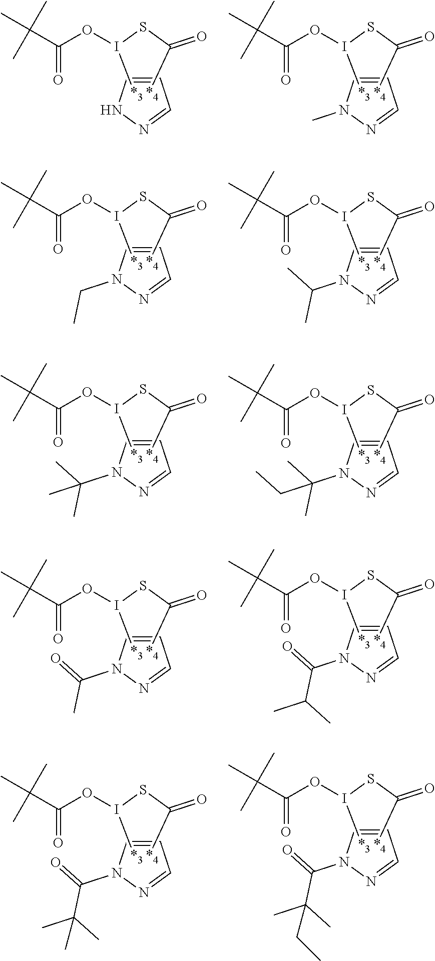

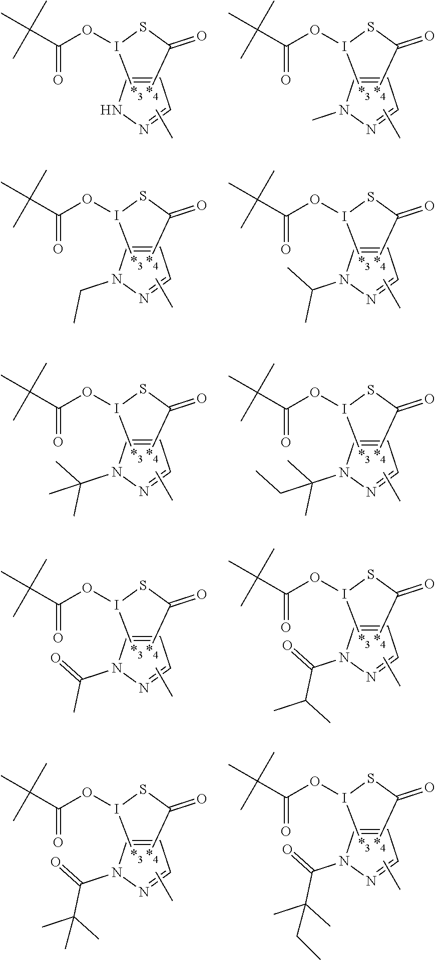

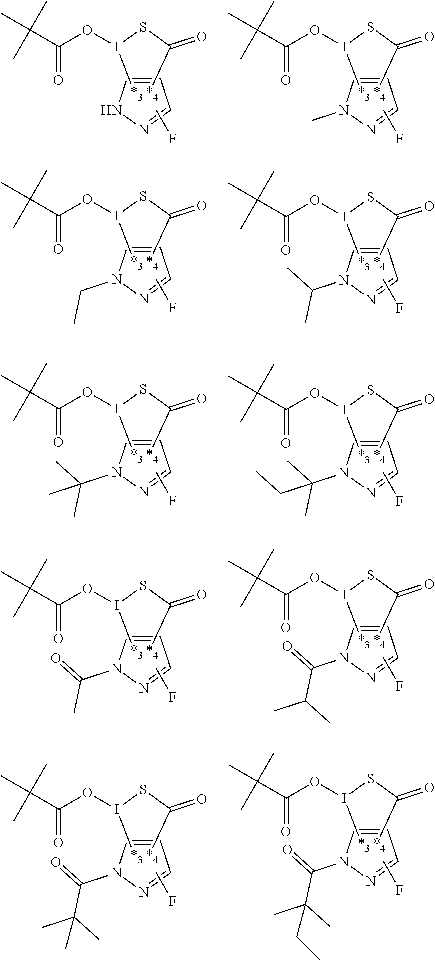

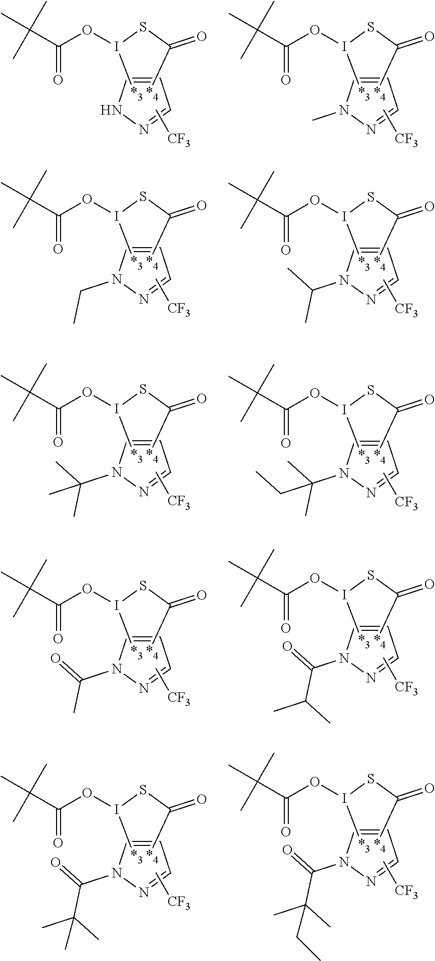

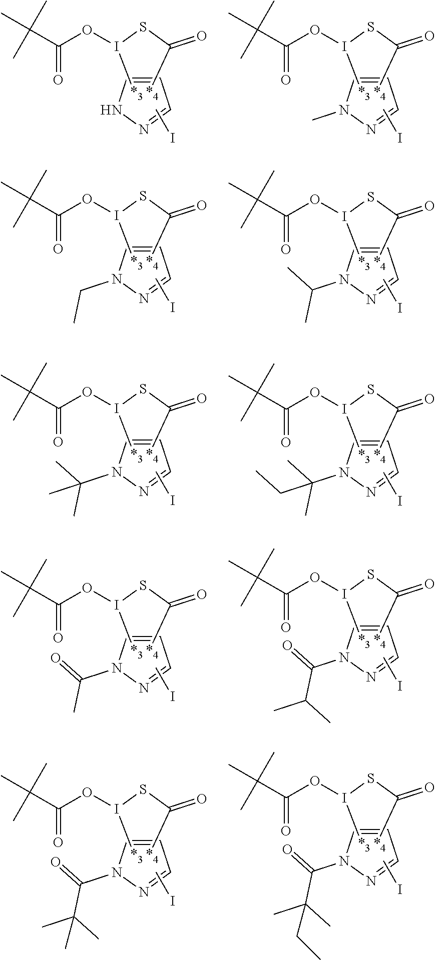

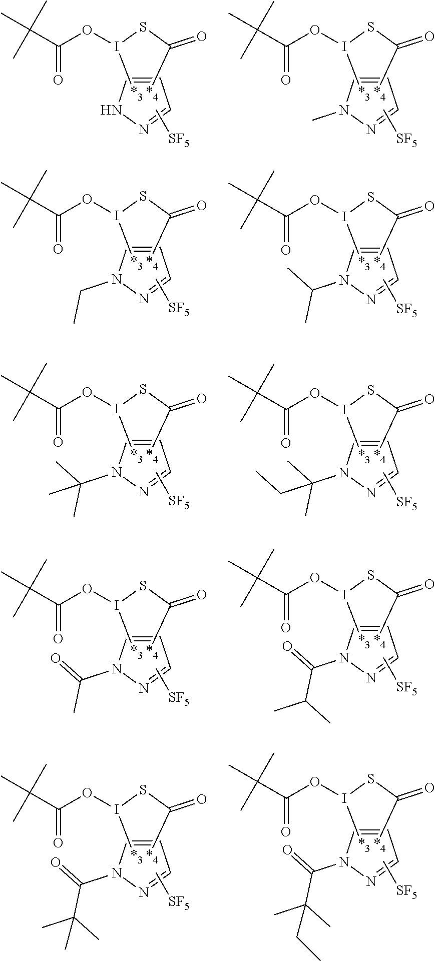

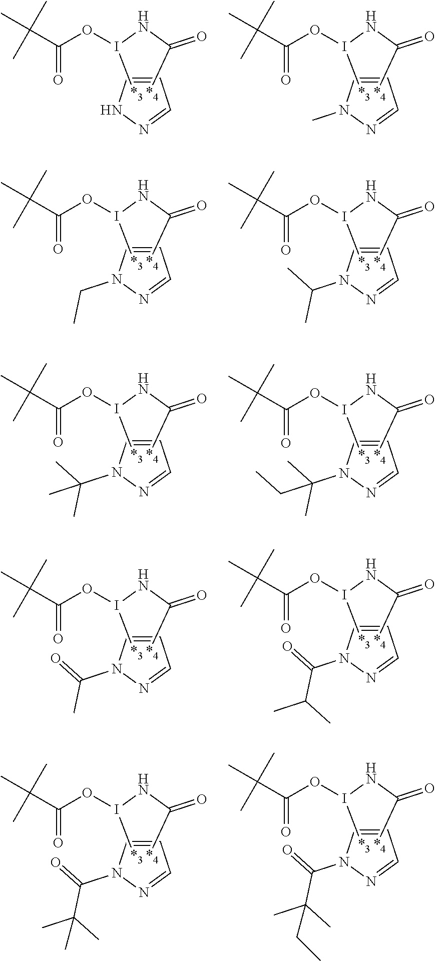

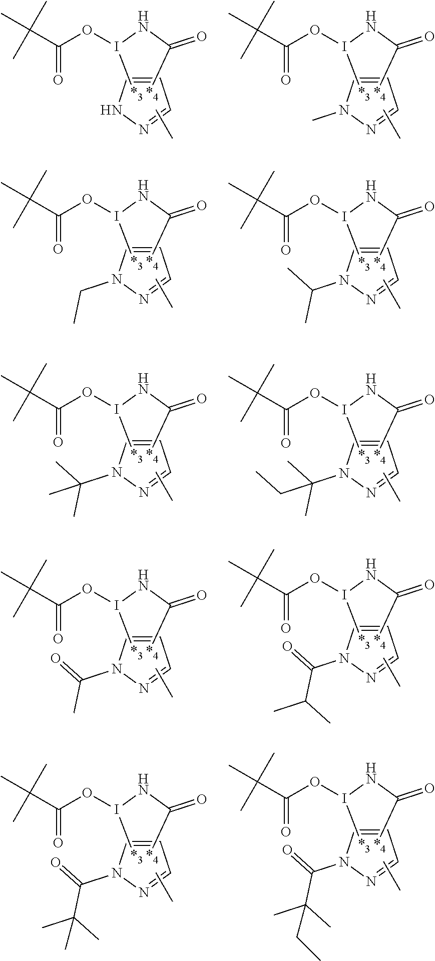

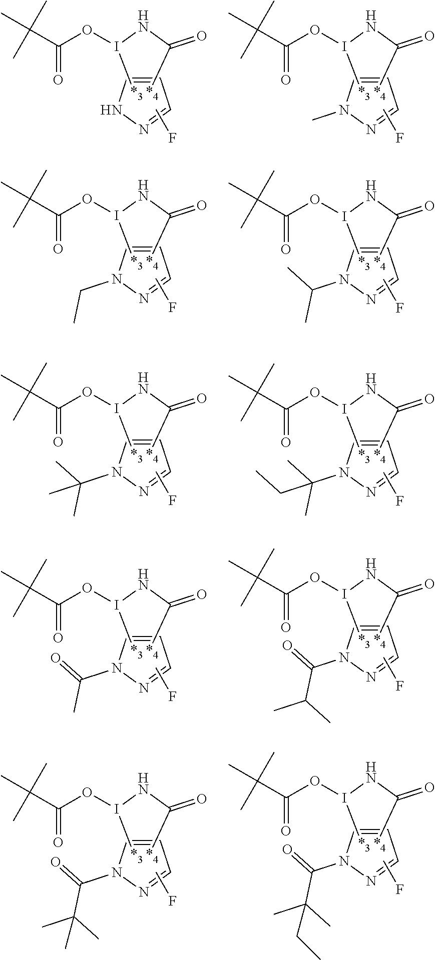

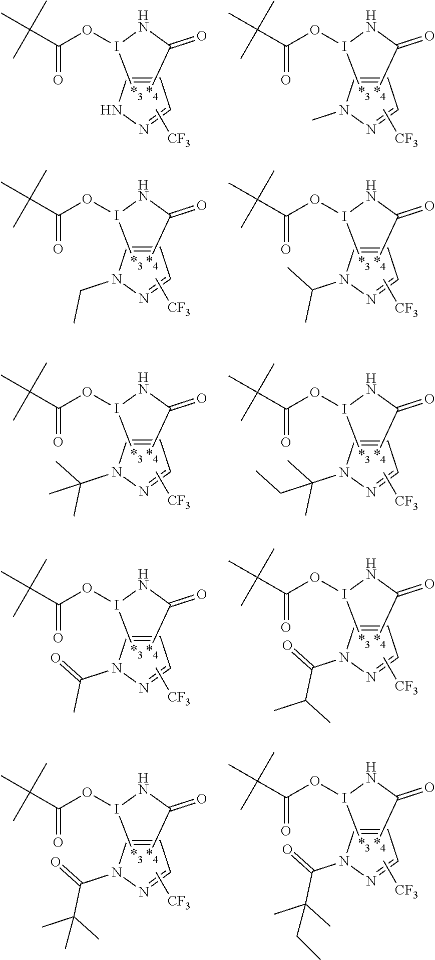

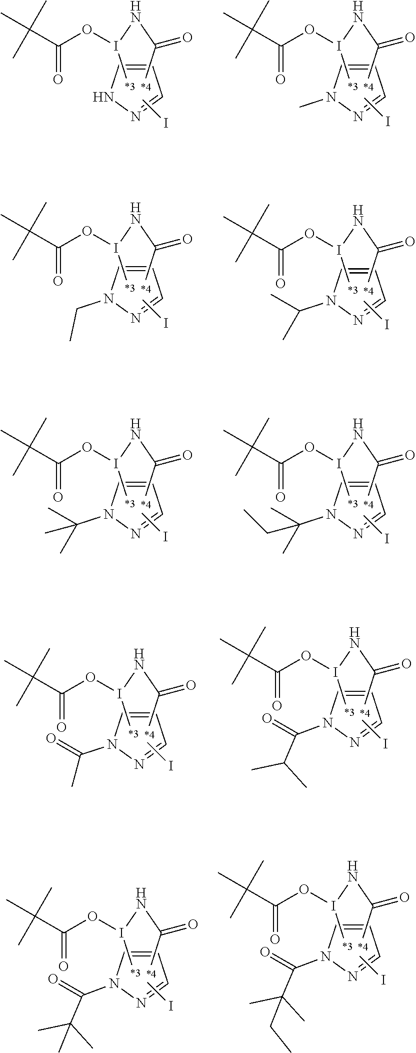

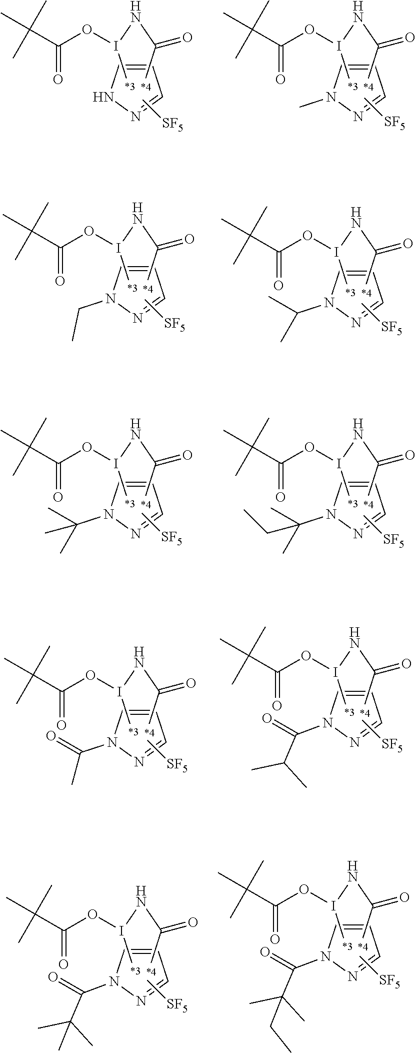

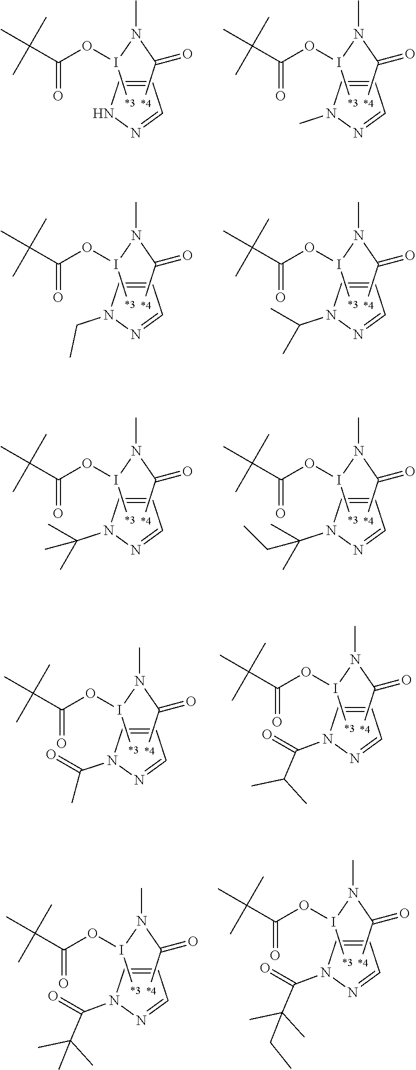

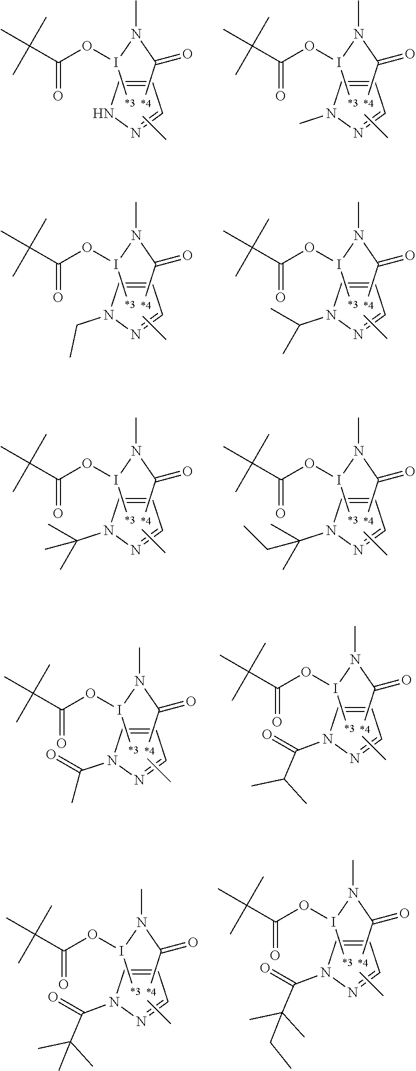

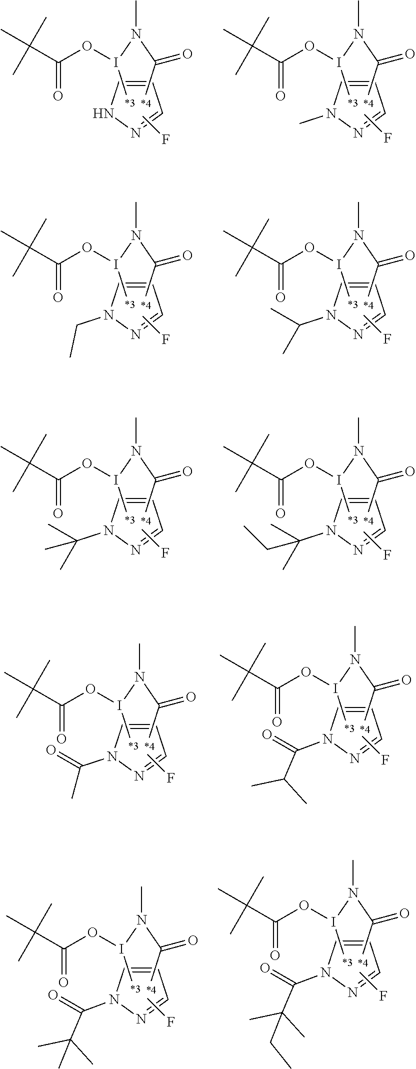

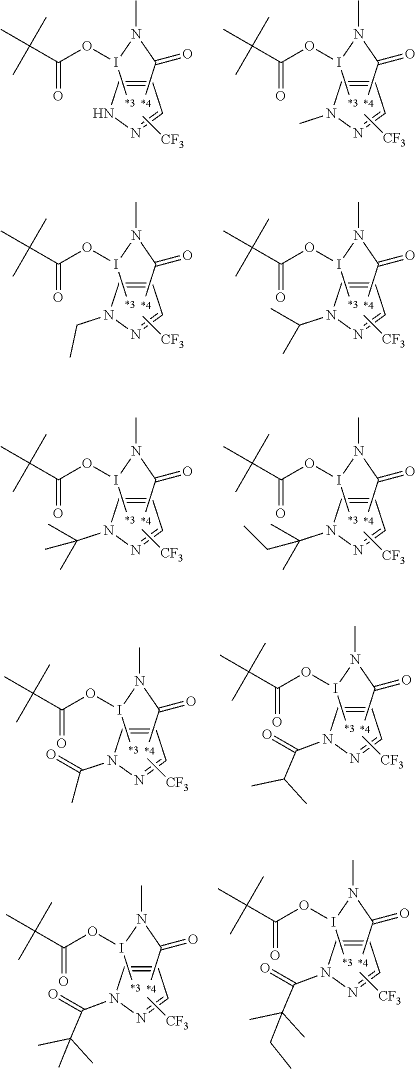

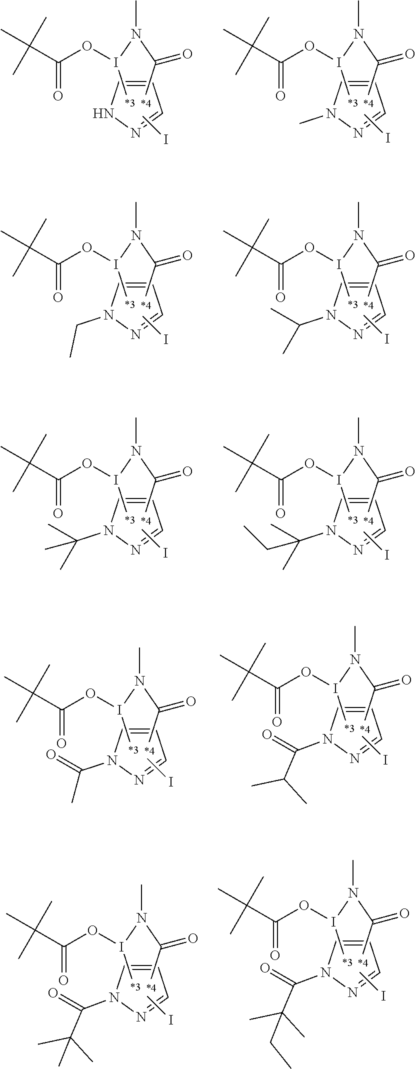

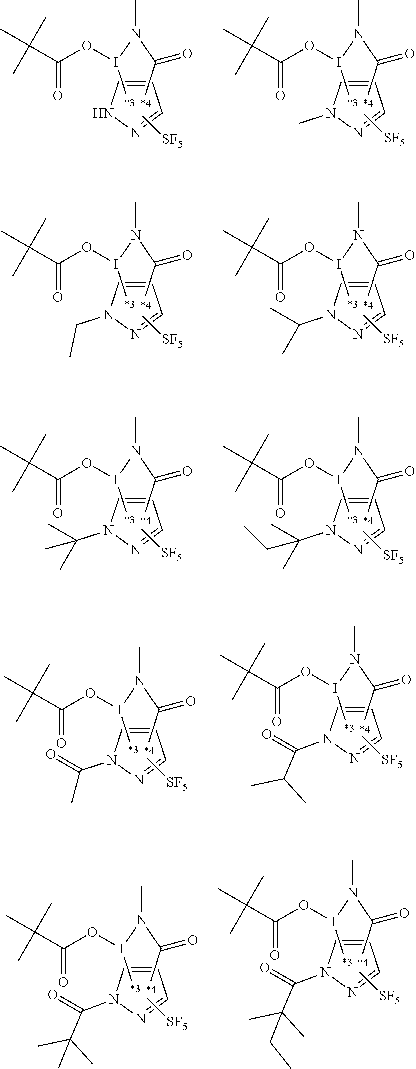

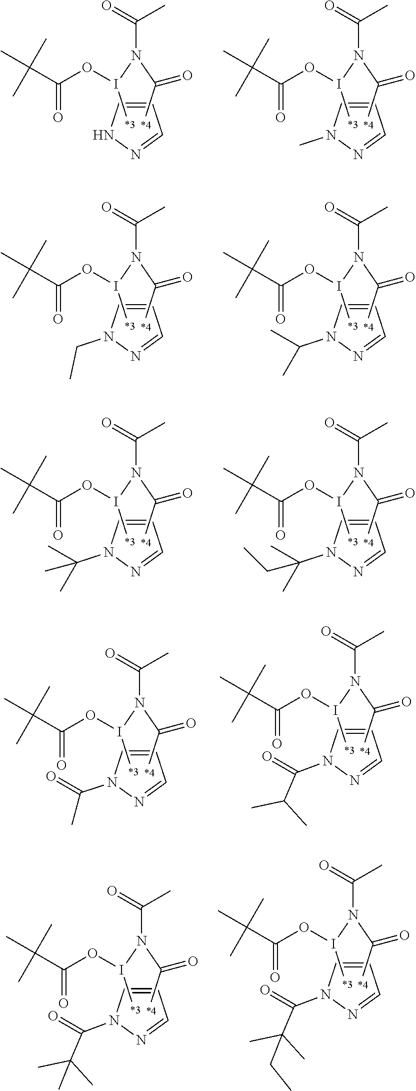

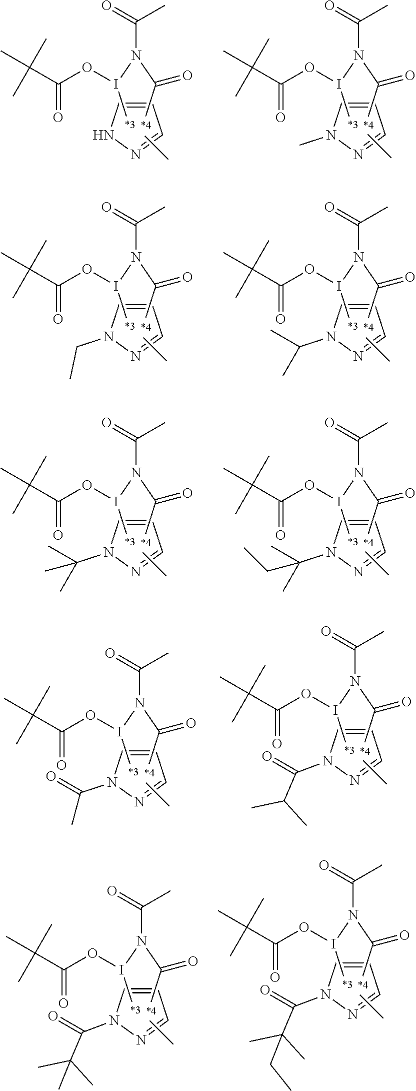

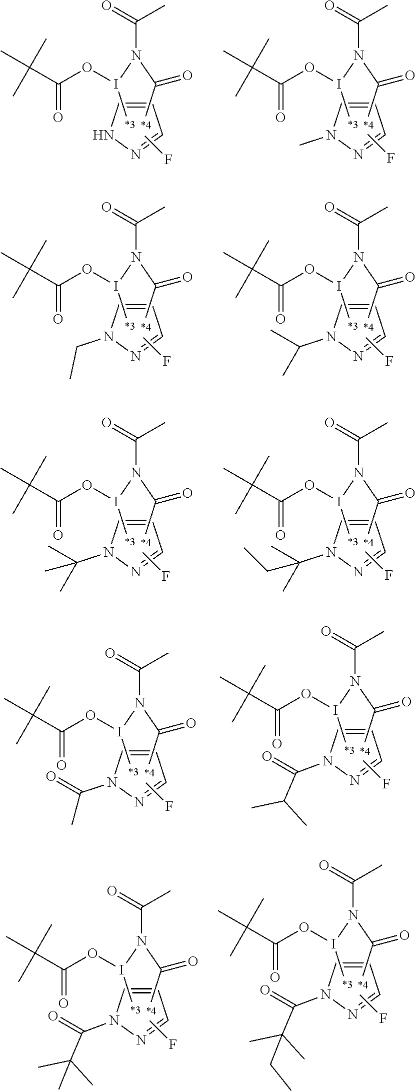

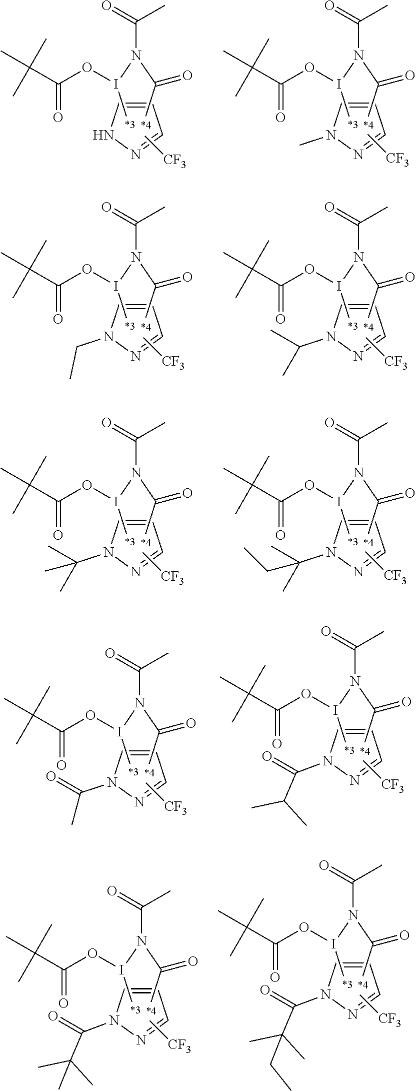

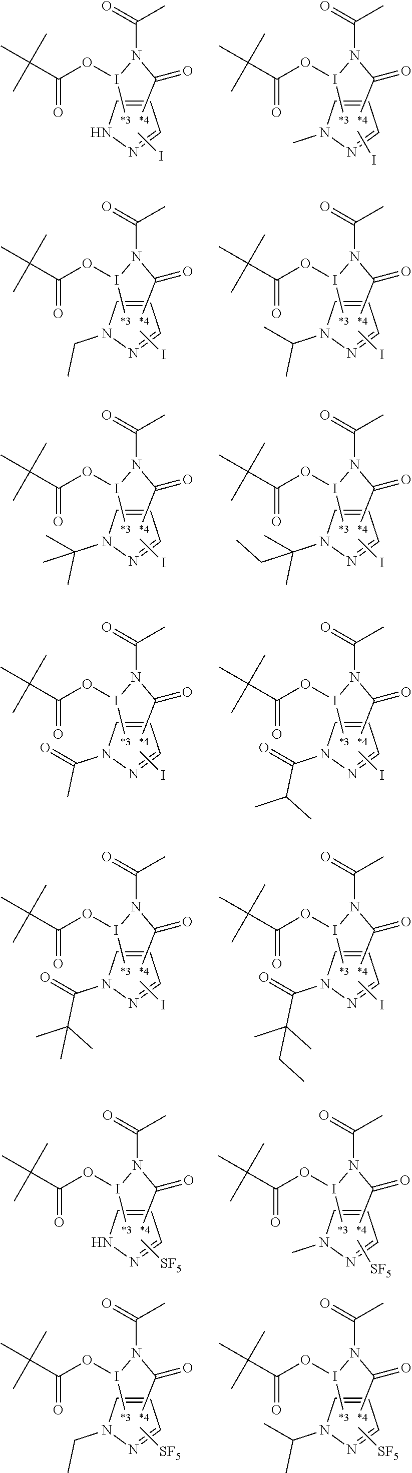

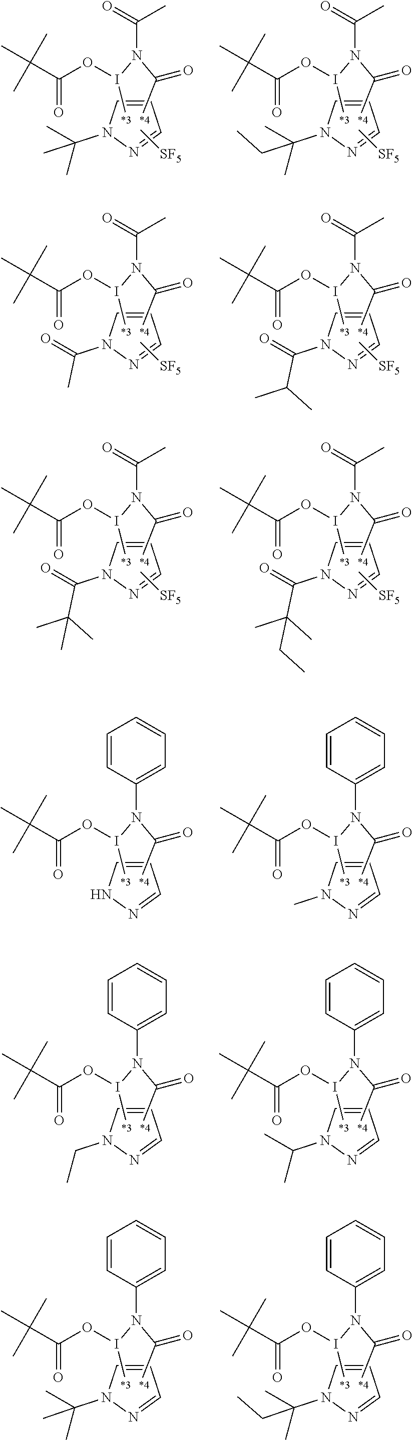

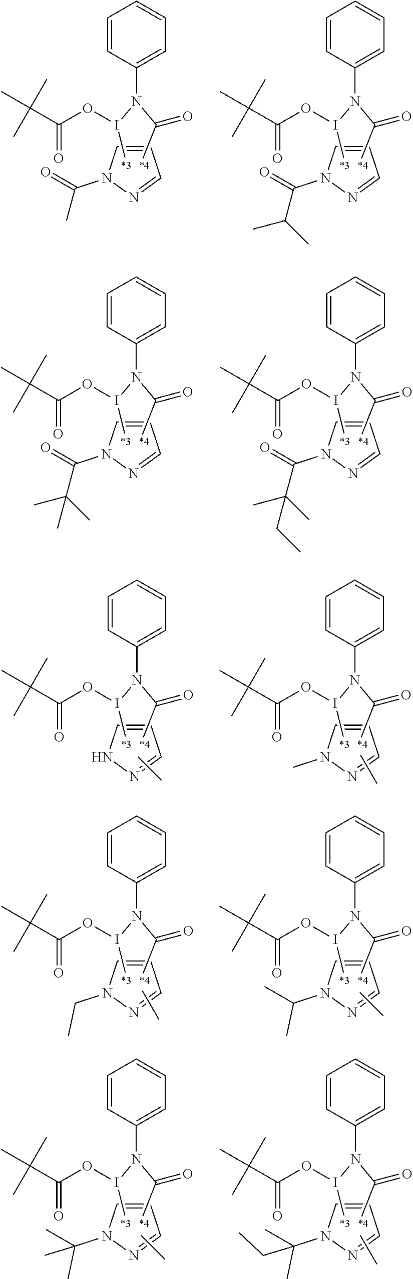

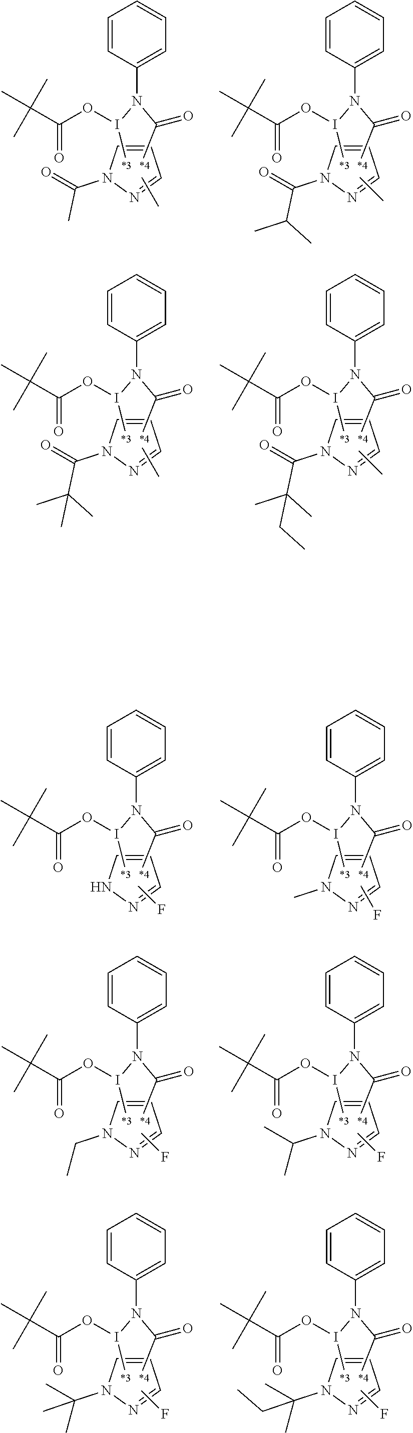

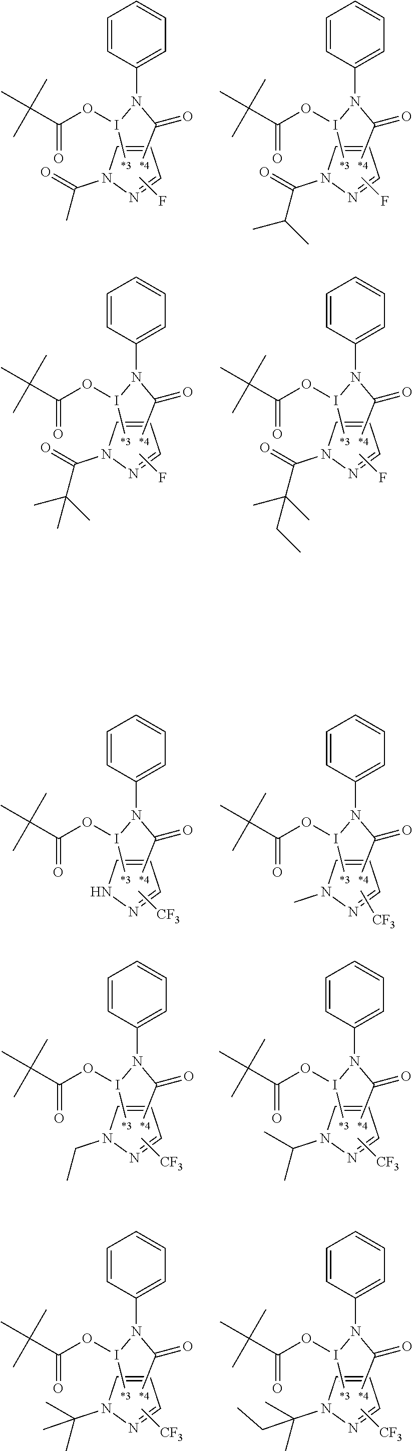

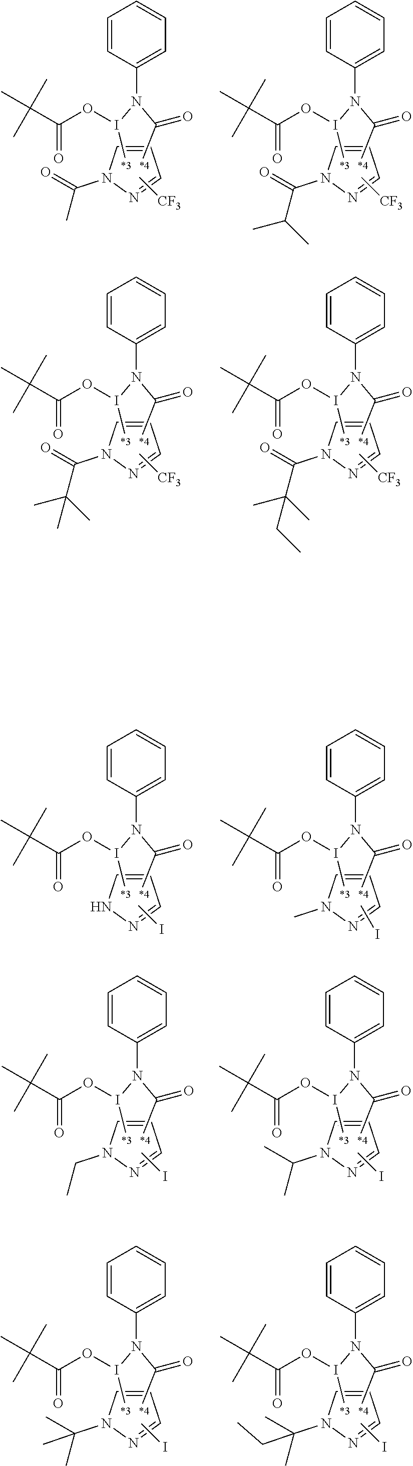

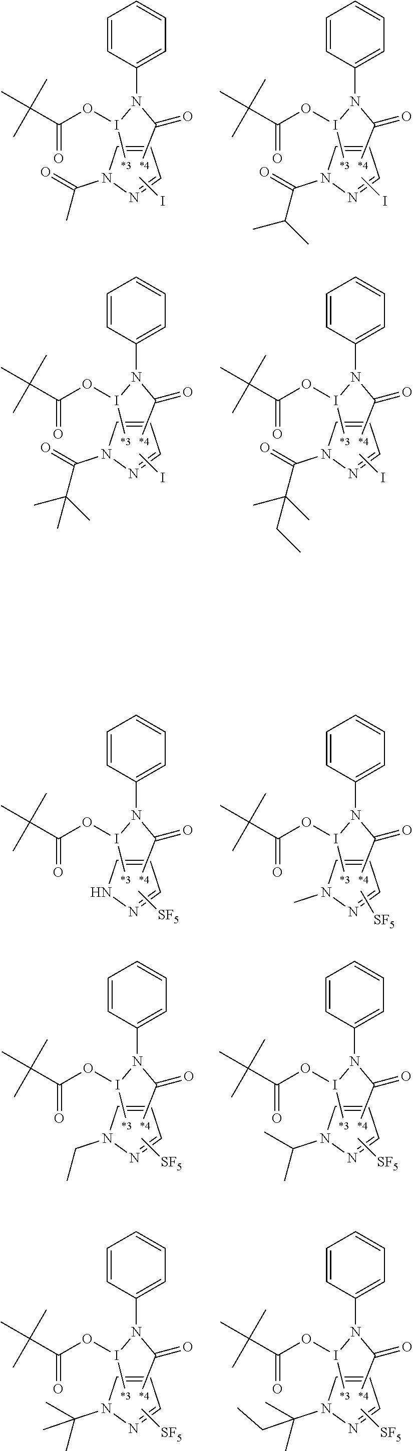

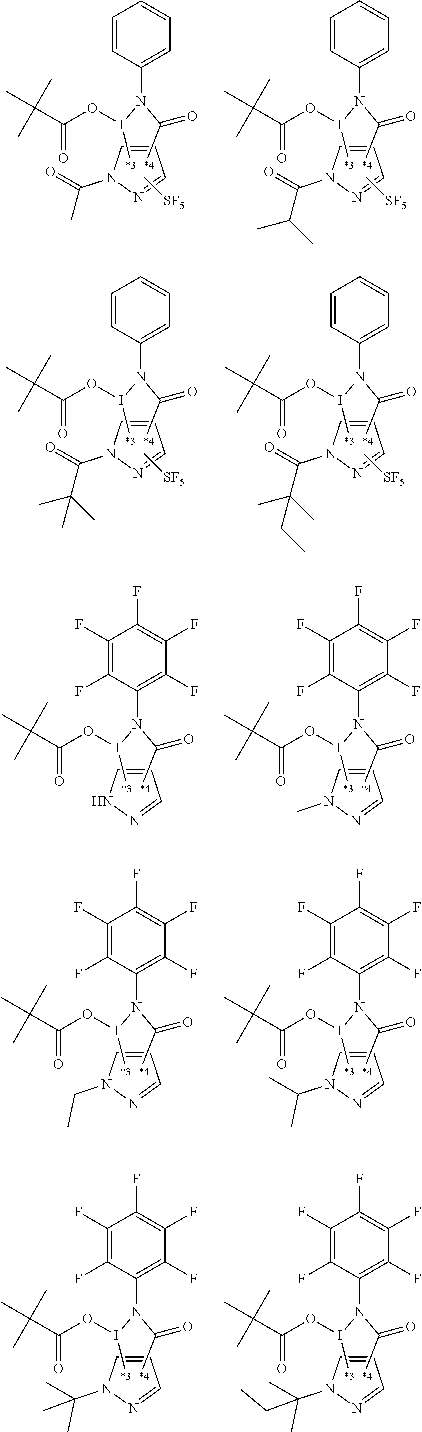

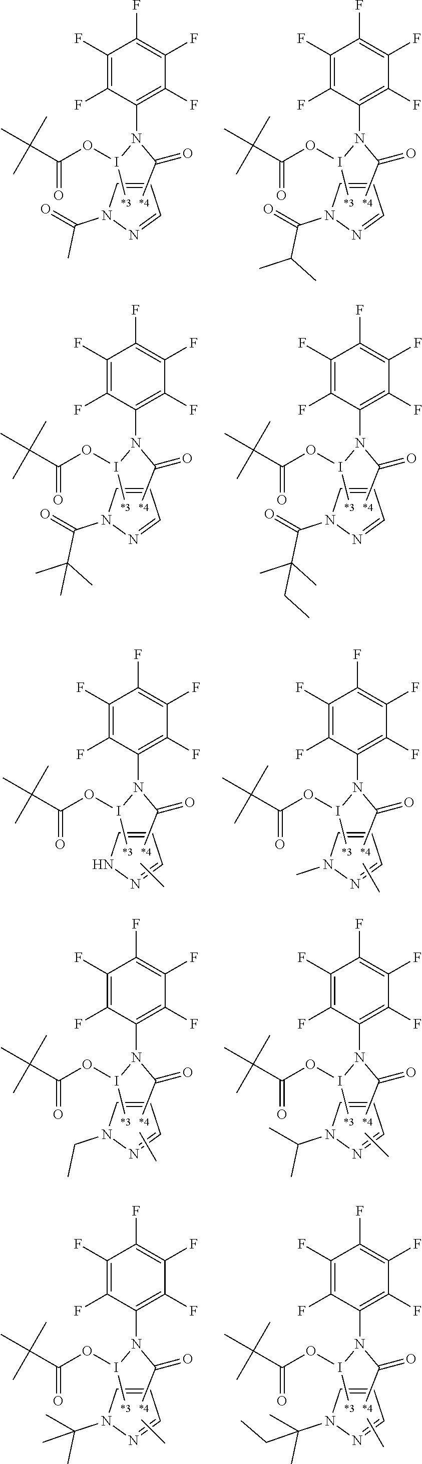

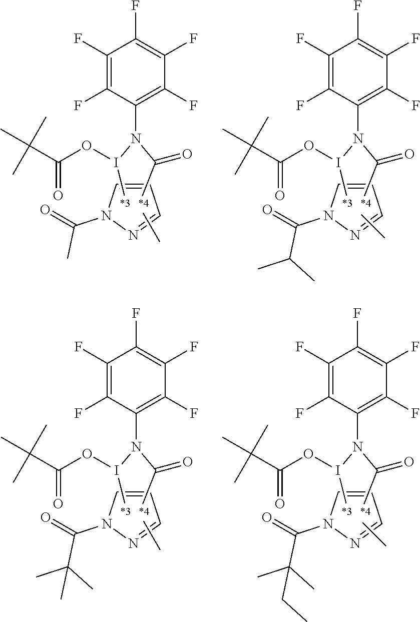

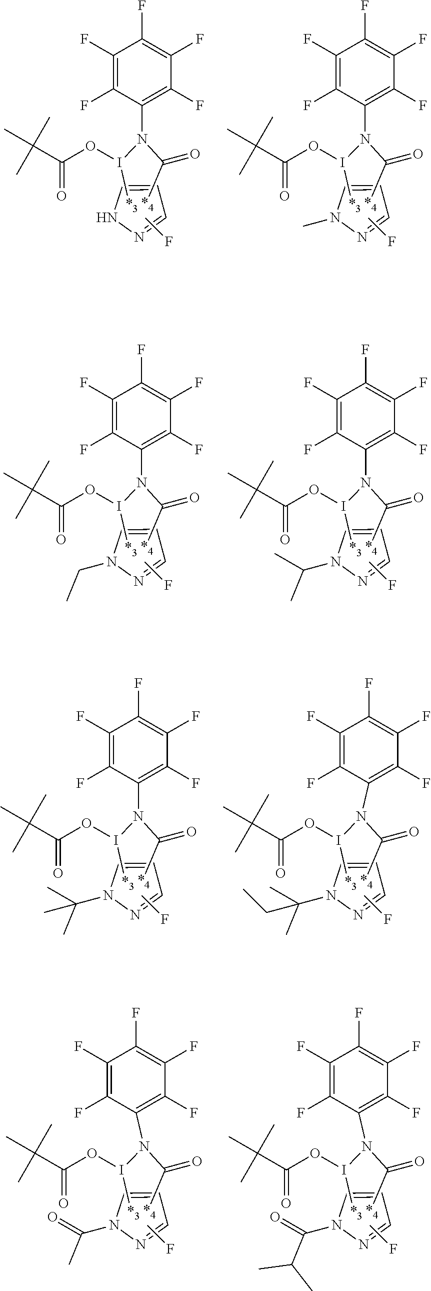

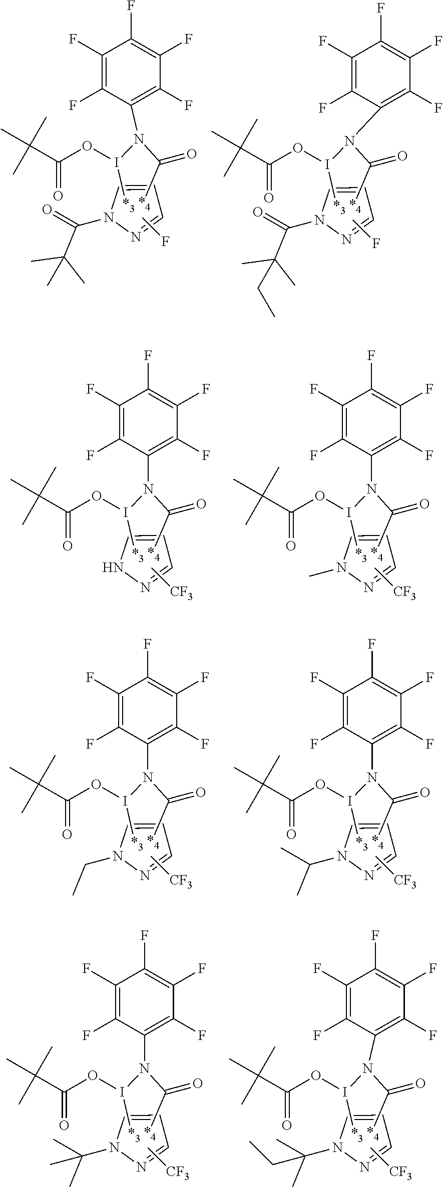

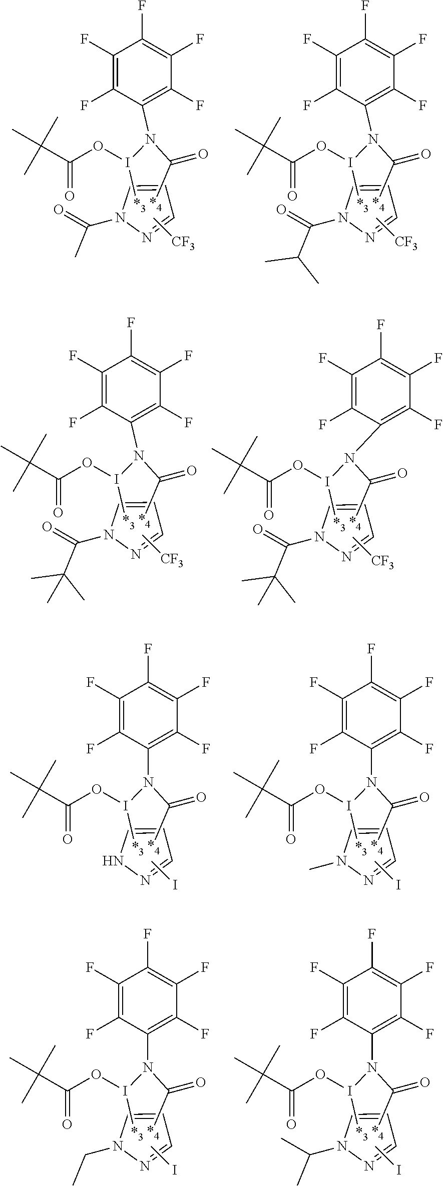

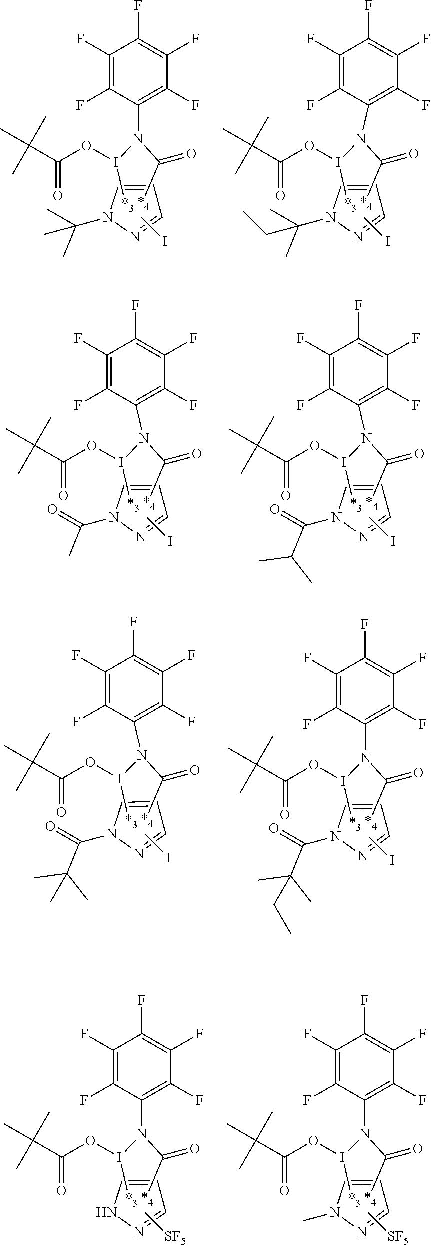

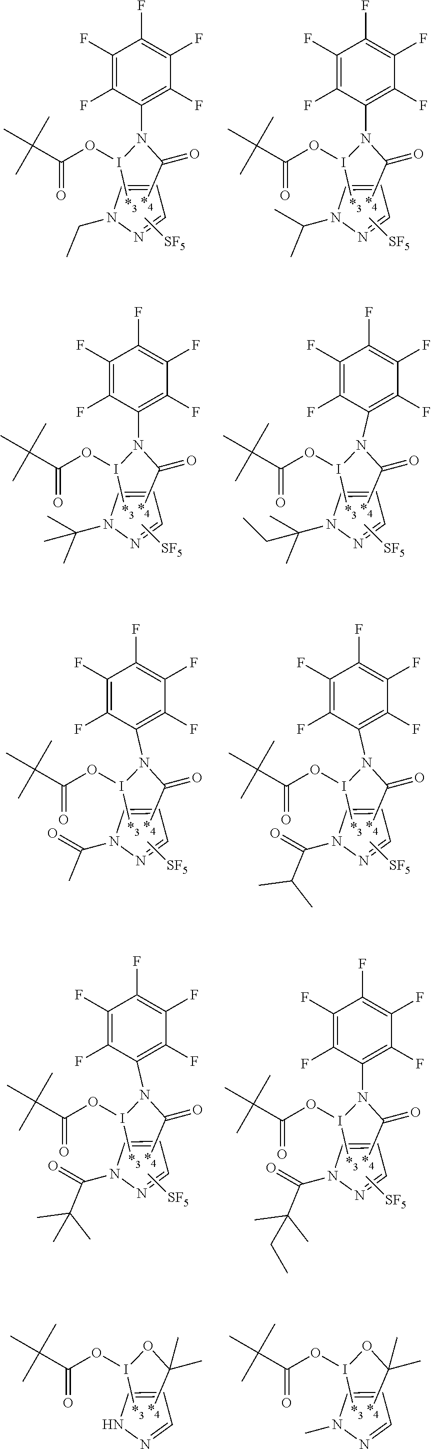

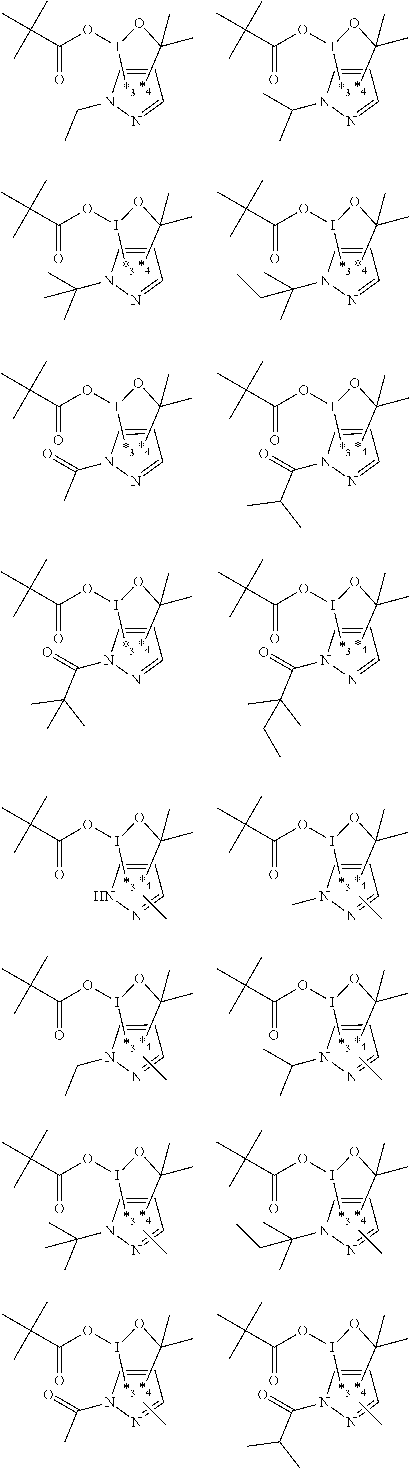

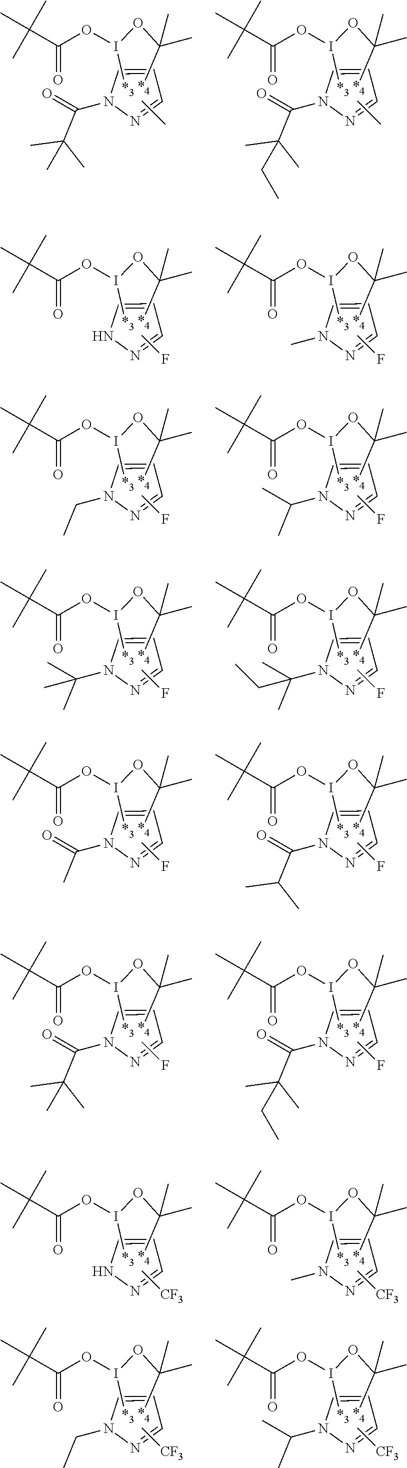

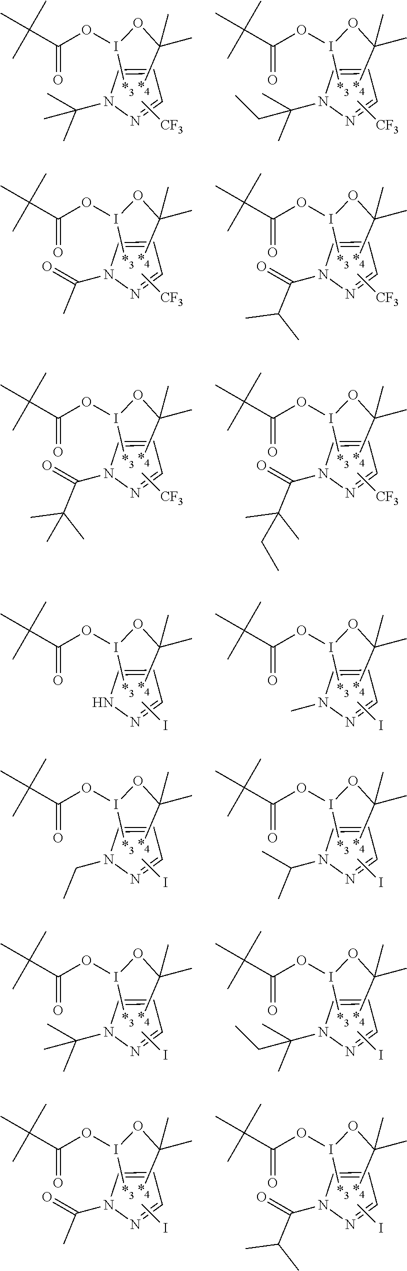

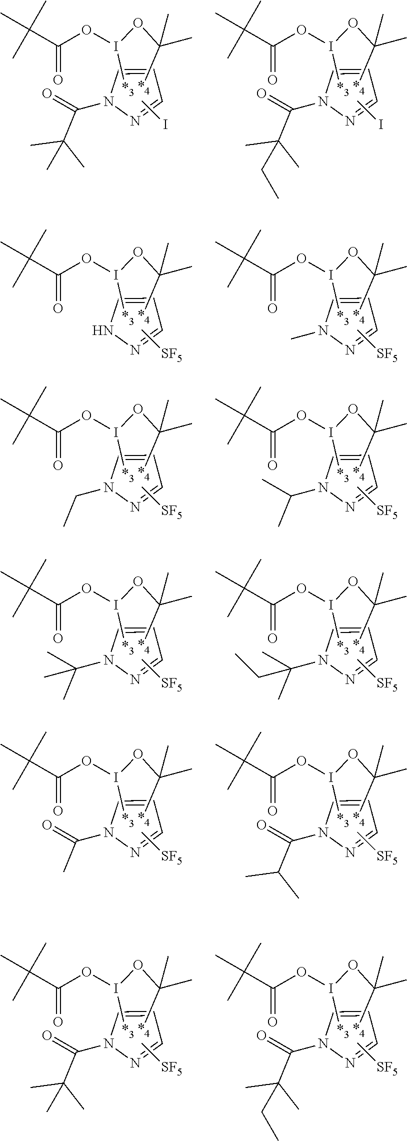

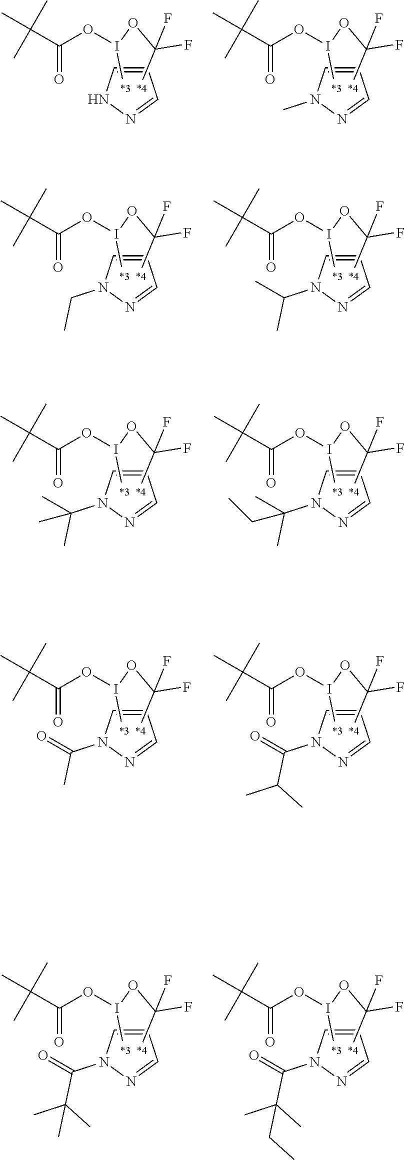

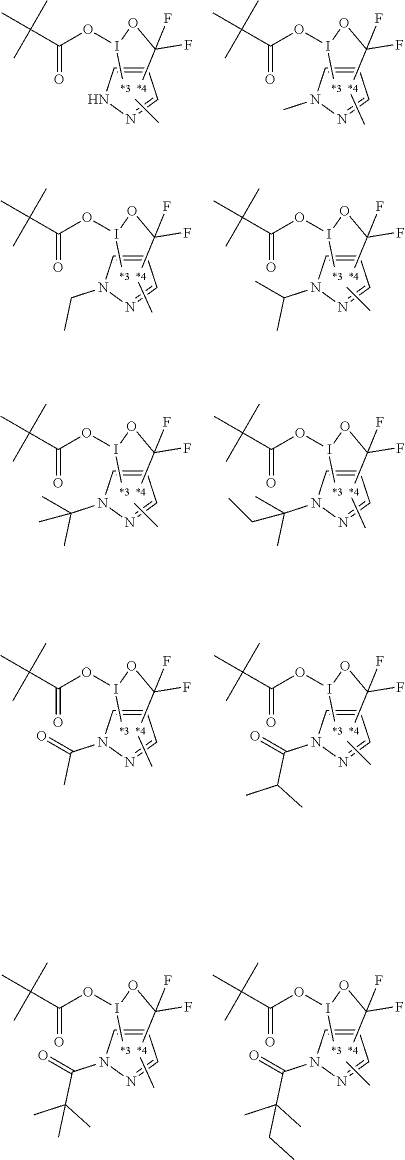

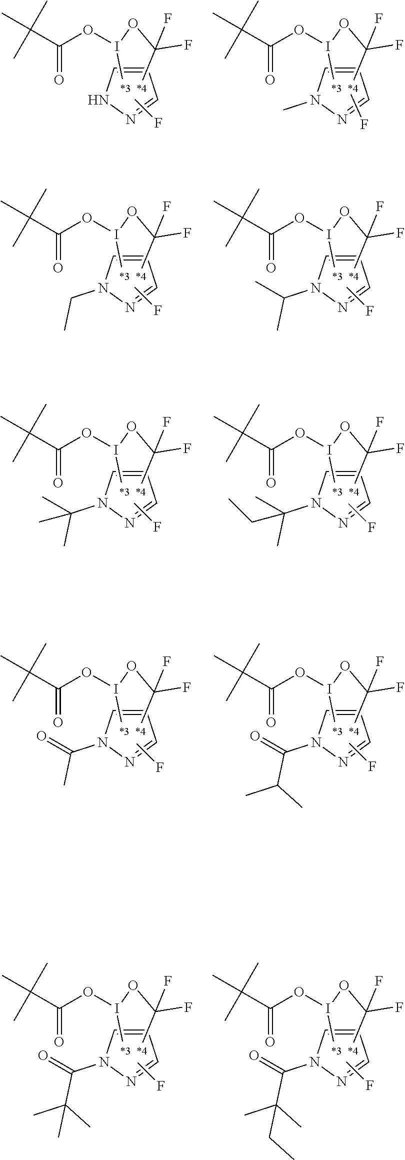

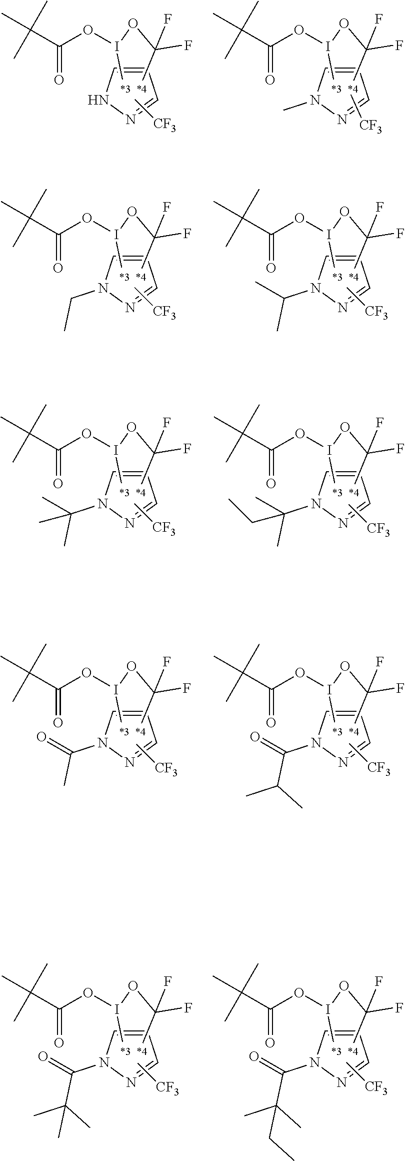

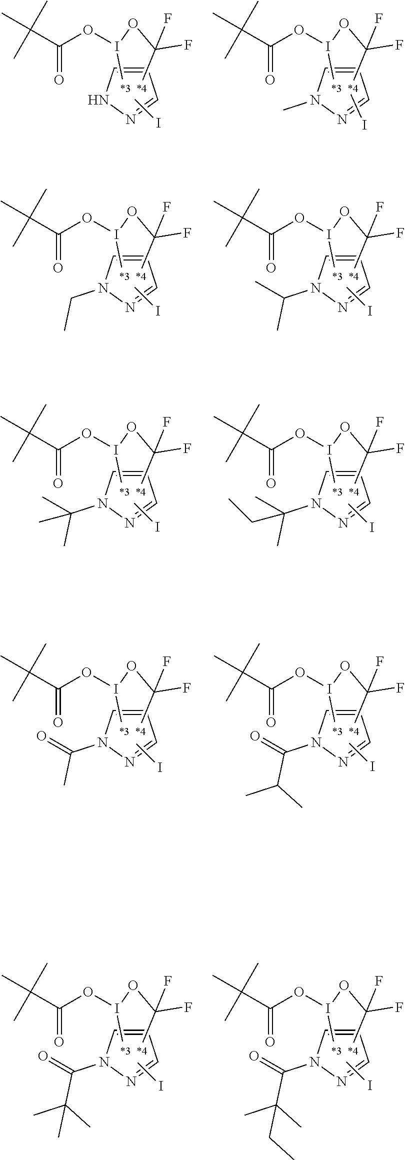

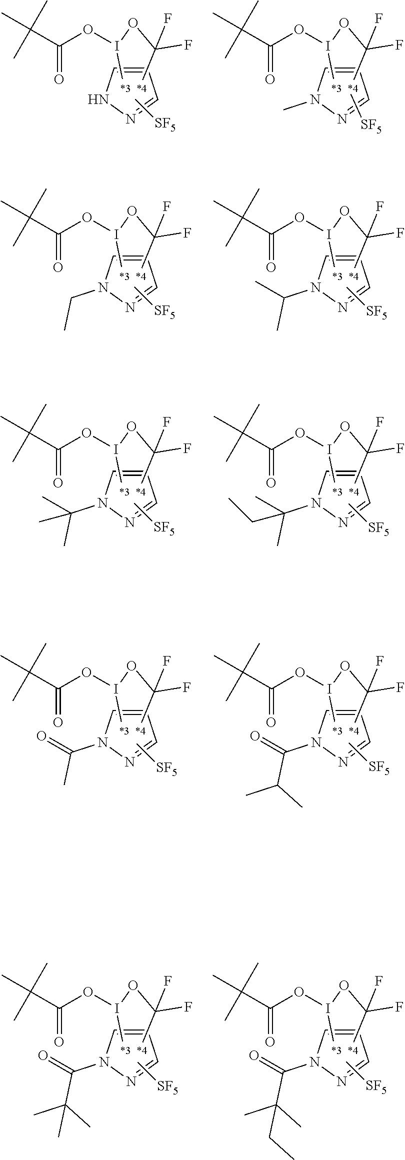

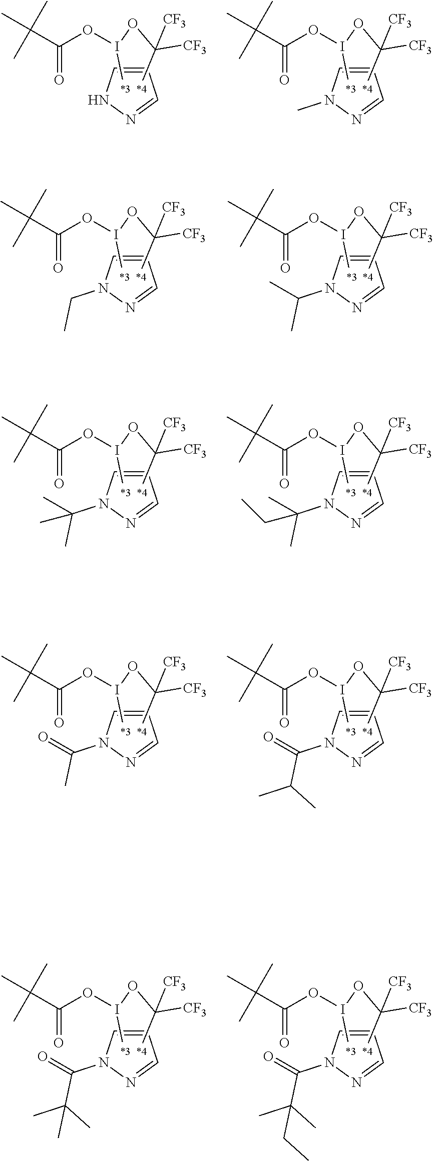

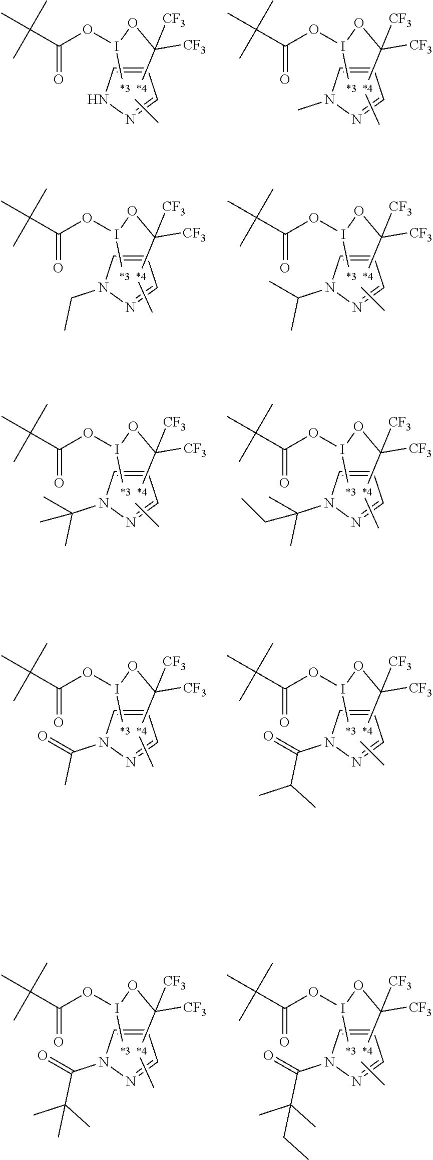

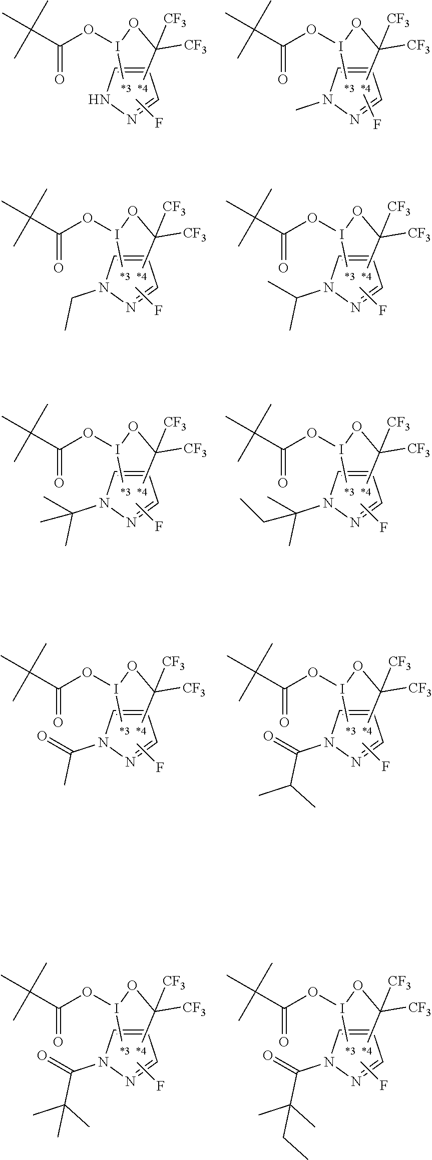

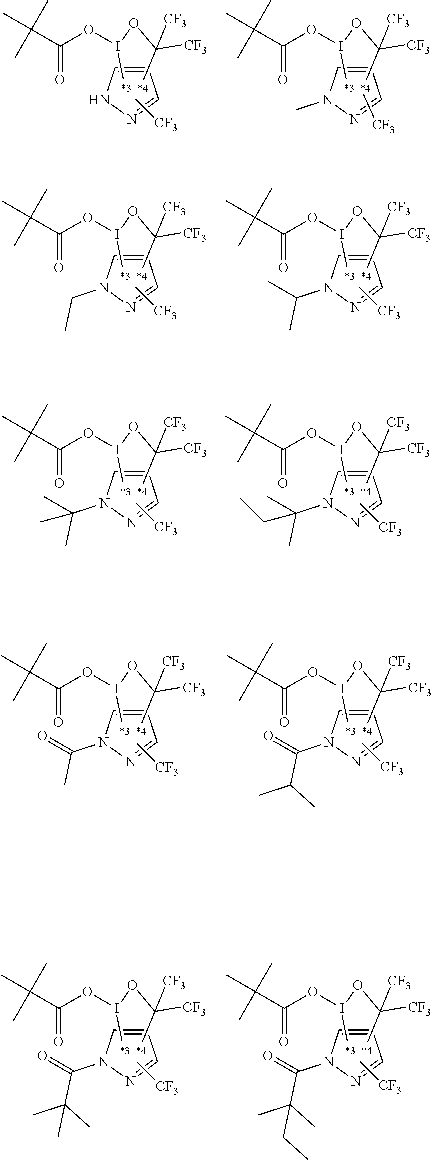

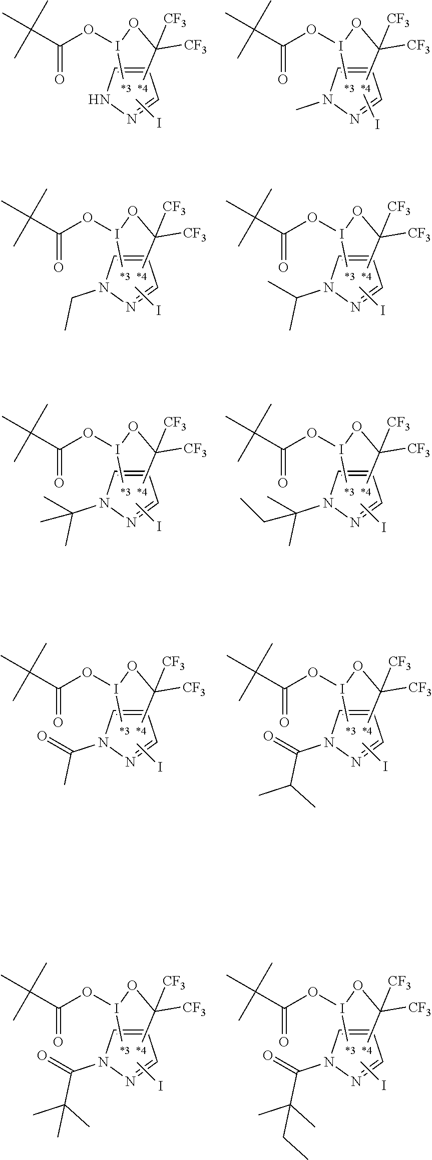

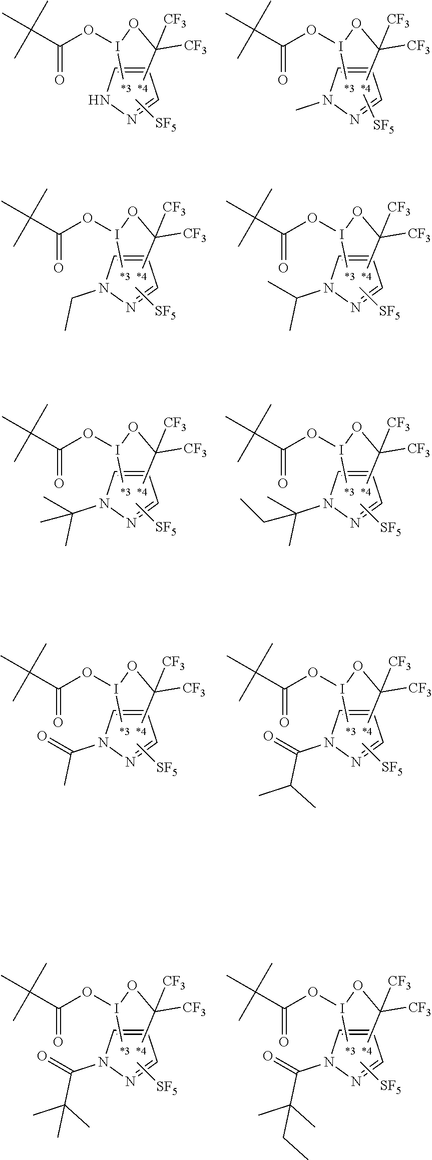

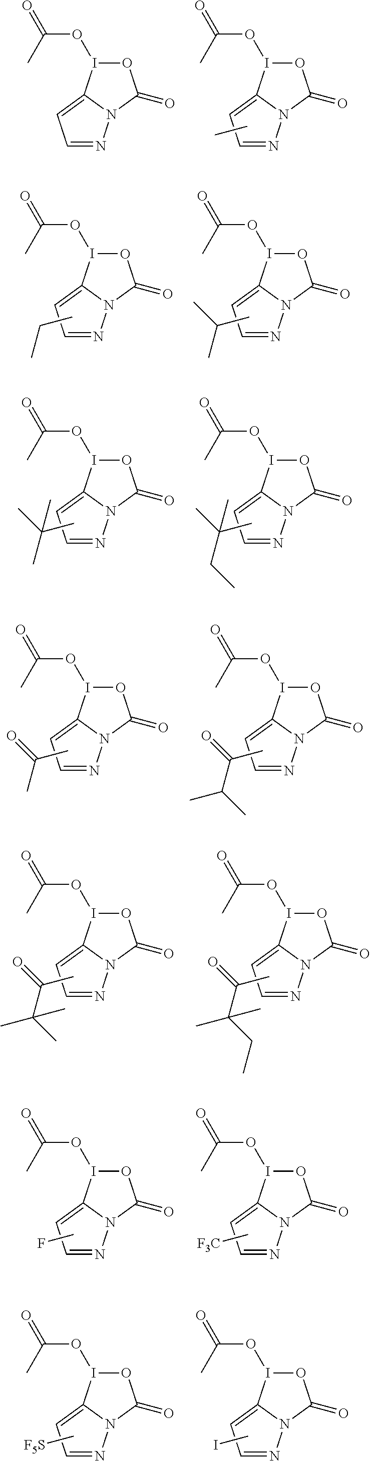

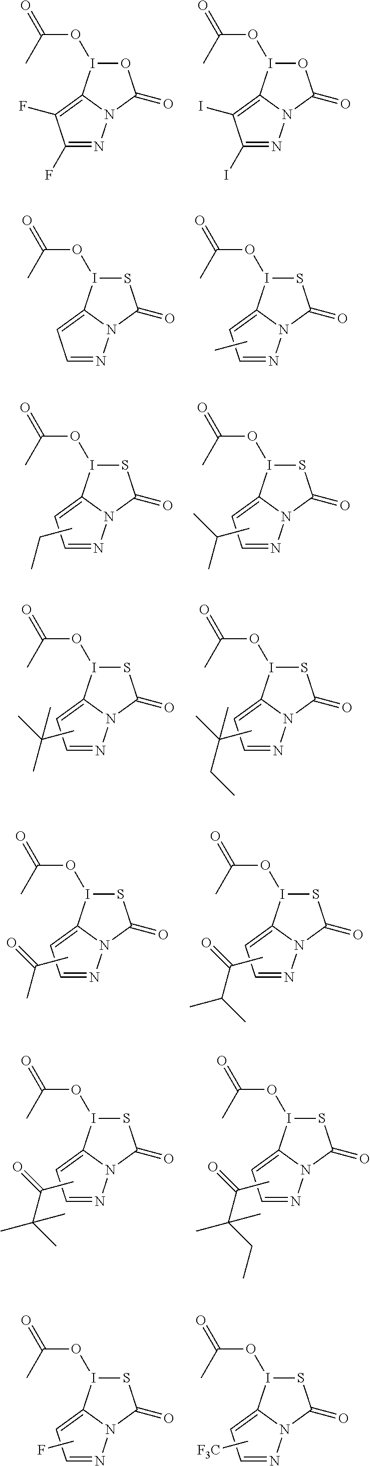

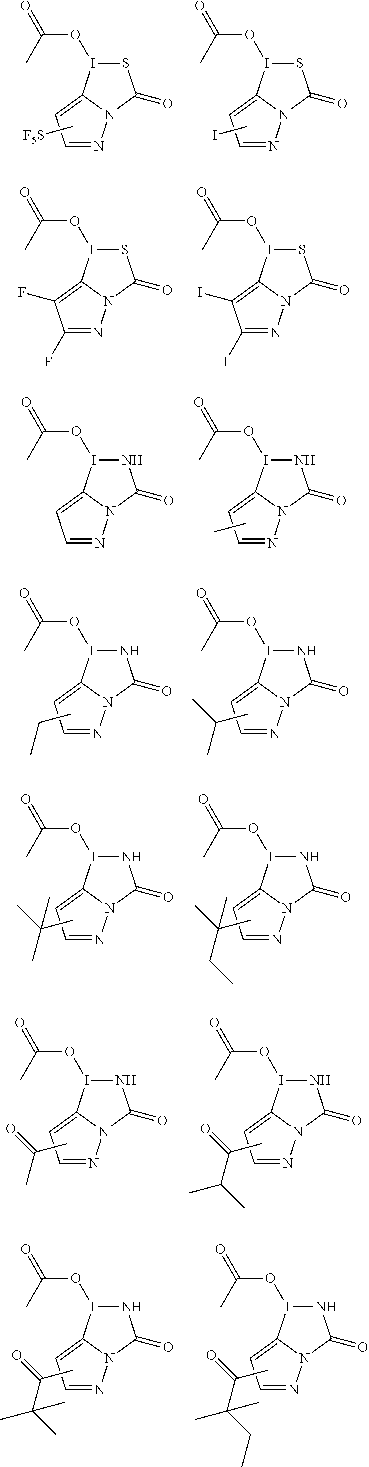

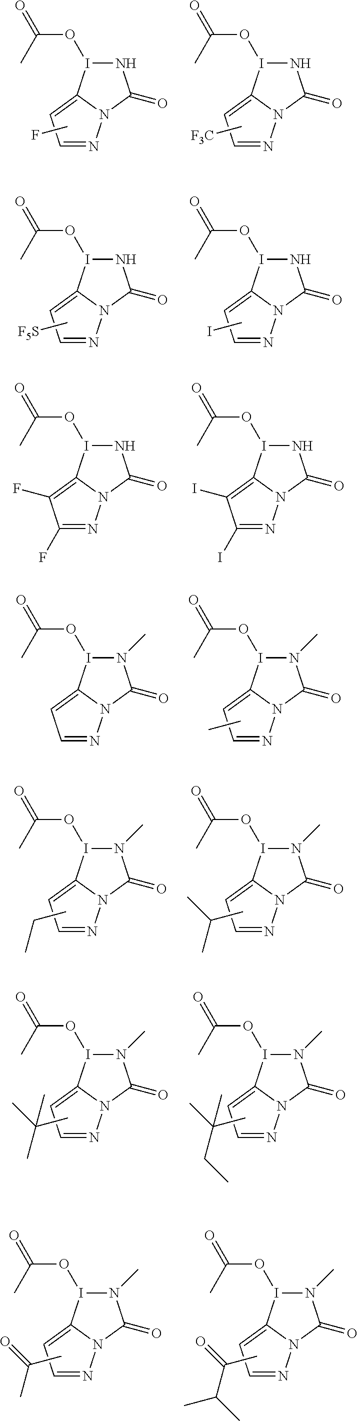

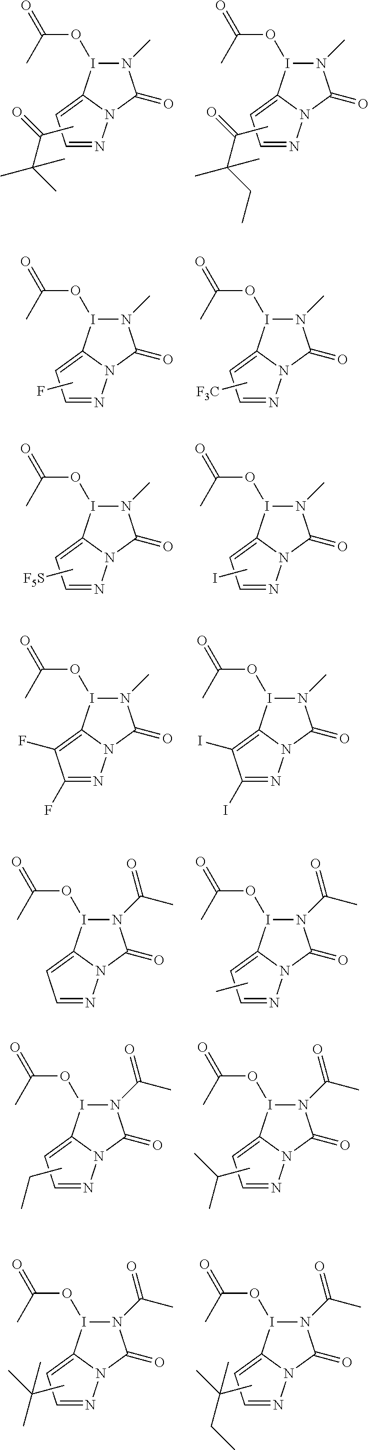

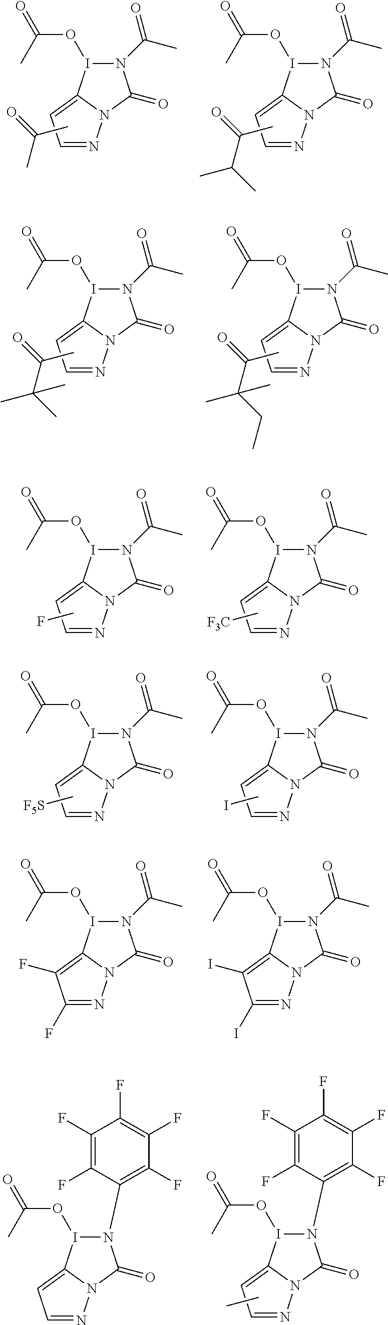

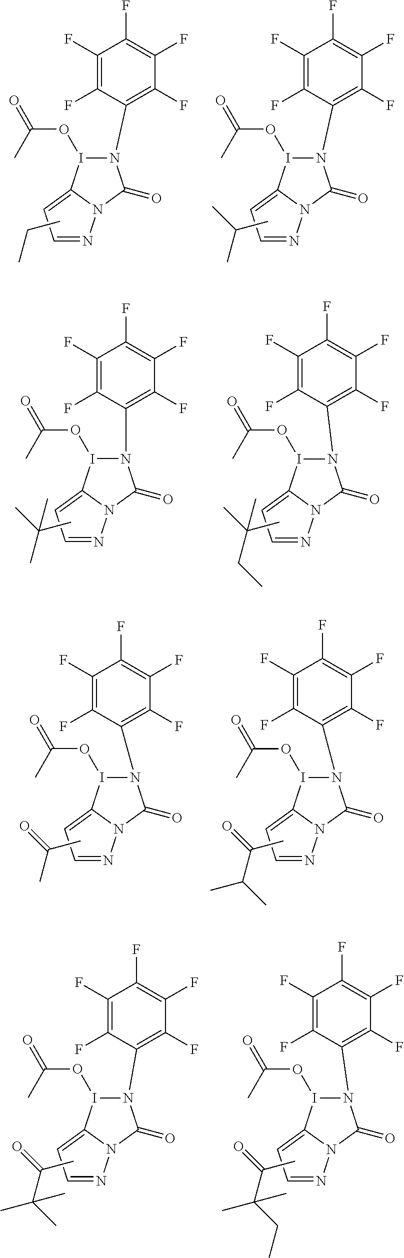

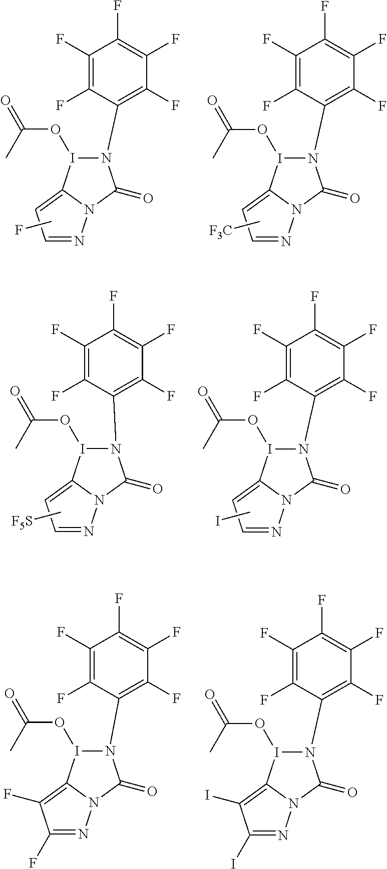

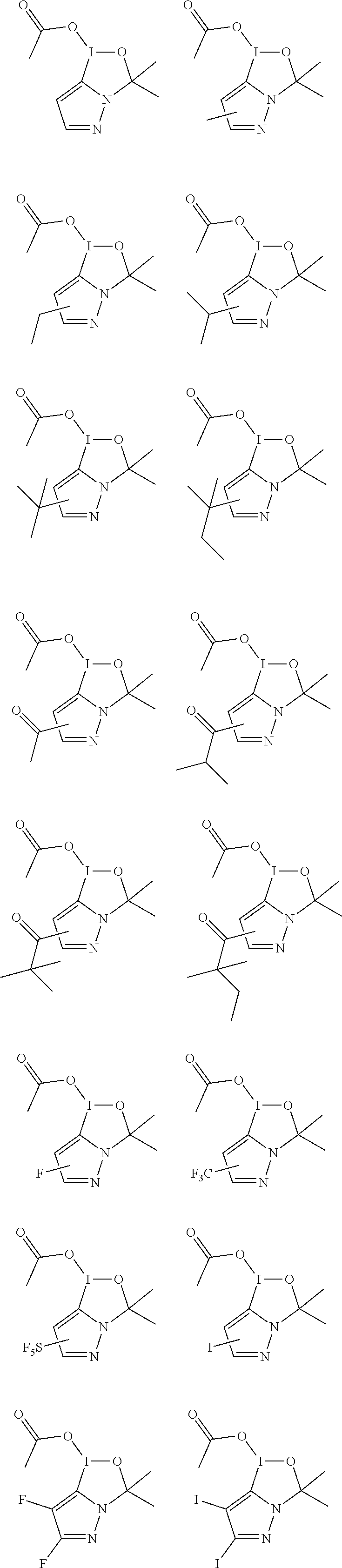

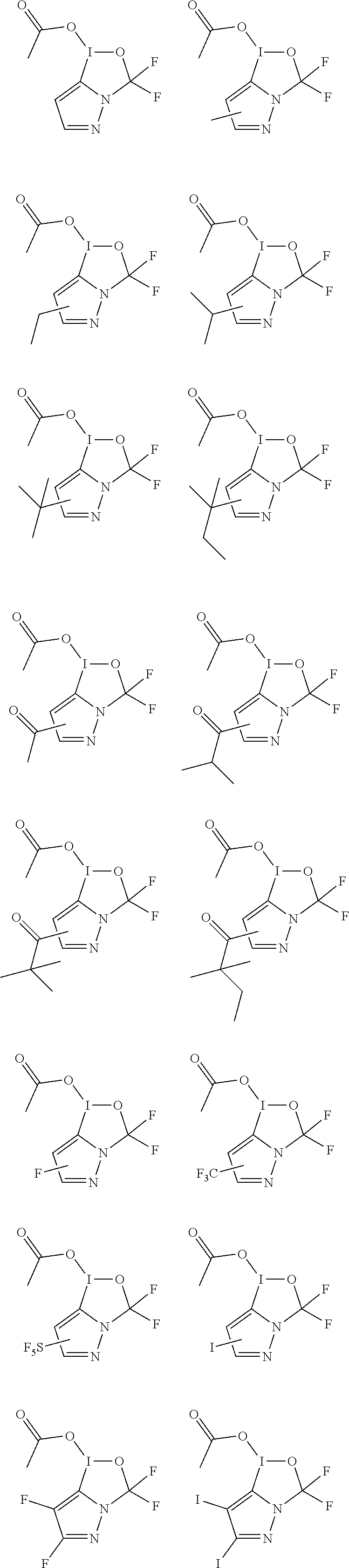

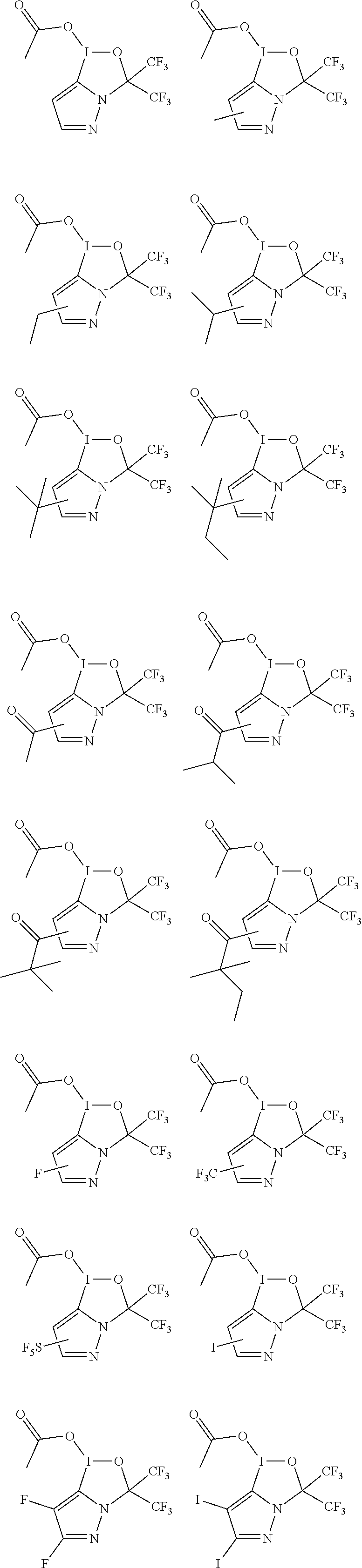

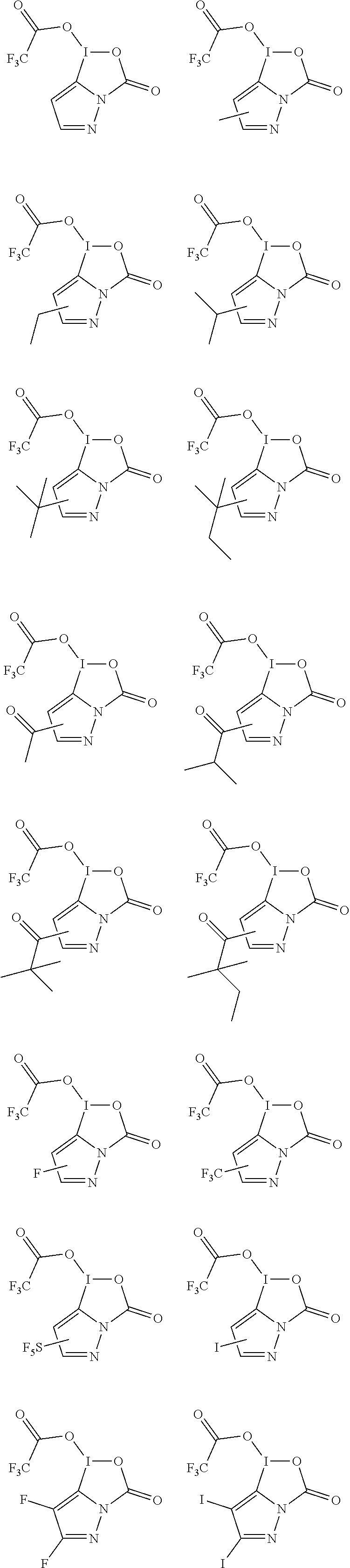

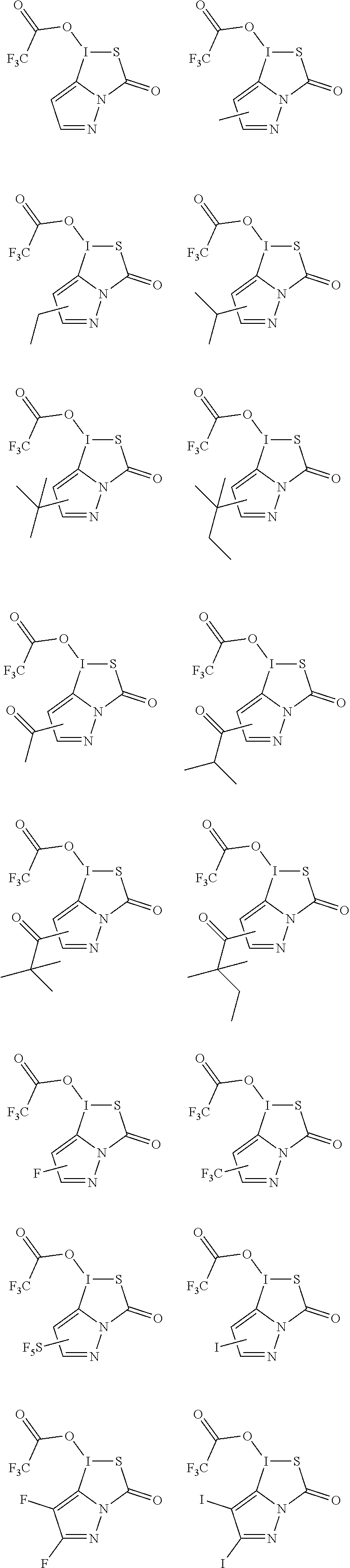

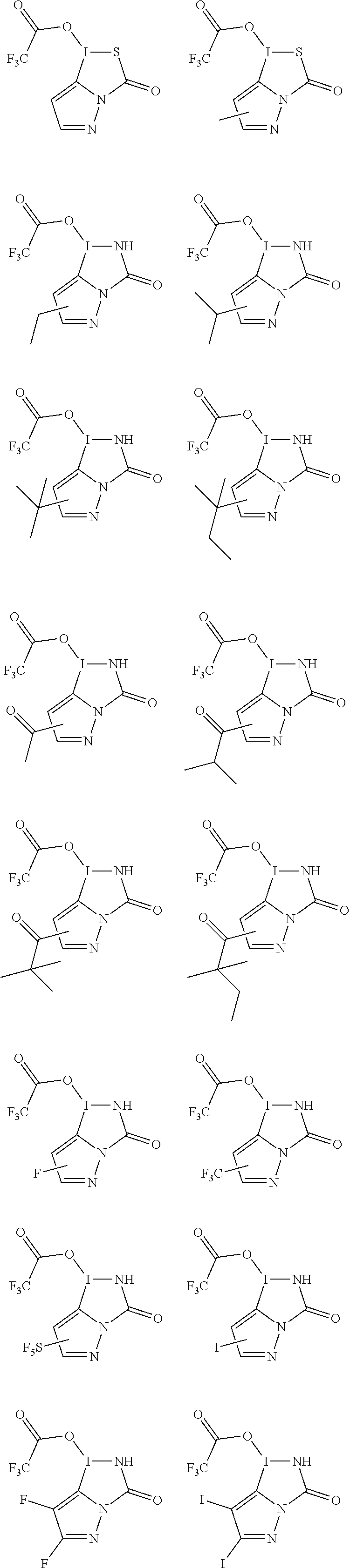

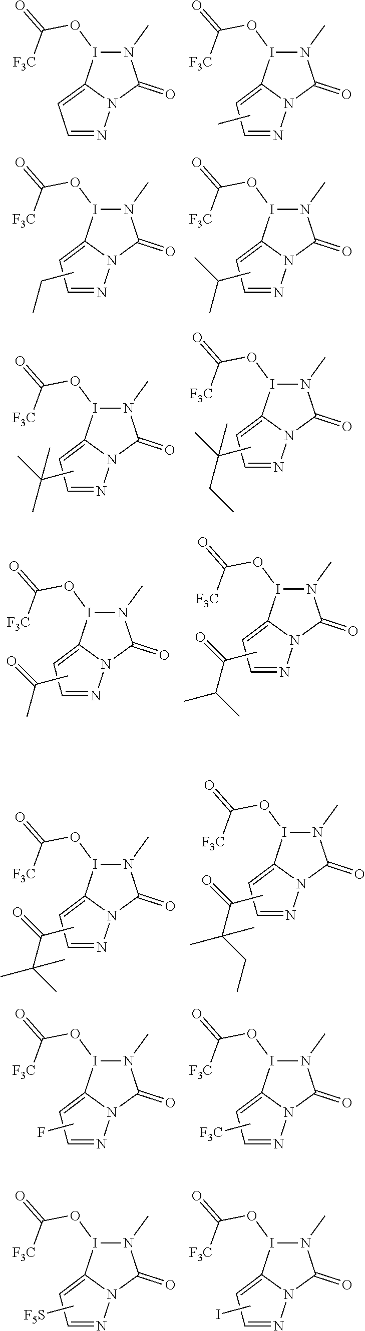

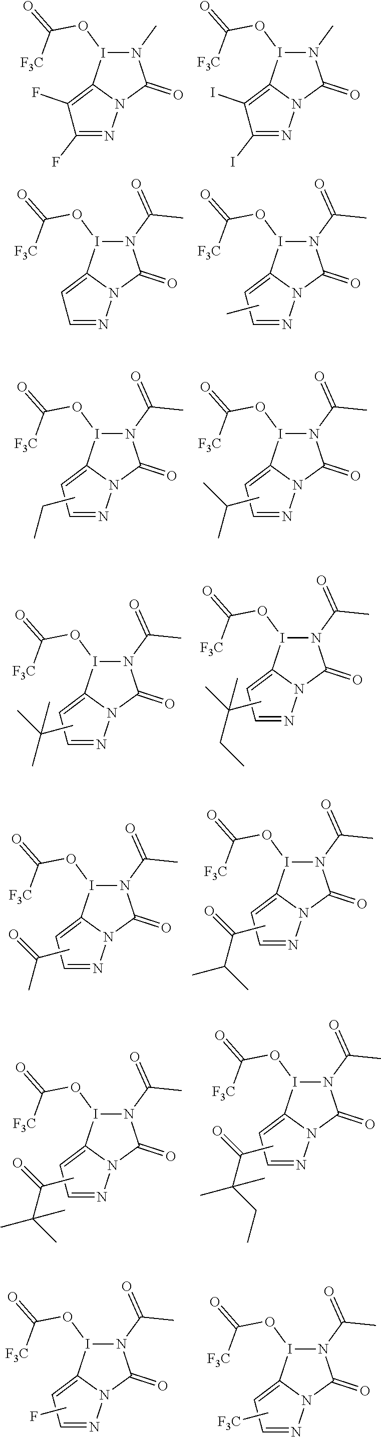

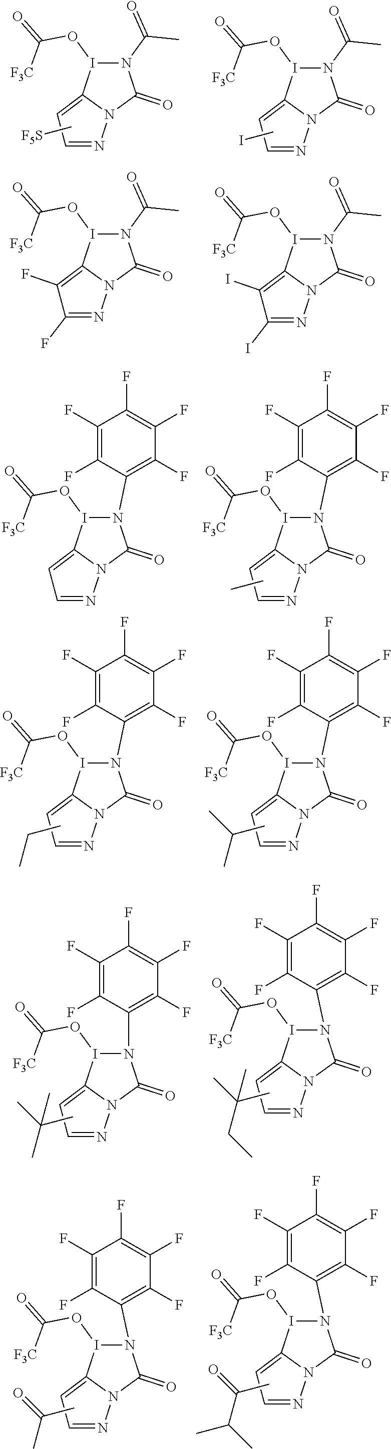

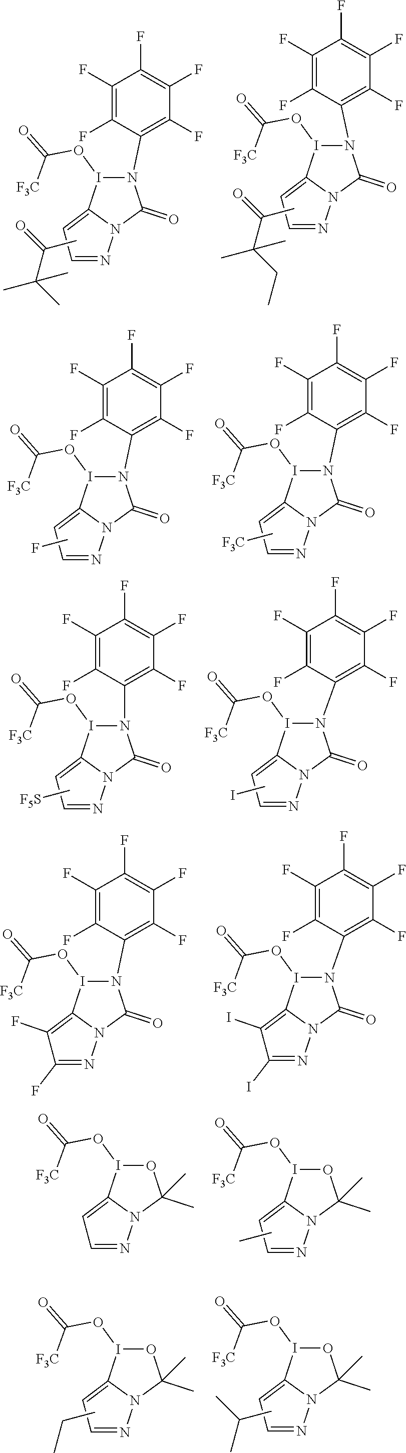

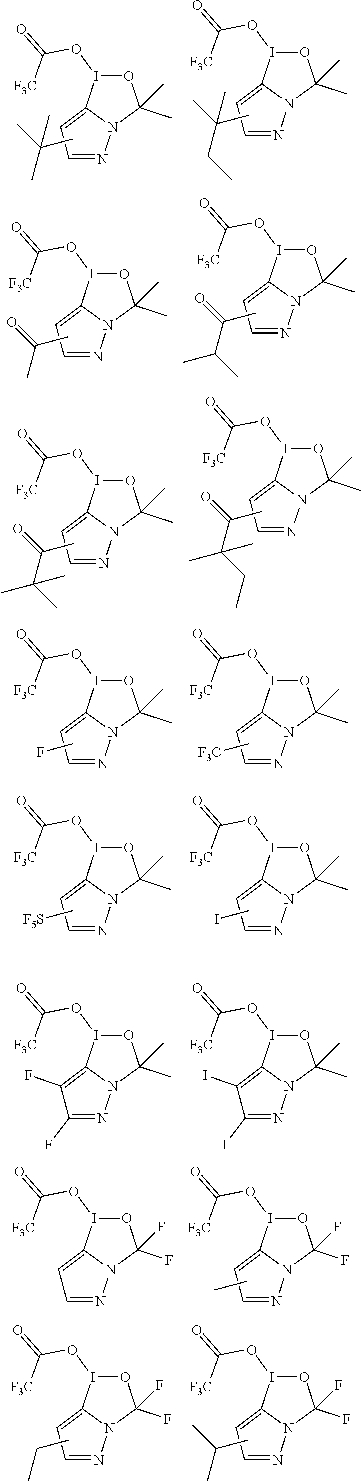

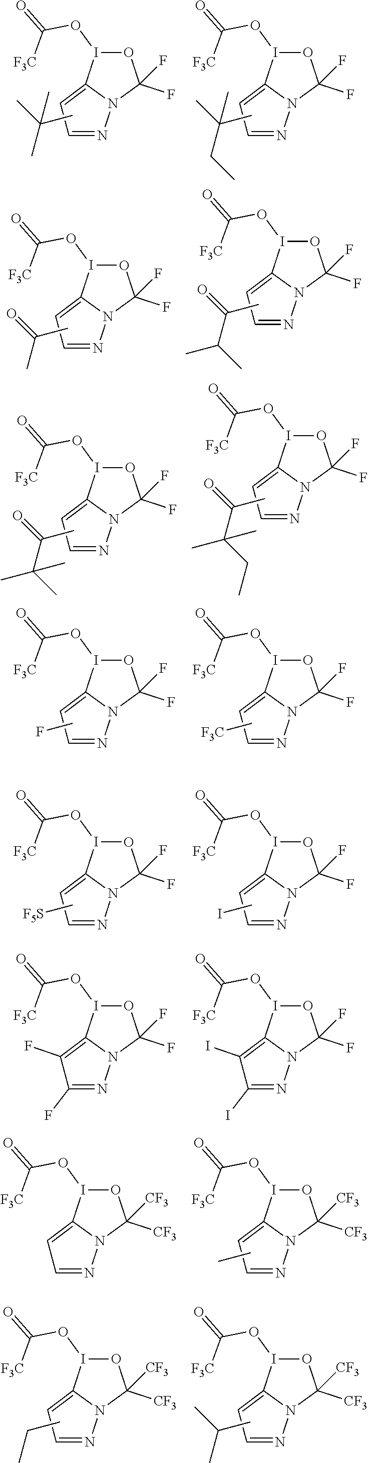

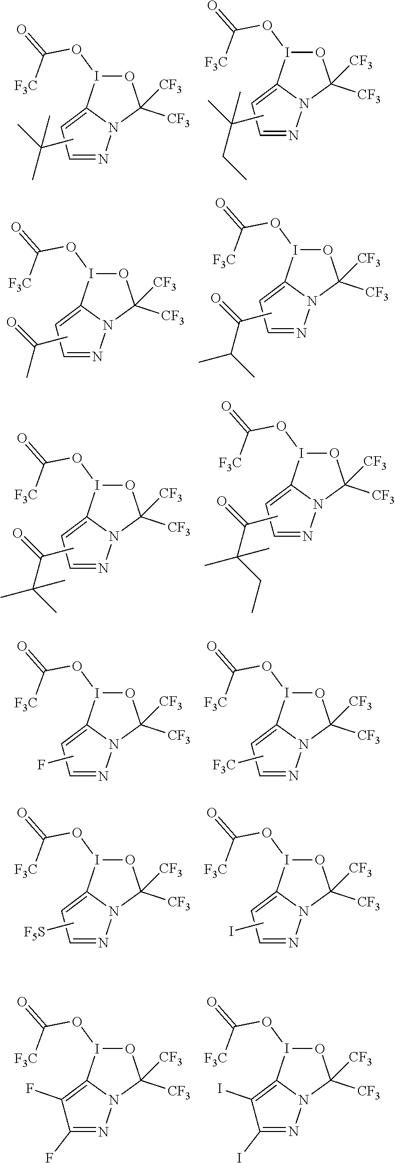

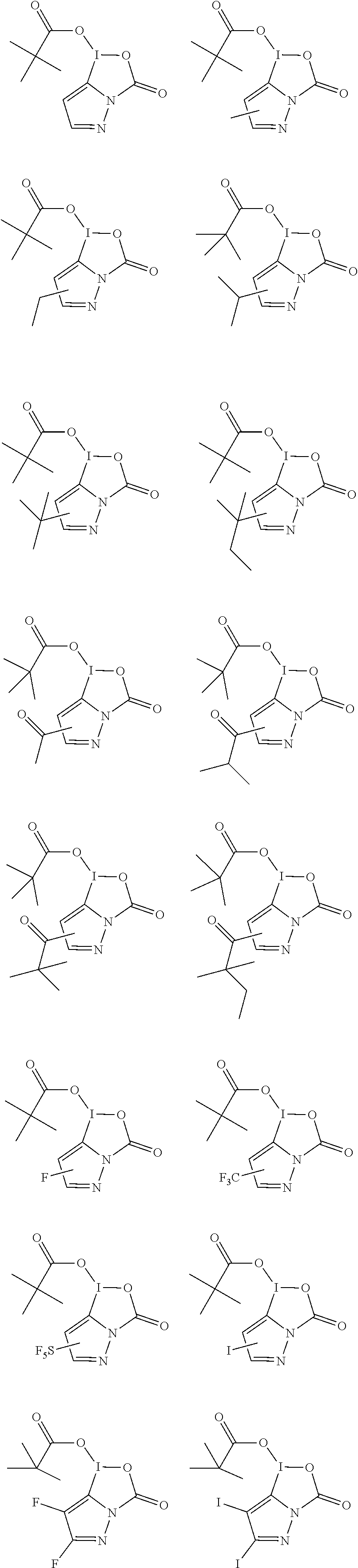

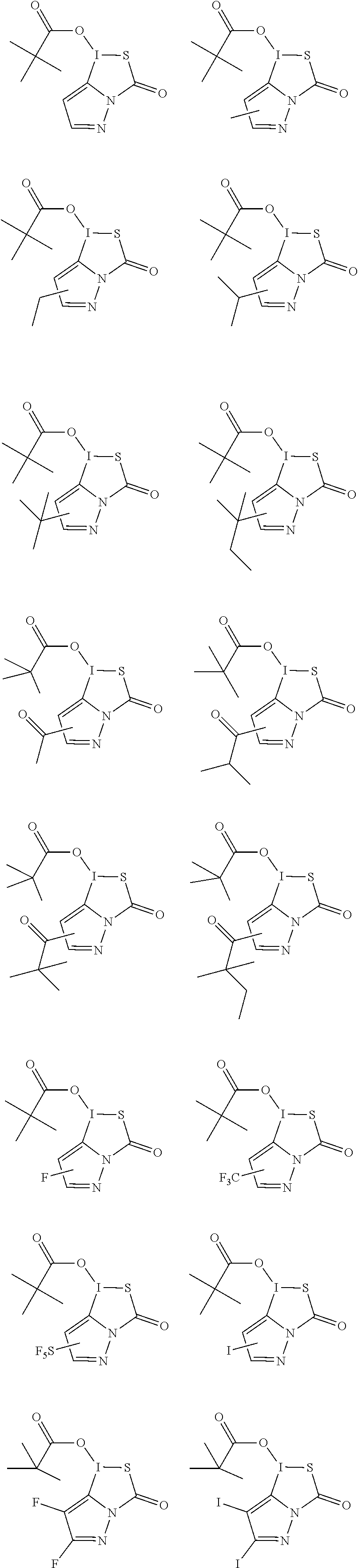

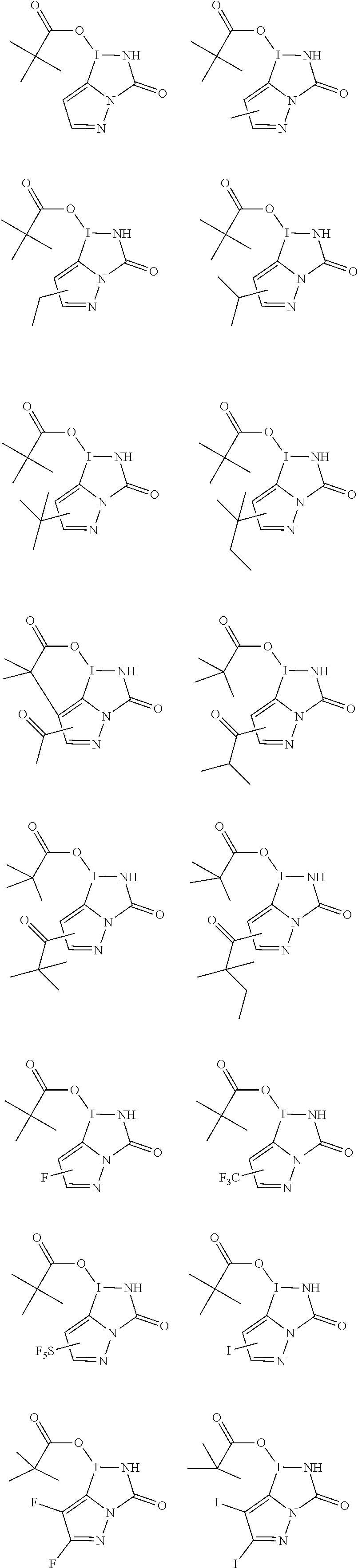

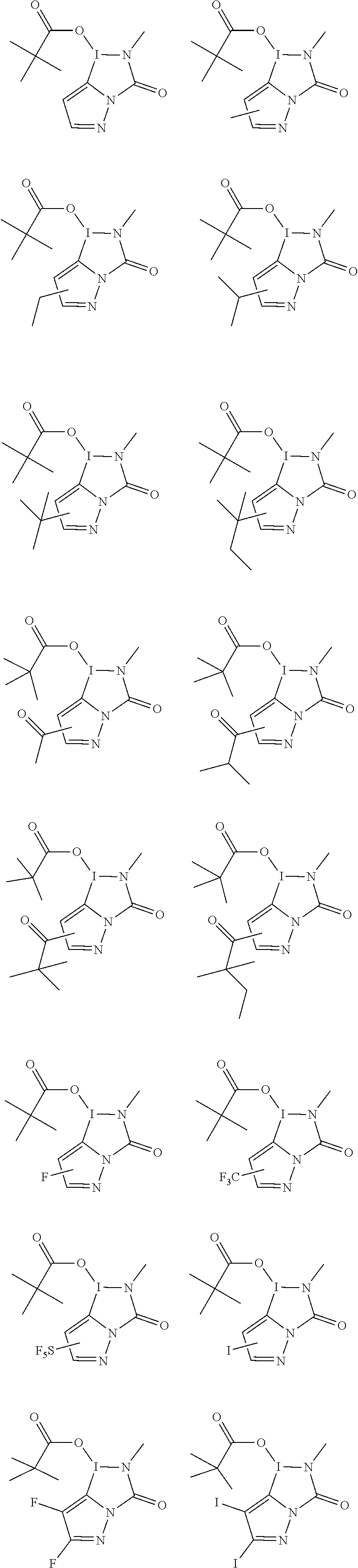

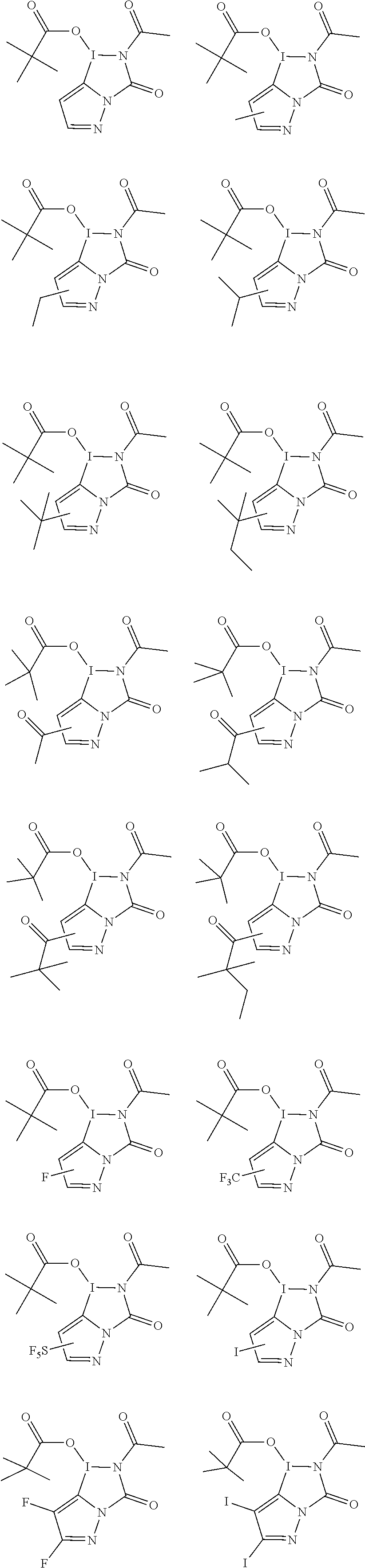

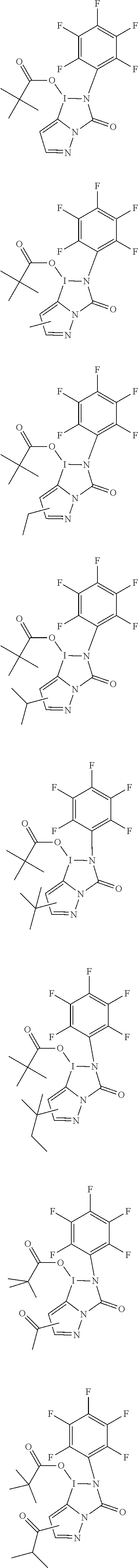

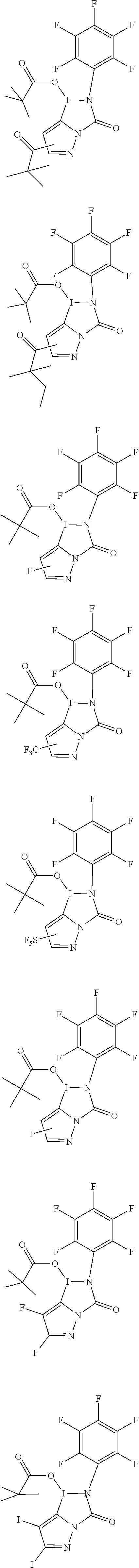

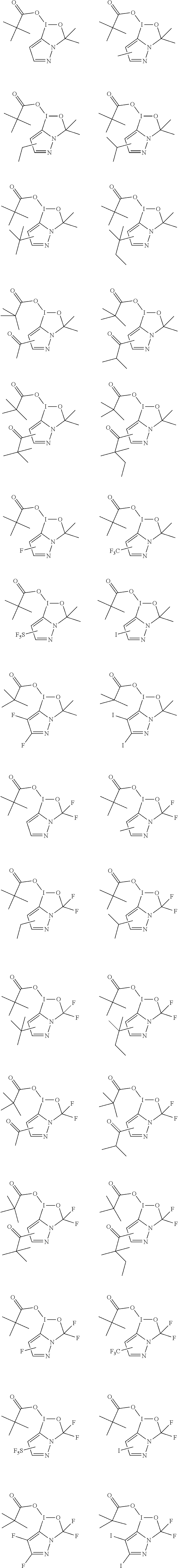

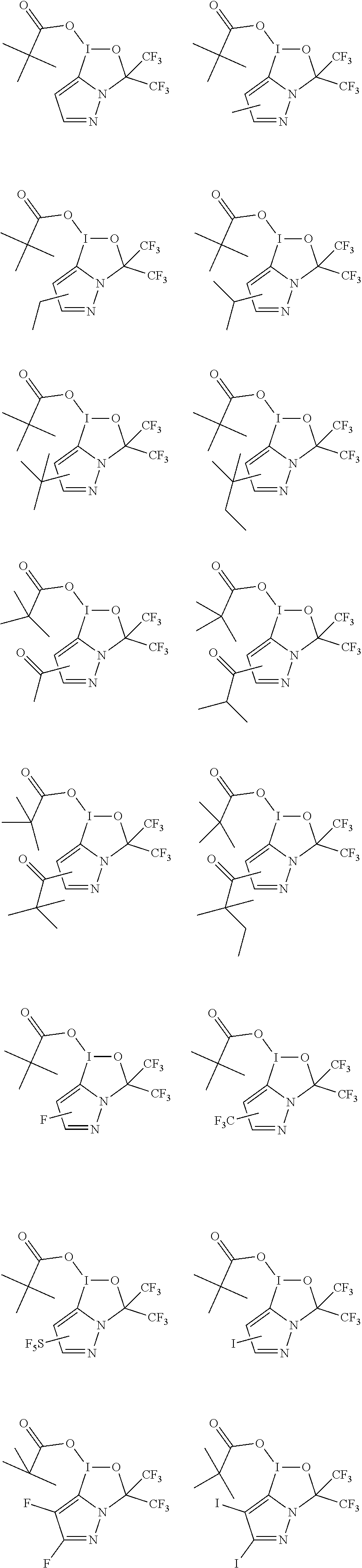

Specific examples of the hypervalent iodine compound represented by the formula (3) include the following, but are not limited thereto. In the following formulae, *5 and *6 represent the same as defined above.

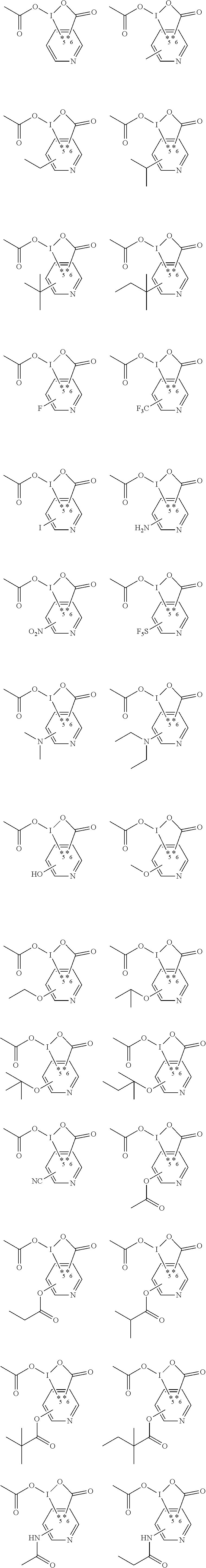

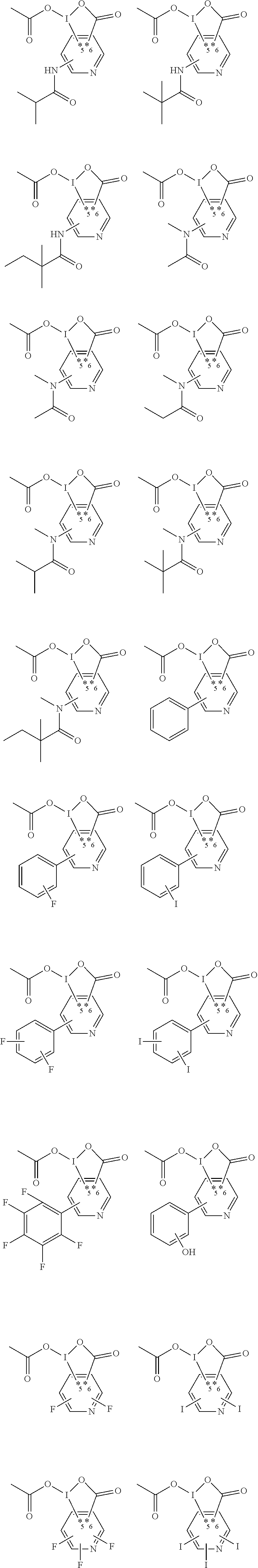

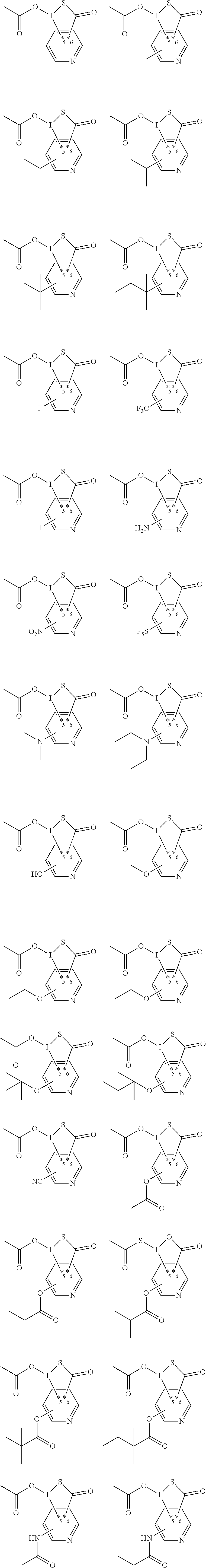

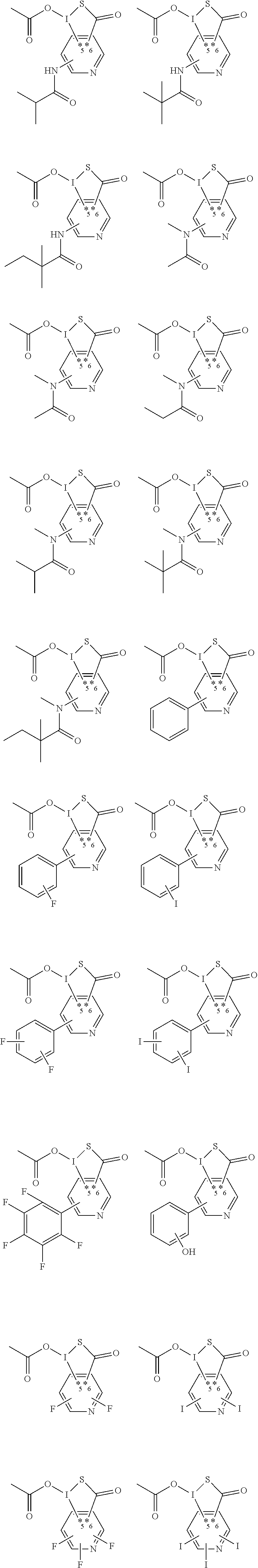

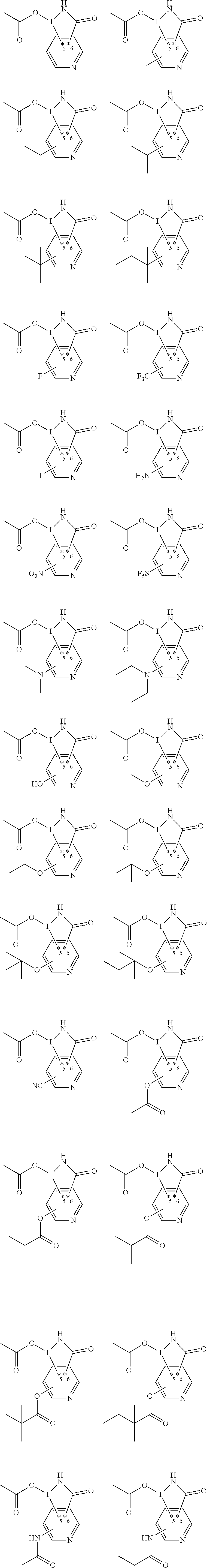

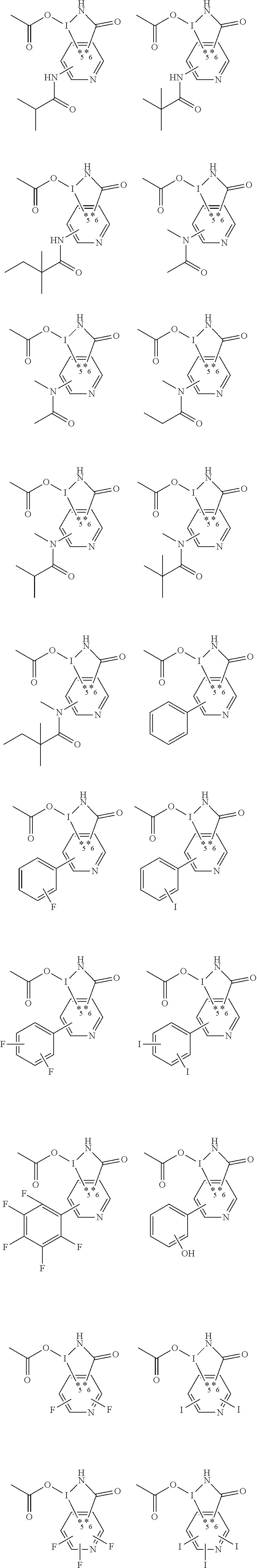

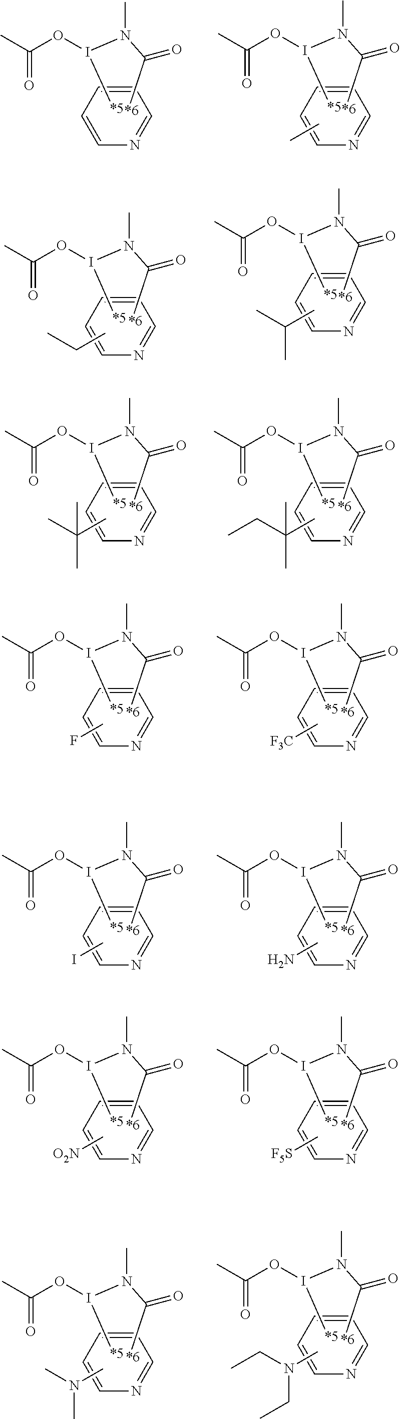

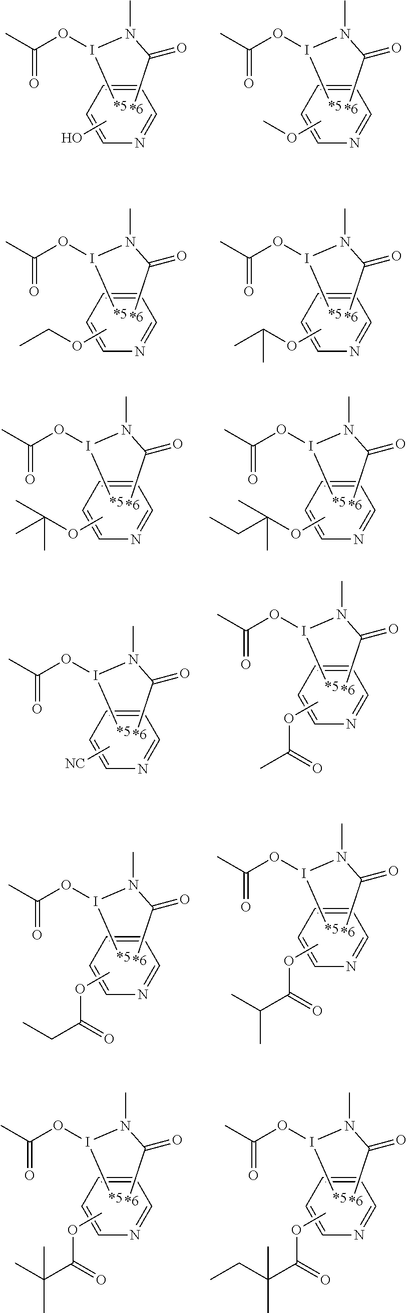

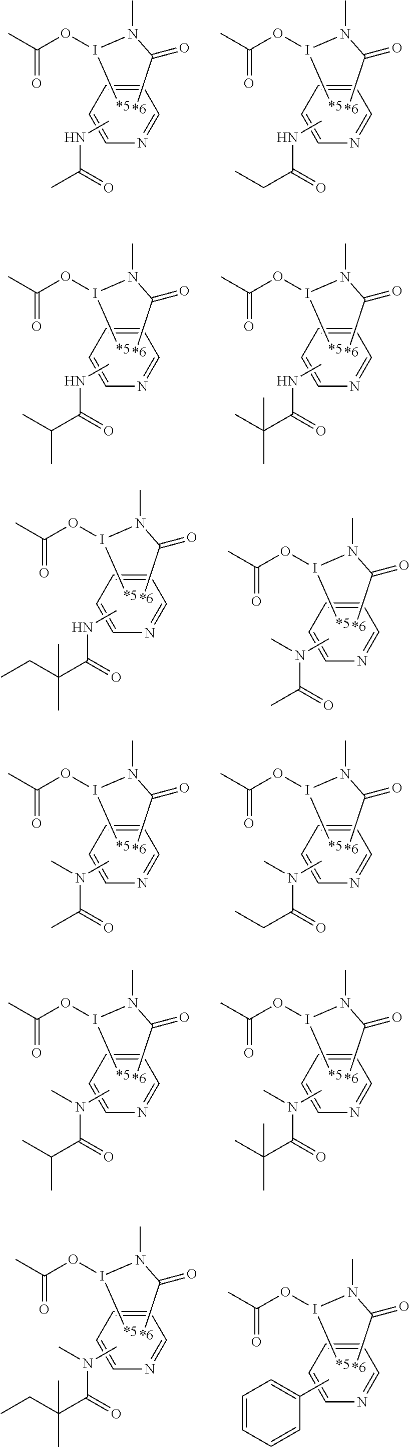

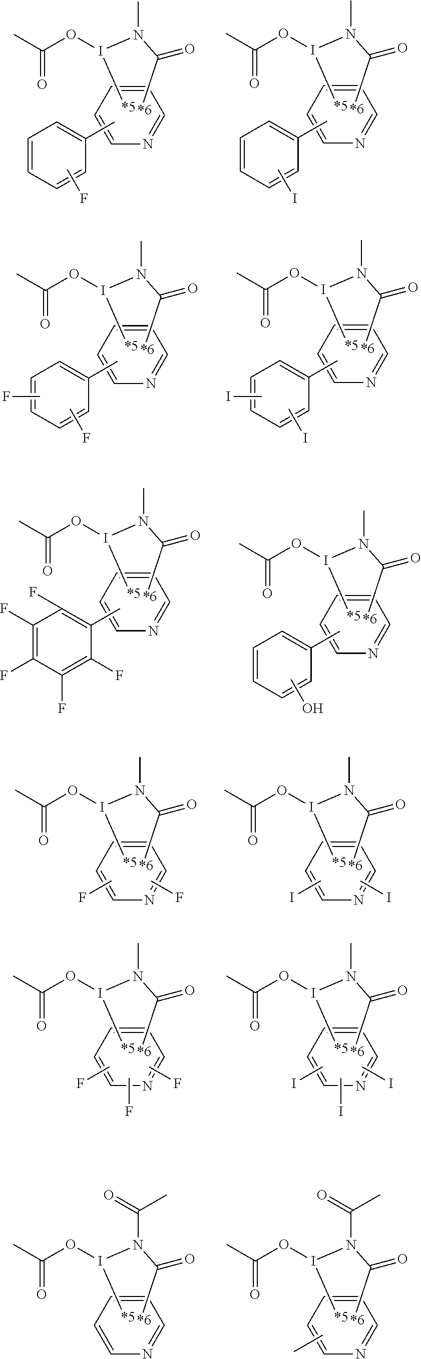

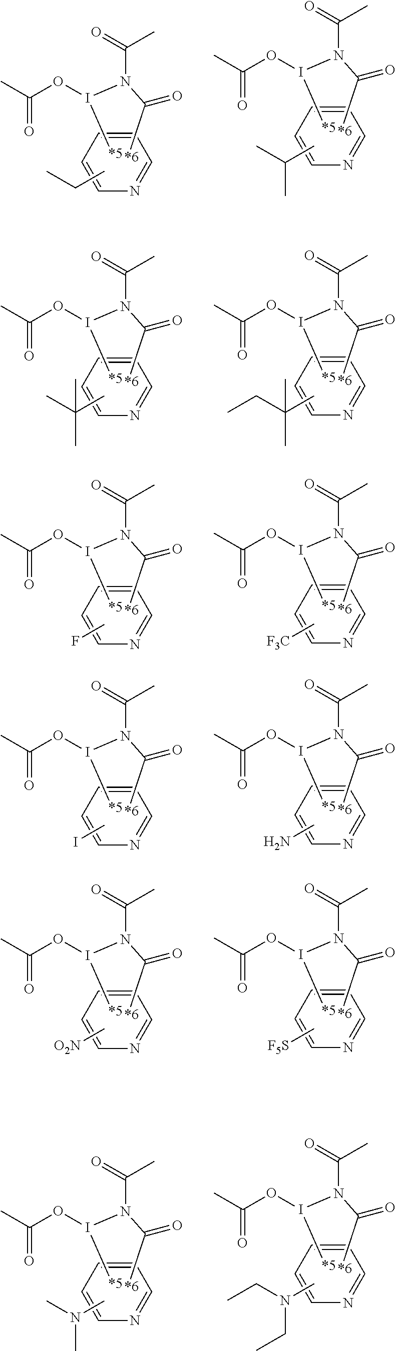

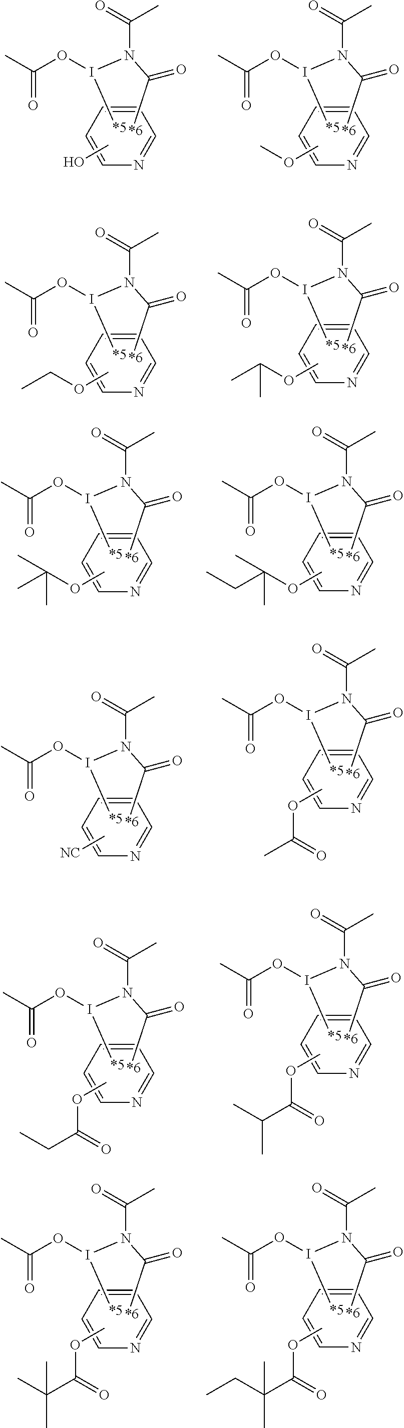

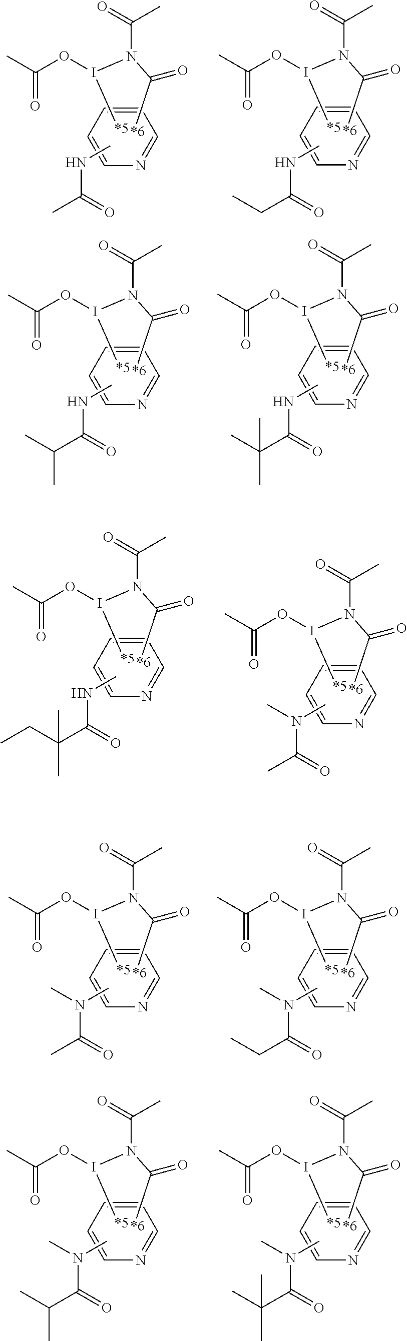

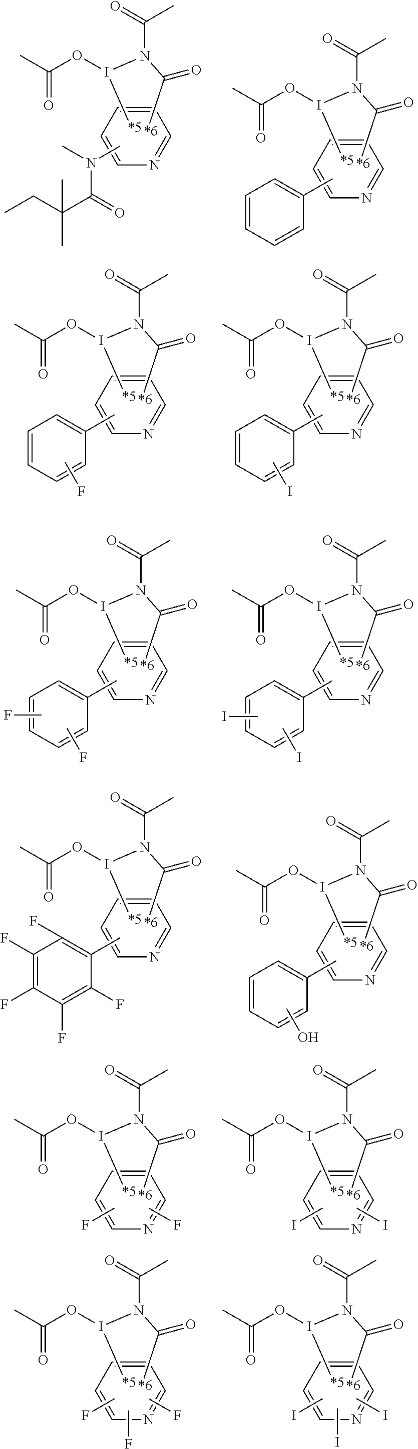

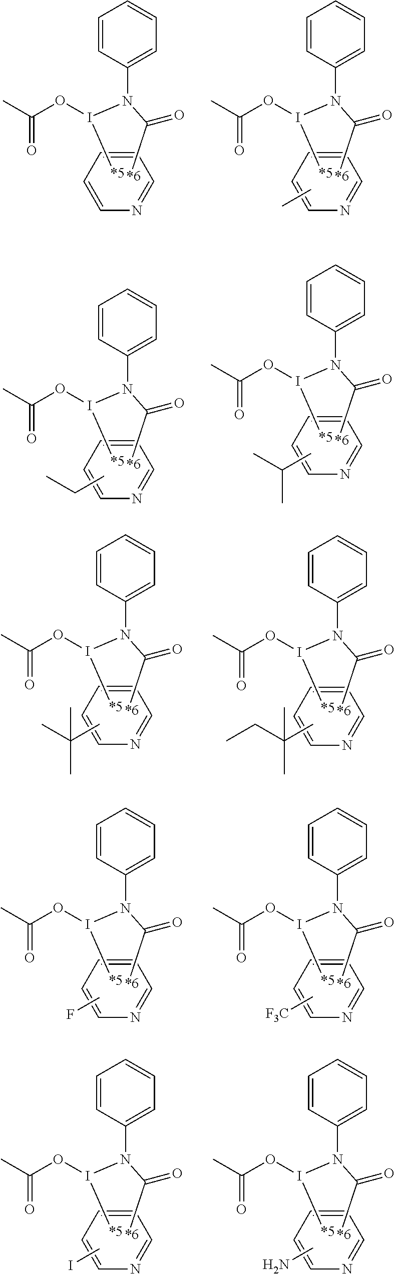

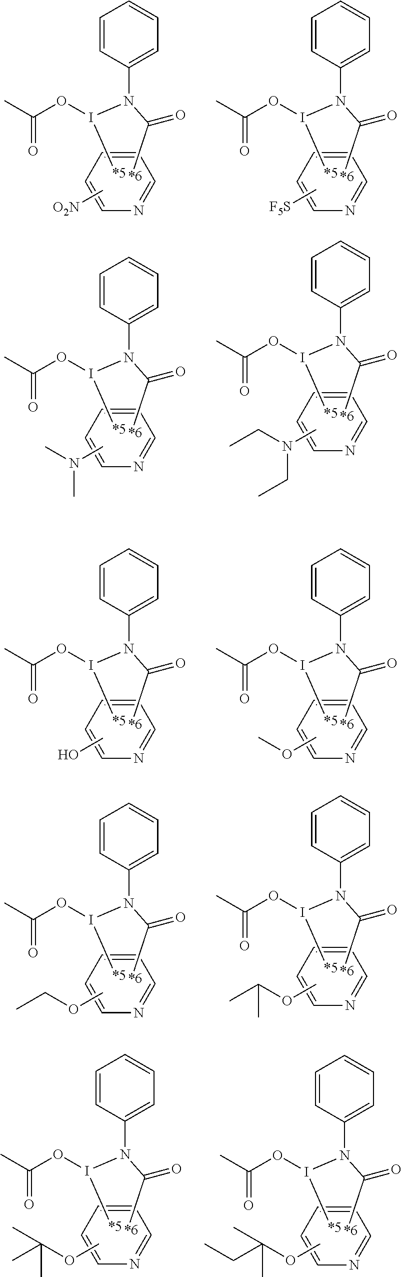

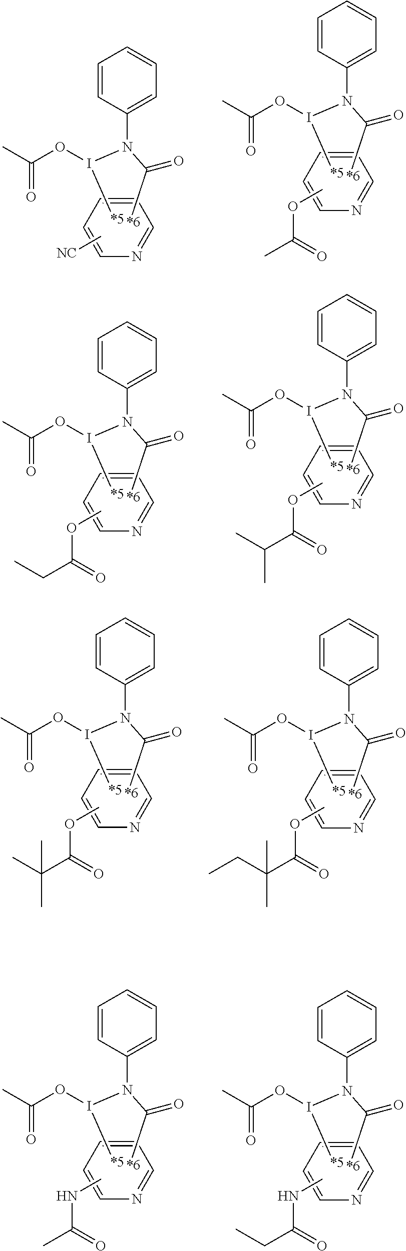

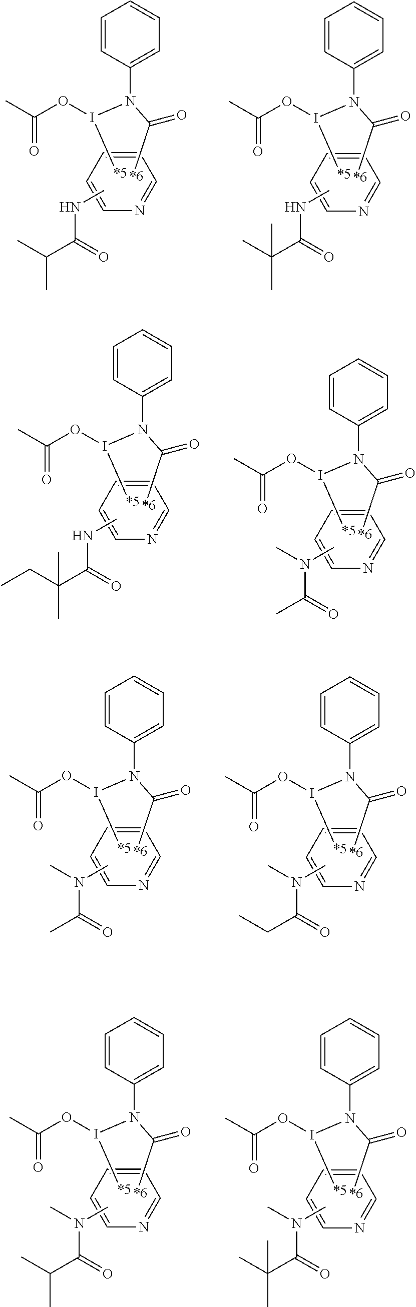

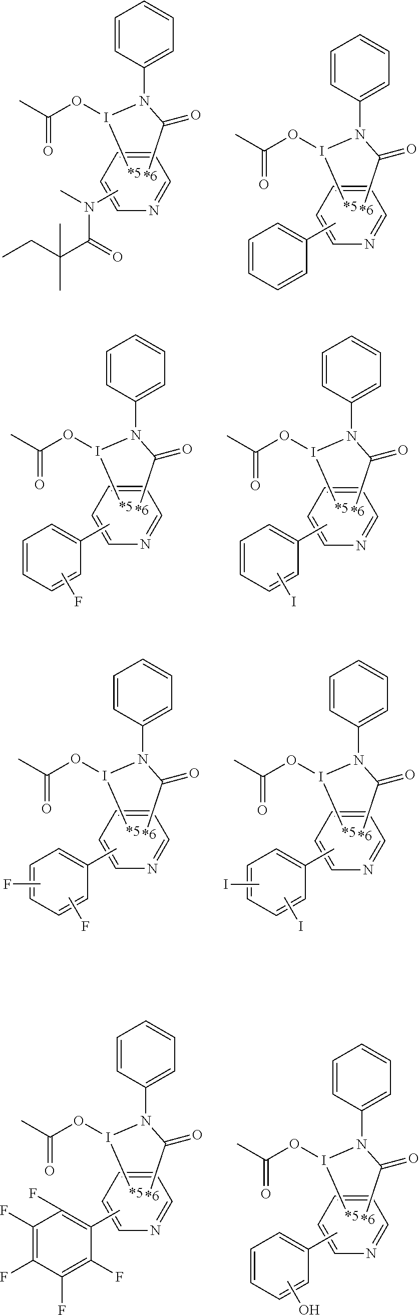

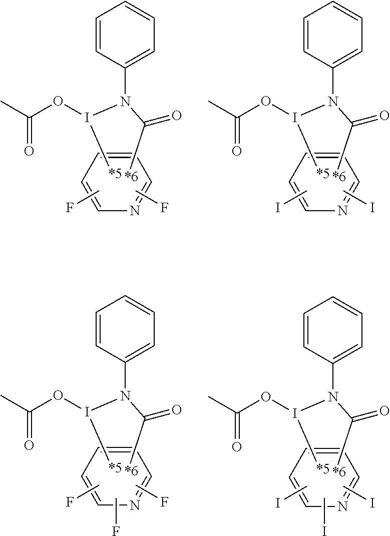

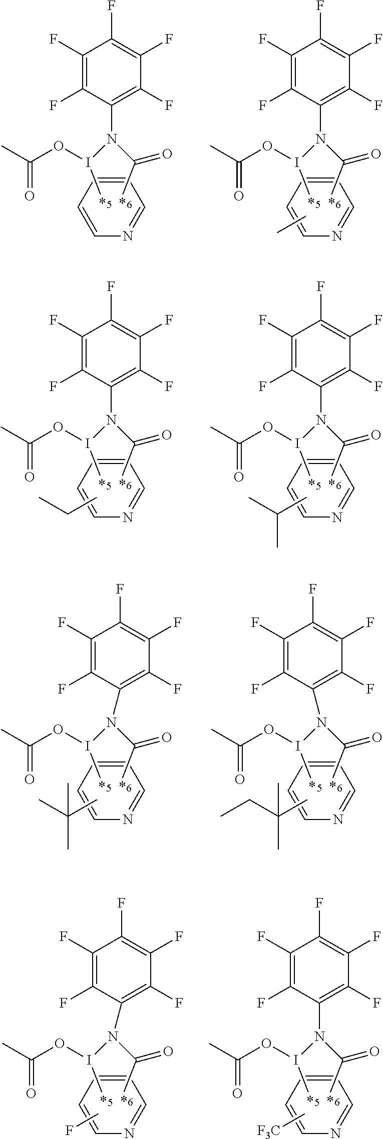

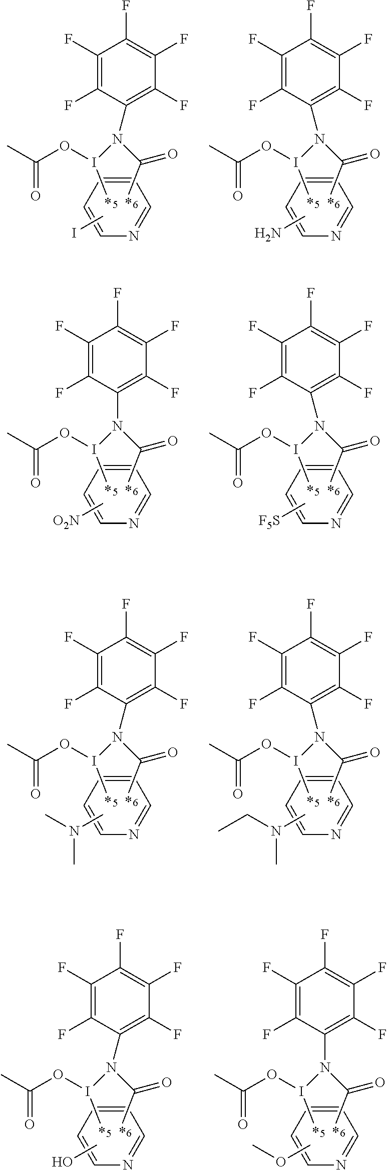

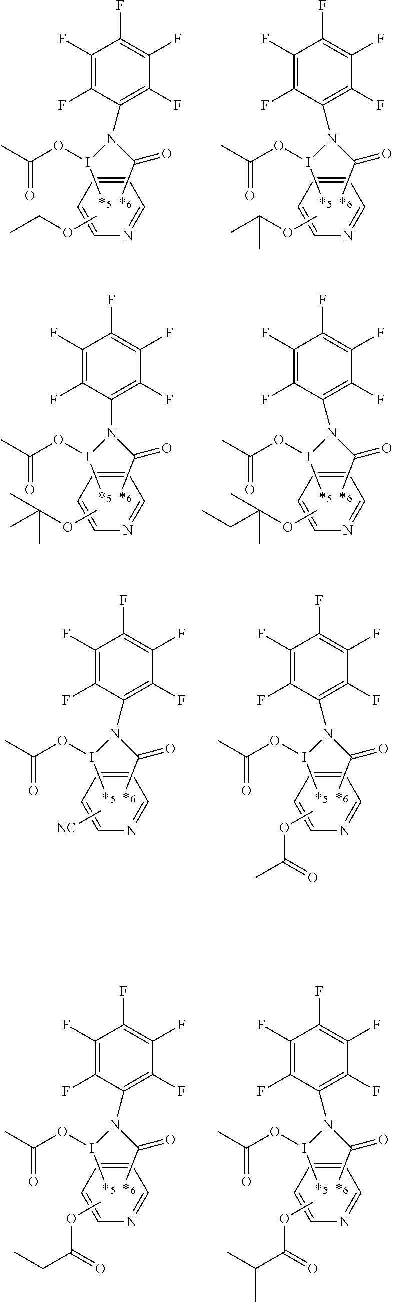

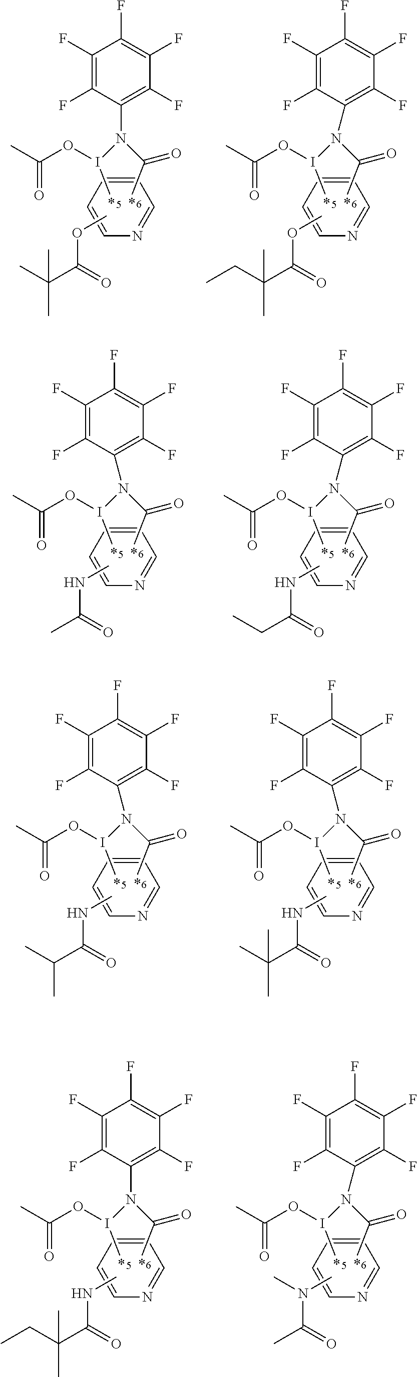

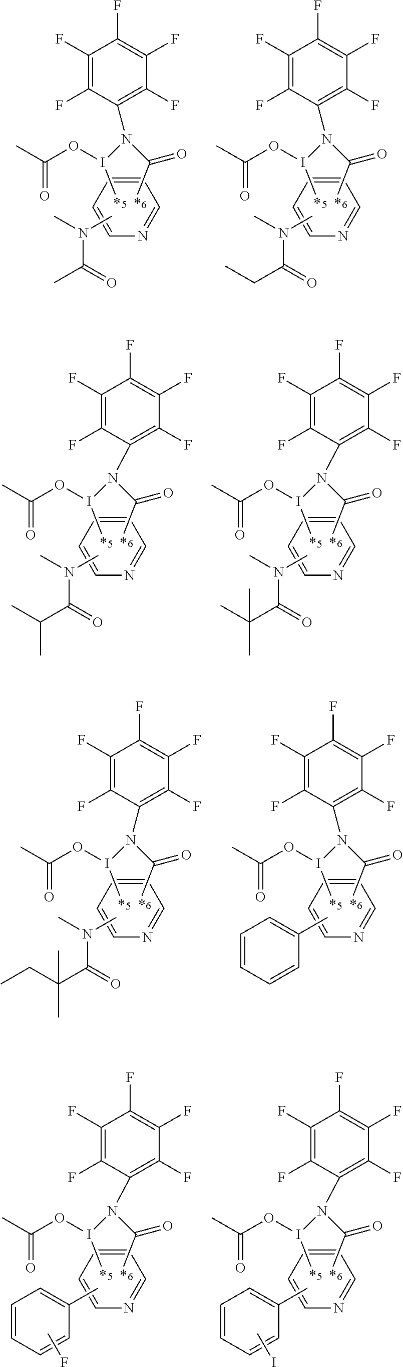

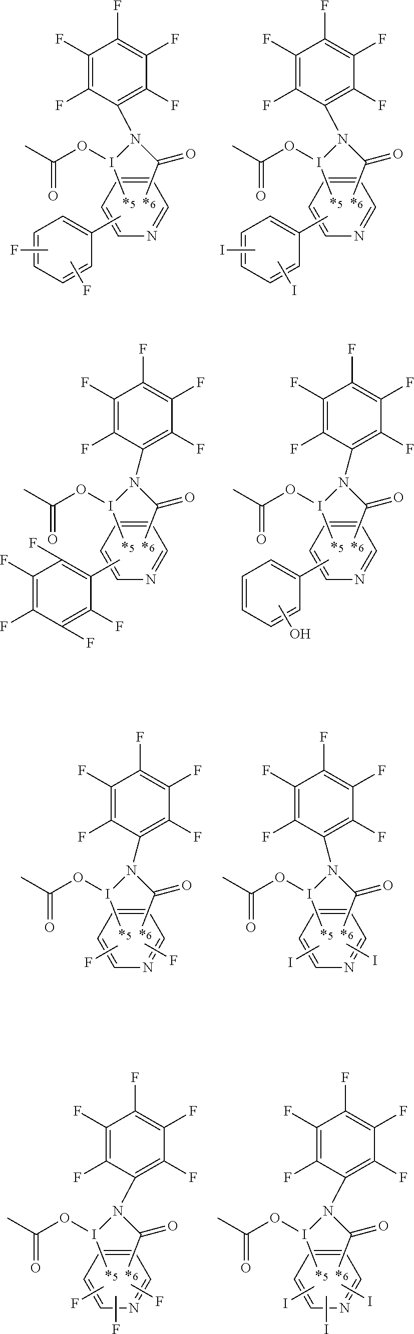

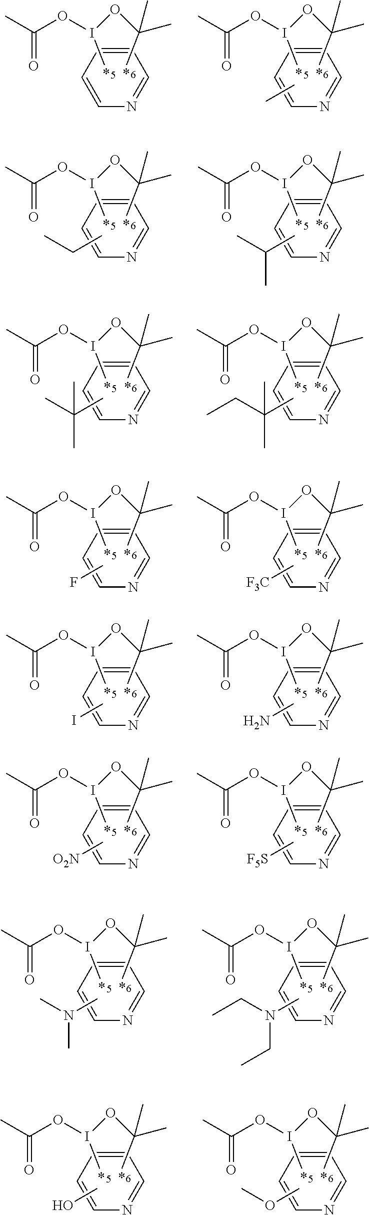

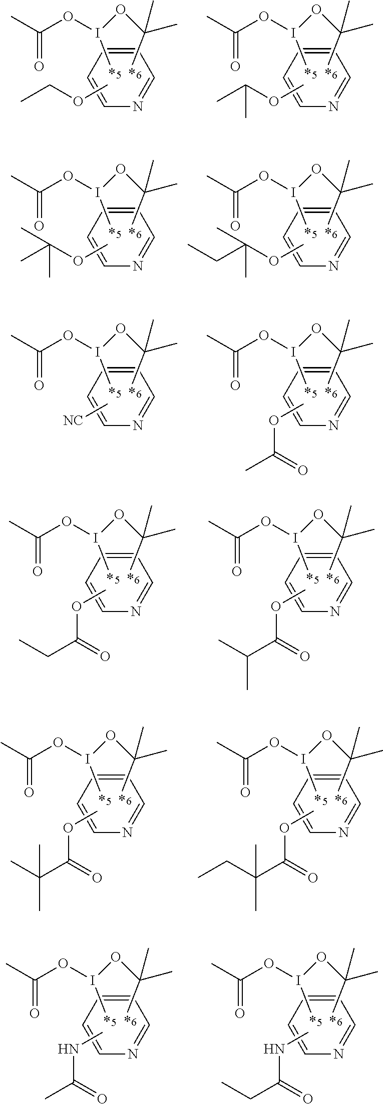

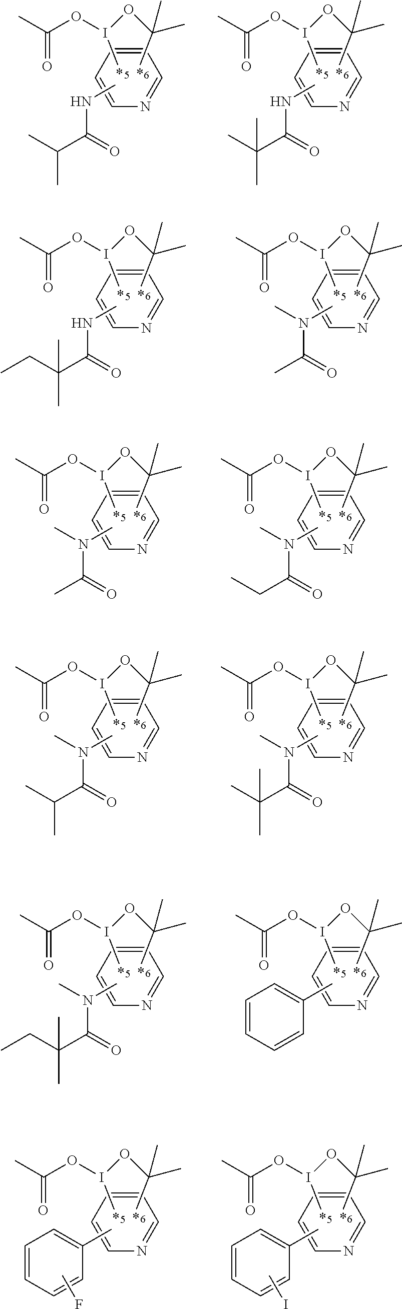

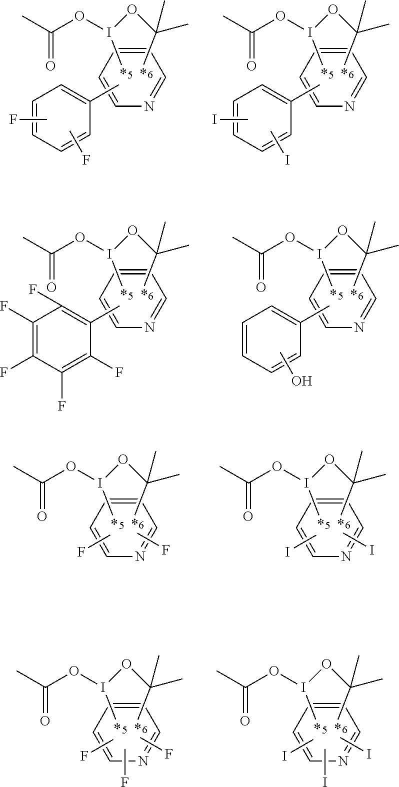

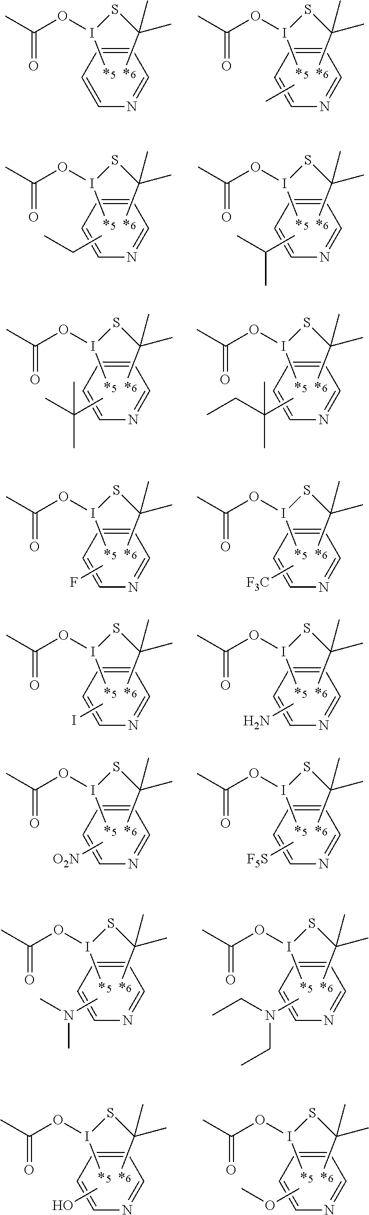

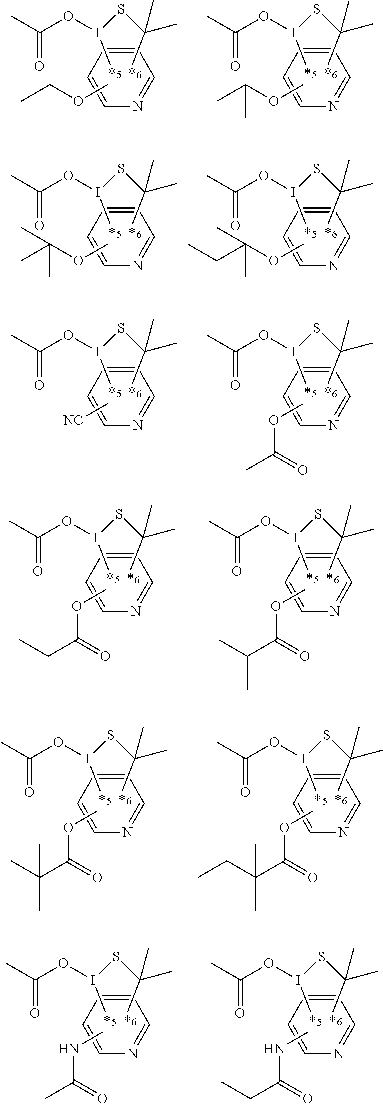

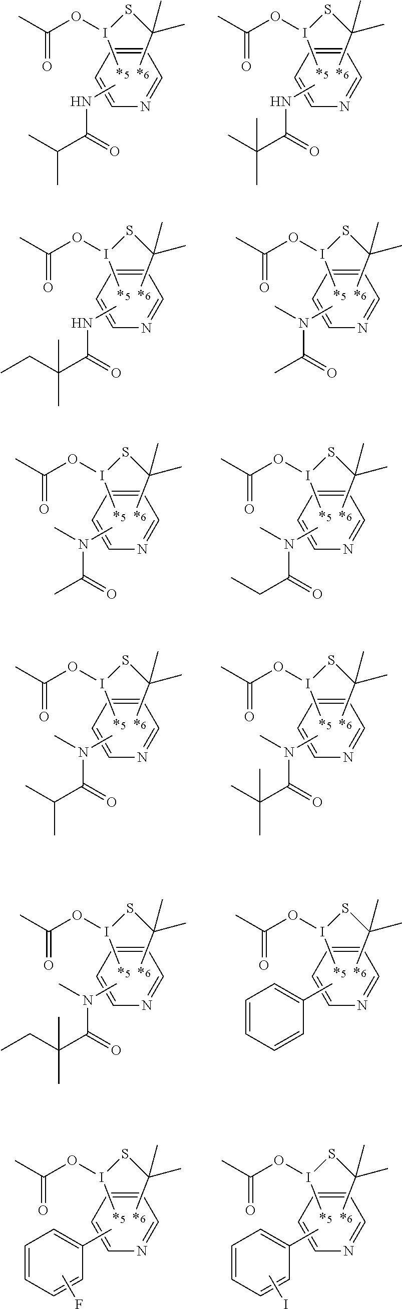

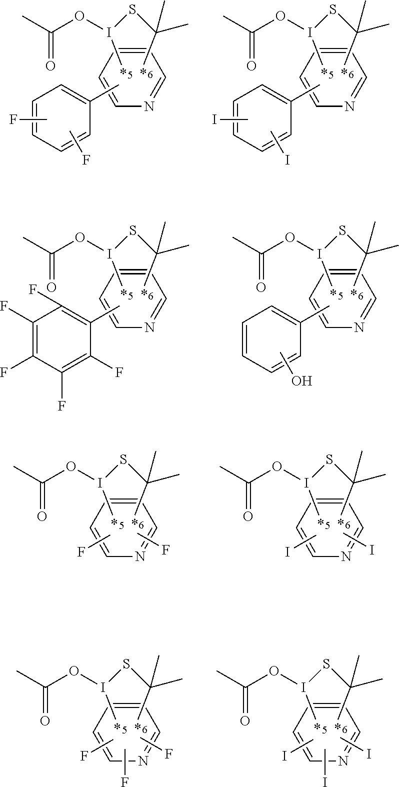

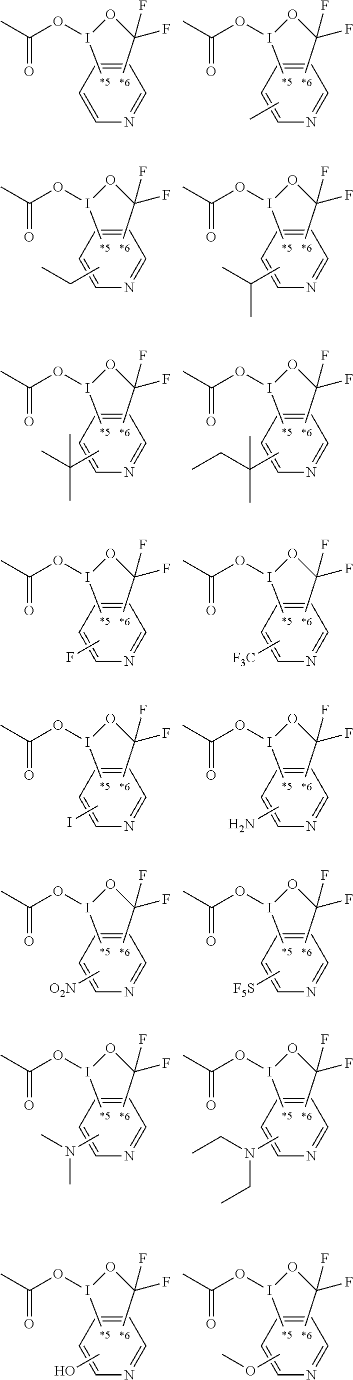

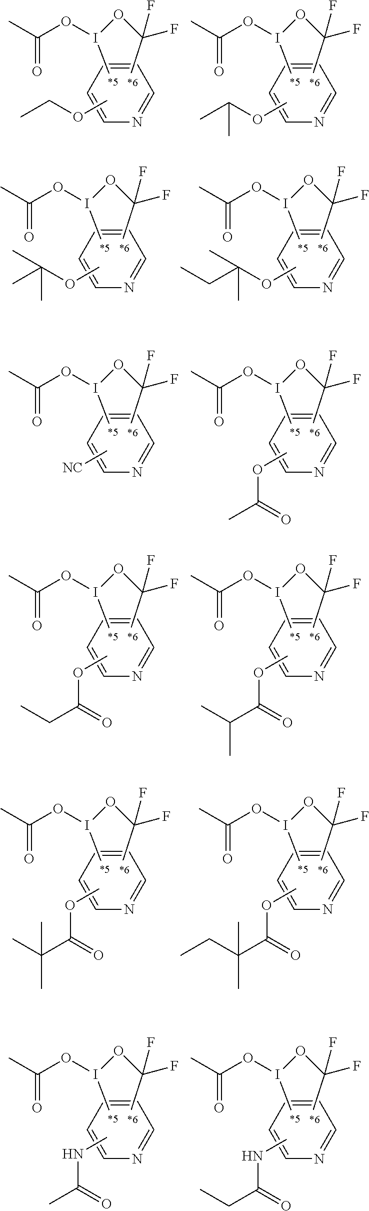

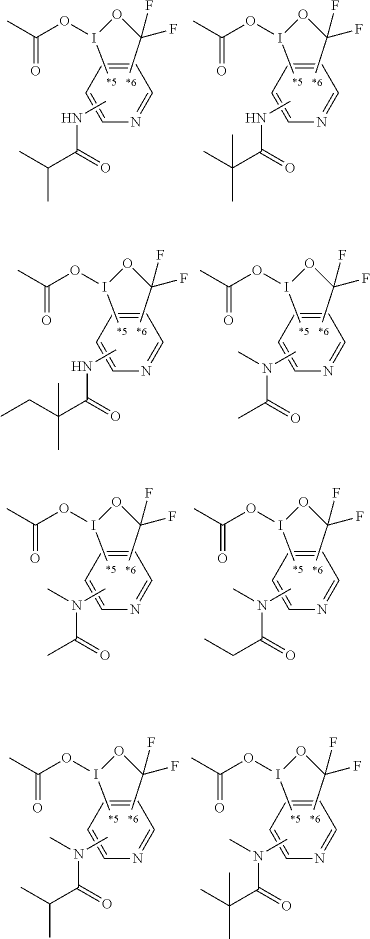

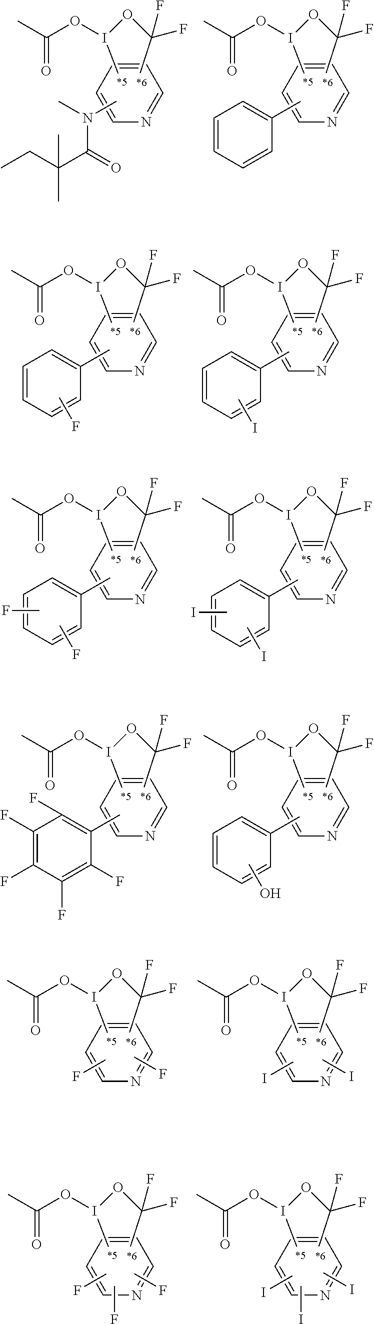

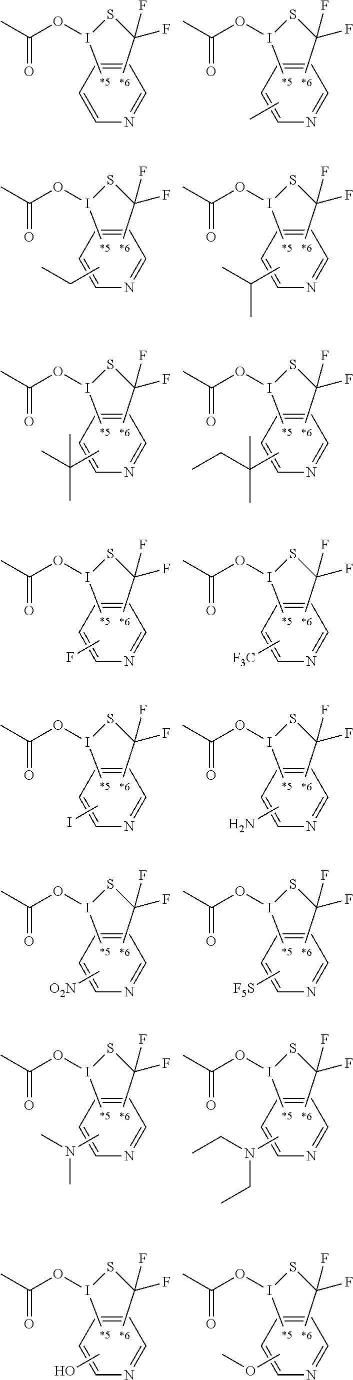

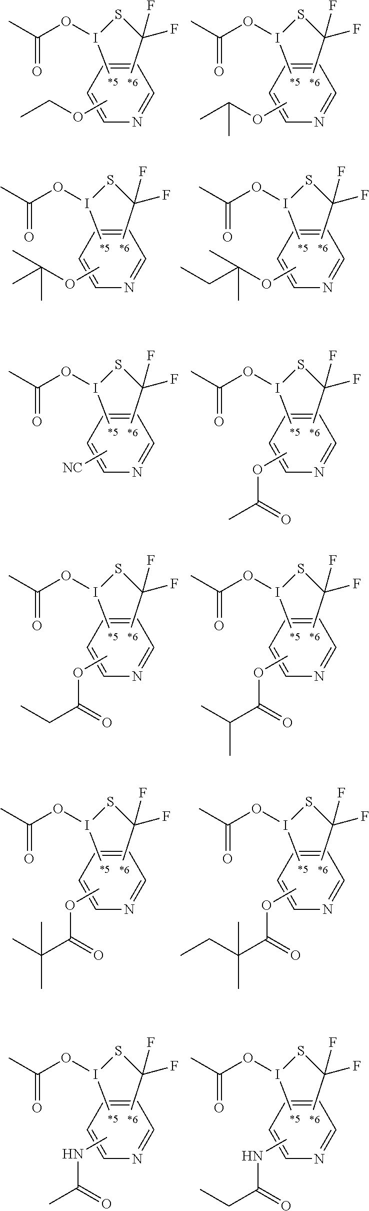

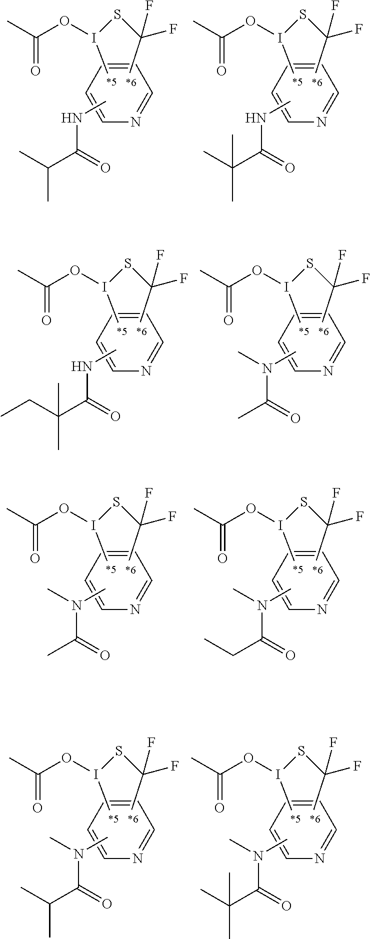

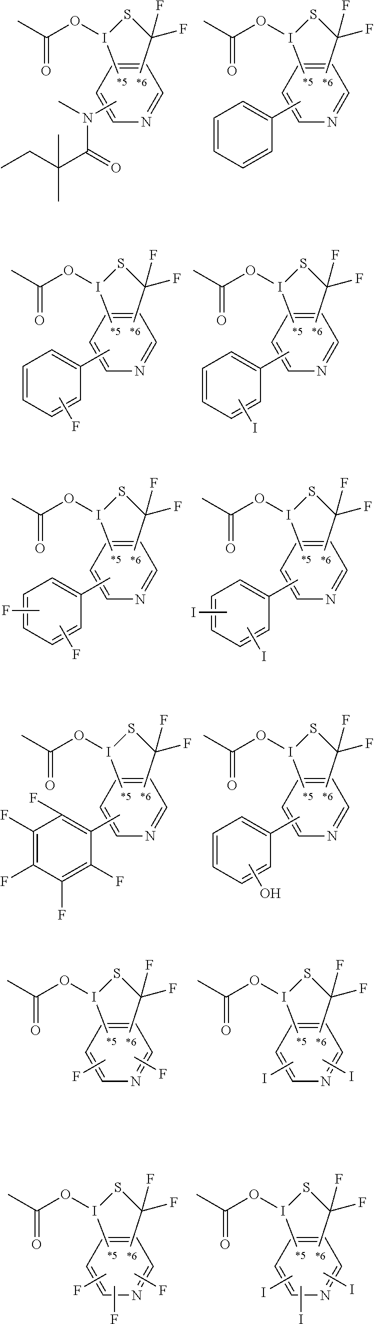

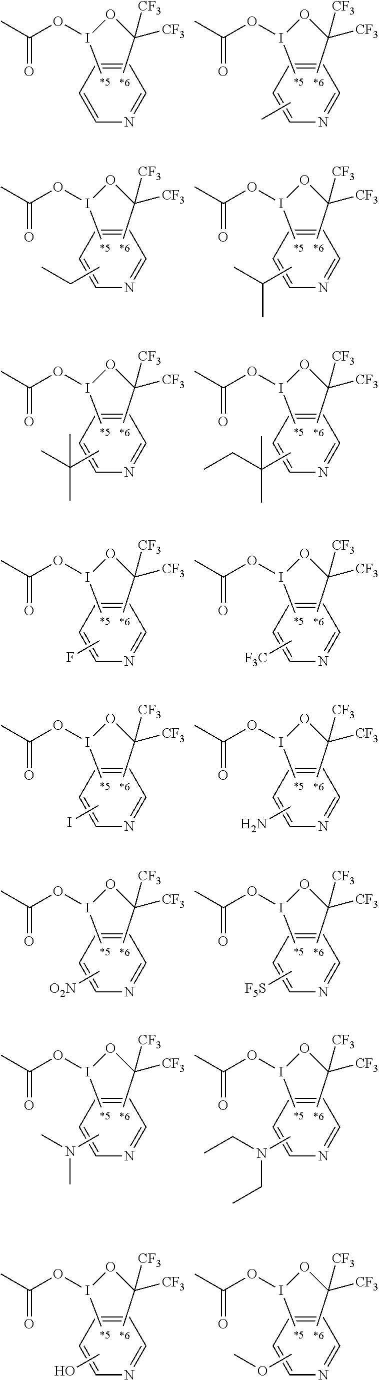

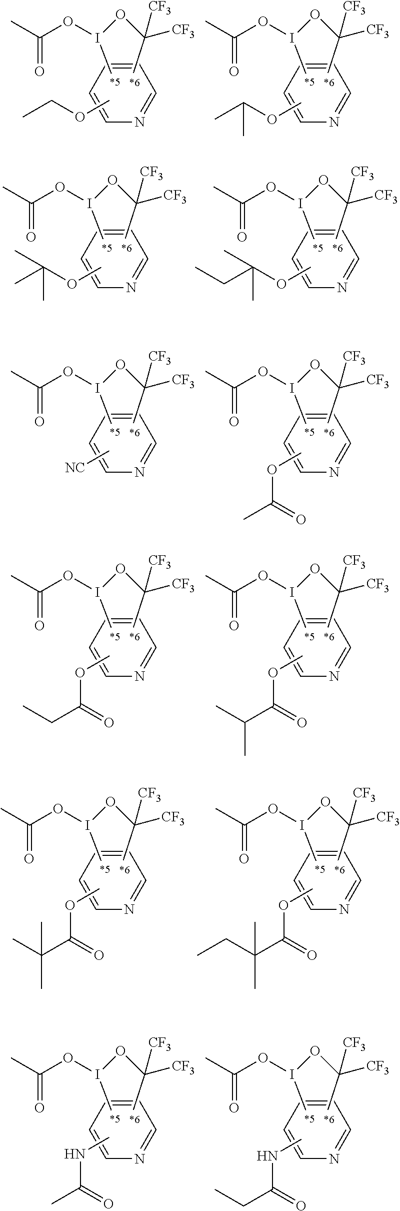

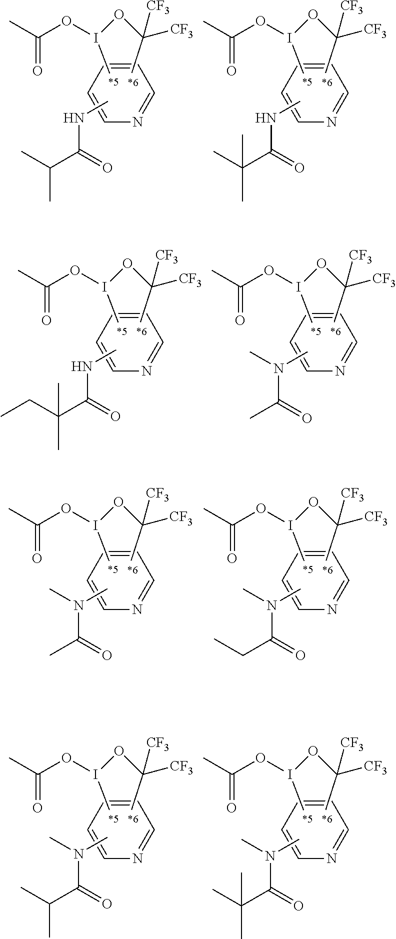

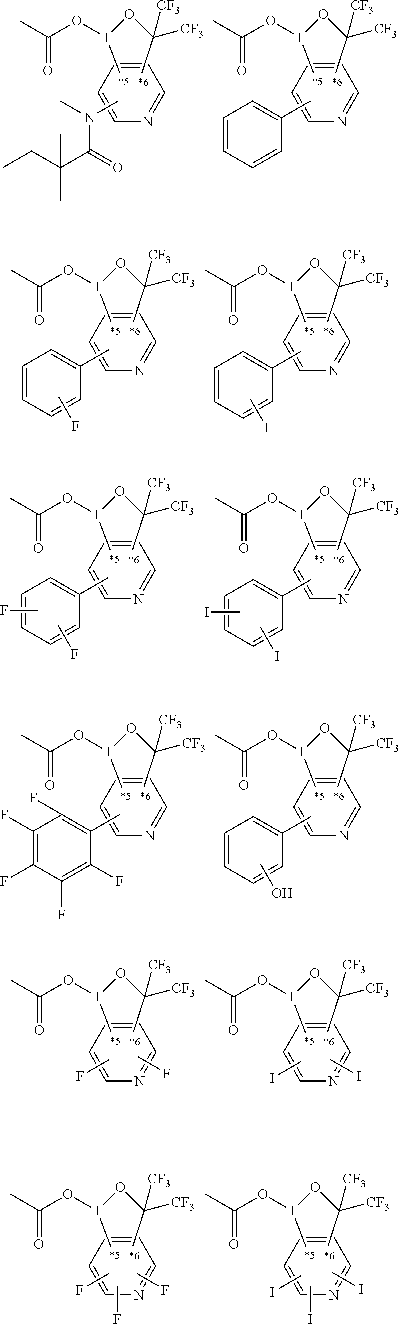

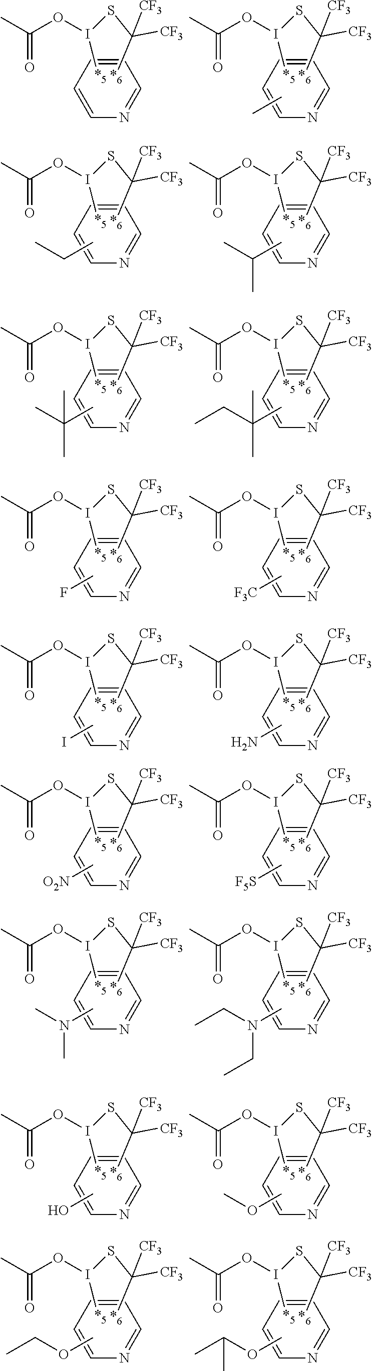

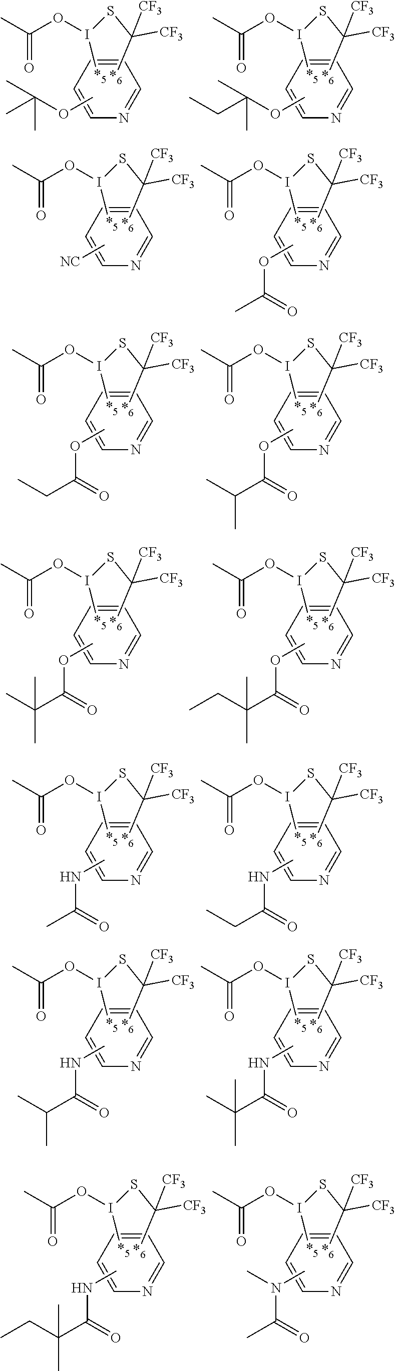

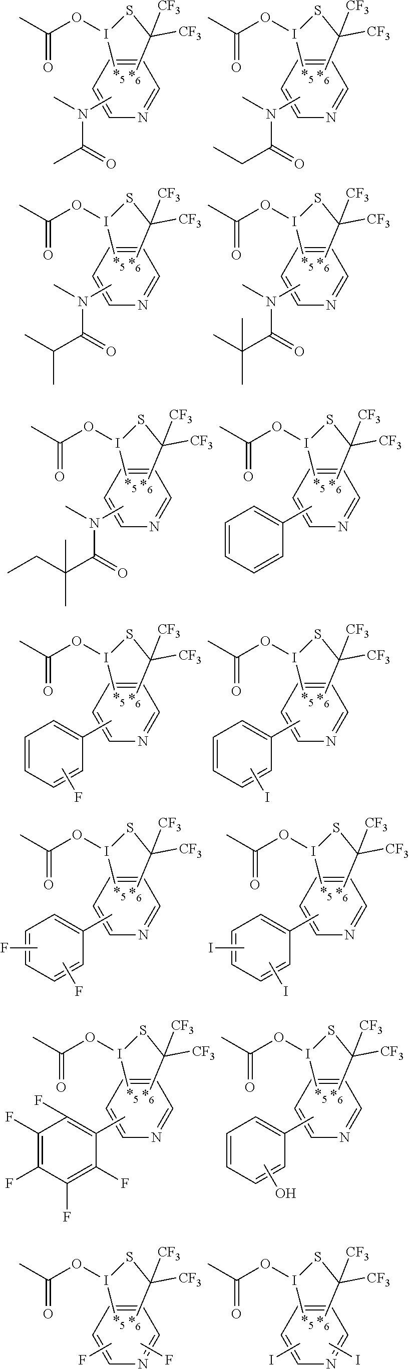

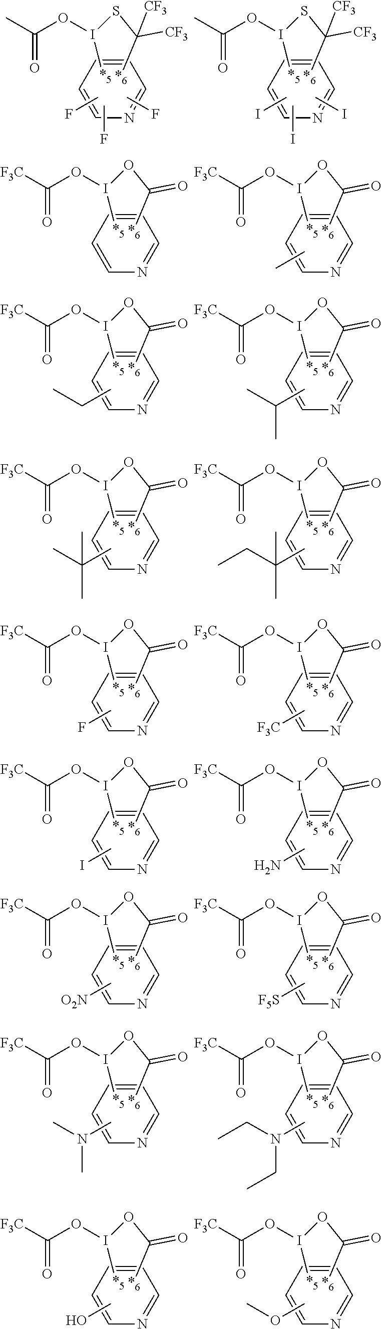

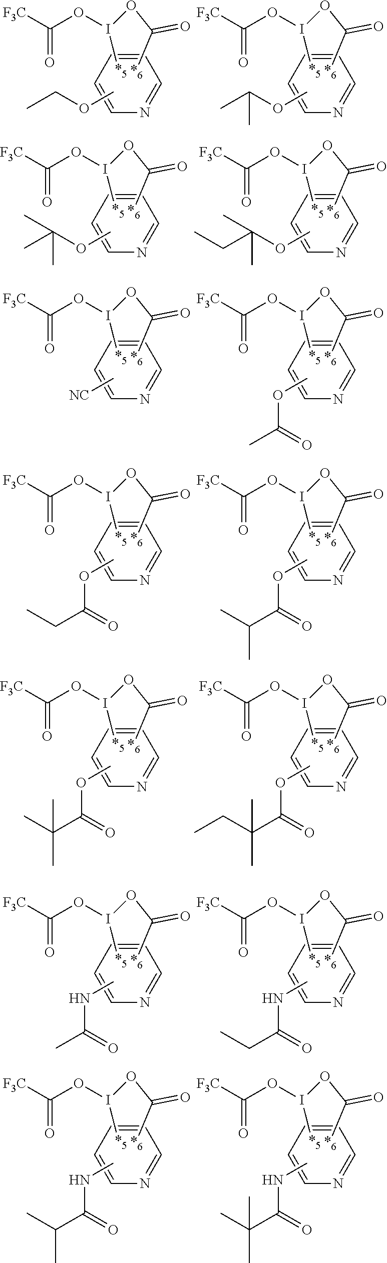

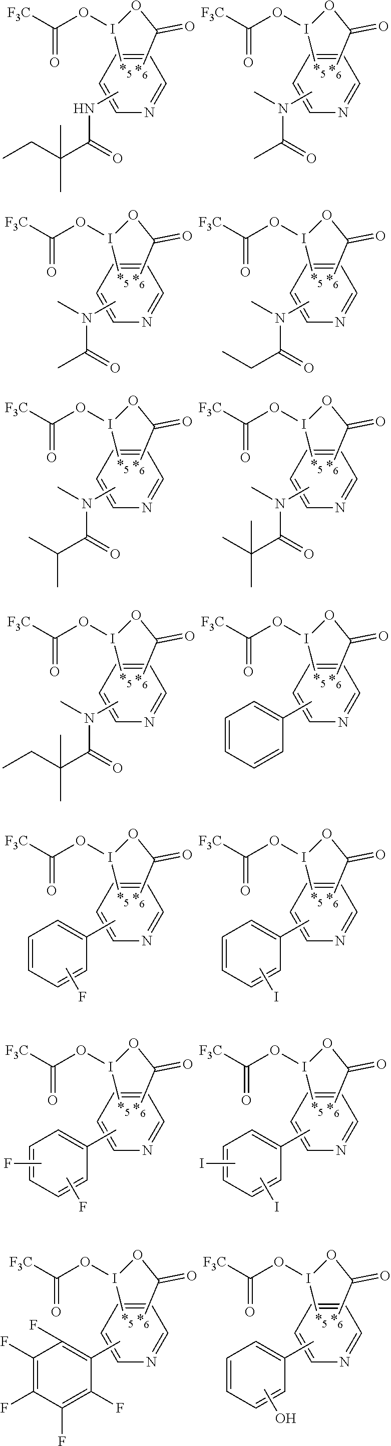

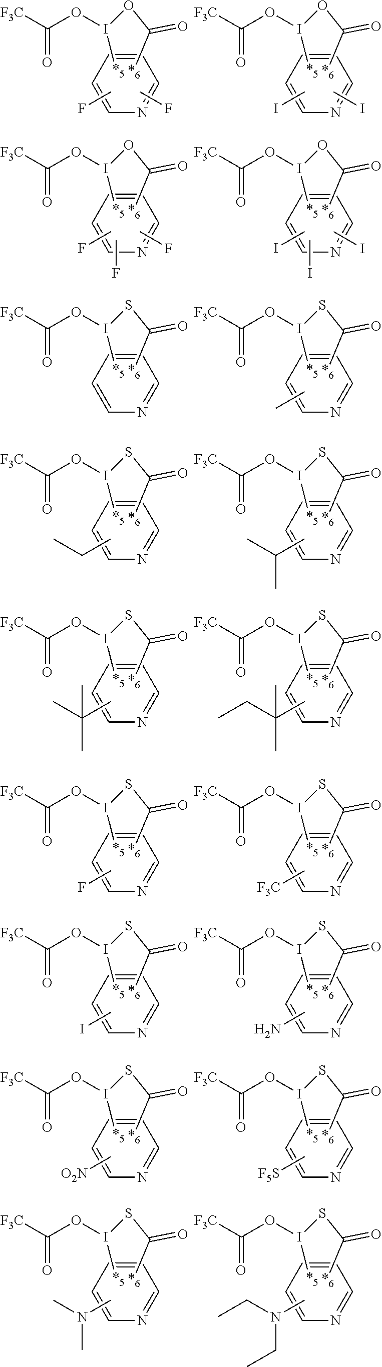

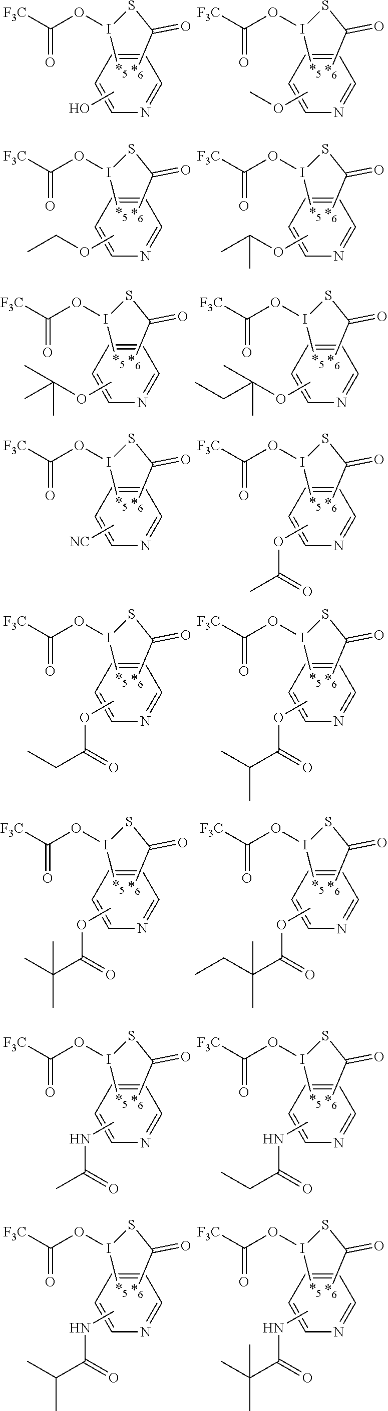

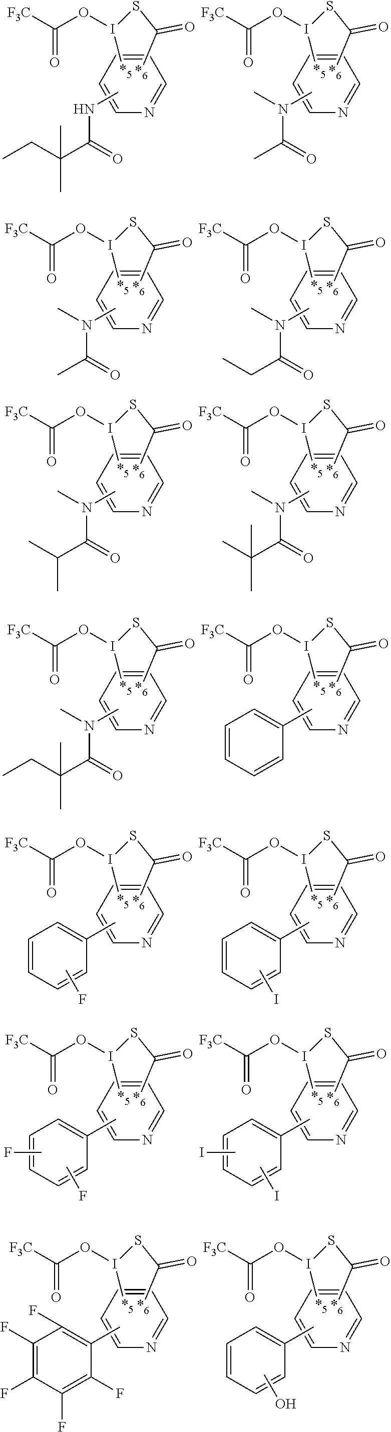

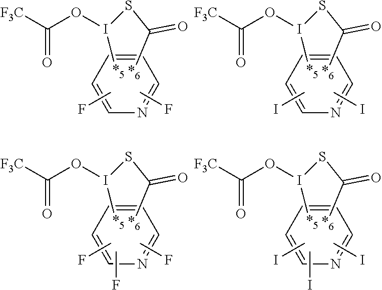

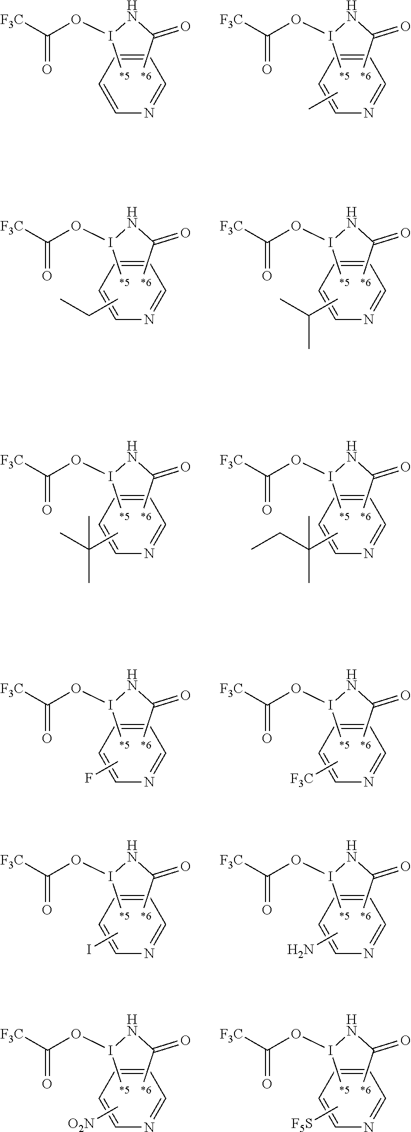

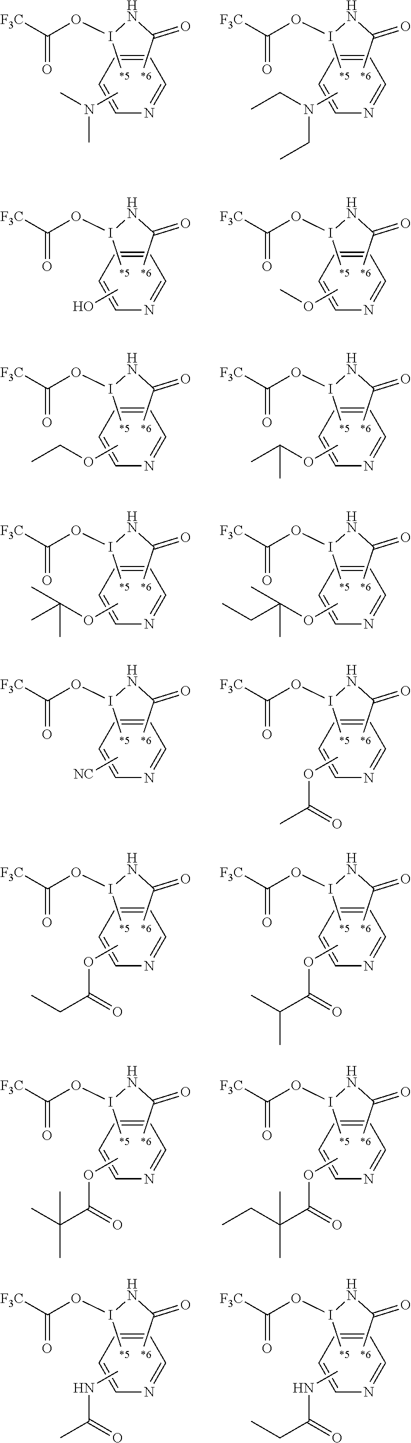

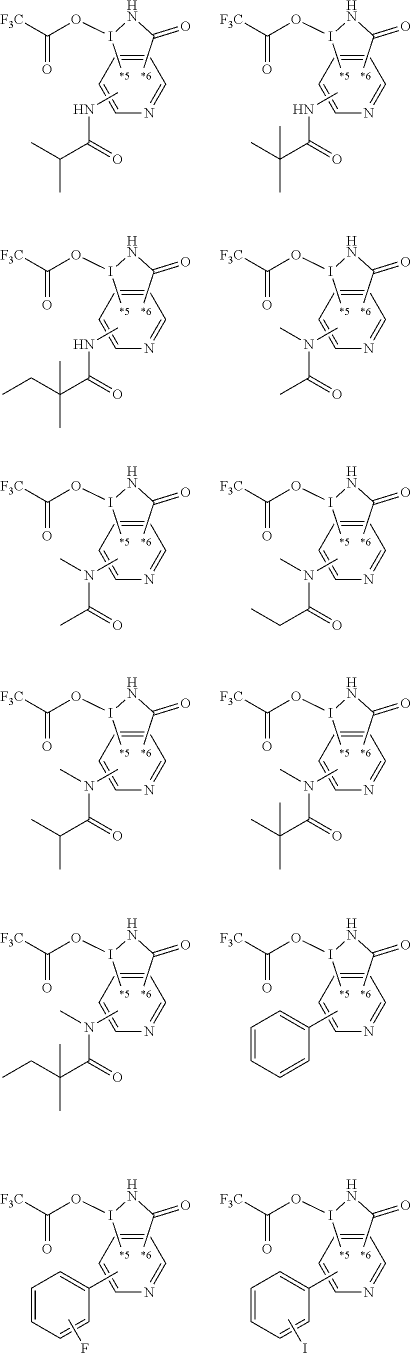

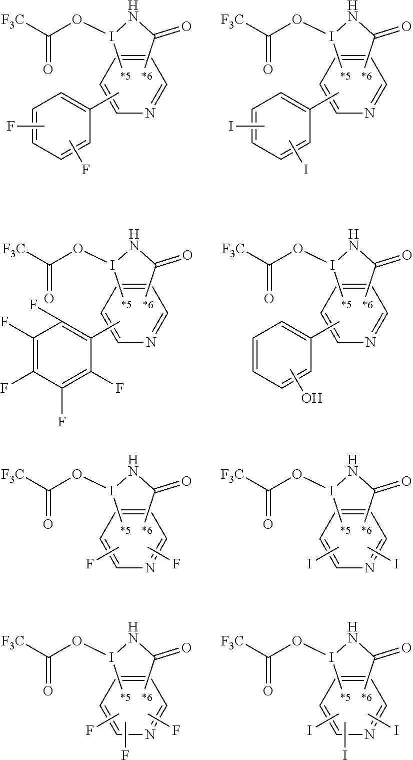

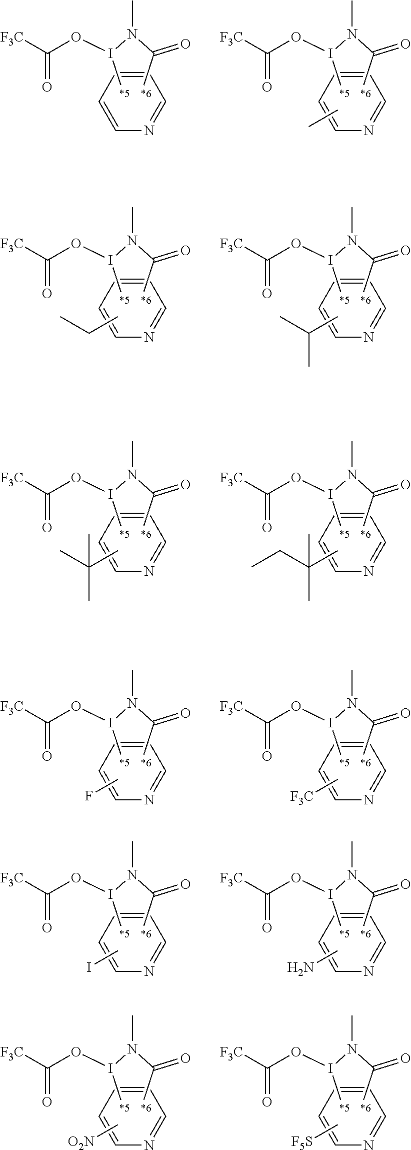

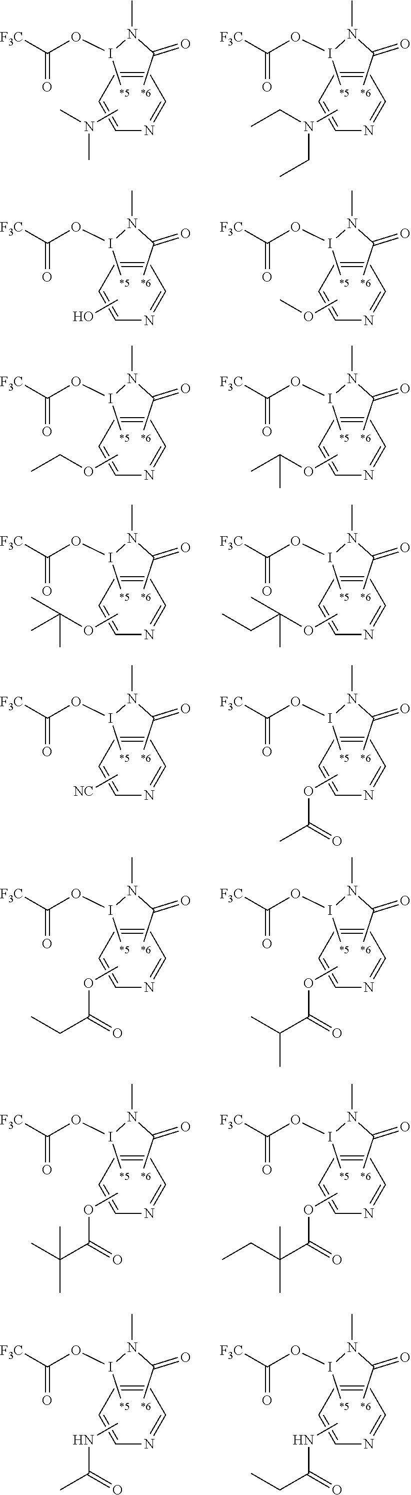

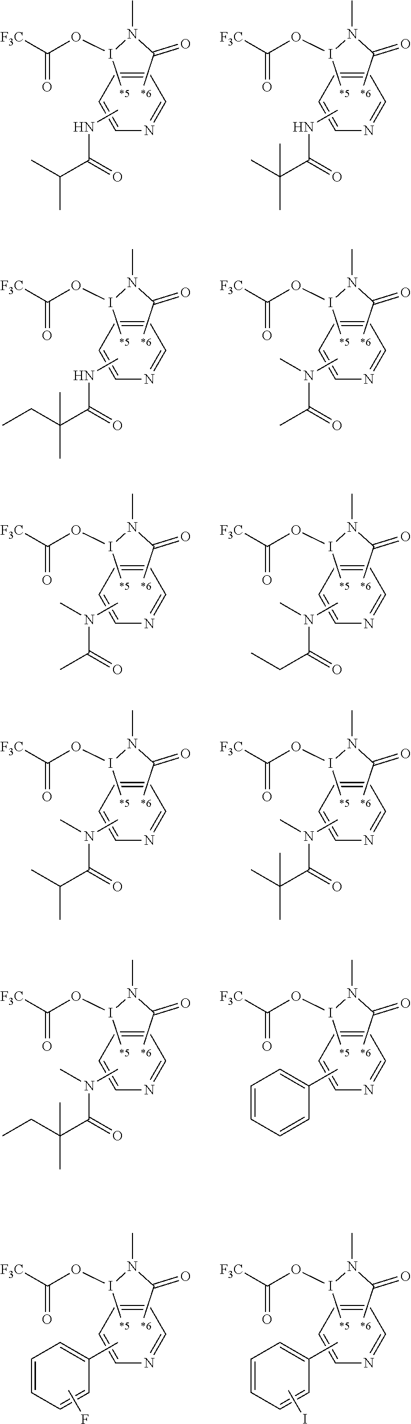

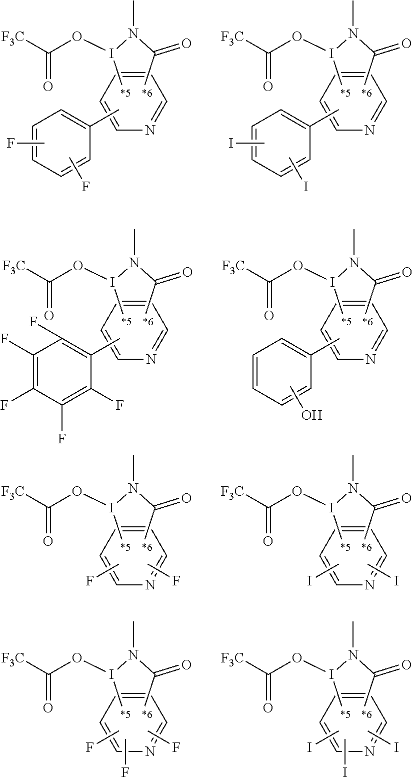

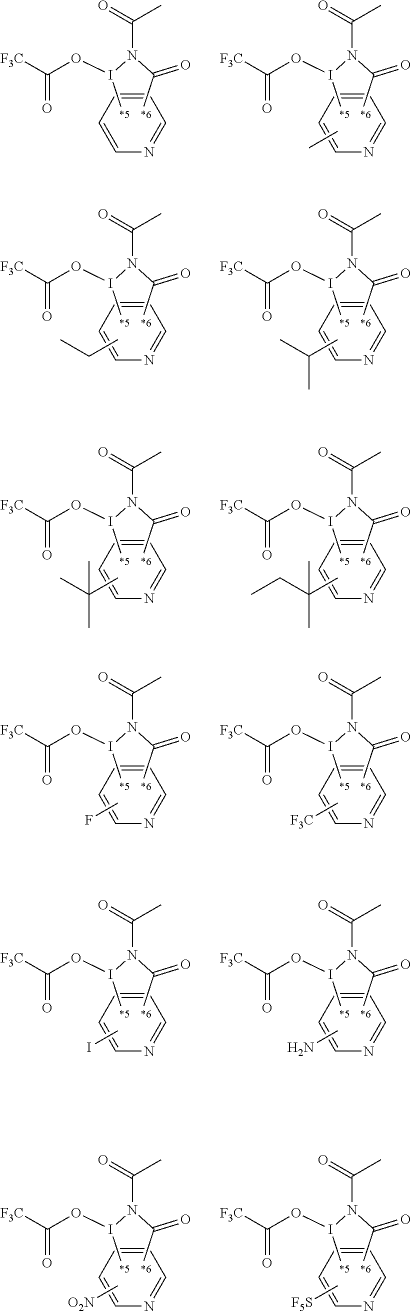

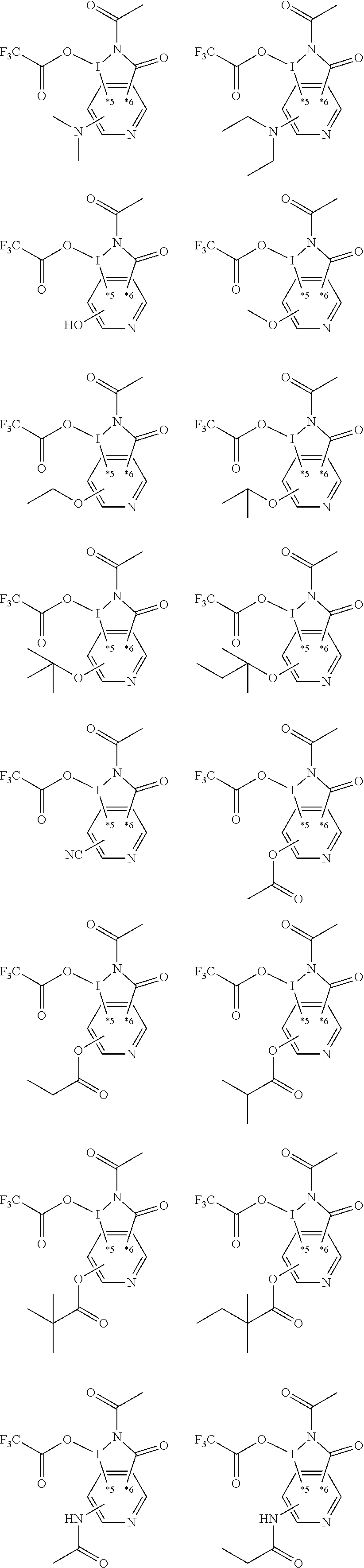

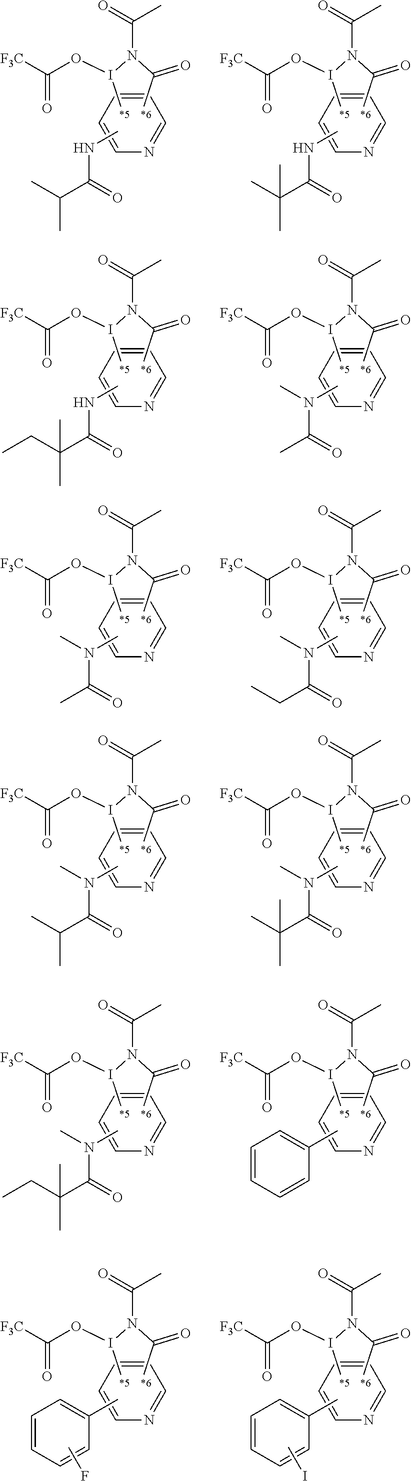

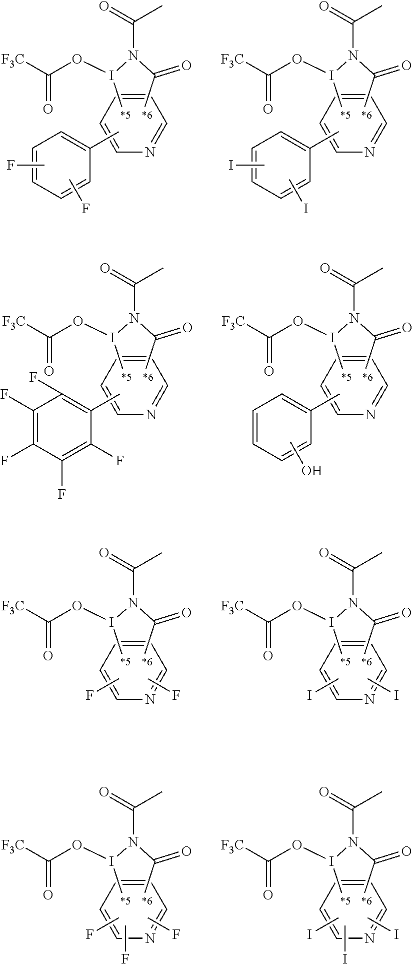

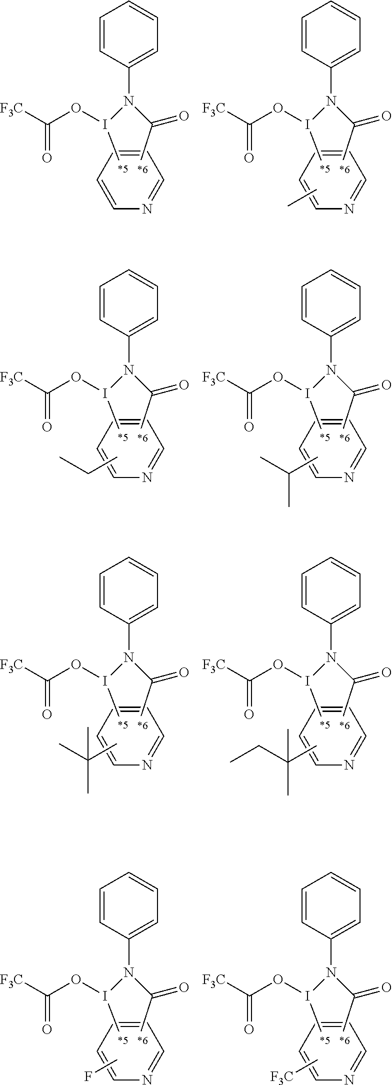

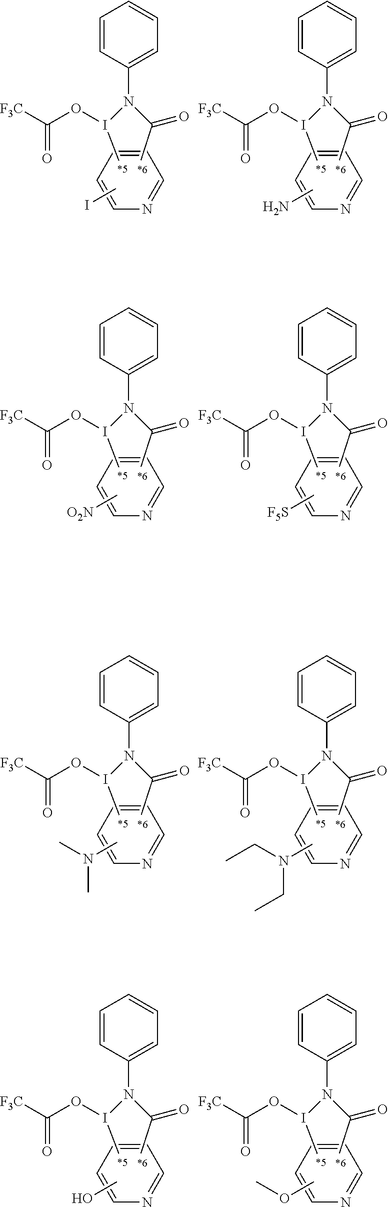

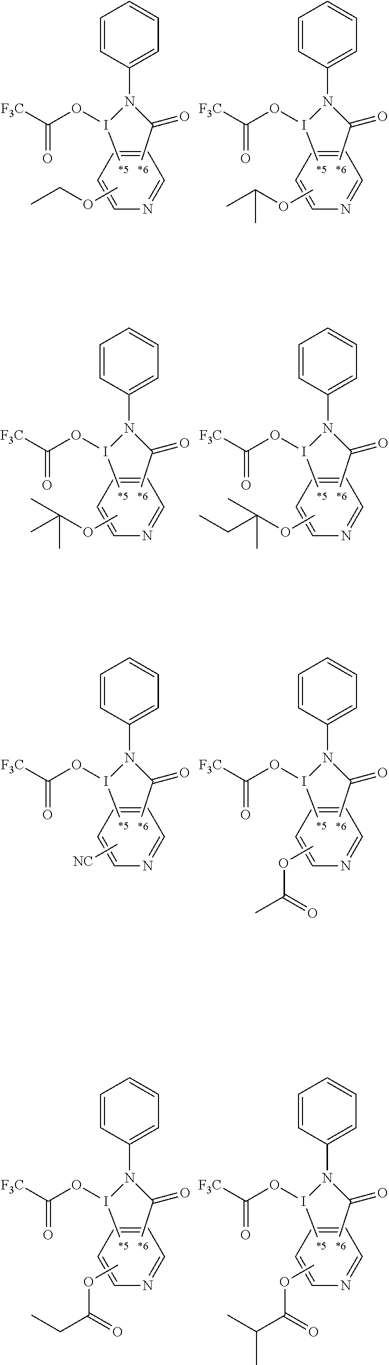

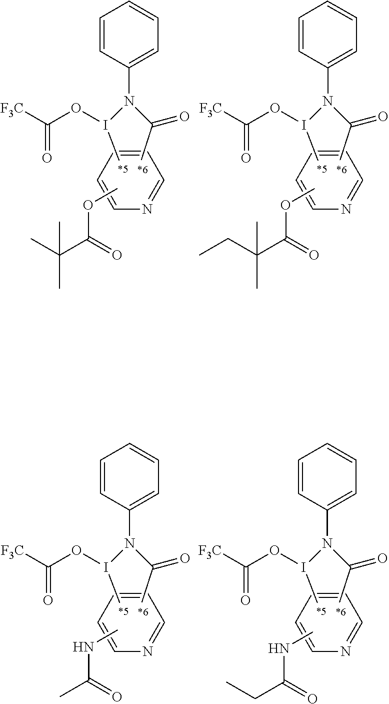

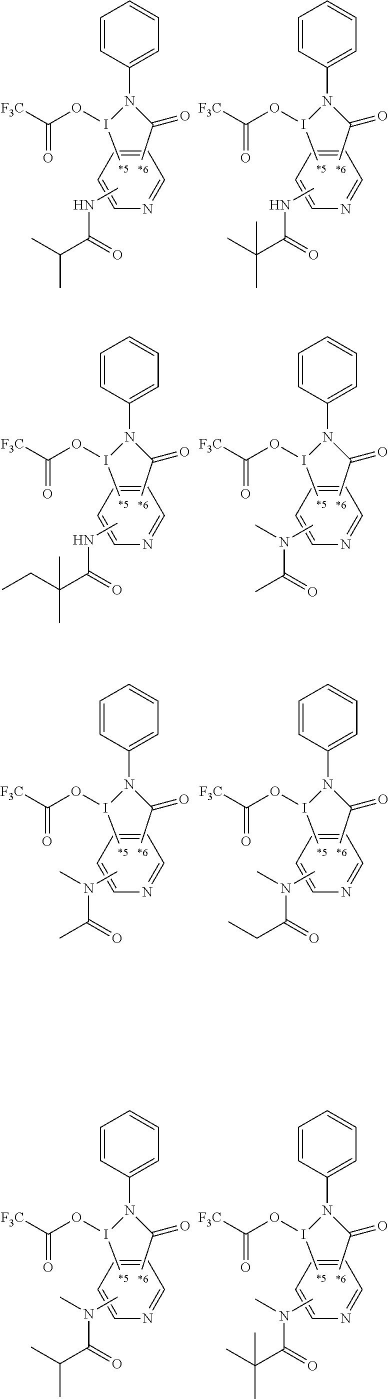

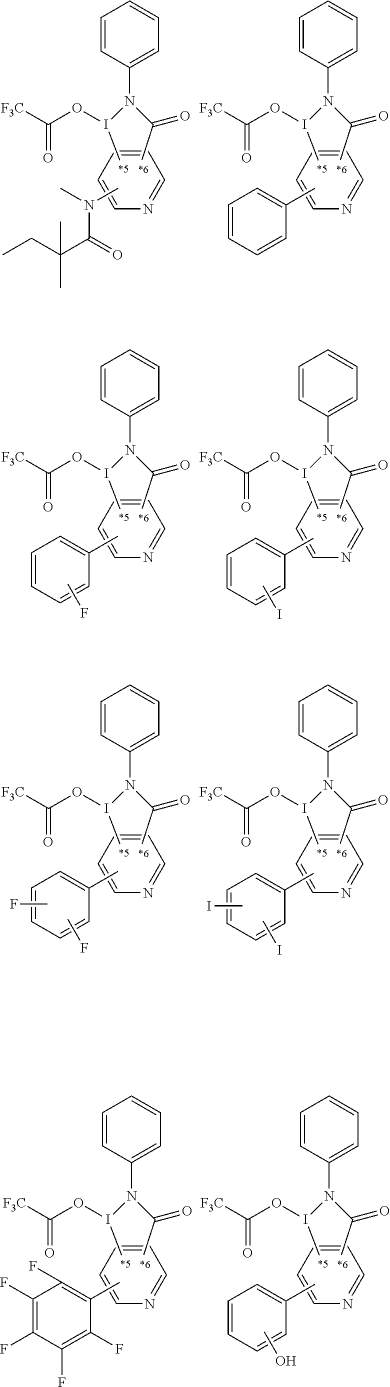

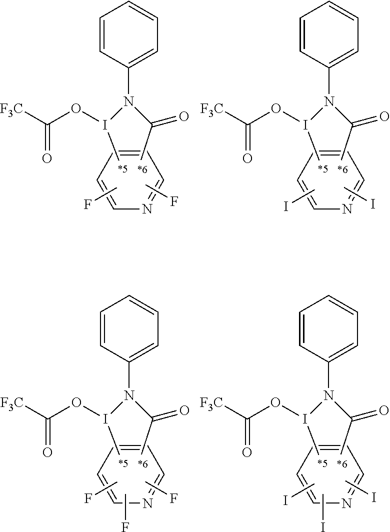

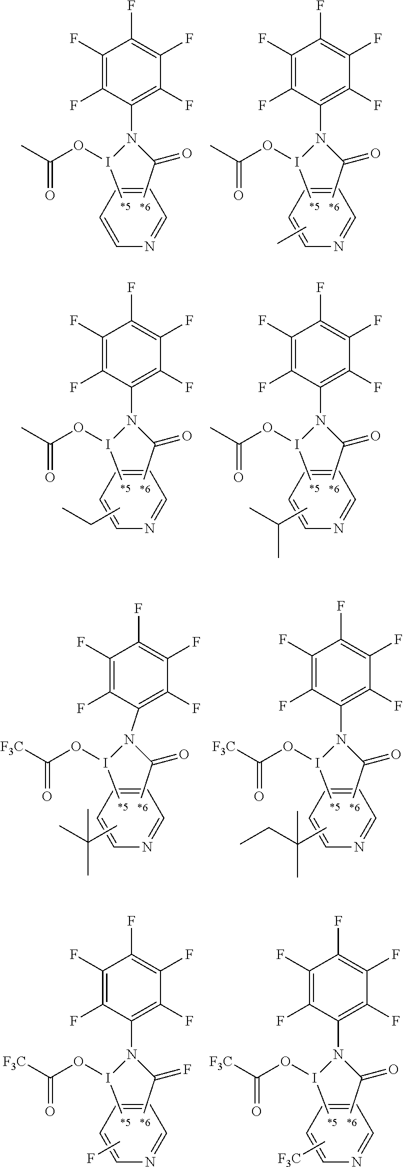

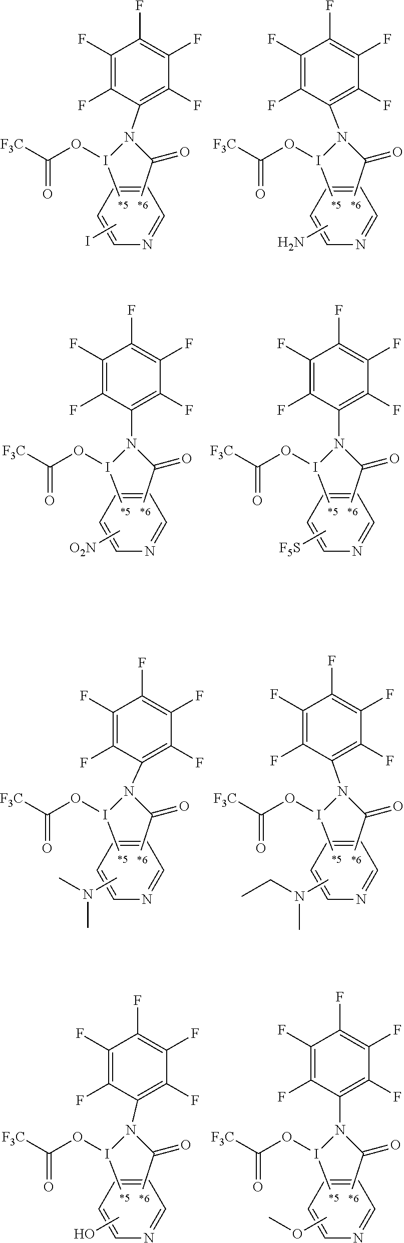

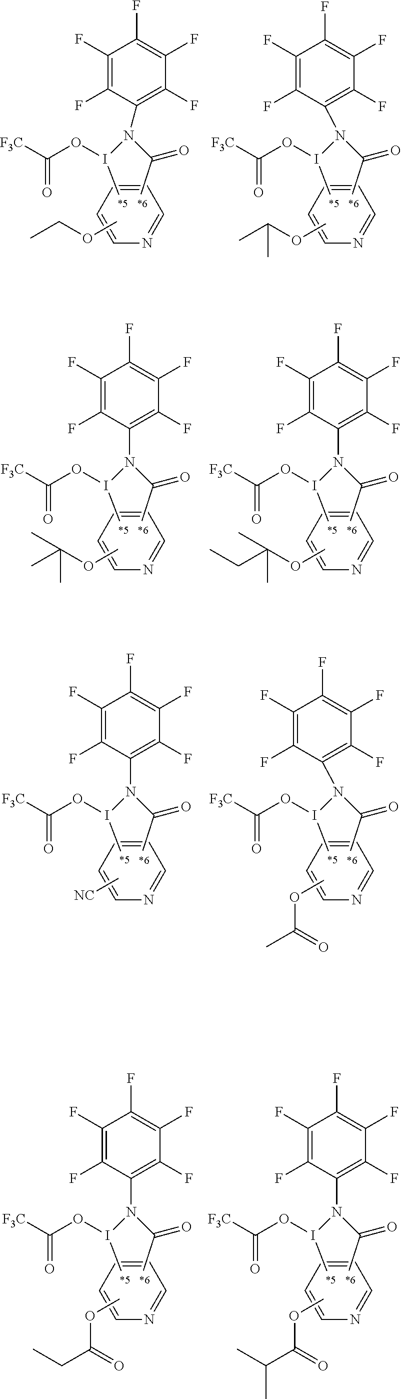

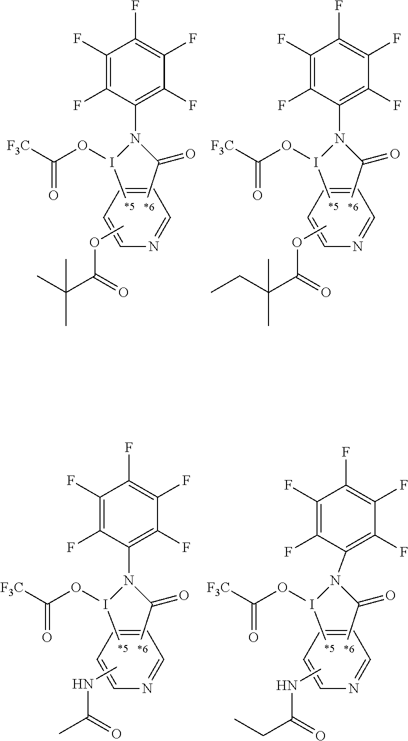

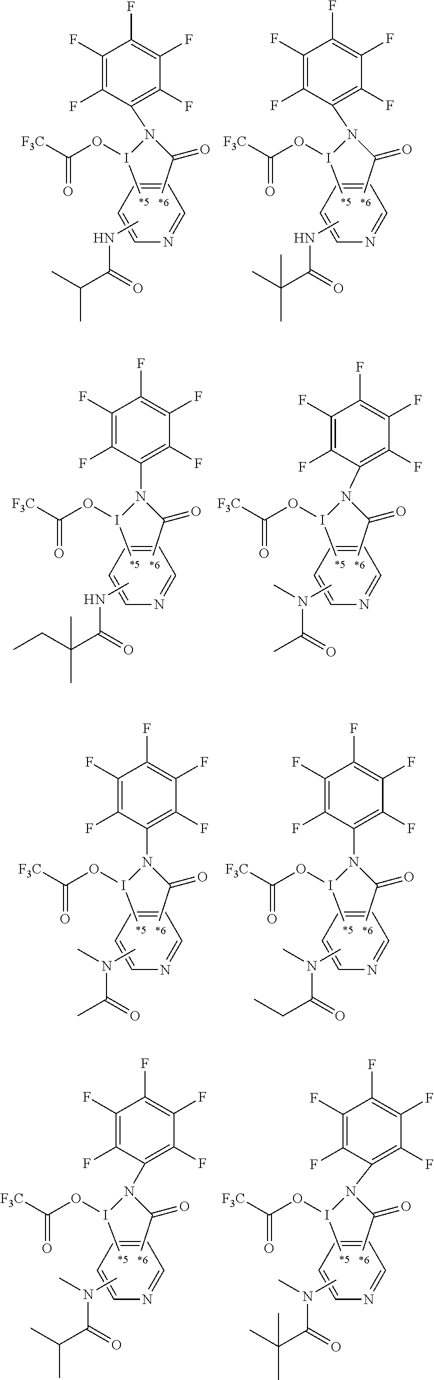

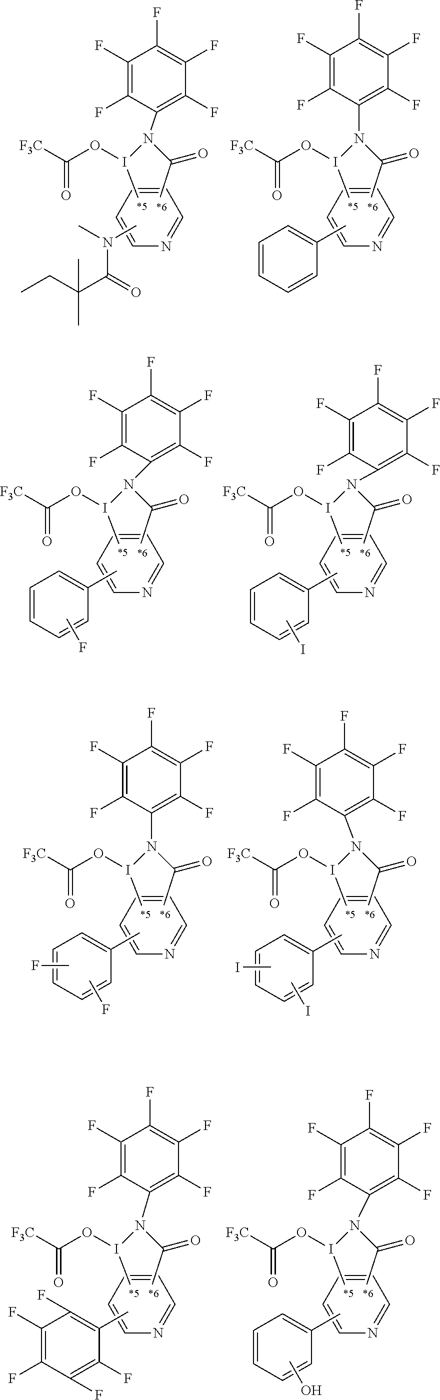

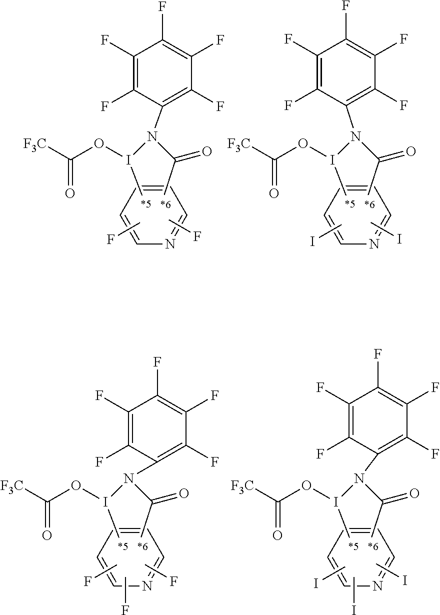

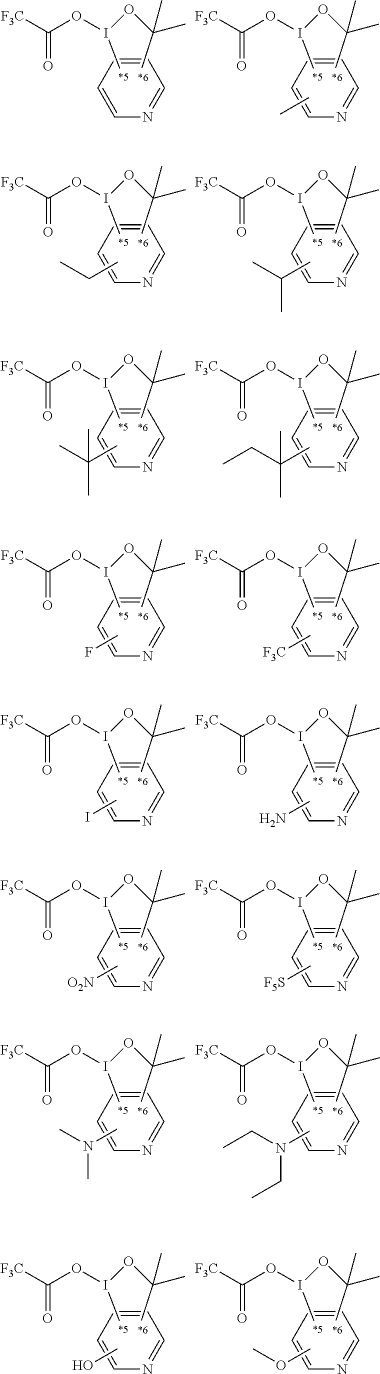

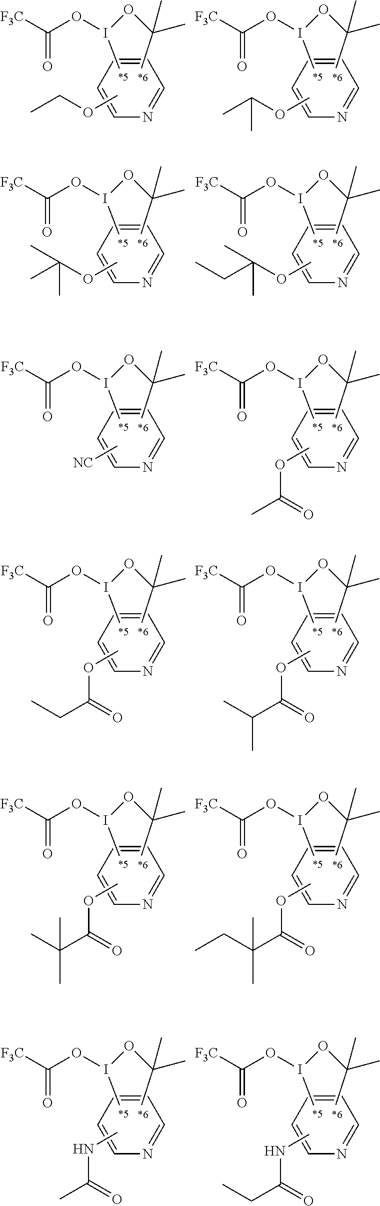

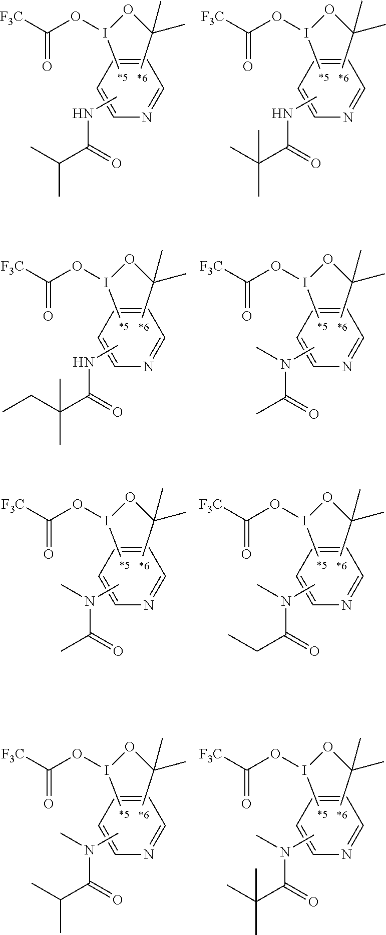

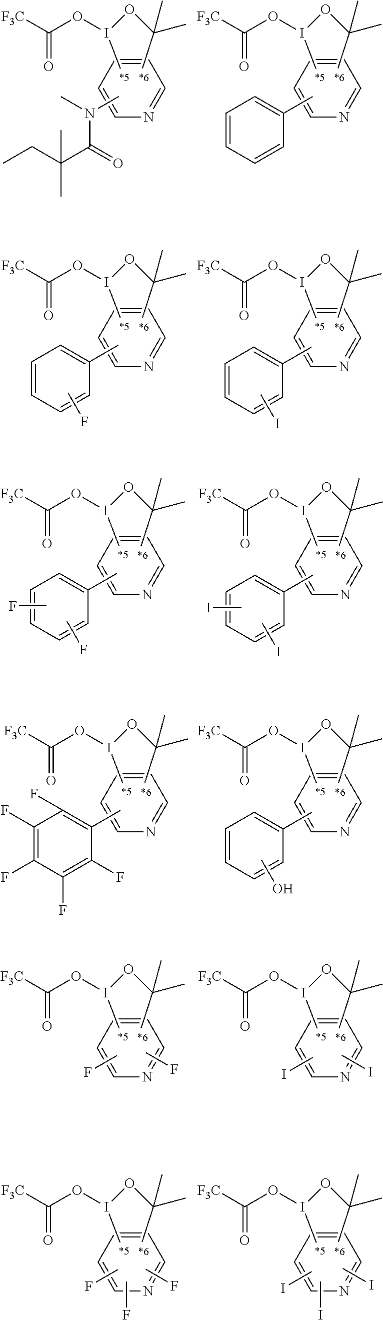

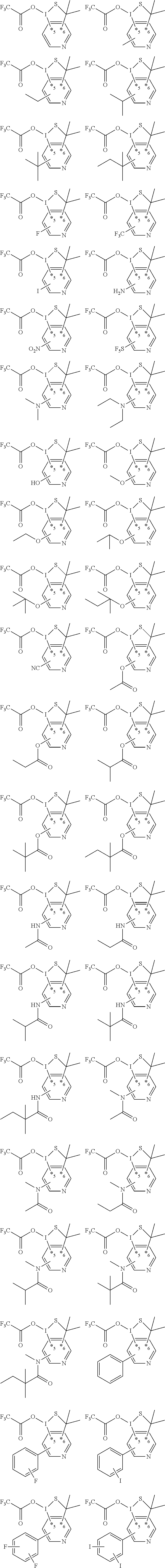

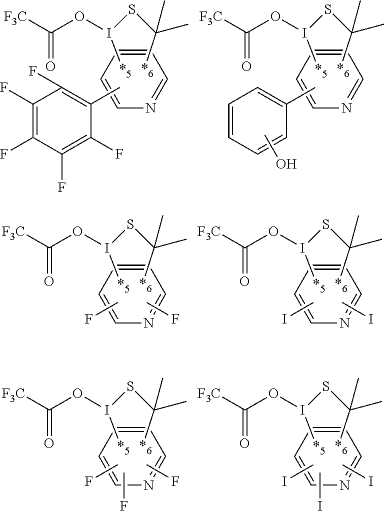

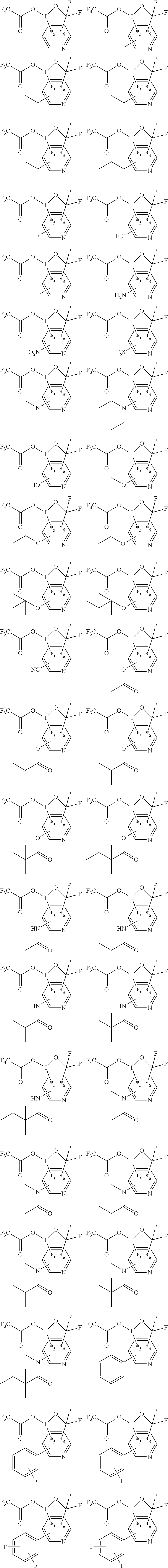

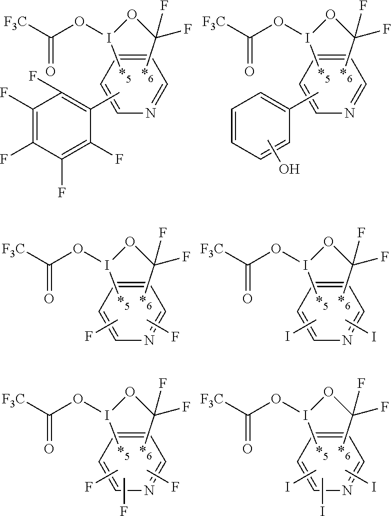

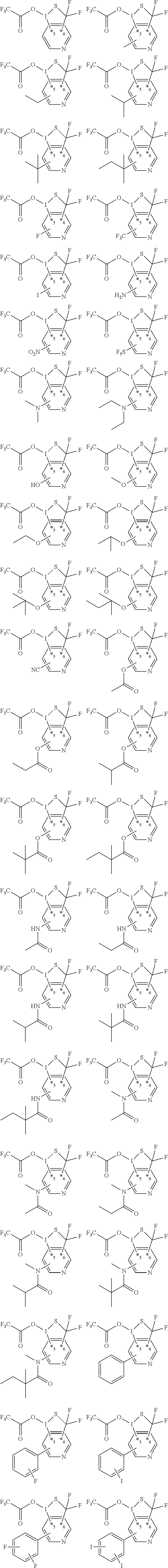

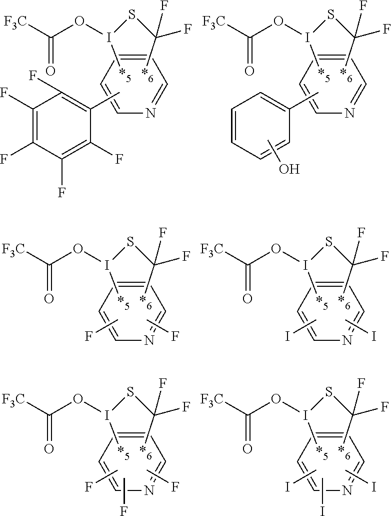

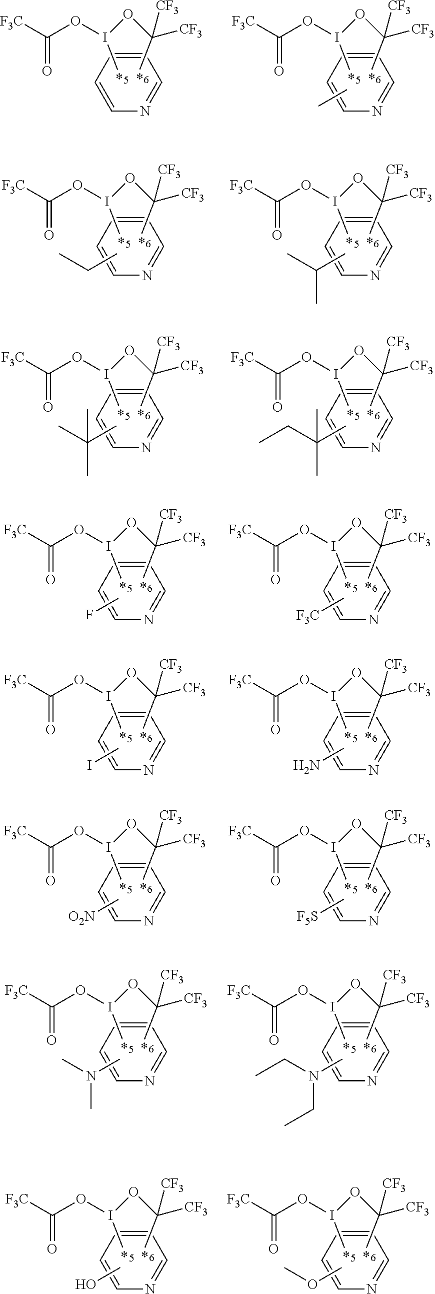

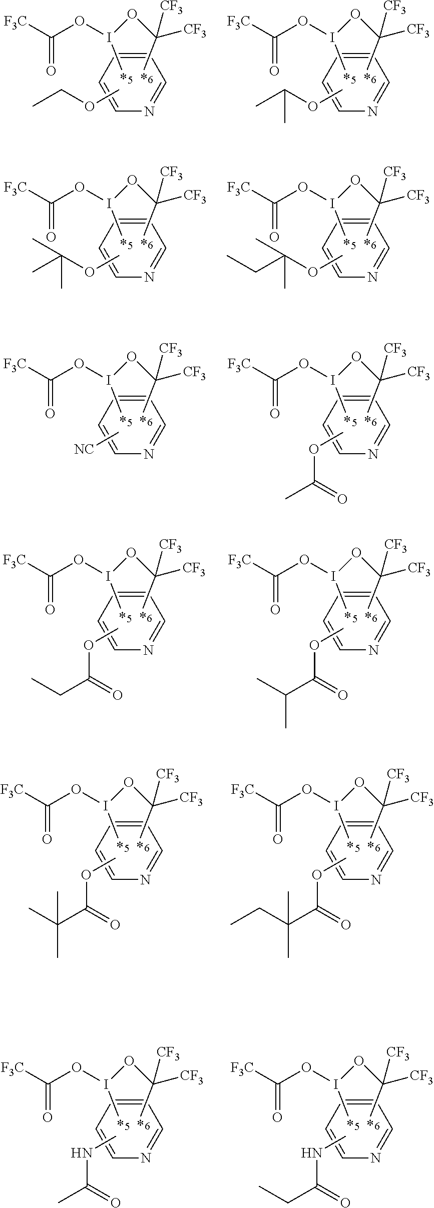

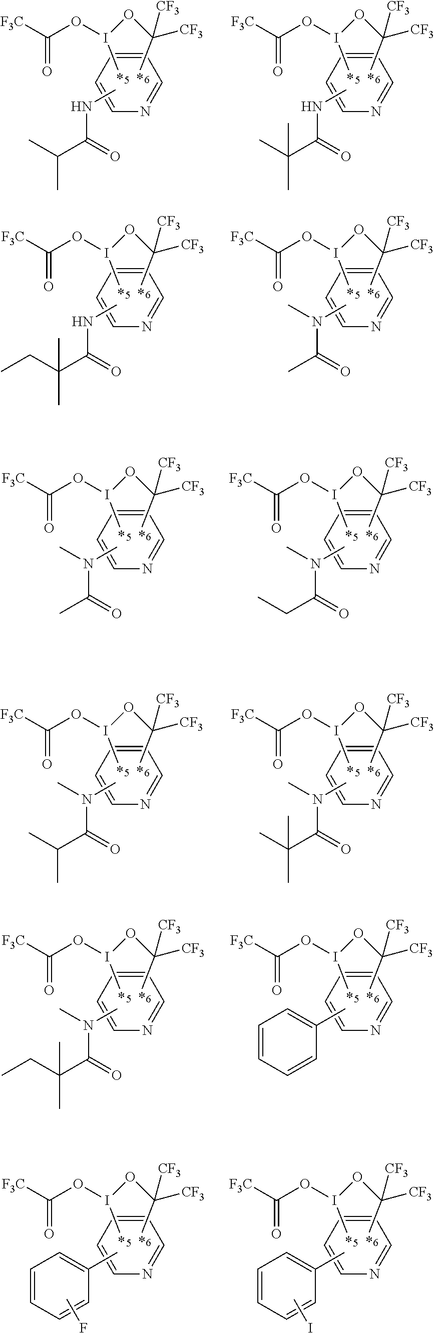

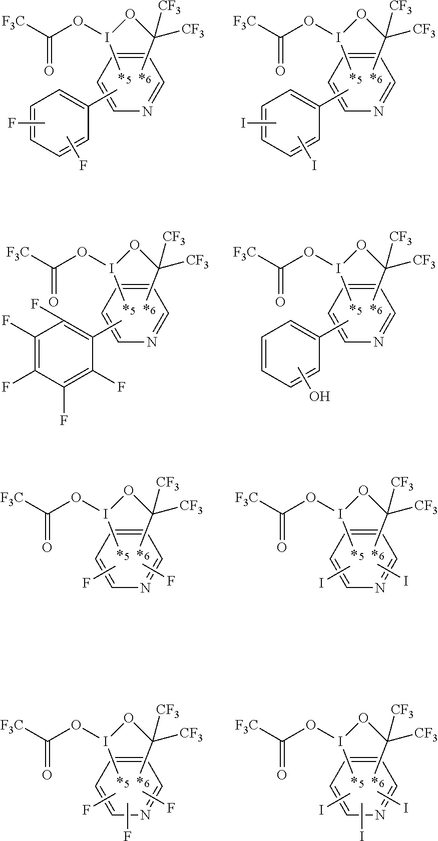

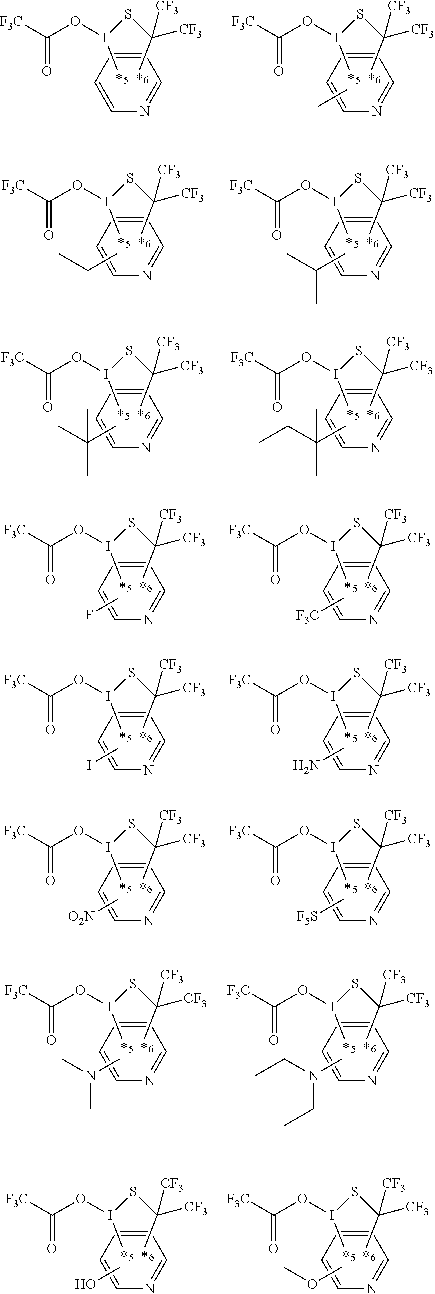

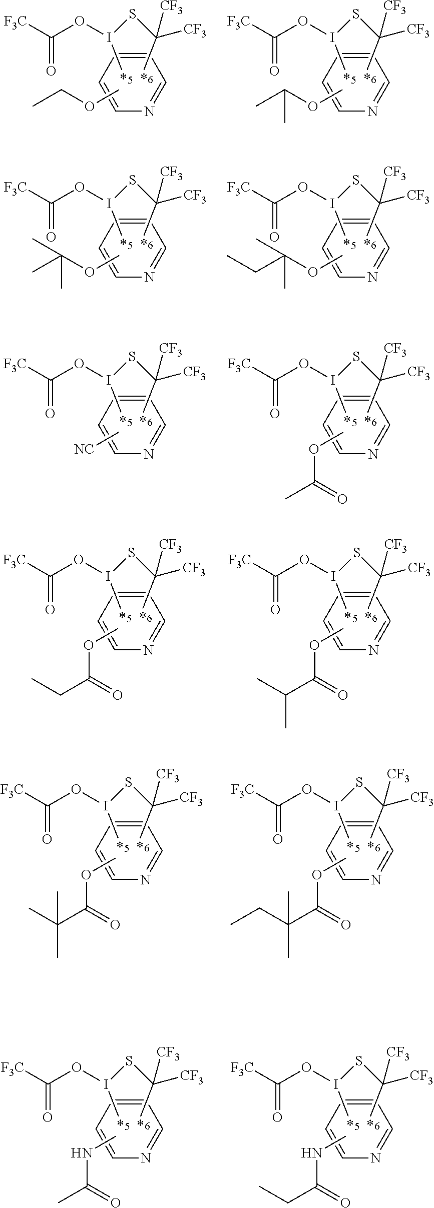

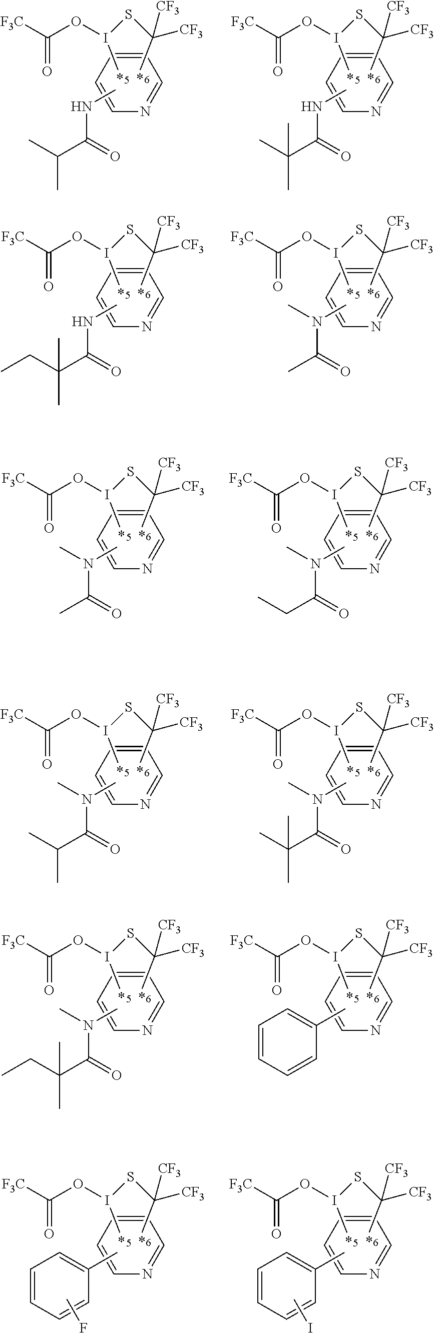

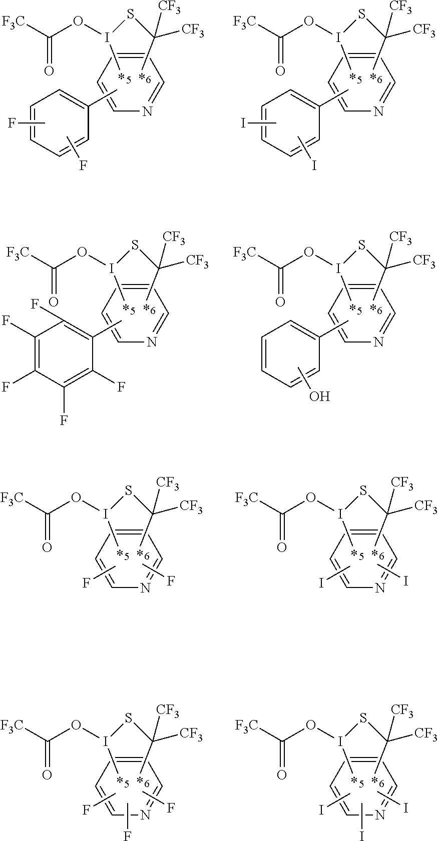

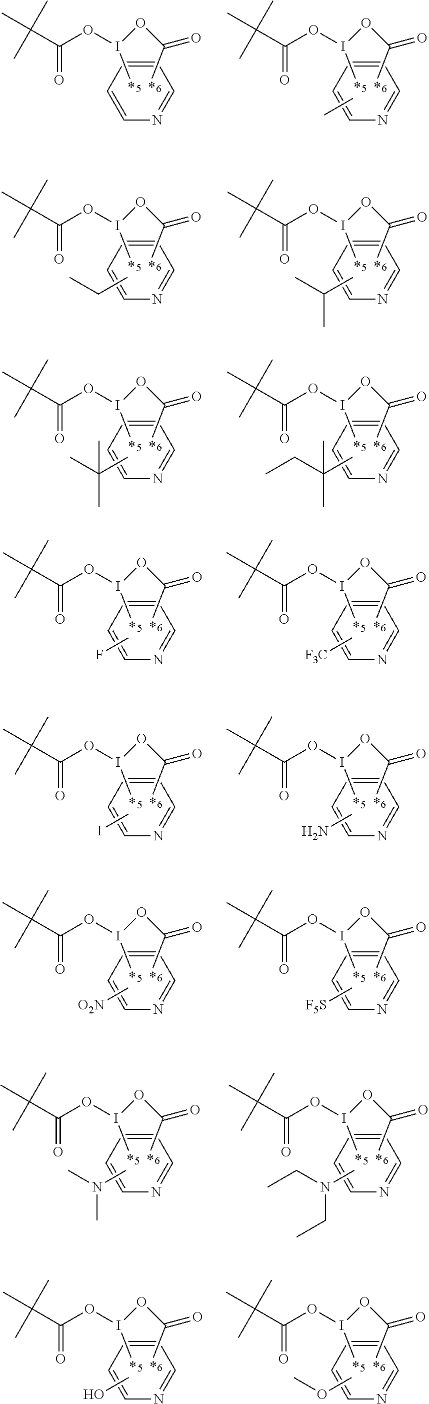

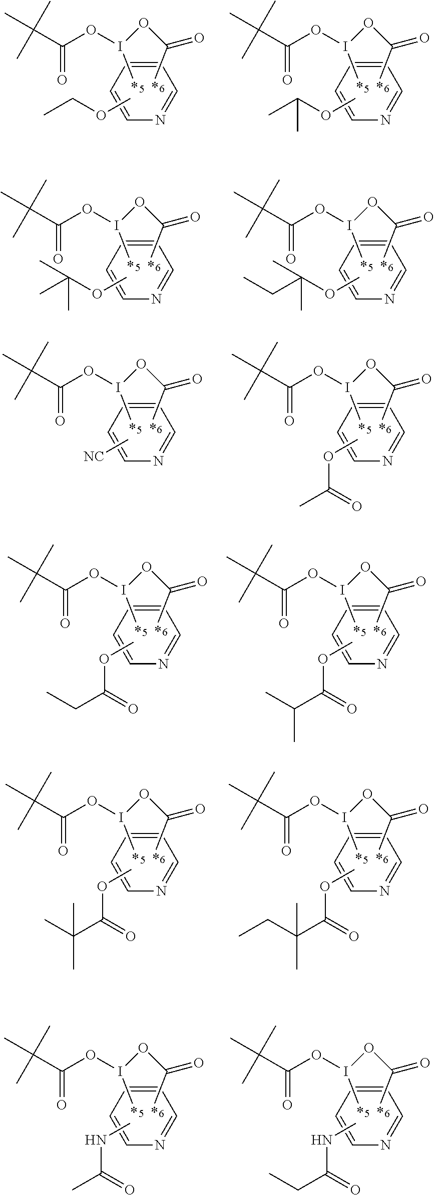

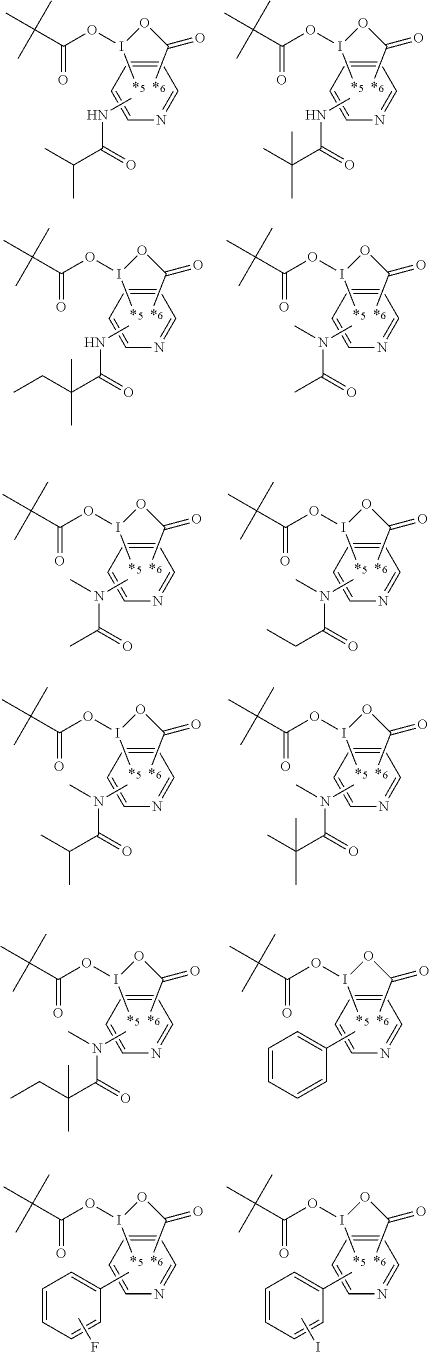

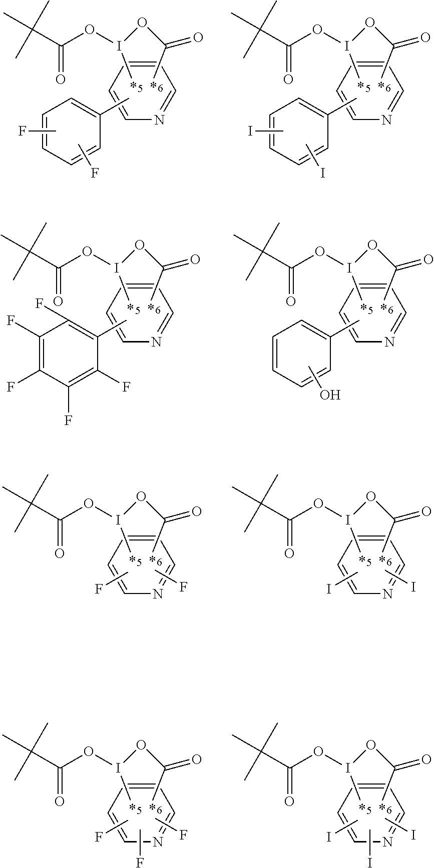

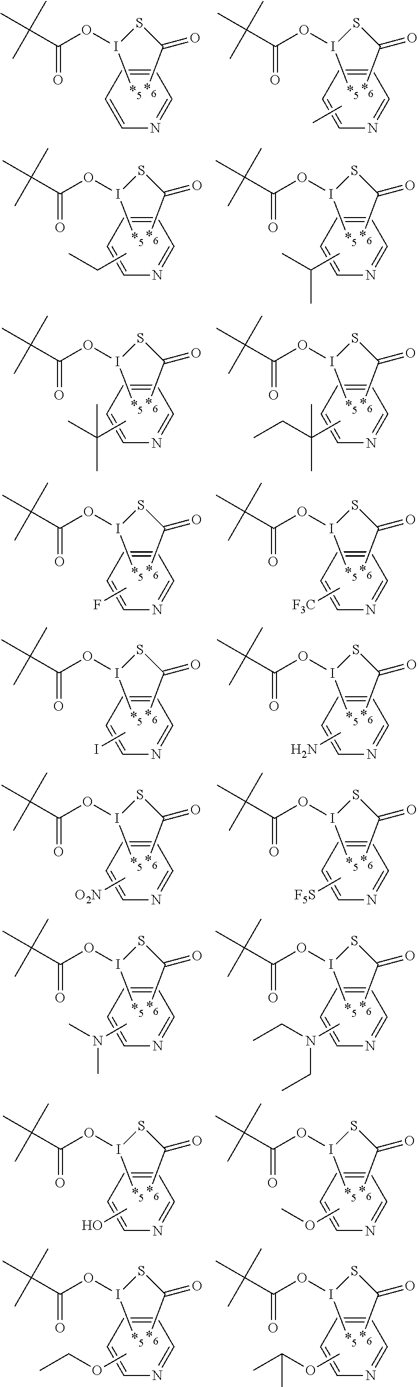

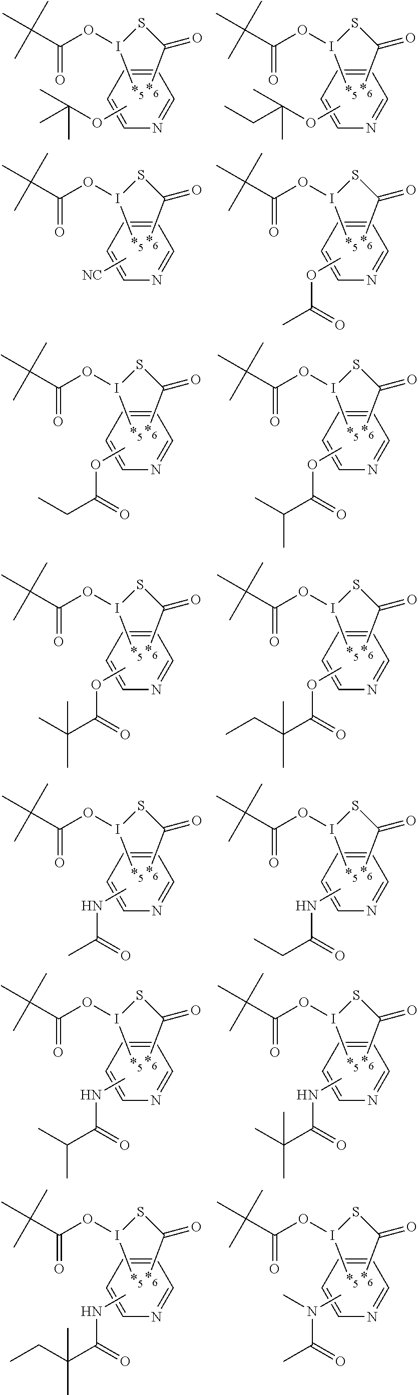

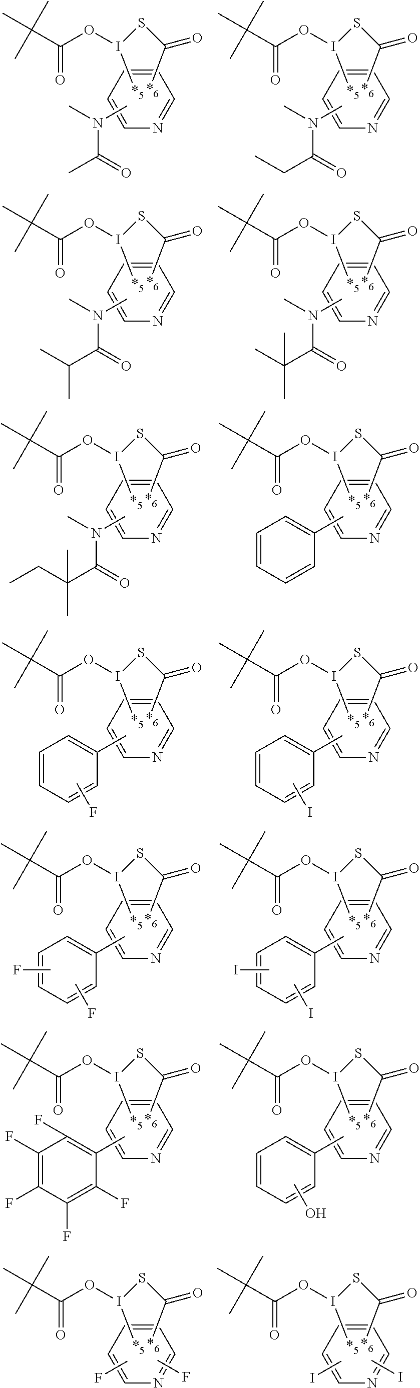

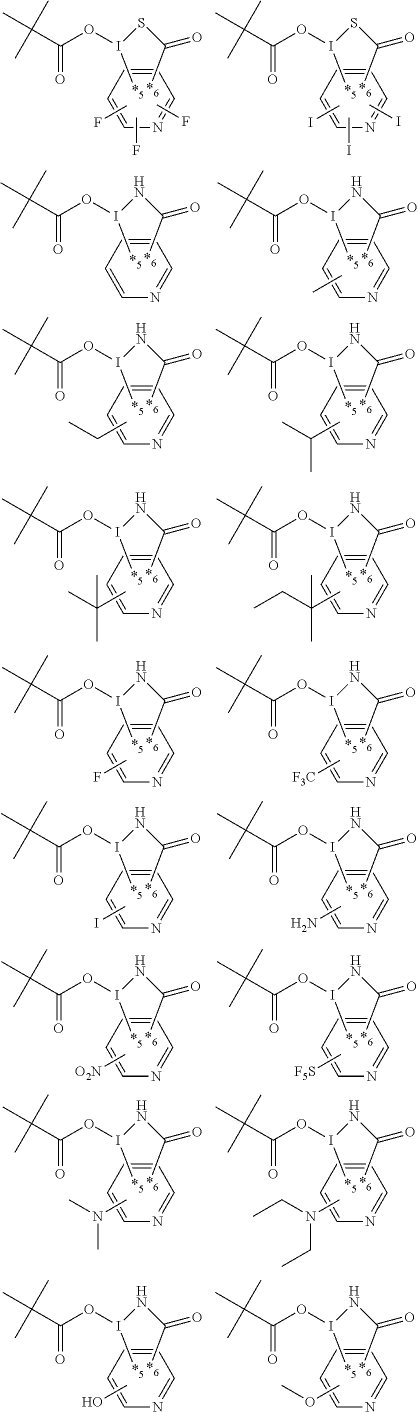

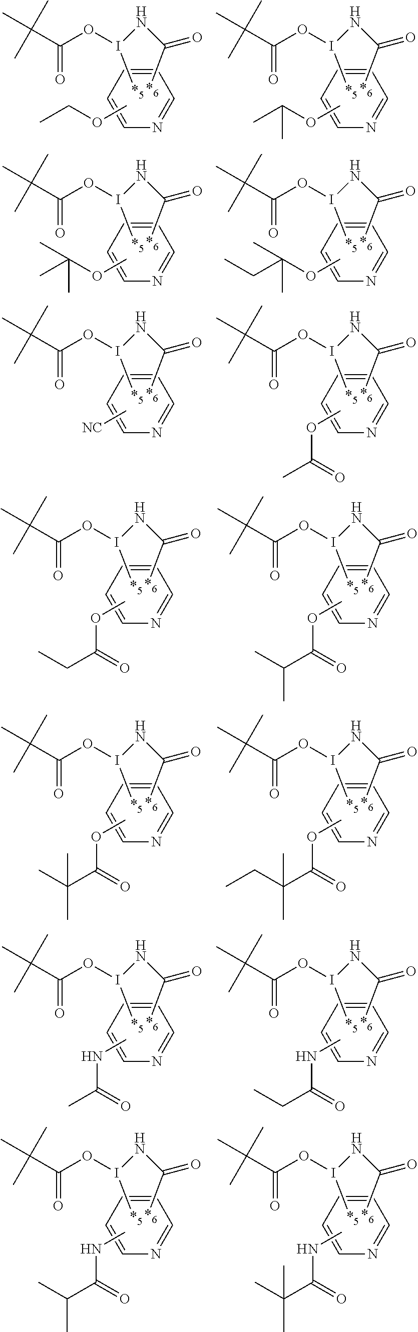

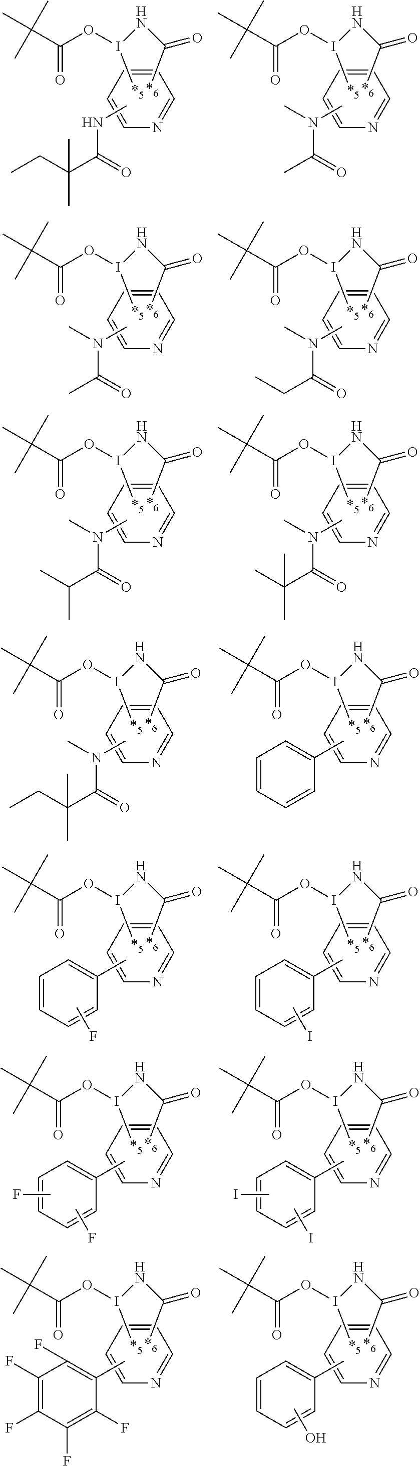

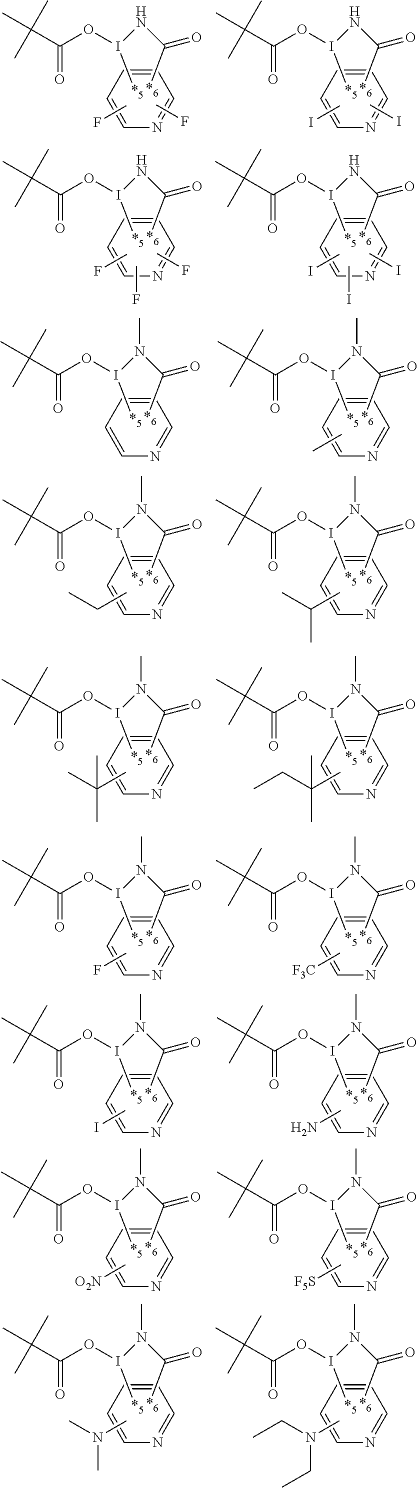

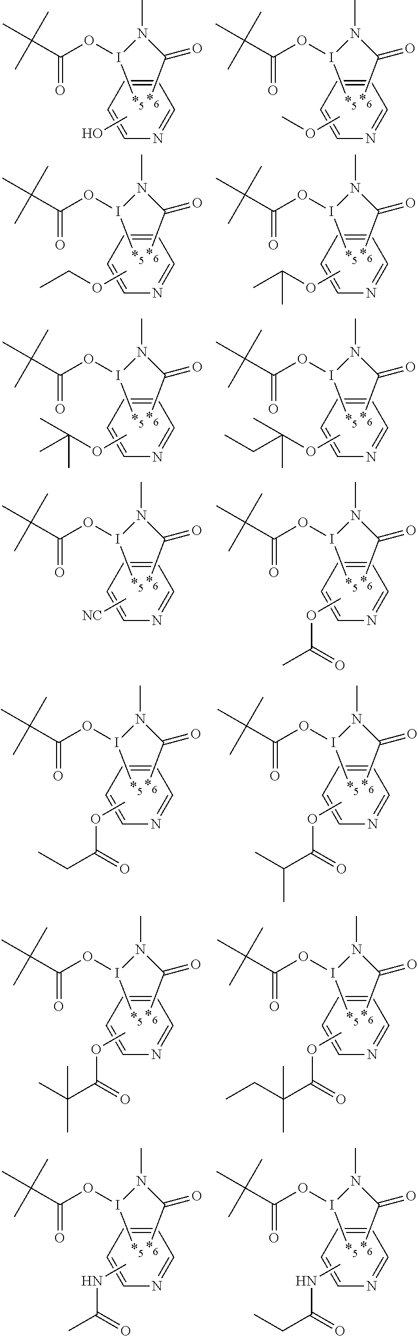

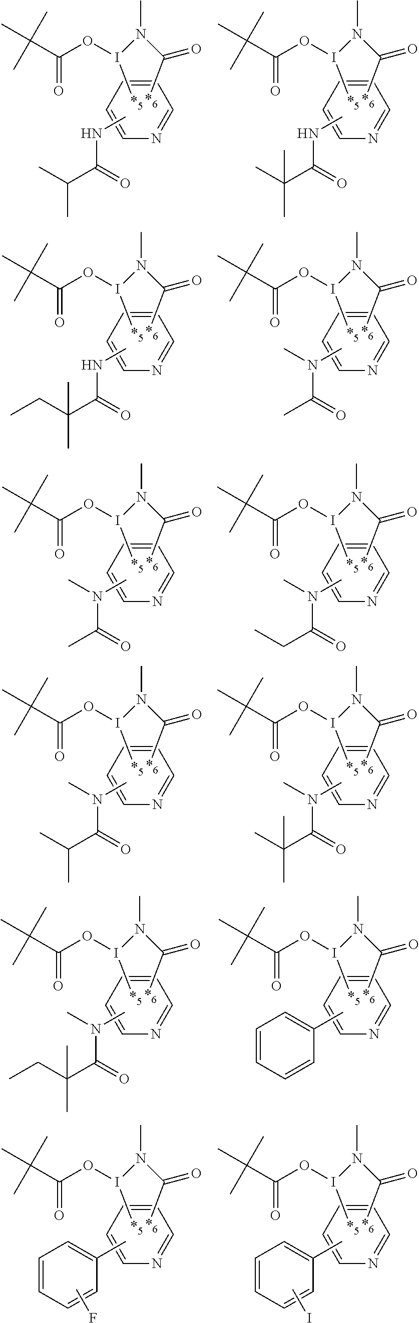

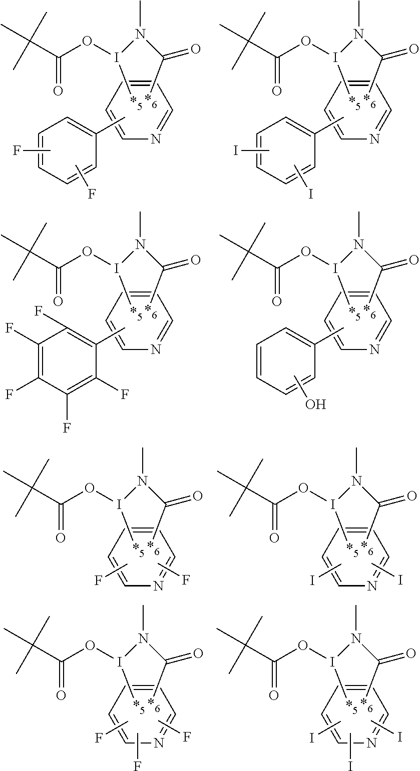

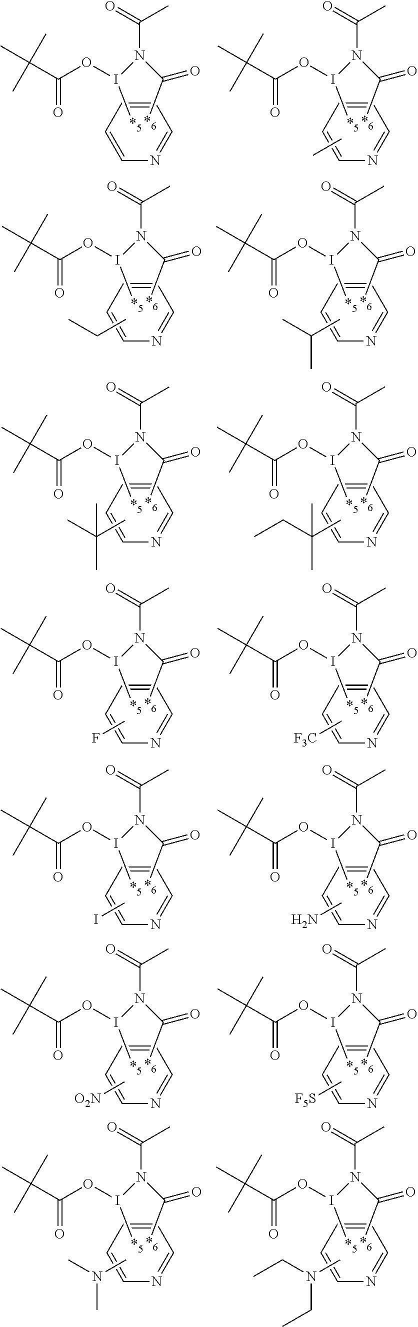

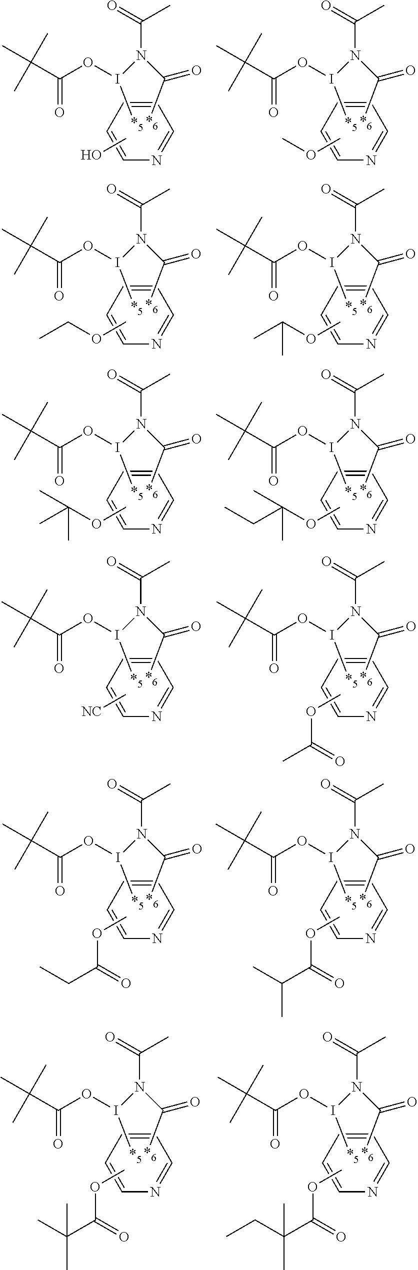

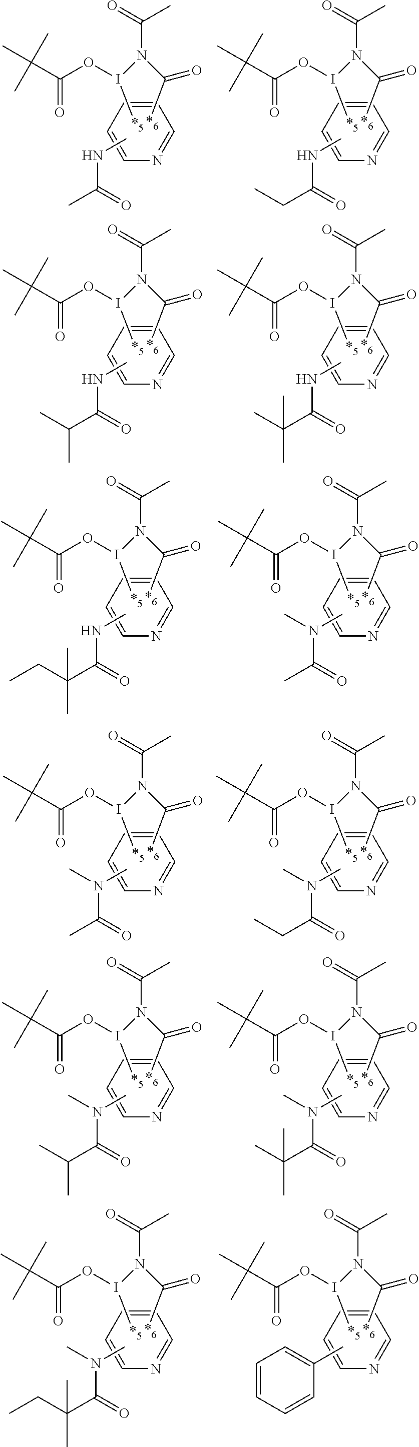

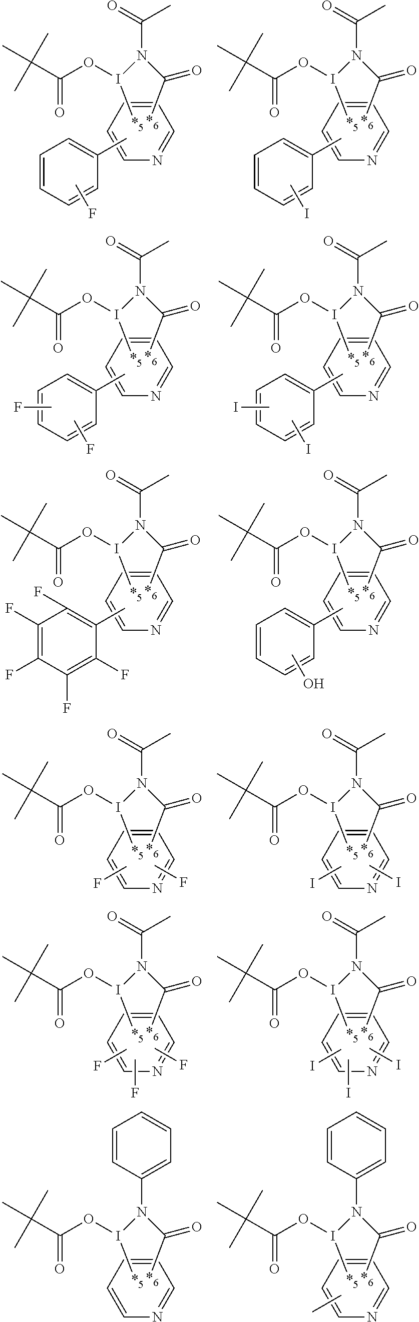

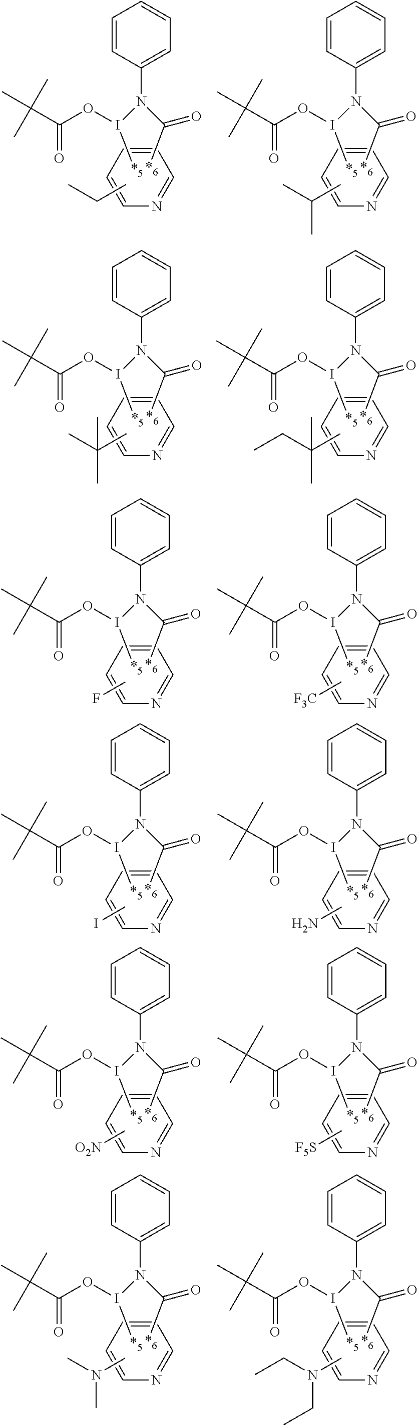

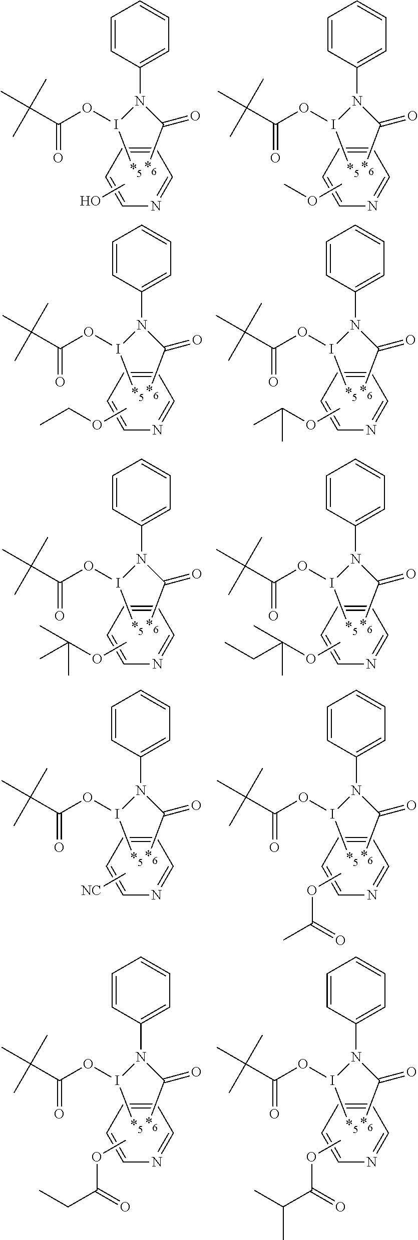

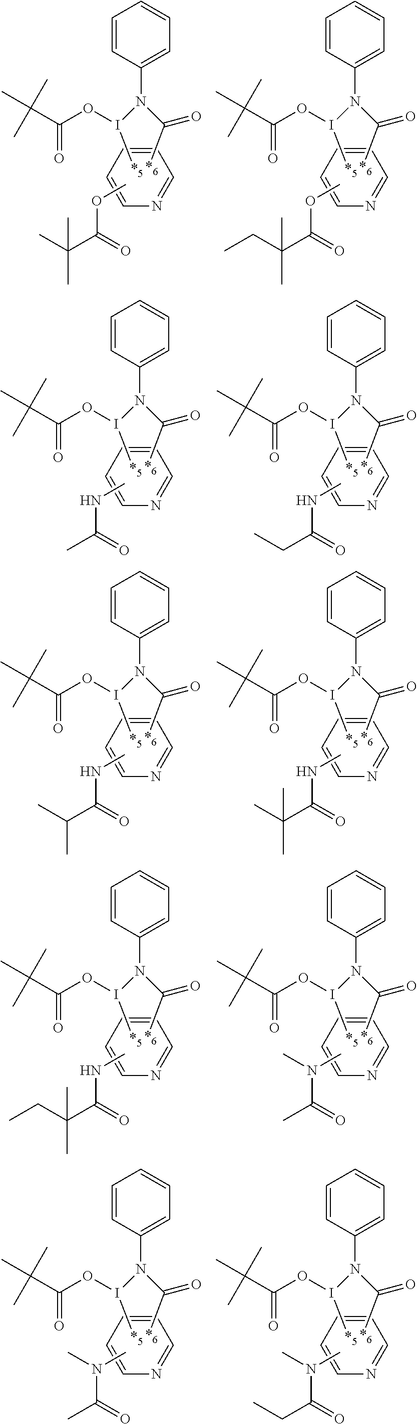

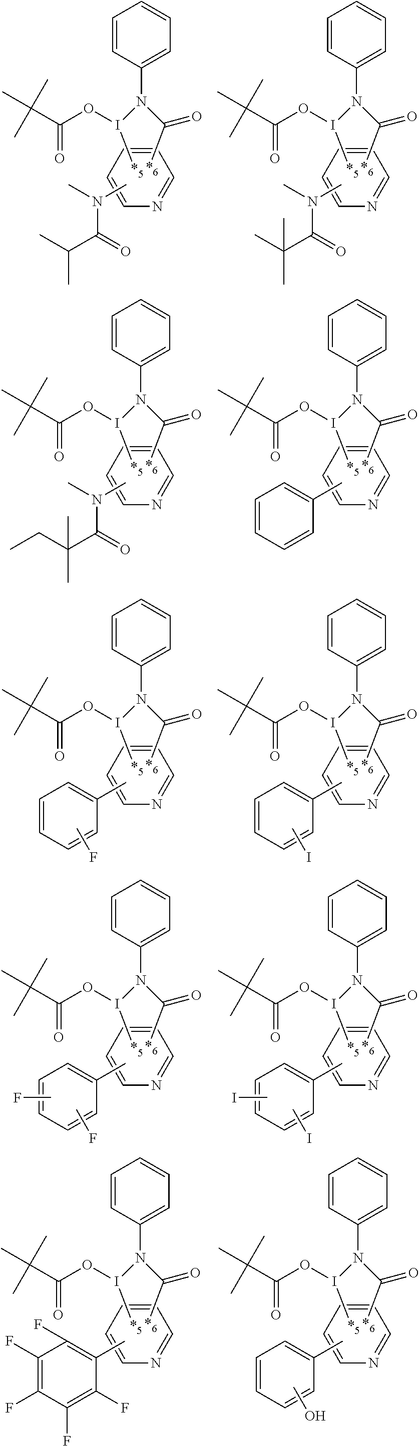

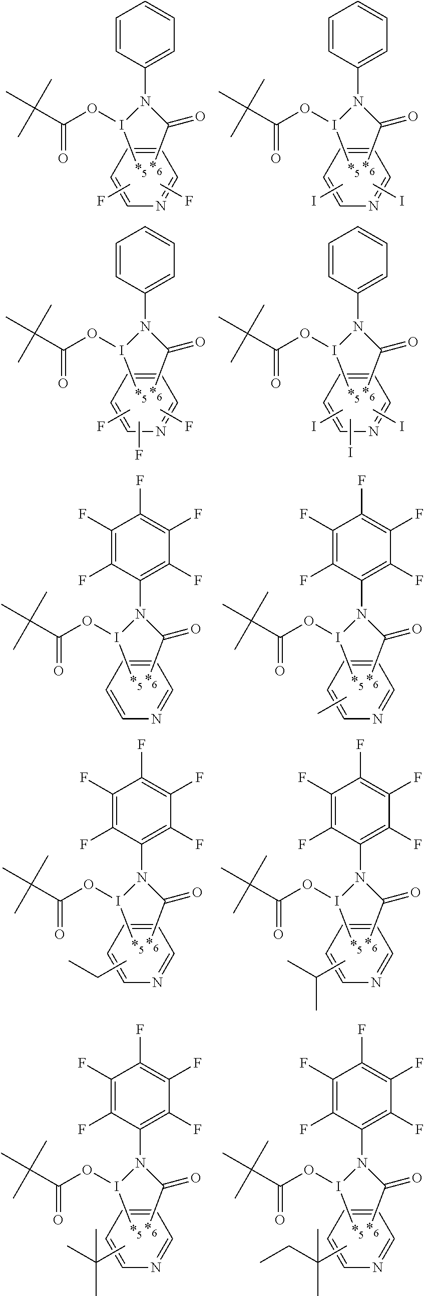

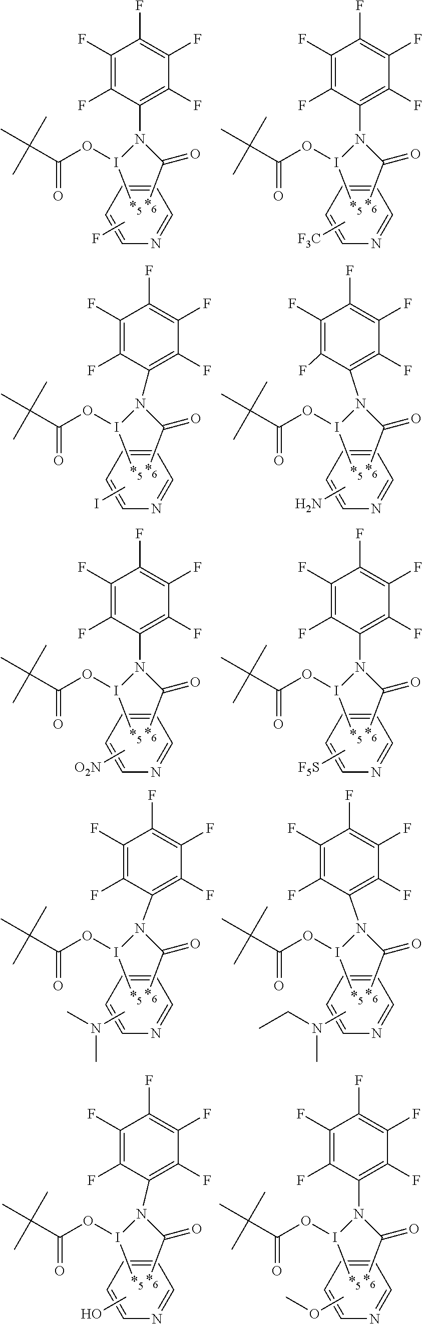

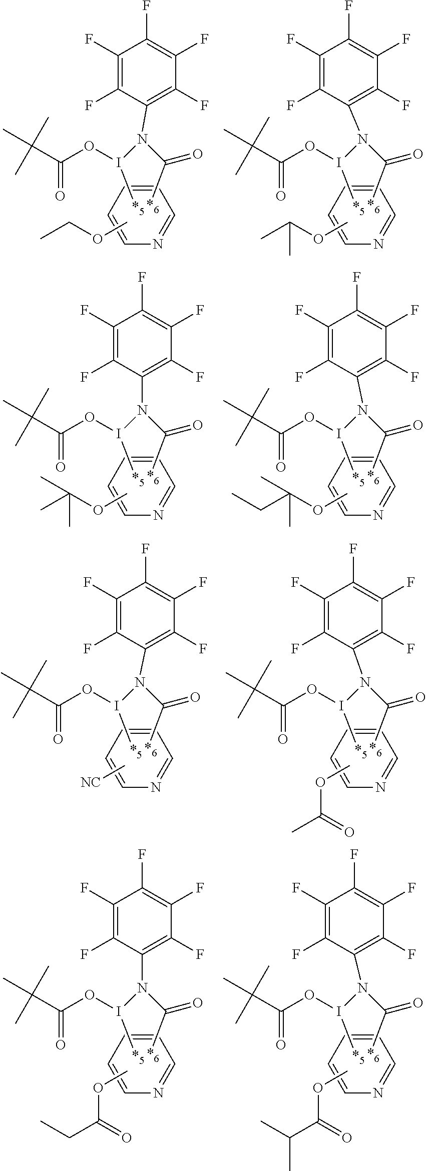

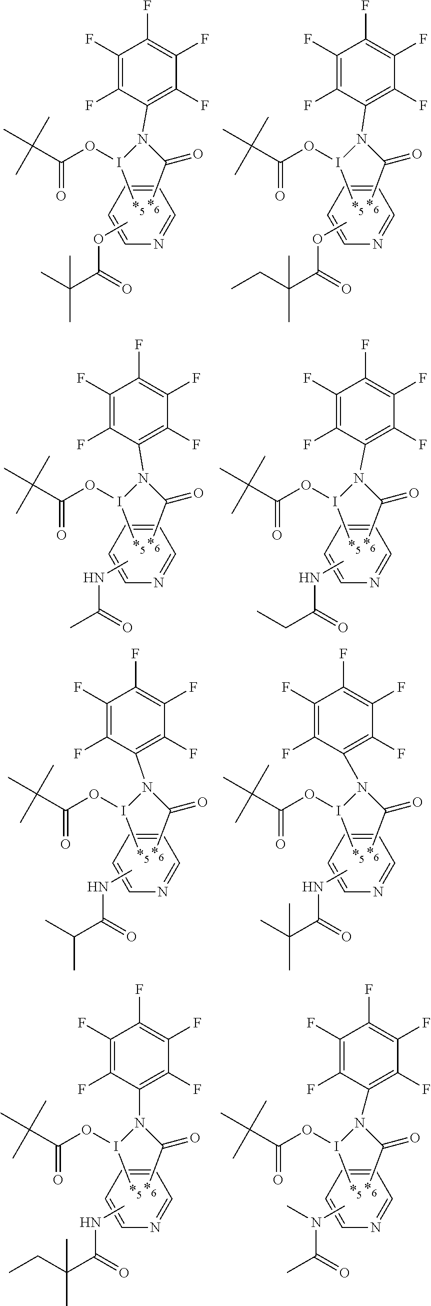

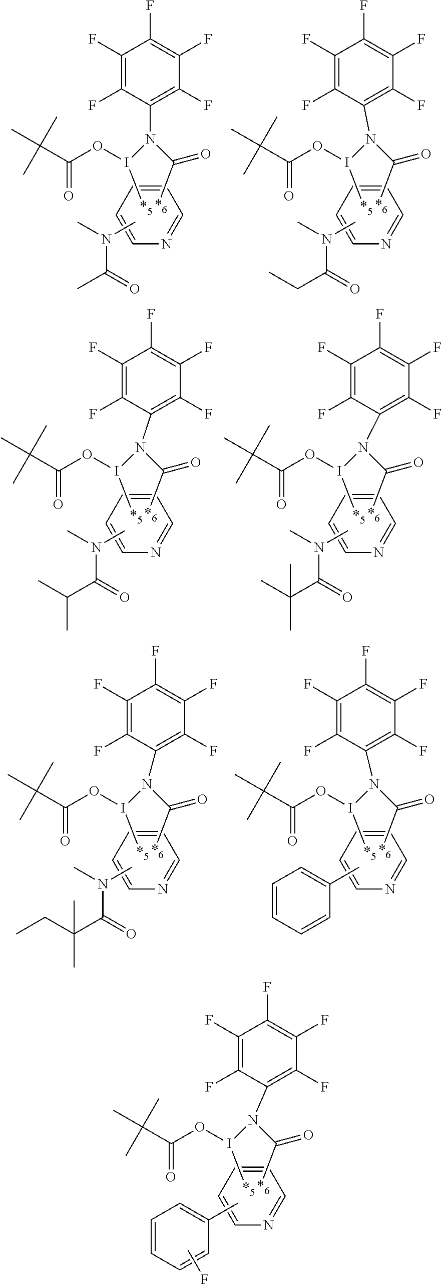

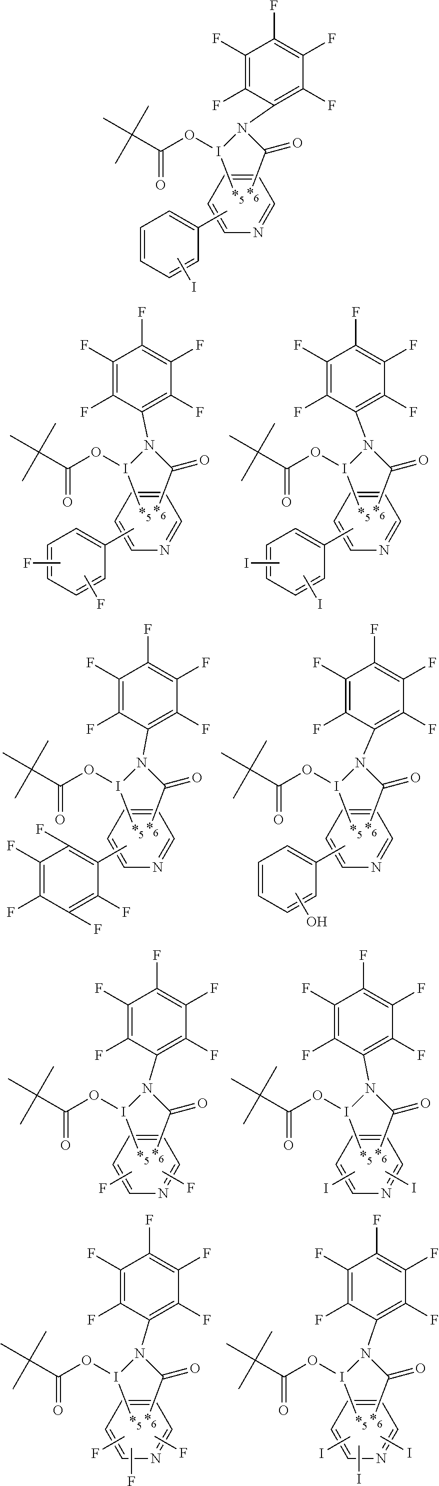

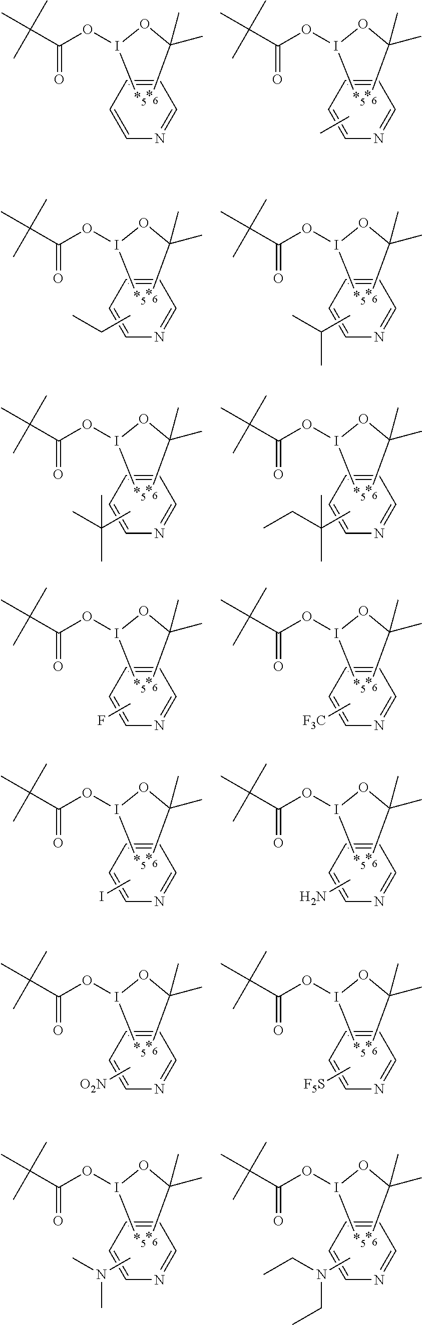

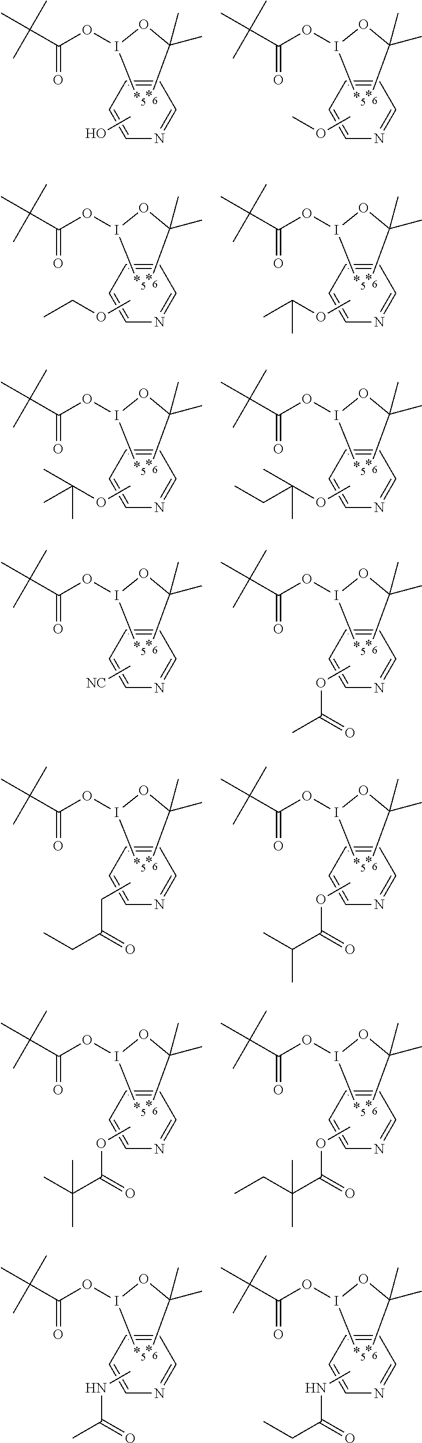

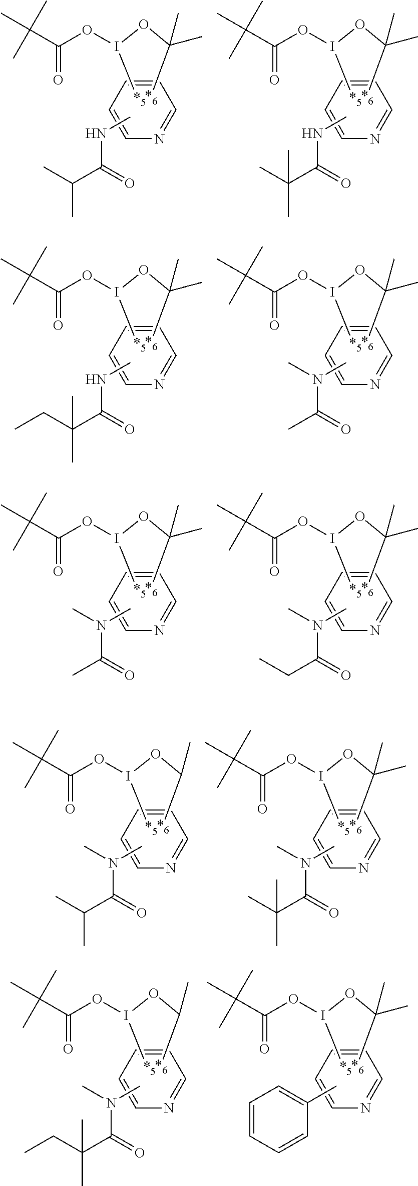

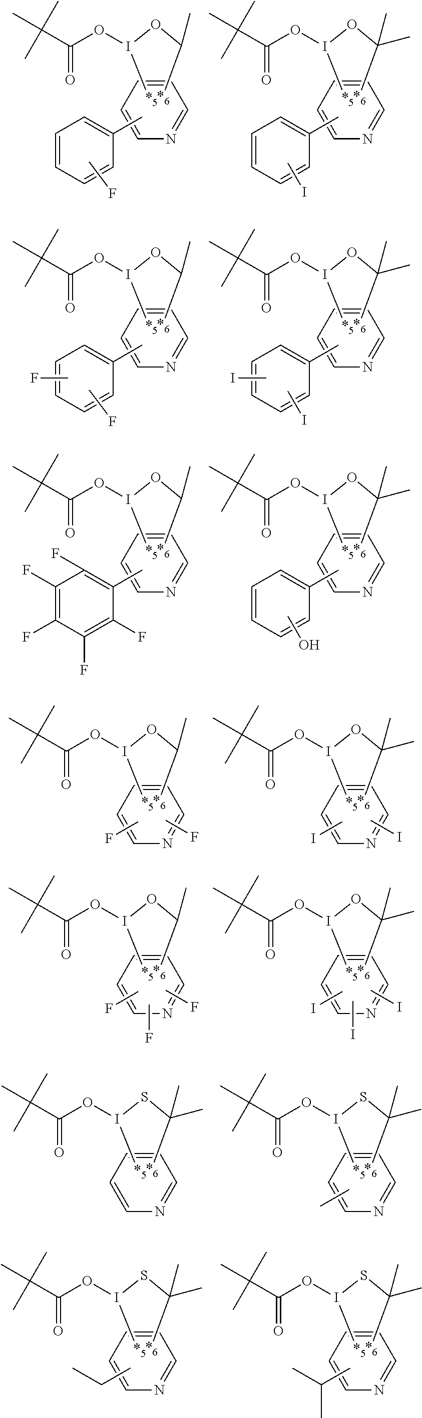

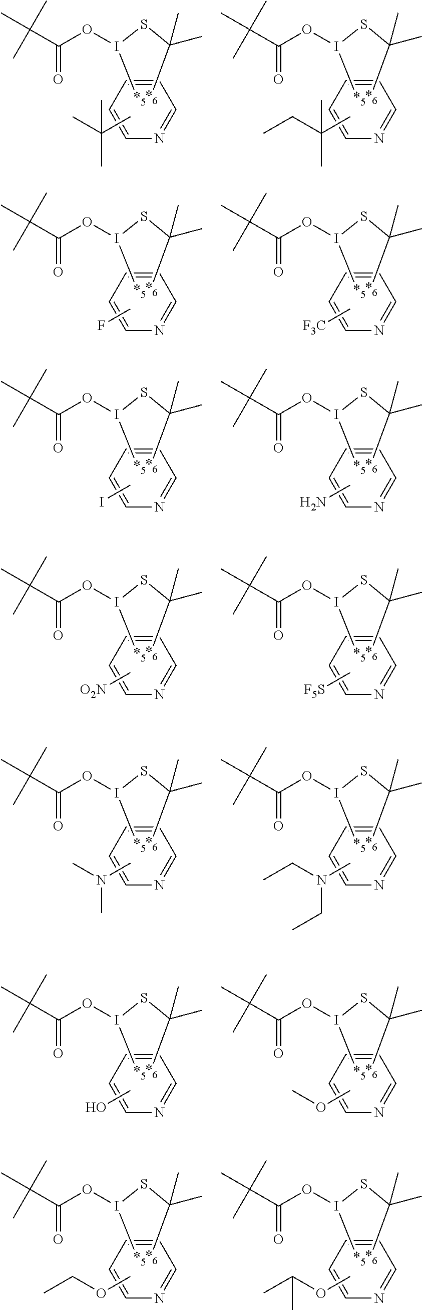

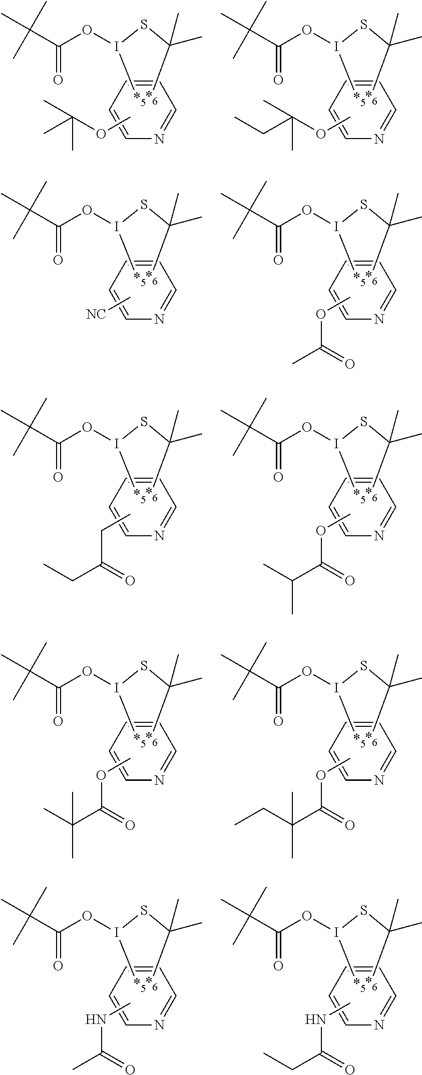

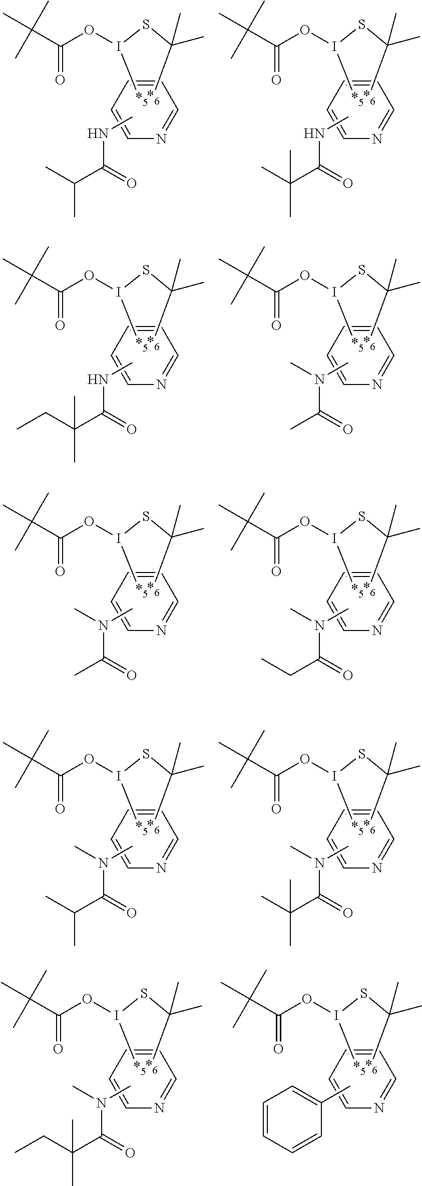

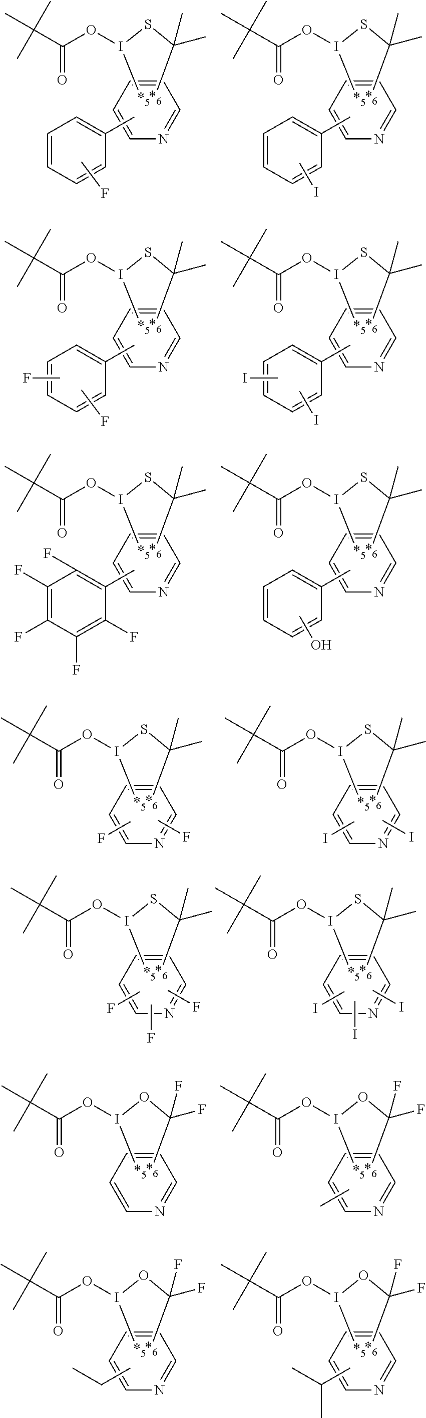

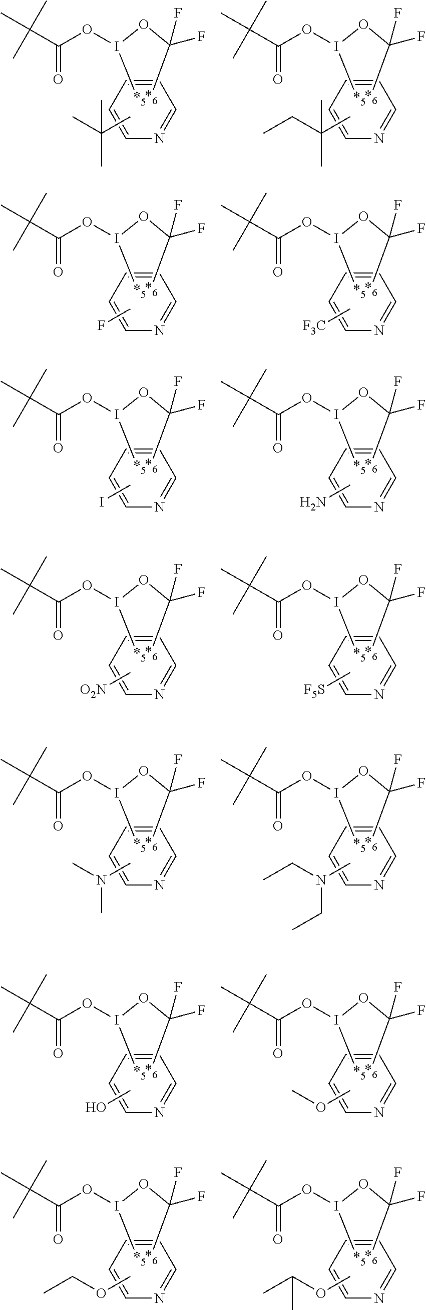

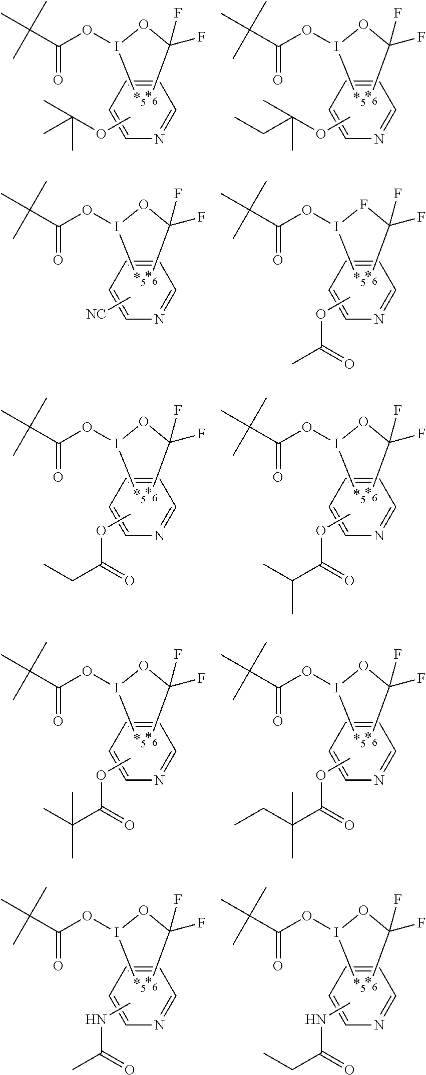

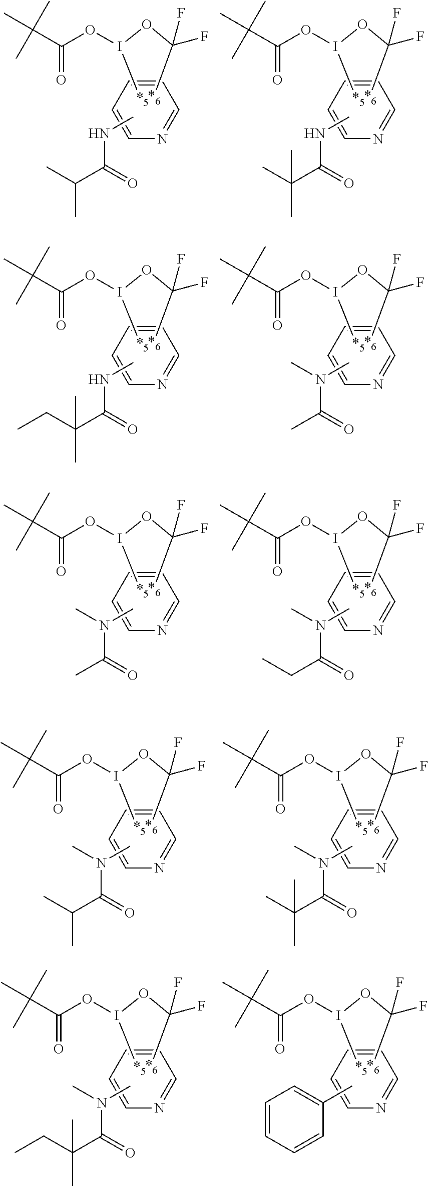

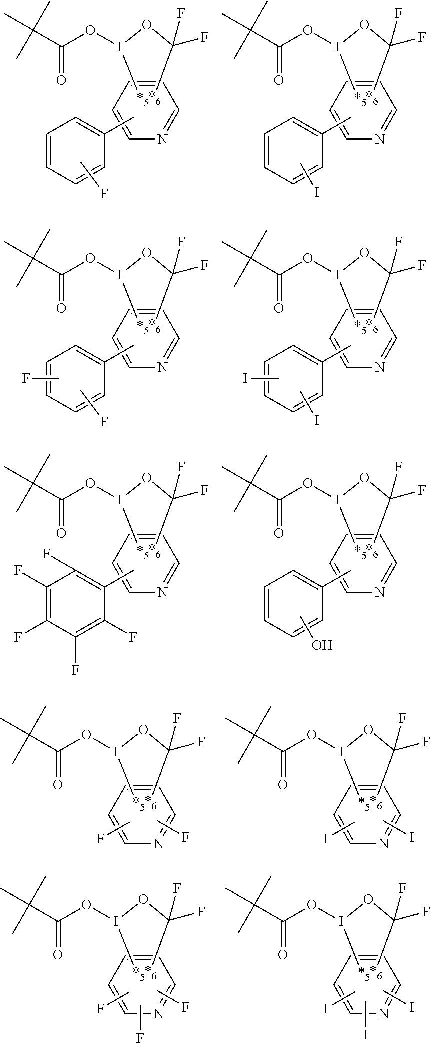

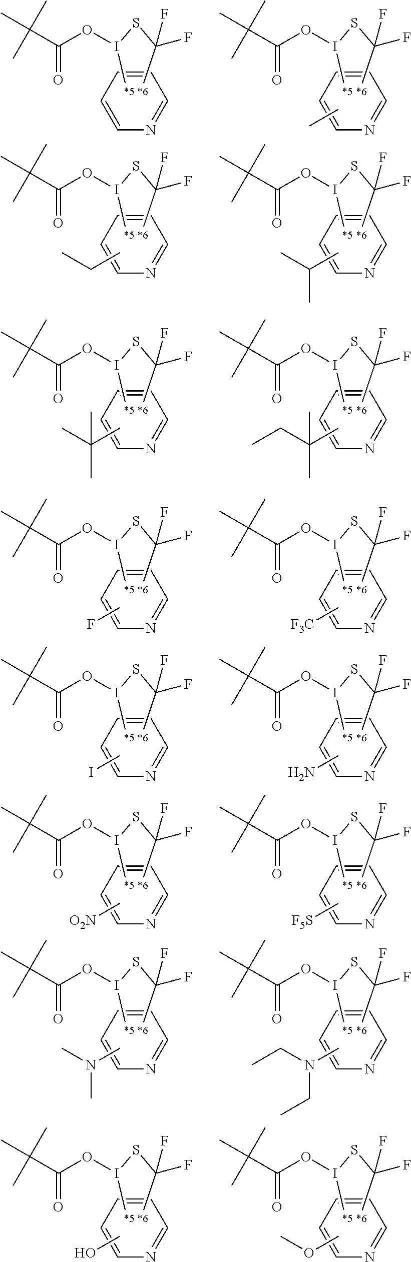

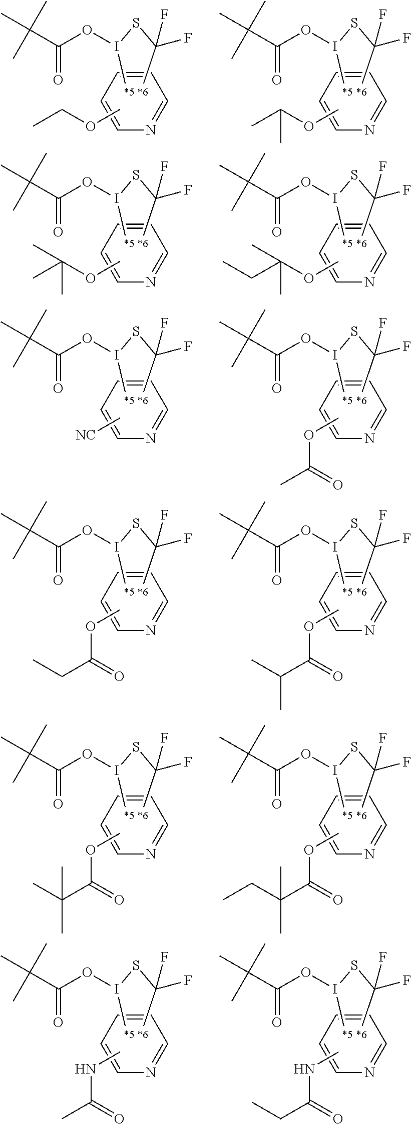

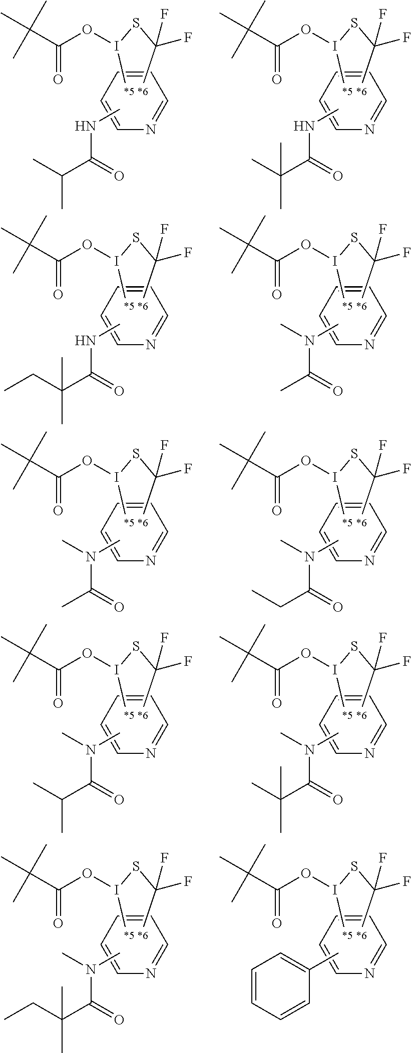

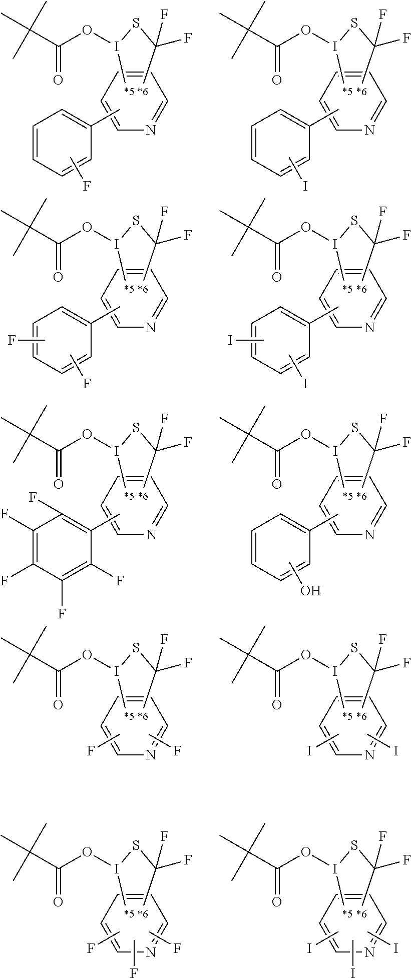

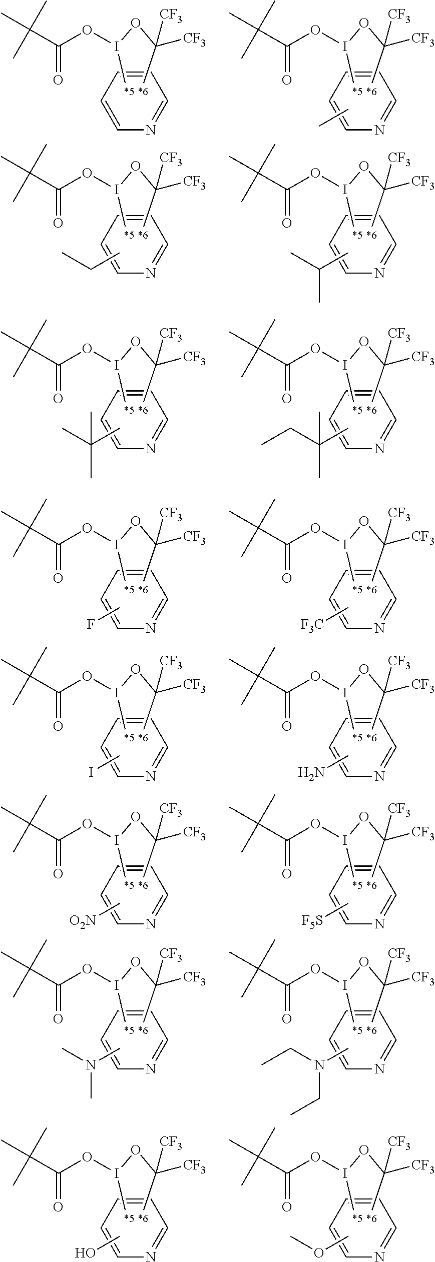

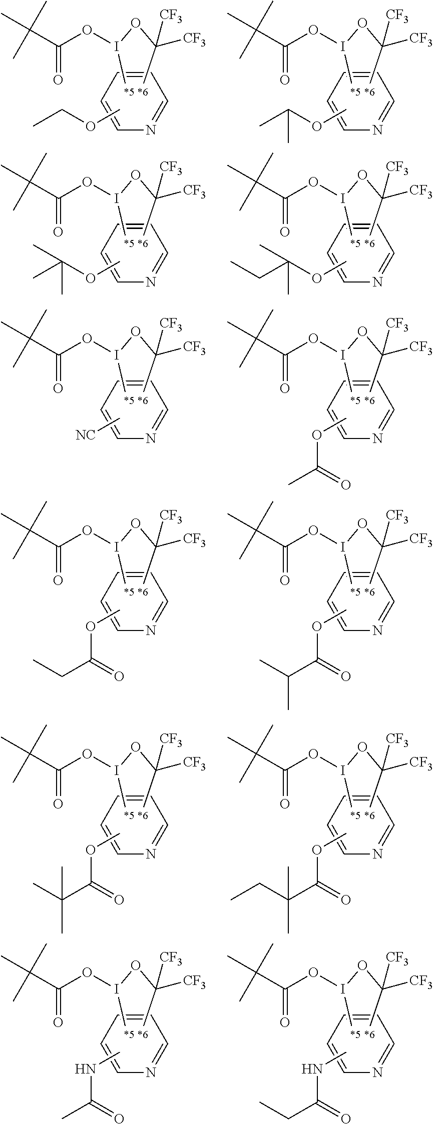

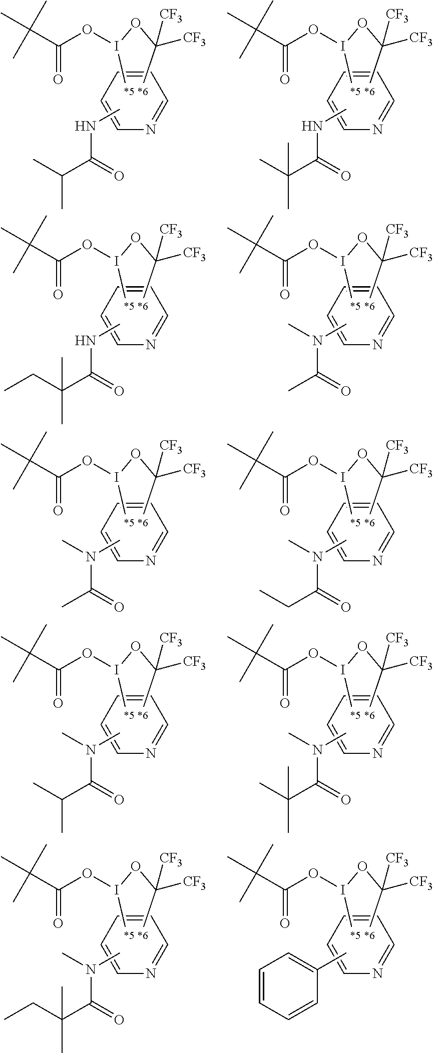

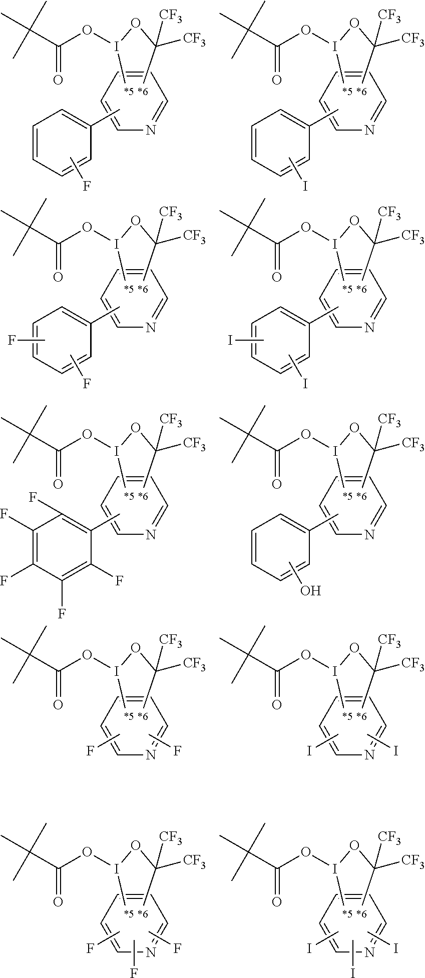

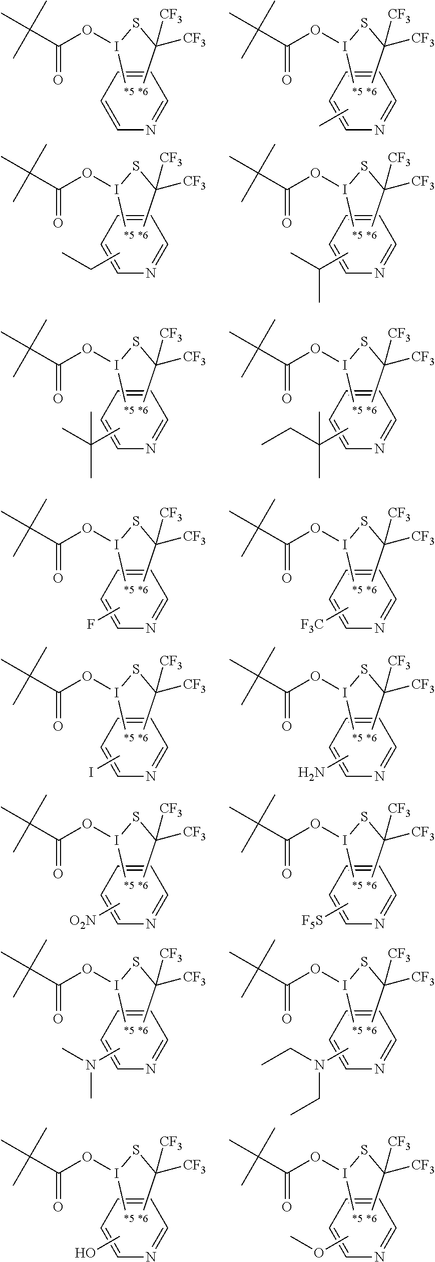

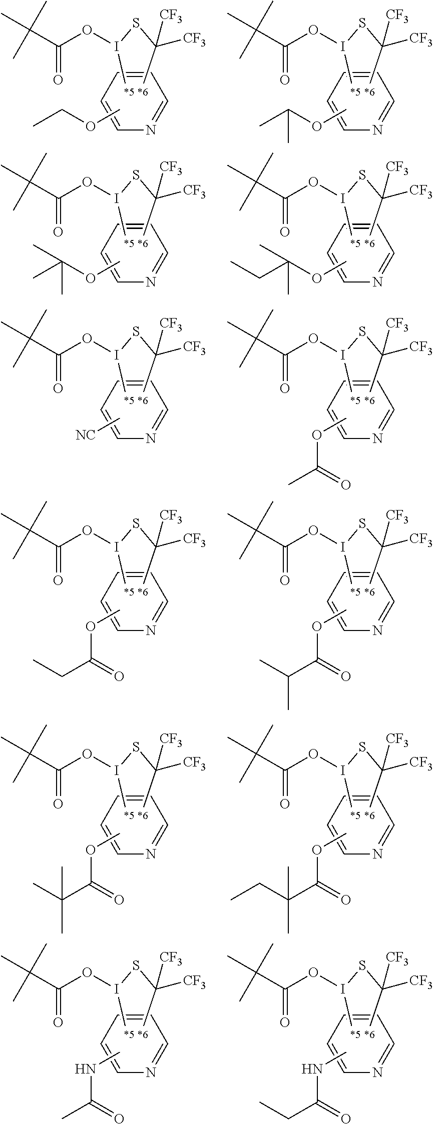

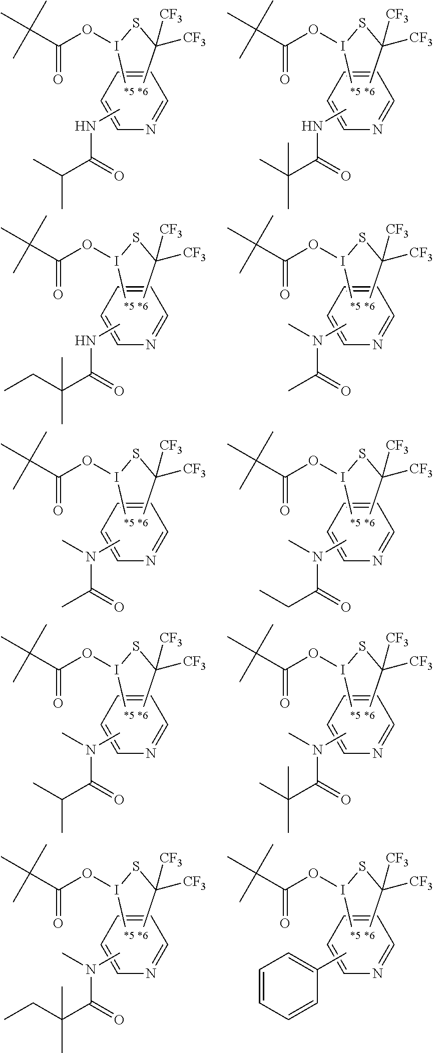

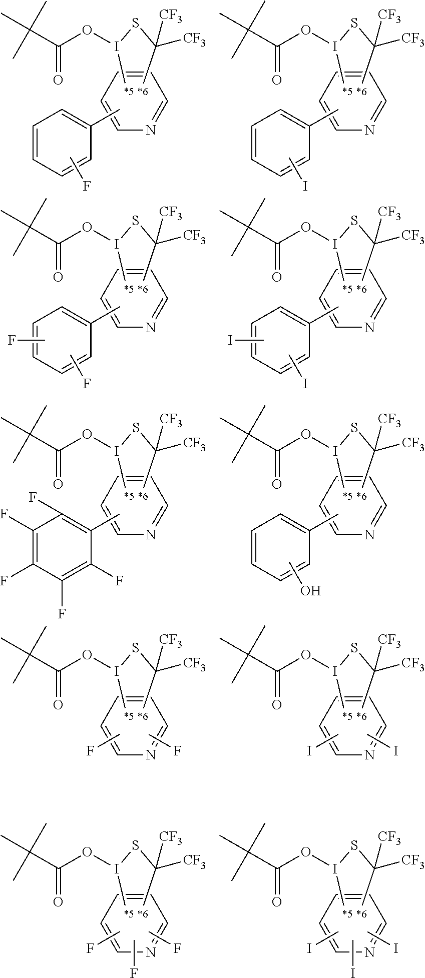

### [Carboxy Group-Containing Compound]

The carboxy group-containing compound is preferably a polymer (carboxy group-containing polymer) having a repeating unit represented by the following formula (4) or a compound (low-molecular compound) represented by the following formula (5).

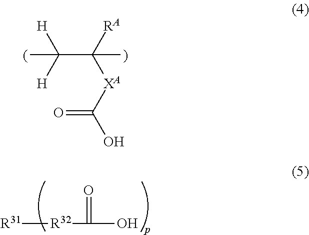

In the formula, R^{A} represents a hydrogen atom, a halogen atom, a methyl group, or a trifluoromethyl group. X^{A} represents a single bond, a phenylene group, a naphthylene group, or *—C(═O)—O—X^{A3}. X^{A3} represents a saturated hydrocarbylene group having 1 to 20 carbon atoms, a phenylene group, or a naphthylene group, and the saturated hydrocarbylene group may have a hydroxy group, an ether bond, an ester bond, or a lactone ring. “*” represents a bond to a carbon atom of the main chain. “p” represents 1, 2, 3, or 4. R³¹ represents a p-valent hydrocarbon group having 1 to 40 carbon atoms or a p-valent heterocyclic group having 2 to 40 carbon atoms, and when “p” is 2, R³¹ may be an ether bond, a carbonyl group, an azo group, a thioether bond, a carbonate bond, a carbamate bond, a sulfinyl group, or a sulfonyl group. In addition, part or all of the hydrogen atoms of the p-valent hydrocarbon group or the p-valent heterocyclic group may be substituted with a group having a heteroatom, and part of the —CH₂— groups of the p-valent hydrocarbon group may be substituted with a group having a heteroatom. R³² represents a single bond or a hydrocarbylene group having 1 to 20 carbon atoms, part or all of the hydrogen atoms of the hydrocarbylene group may be substituted with a group having a heteroatom, and part of the —CH₂— groups of the hydrocarbylene group may be substituted with a group having a heteroatom. When “p” is 2, 3, or 4, R³²s each may be identical or different.

In the formula (4), R^{A} represents a hydrogen atom, a halogen atom, a methyl group, or a trifluoromethyl group. X^{A} represents a single bond, a phenylene group, a naphthylene group, or *—C(═O)—O—X^{A3}—. X^{A3} represents a saturated hydrocarbylene group having 1 to 20 carbon atoms, preferably 1 to 10 carbon atoms, a phenylene group, or a naphthylene group, and the saturated hydrocarbylene group may contain a hydroxy group, an ether bond, an ester bond, or a lactone ring. “*” represents a bond to a carbon atom of the main chain.

In the formula (5), “p” represents 1, 2, 3 or 4.

In the formula (5), R³¹ is a p-valent hydrocarbon group having 1 to 40 carbon atoms or a p-valent heterocyclic group having 2 to 40 carbon atoms, and when “p” is 2, R³¹ may be an ether bond, a carbonyl group, an azo group, a thioether bond, a carbonate bond, a carbamate bond, a sulfinyl group or a sulfonyl group. Part or all of the hydrogen atoms of the p-valent hydrocarbon group or the p-valent heterocyclic group may be substituted with a group having a heteroatom, and part of the —CH₂— groups of the p-valent hydrocarbon group may be substituted with a group having a heteroatom.

In the formula (5), R³² represents a single bond or a hydrocarbylene group having 1 to 20 carbon atoms, part or all of the hydrogen atoms of the hydrocarbylene group may be substituted with a group having a heteroatom, and part of the —CH₂— groups of the hydrocarbylene group may be substituted with a group having a heteroatom. When “p” is 2, 3, or 4, R³²s may be identical to or different from each other.

The p-valent hydrocarbon group represented by R³¹ may be saturated or unsaturated, and may be linear, branched, or cyclic. The p-valent hydrocarbon group is a group obtained by eliminating p hydrogen atoms from a hydrocarbon. Examples of the hydrocarbon include alkanes having 1 to 40 carbon atoms, alkenes having 2 to 40 carbon atoms, alkynes having 2 to 40 carbon atoms, saturated cyclic hydrocarbons having 3 to 40 carbon atoms, unsaturated cyclic hydrocarbons having 3 to 40 carbon atoms, and aromatic hydrocarbons having 6 to 40 carbon atoms.

Examples of the alkanes having 1 to 40 carbon atoms include methane, ethane, propane, butane, pentane, hexane, heptane, octane, nonane, decane, and structural isomers thereof.

Examples of the alkenes having 2 to 40 carbon atoms include ethylene, propylene, butene, pentene, hexene, heptene, octene, nonene, decene, and structural isomers thereof.

Examples of the alkyne having 2 to 40 carbon atoms include acetylene, propyne, butyne, pentyne, hexyne, heptyne, octyne, nonyne, decyne, and structural isomers thereof.

Examples of the saturated cyclic hydrocarbon having 3 to 40 carbon atoms include cyclopropane, cyclobutane, cyclohexane, cycloheptane, cyclooctane, adamantane, and norbornane.

Examples of the unsaturated cyclic hydrocarbon having 3 to 40 carbon atoms include cyclopropene, cyclobutene, cyclopentene, cyclohexene, cycloheptene, cyclooctene, and norbornene.

Examples of the aromatic hydrocarbons having 6 to 40 carbon atoms include benzene, naphthalene, and biphenyl.

The p-valent heterocyclic group represented by R³¹ is a group obtained by eliminating p hydrogen atoms from a heterocyclic compound. Examples of the heterocyclic compound include furan, pyridine, pyrazole, and thiazolidine.

In the p-valent hydrocarbon group or the p-valent heterocyclic group, part or all of its hydrogen atoms may be substituted with a group having a heteroatom, such as an oxygen atom, a sulfur atom, a nitrogen atom, or a halogen atom. As a result, the p-valent hydrocarbon group or the p-valent heterocyclic group may have a hydroxy group, a cyano group, a fluorine atom, a chlorine atom, a bromine atom, an iodine atom, etc. Furthermore, part of the —CH₂— groups constituting the p-valent hydrocarbon group may be substituted with a group having a heteroatom, such as an oxygen atom, a sulfur atom, or a nitrogen atom. As a result, the p-valent hydrocarbon group may have a carbonyl group, an ether bond, a thioether bond, an ester bond, a sulfonic acid ester bond, a carbonate bond, a carbamate bond, a lactone ring, a sultone ring, a carboxylic anhydride (—C(═O)—O—C(═O)—), etc.

The hydrocarbylene group represented by R³² may be saturated or unsaturated, and may be linear, branched, or cyclic. Specific examples thereof include: alkanediyl groups having 1 to 20 carbon atoms, such as a methanediyl group, an ethane-1,1-diyl group, an ethane-1,2-diyl group, propane-1,2-diyl group, a propane-1,3-diyl group, a butane-1,4-diyl group, a pentane-1,5-diyl group, a hexane-1,6-diyl group, a heptane-1,7-diyl group, an octane-1,8-diyl group, a nonane-1,9-diyl group, a decane-1,10-diyl group, an undecane-1,11-diyl group, and a dodecane-1,12-diyl group; saturated cyclic hydrocarbylene groups having 3 to 20 carbon atoms, such as a cyclopentanediyl group, a cyclohexanediyl group, a norbornanediyl group, and an adamantanediyl group; unsaturated aliphatic hydrocarbylene groups having 2 to 20 carbon atoms, such as a vinylene group and a propene-1,3-diyl group; arylene groups having 6 to 20 carbon atoms, such as a phenylene group and a naphthylene group; and groups obtained by combining these groups. Furthermore, part or all of the hydrogen atoms of the hydrocarbylene group may be substituted with a group having a heteroatom, such as an oxygen atom, a sulfur atom, a nitrogen atom, or a halogen atom, or part of the —CH₂— groups constituting the hydrocarbylene group may be substituted with a group having a heteroatom, such as an oxygen atom, a sulfur atom, or a nitrogen atom. As a result, the hydrocarbylene group may have a hydroxy group, a cyano group, a fluorine atom, a chlorine atom, a bromine atom, an iodine atom, a carbonyl group, an ether bond, a thioether bond, an ester bond, a sulfonic acid ester bond, a carbonate bond, a carbamate bond, a lactone ring, a sultone ring, a carboxylic anhydride, etc.

Among the carboxylic acid compounds represented by the formula (5), those in which “p” is 2, 3, or 4 are preferable. In these cases, when the carboxylic acid compound is mixed with a hypervalent iodine compound, a strong, high-molecular-weight resist film can be readily formed, which is preferable from the viewpoints of etching resistance and developer resistance.

Specific examples of the repeating unit, containing a carboxy group, represented by the formula (4), include, but are not limited to, those shown below. In the following formulae, R^{A} represents the same as defined above.

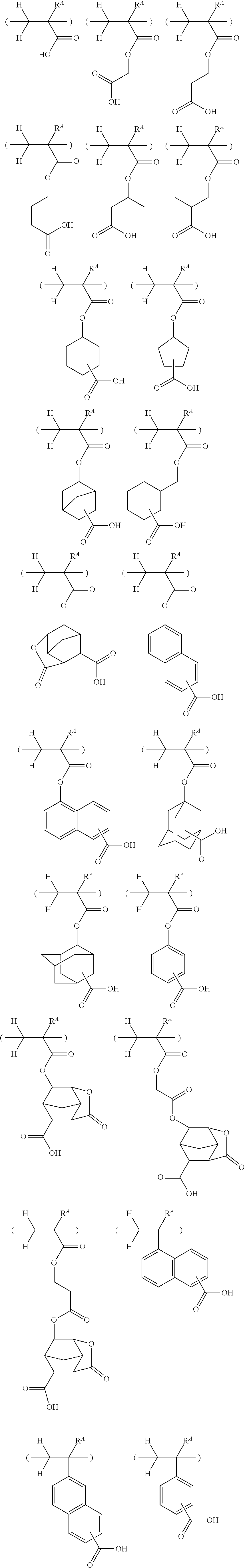

Examples of the carboxylic acid compound represented by the formula (5) include those shown below, but are not limited thereto. The carboxylic acid compound may be a commercially available product or may be synthesized.

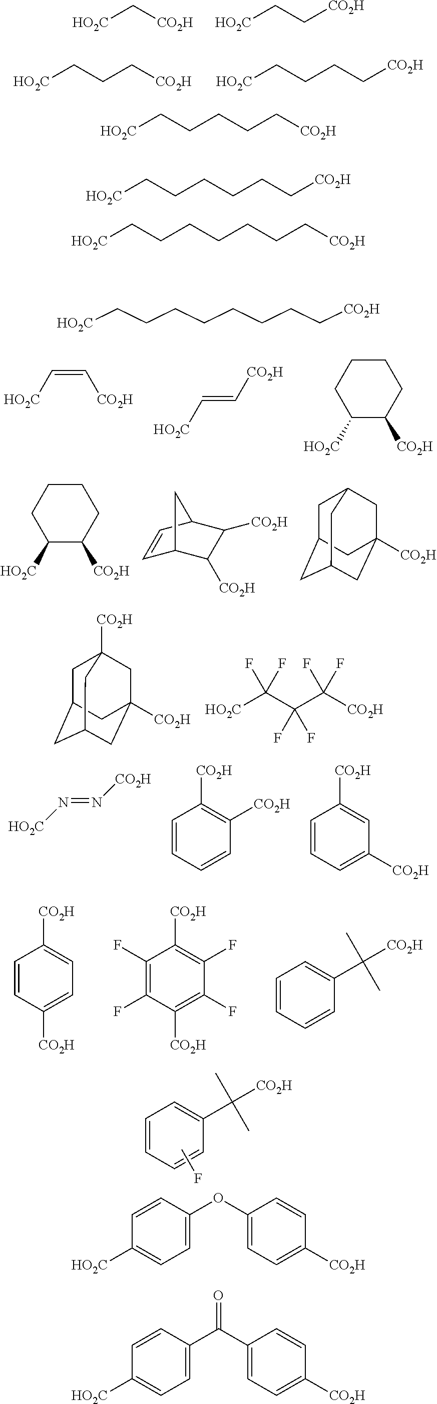

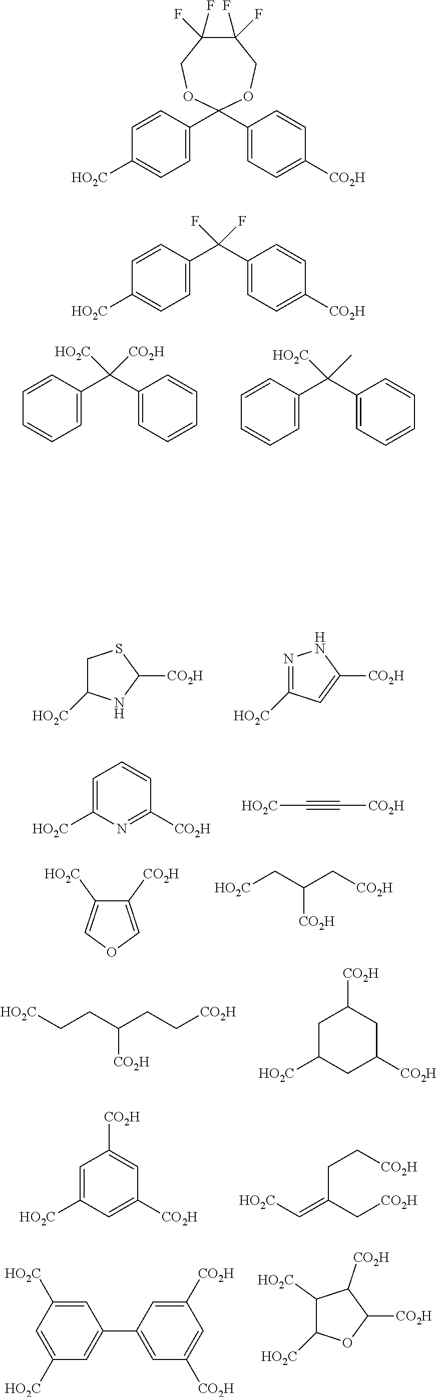

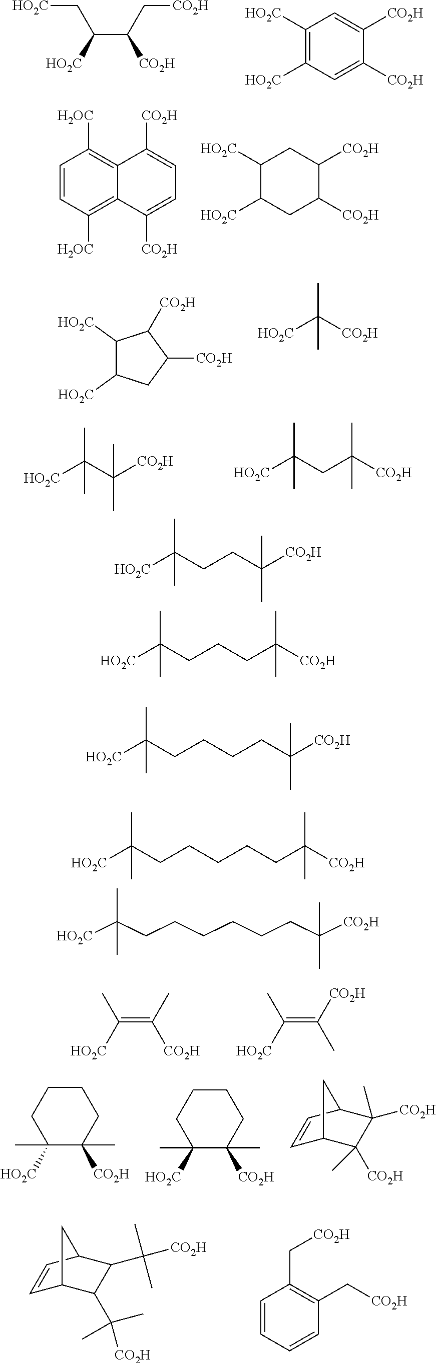

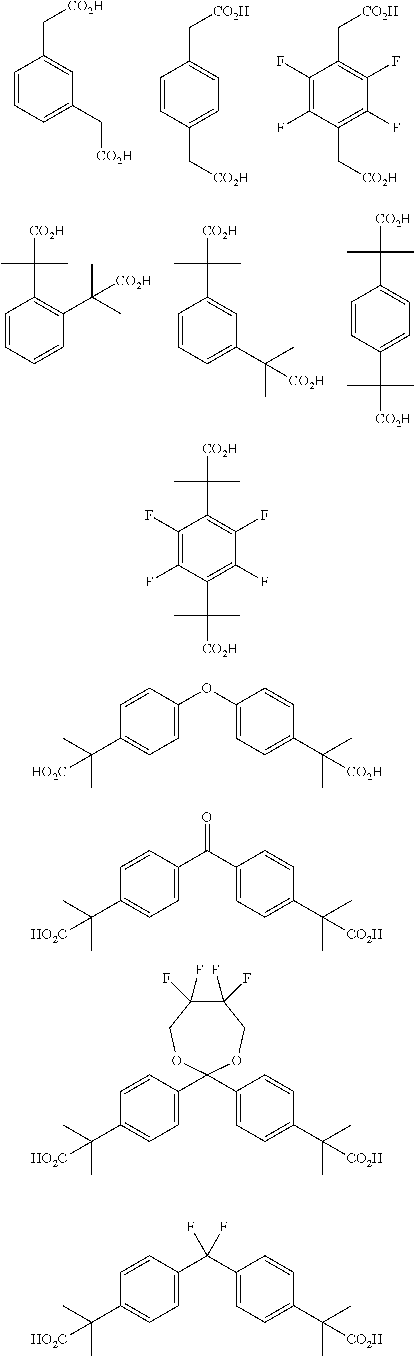

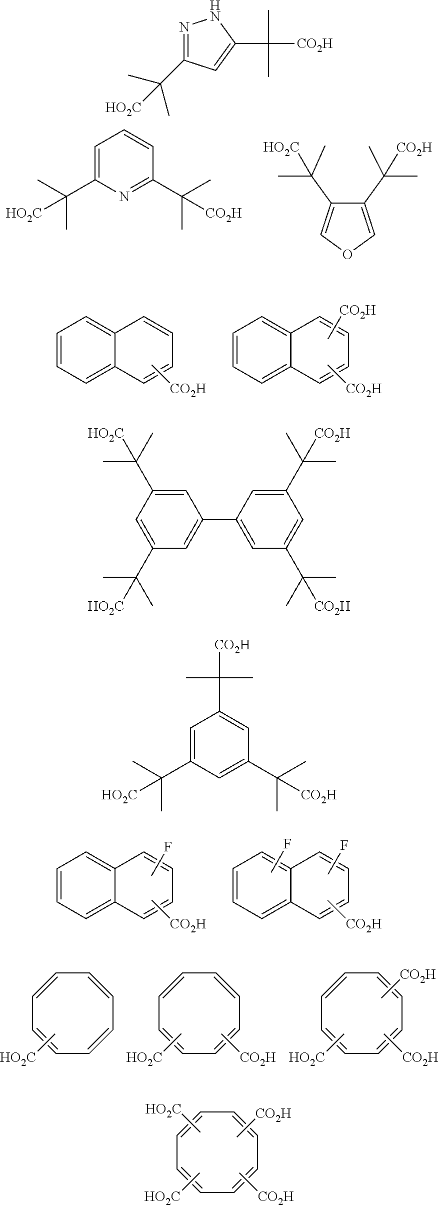

The carboxy group-containing polymer having the repeating unit represented by the formula (4) may further have other repeating units (hereinafter also referred to as “other repeating units”). The other repeating unit is not particularly limited, but is preferably one that can improve the solubility, in a solvent, of a polymer that is poorly soluble in the solvent when it contains only a repeating unit having a carboxyl group. For the other repeating units, those having a rigid skeleton and a cyclic structure that is expected to have high etching resistance, and those having a styrene skeleton are preferable.

Specific examples of the other repeating units include, but are not limited to, those shown below. In the following formulae, R^{A} represents the same as defined above, and X^{B} represents each independently —CH₂— or —O—.

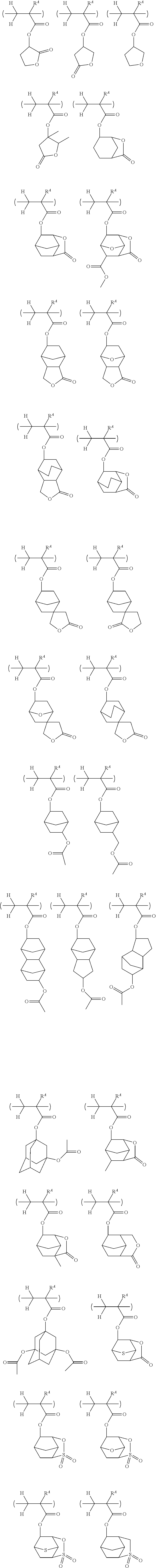

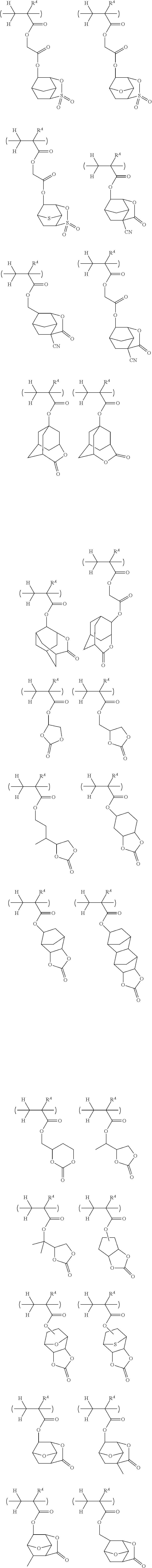

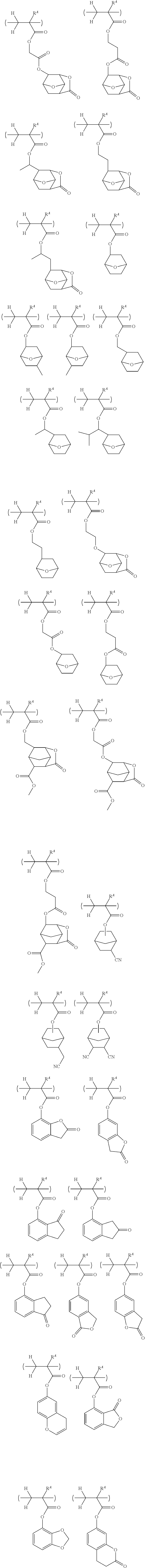

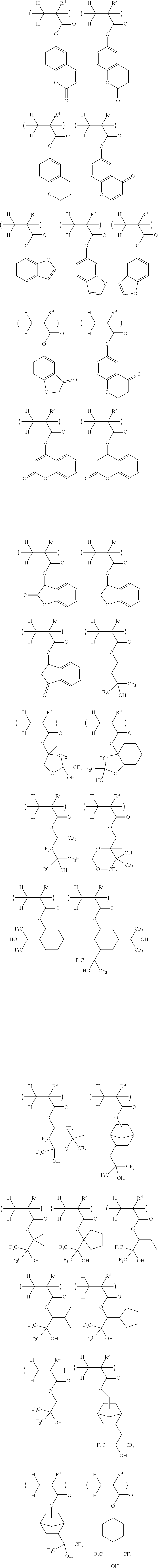

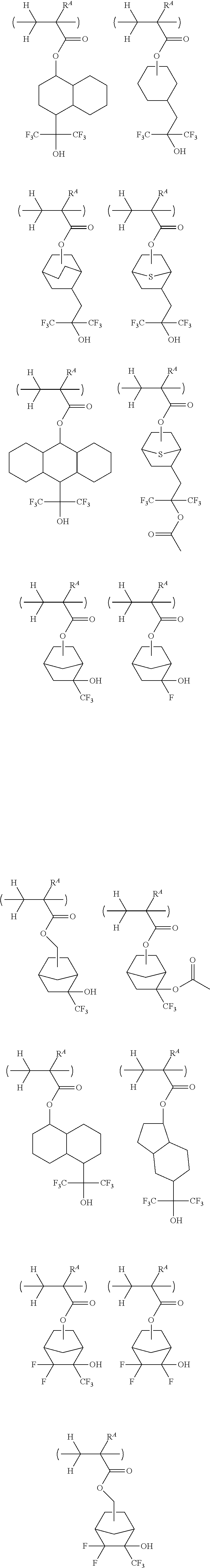

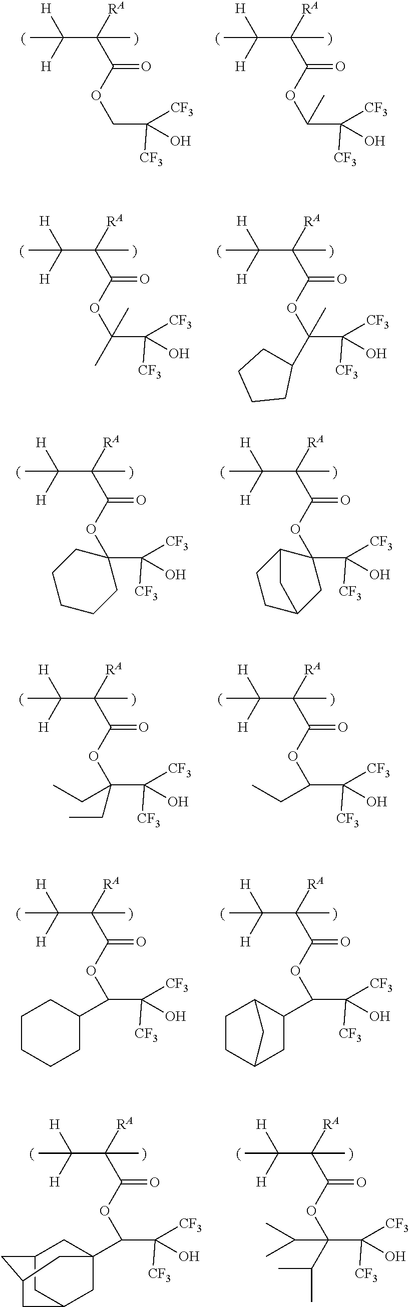

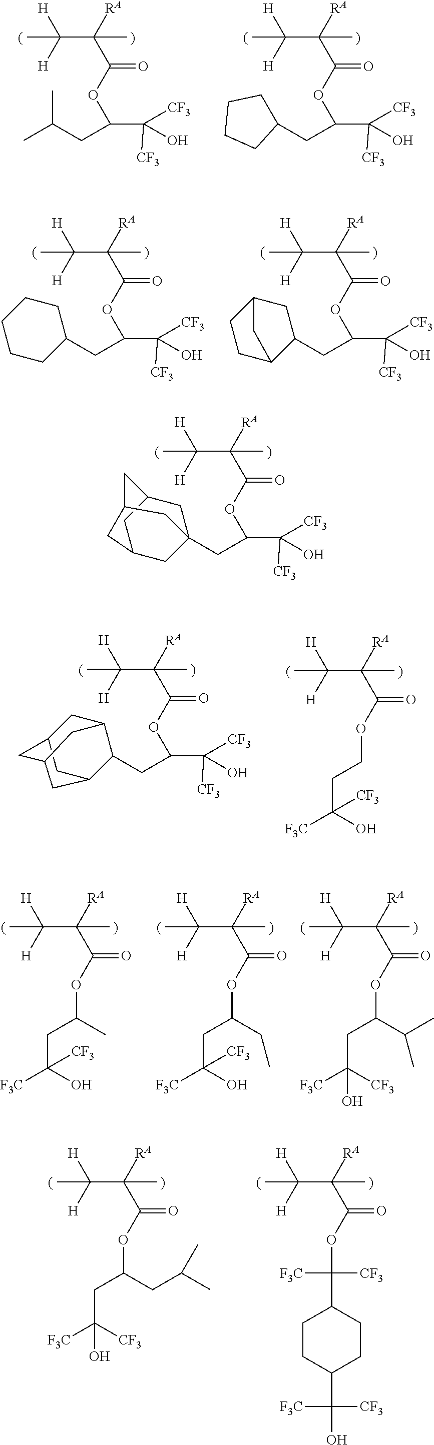

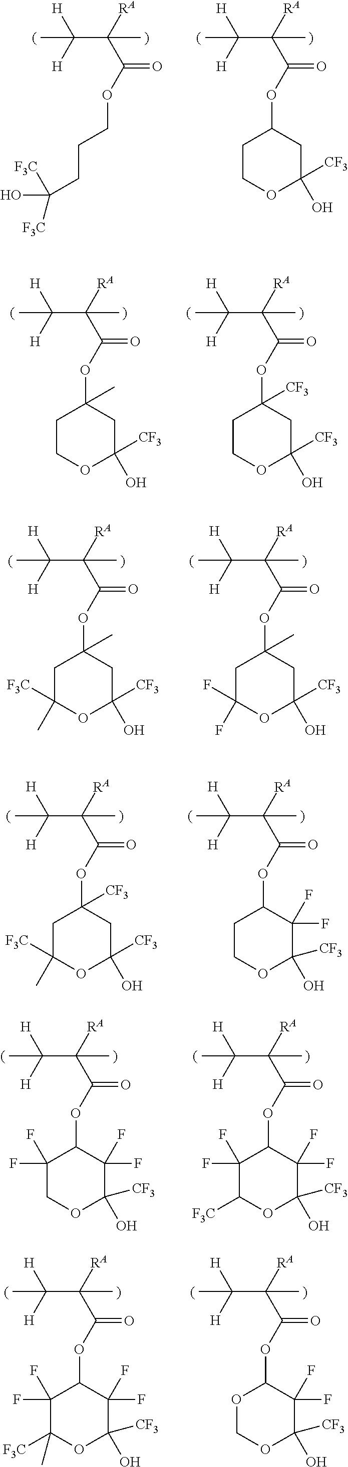

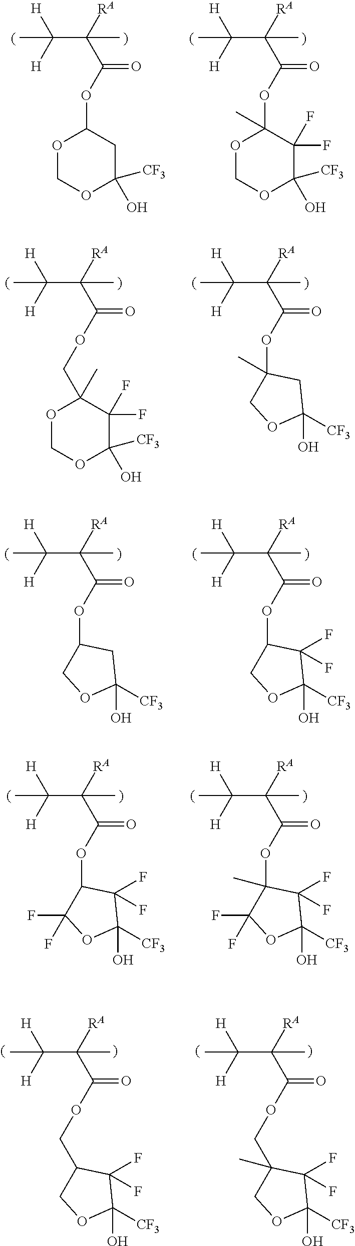

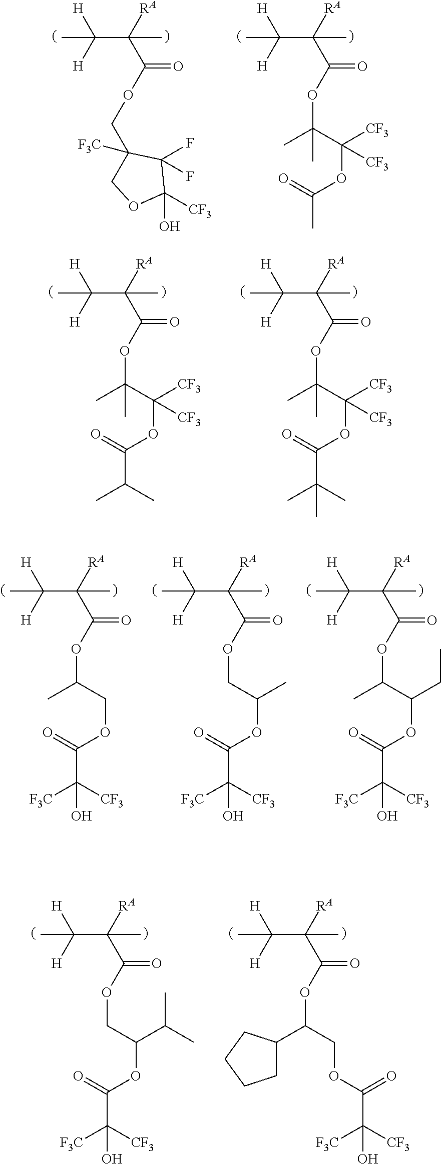

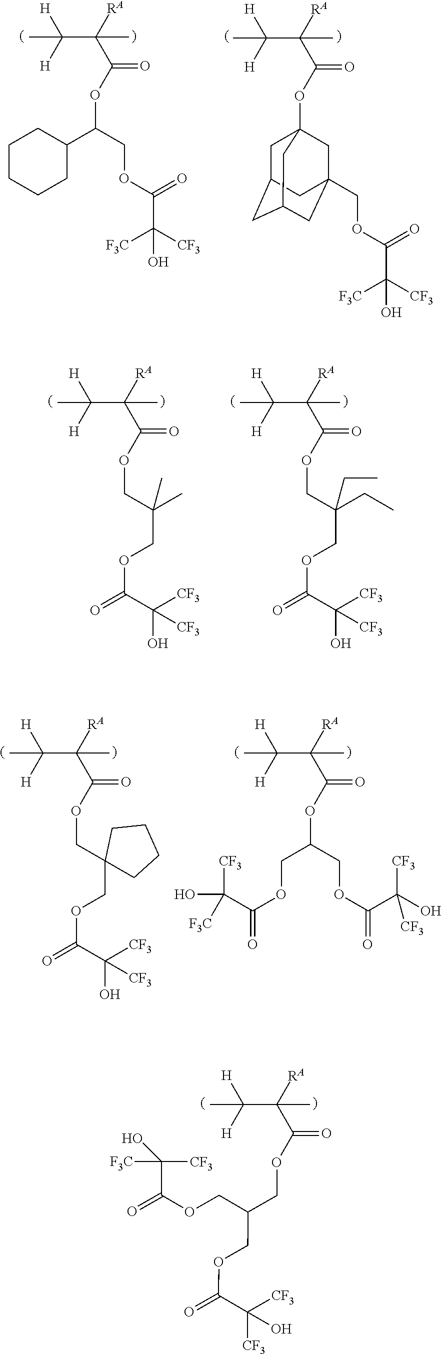

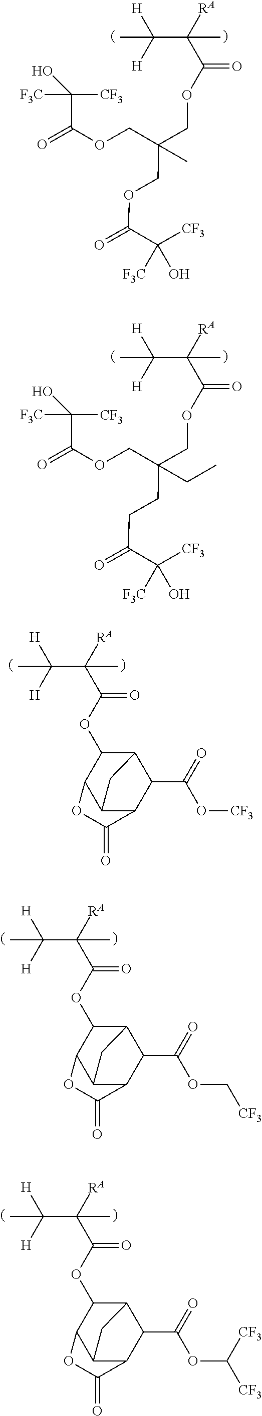

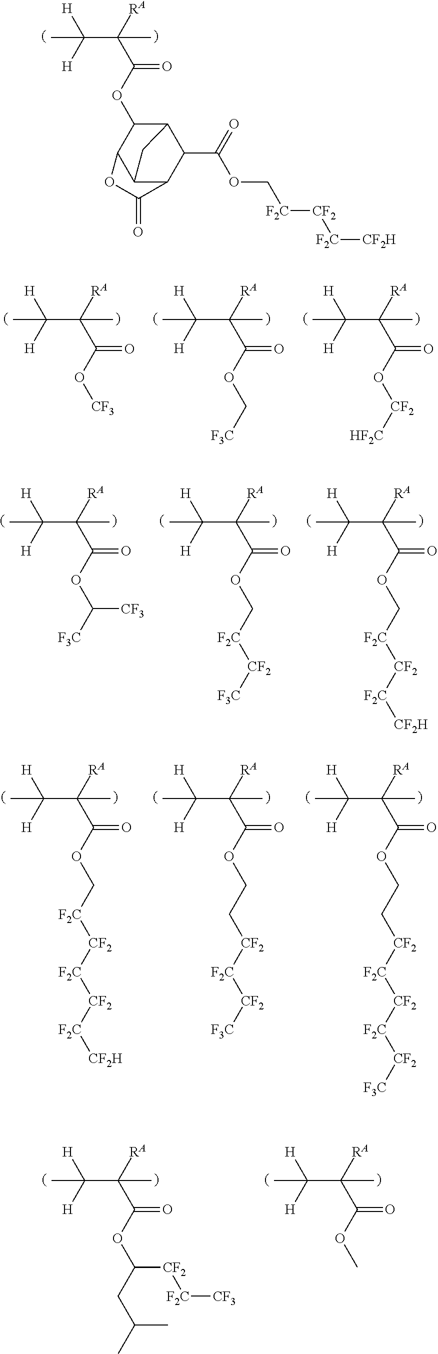

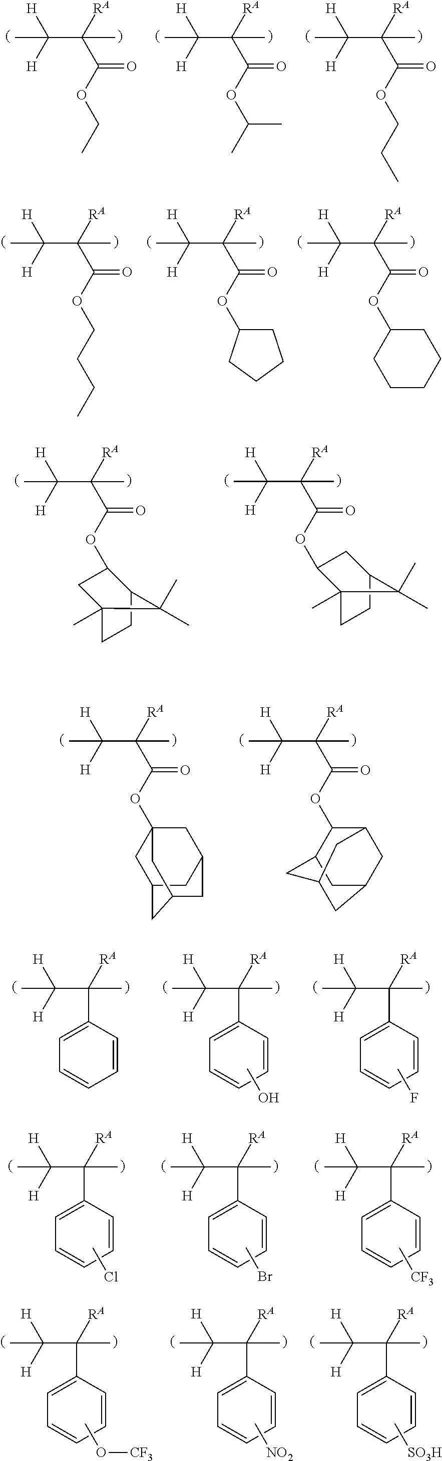

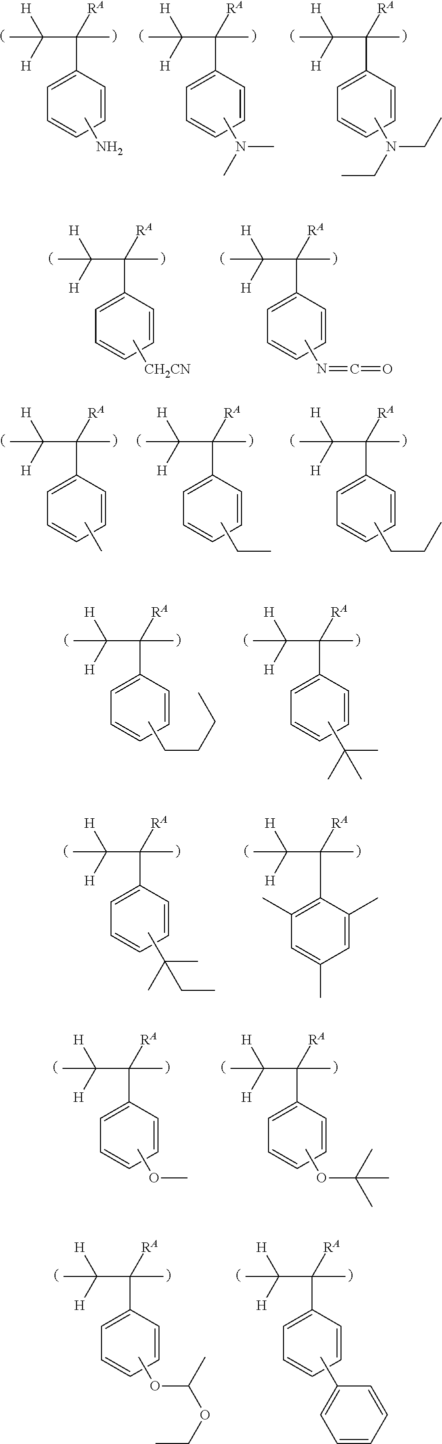

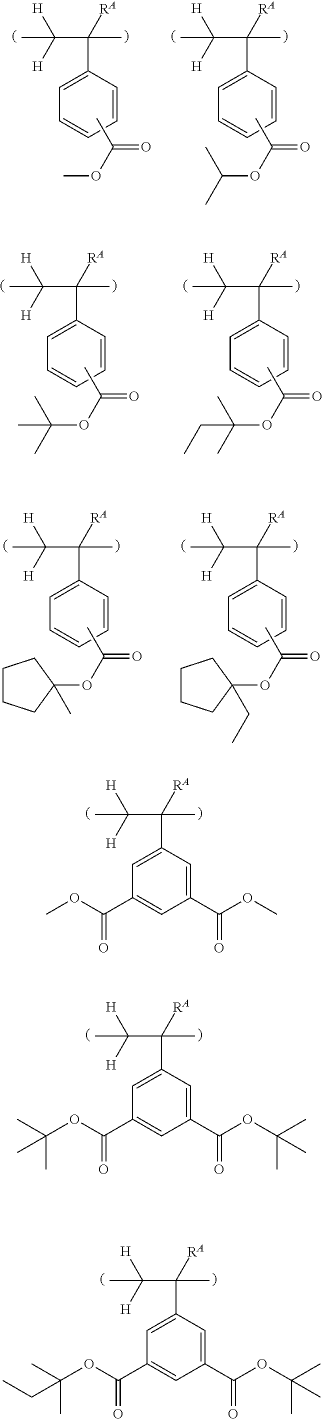

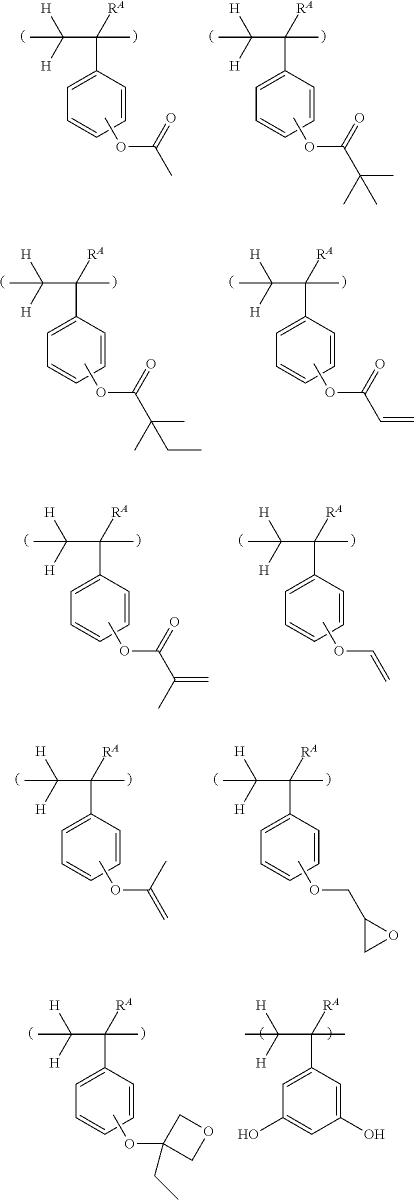

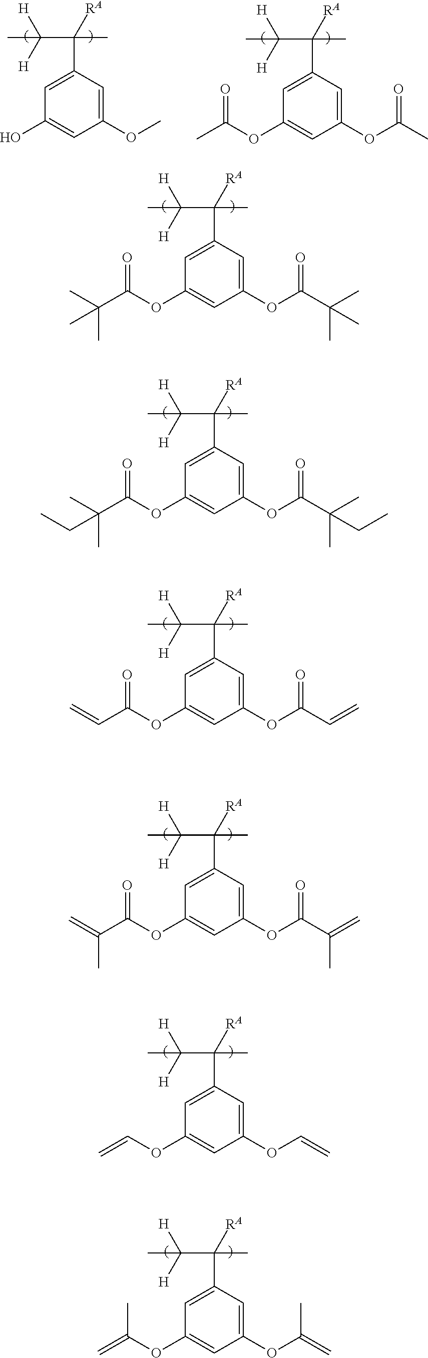

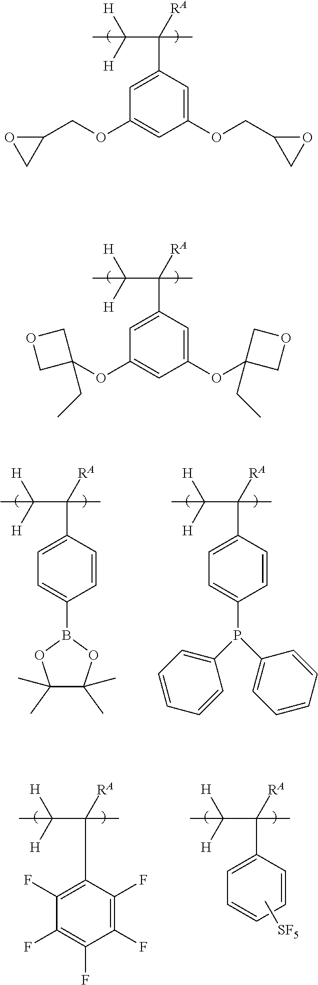

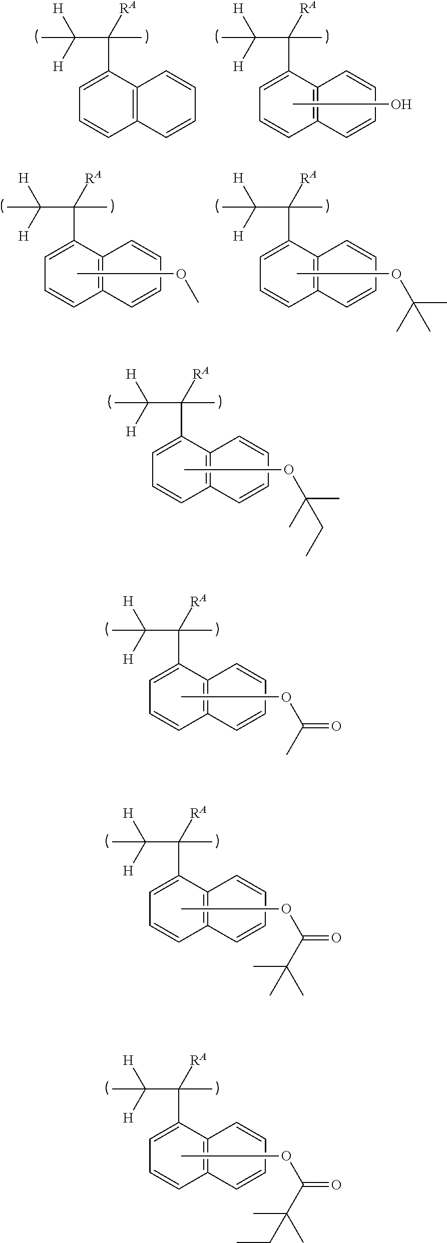

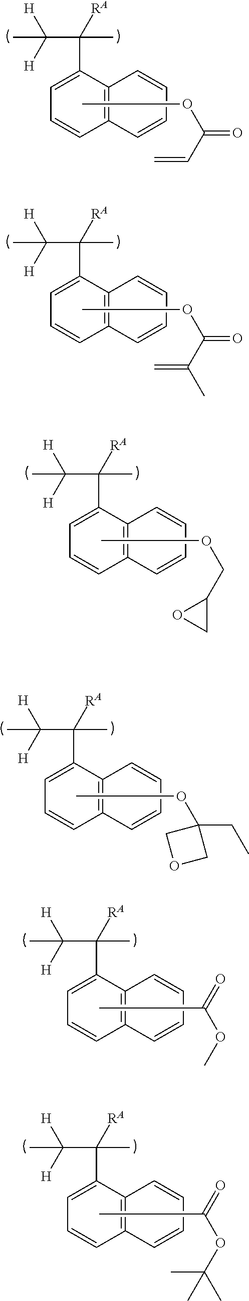

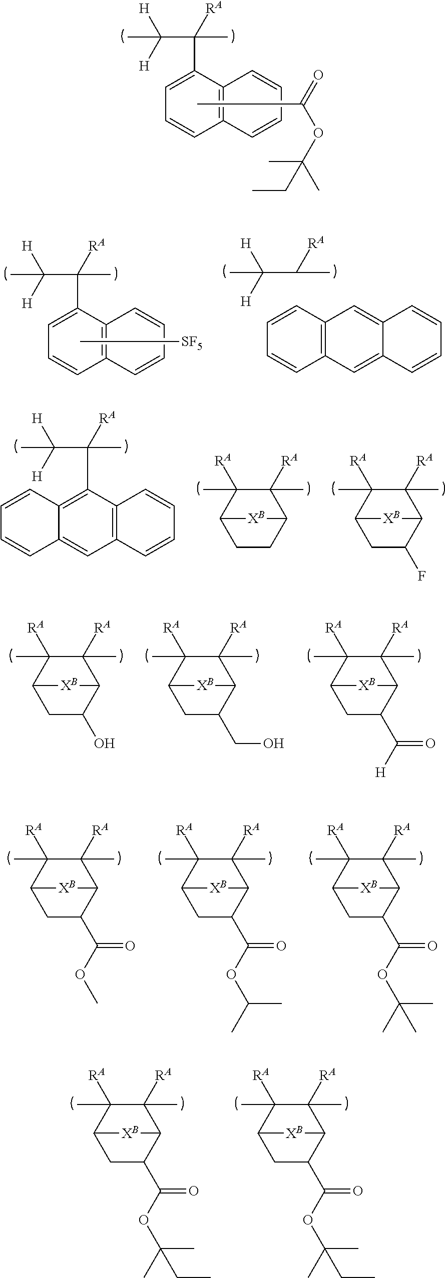

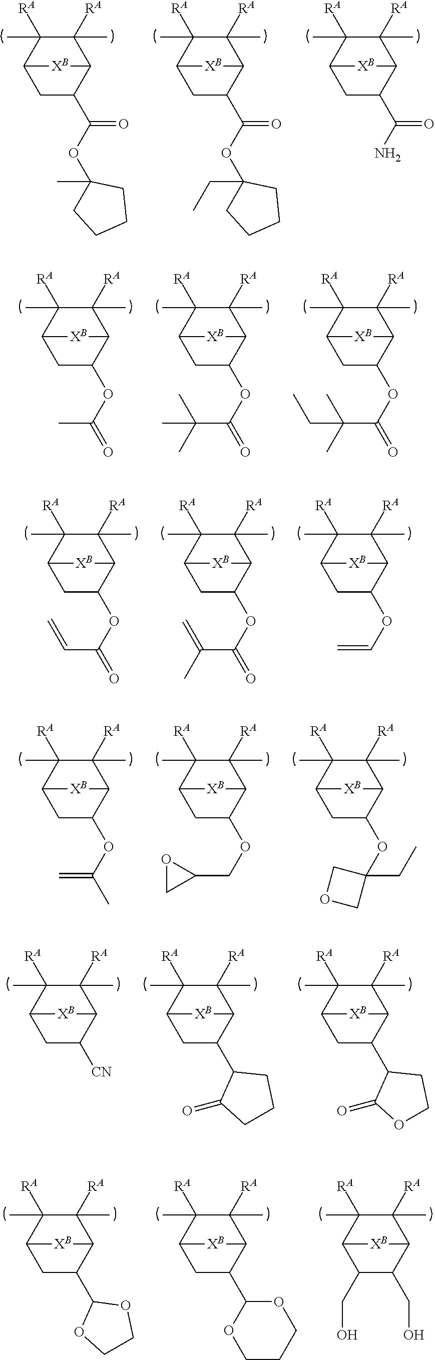

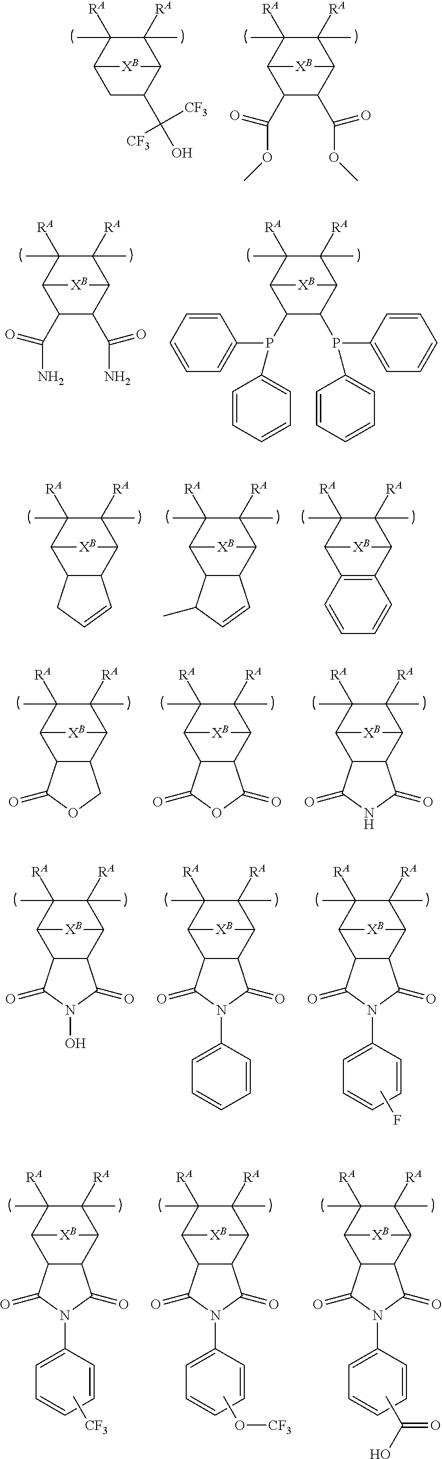

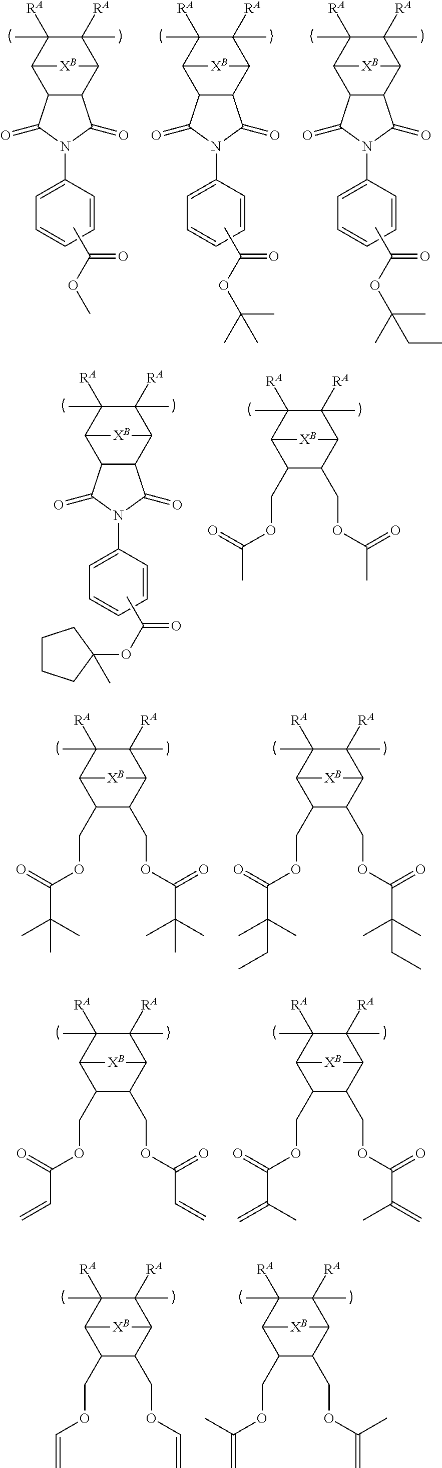

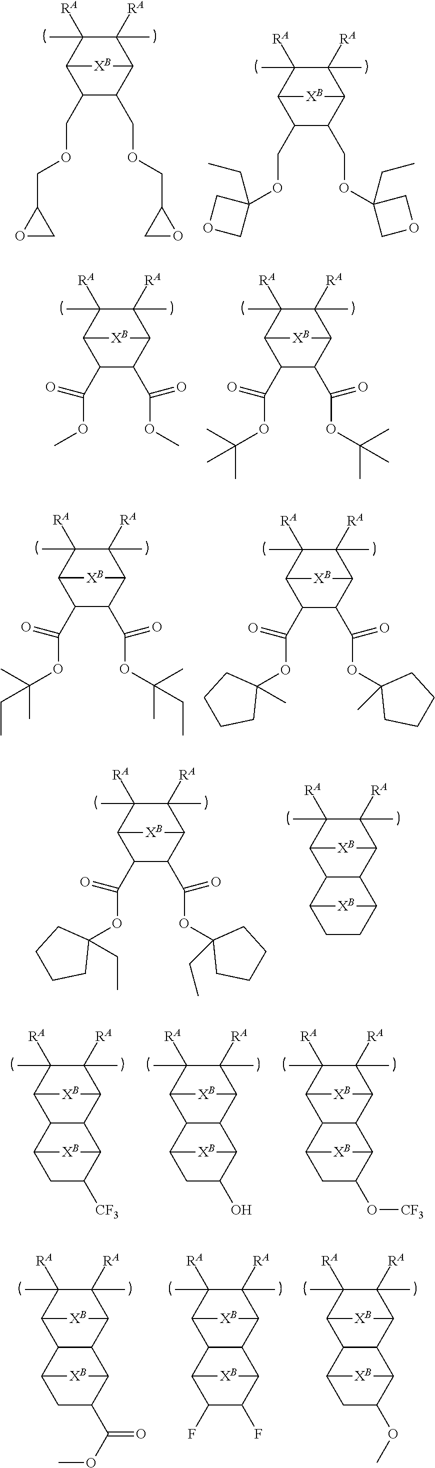

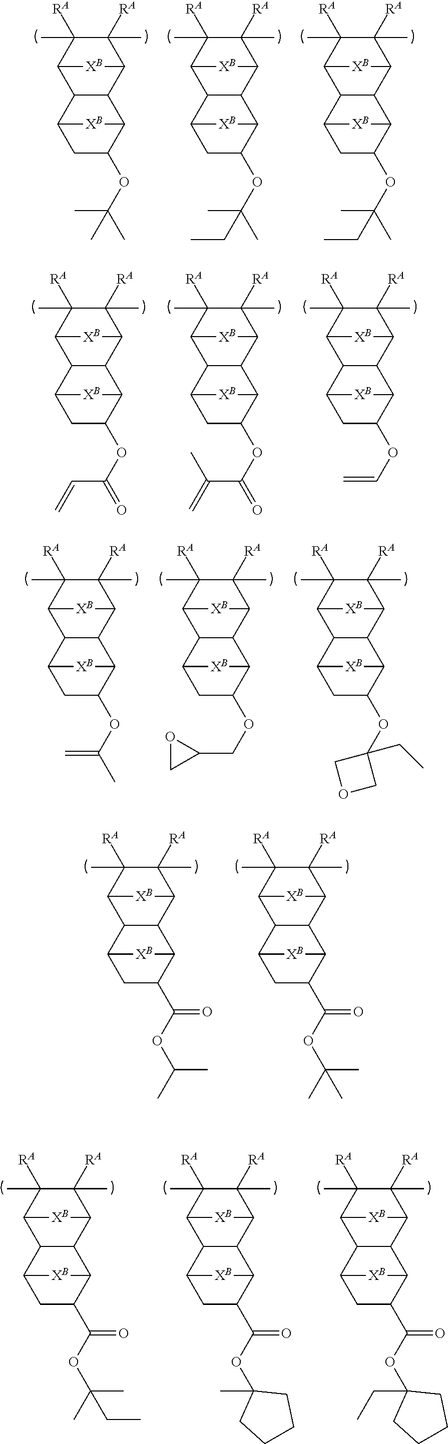

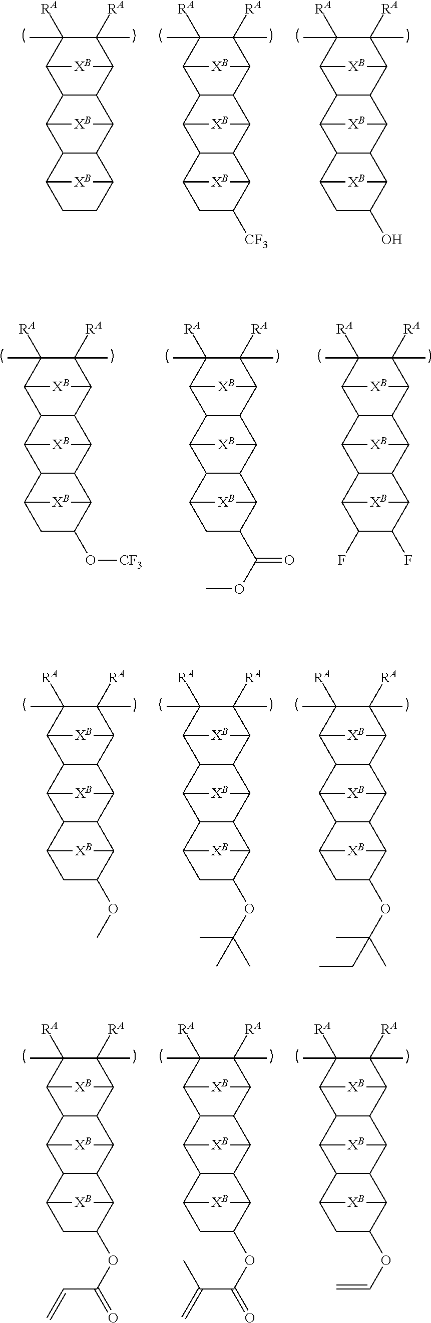

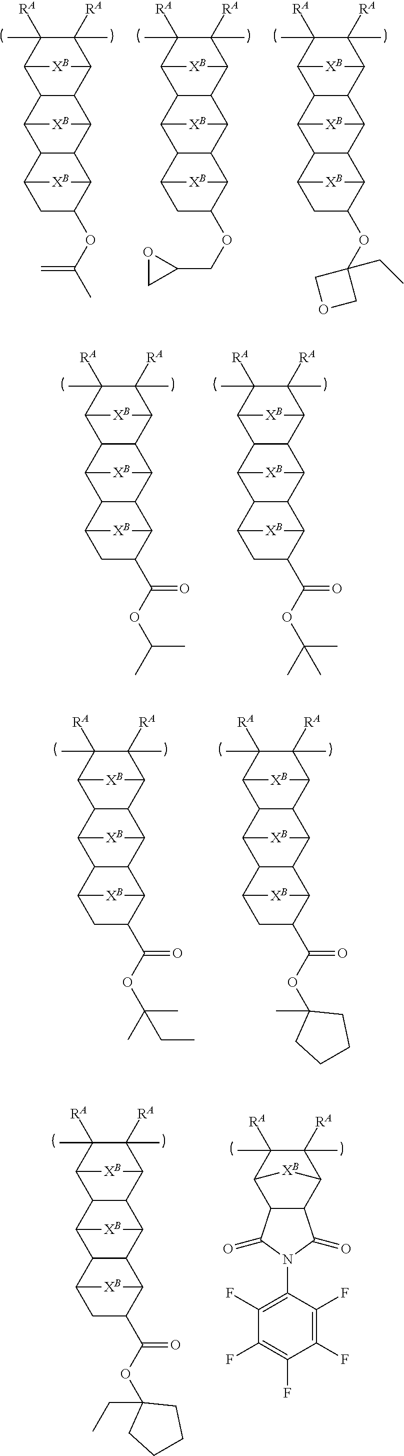

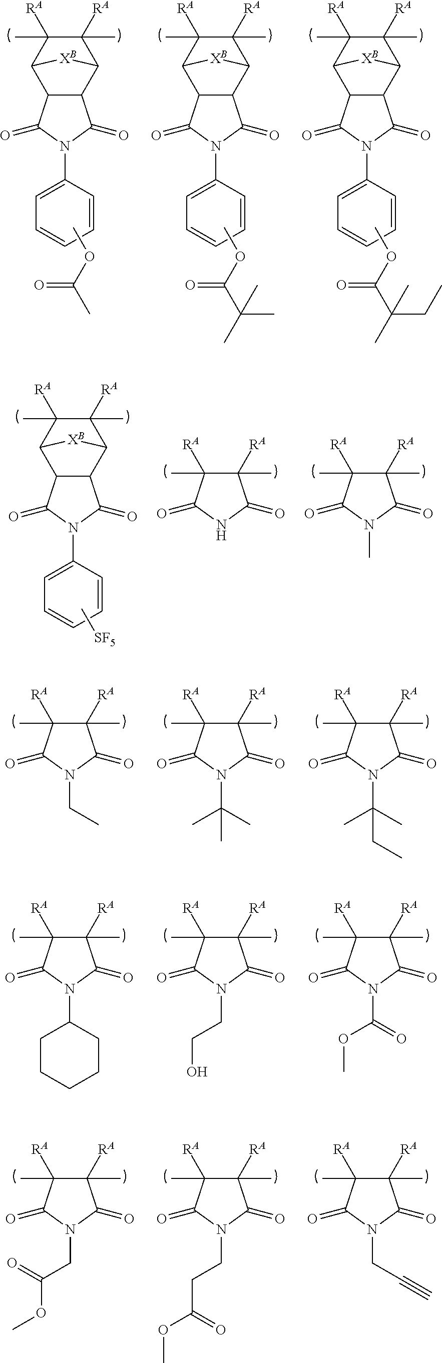

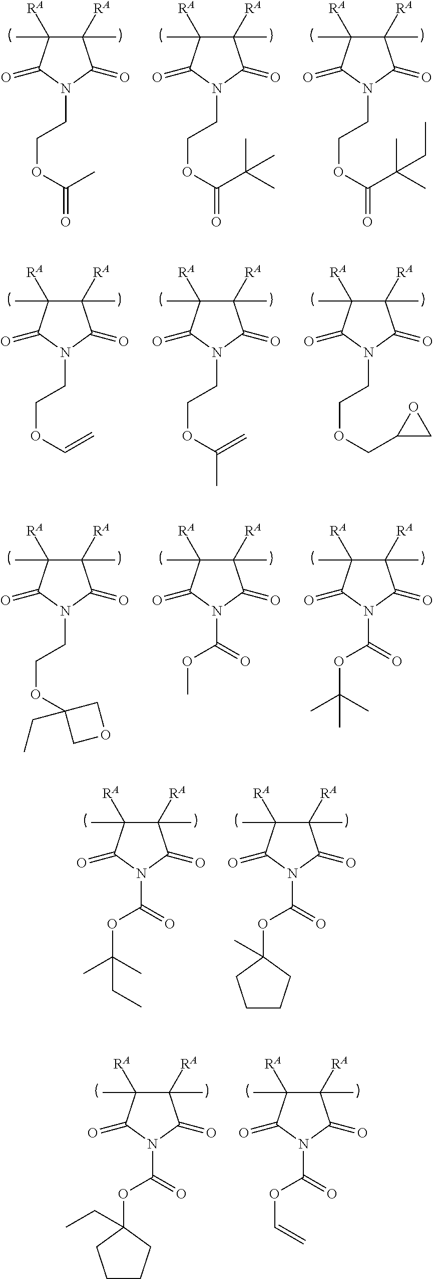

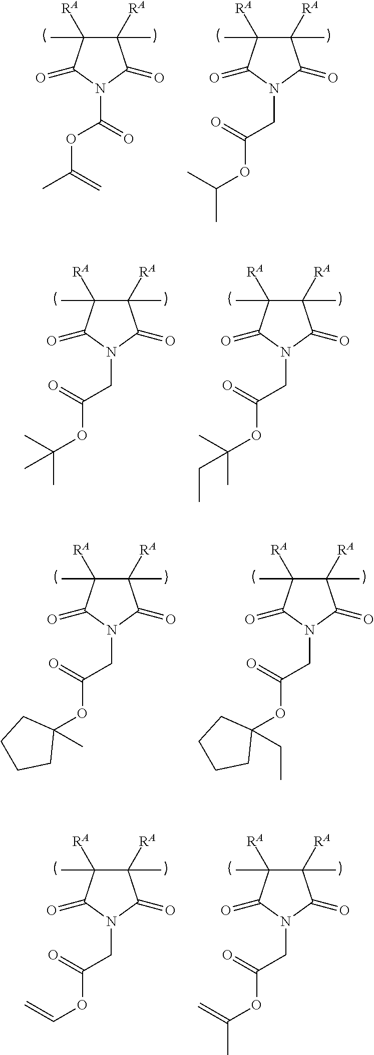

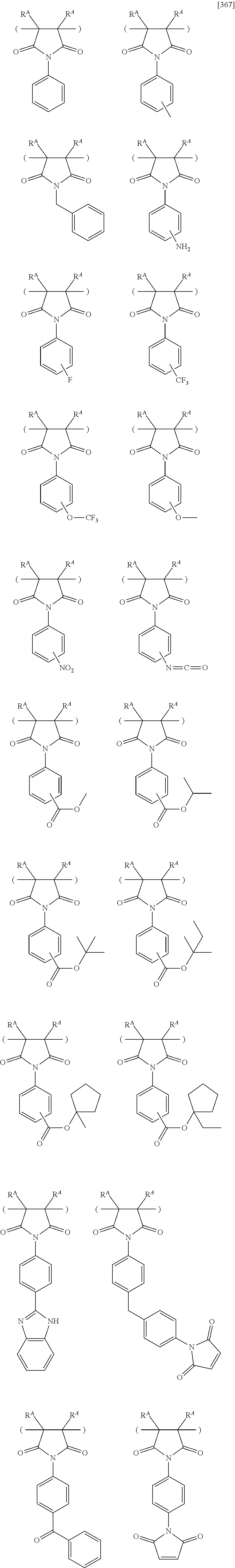

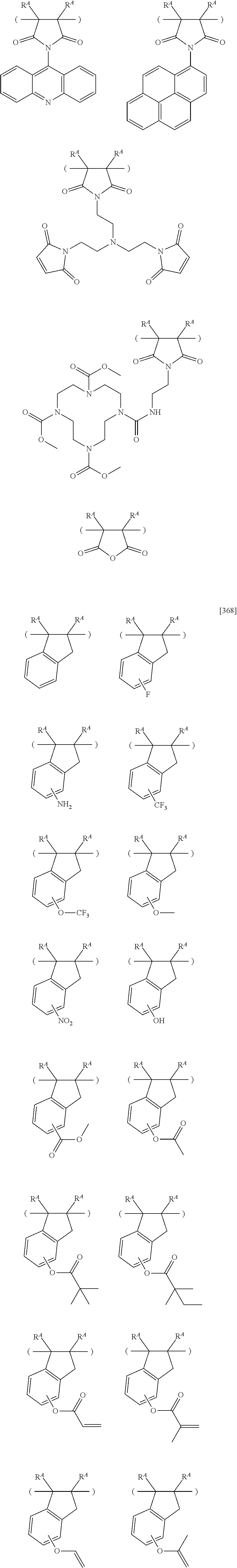

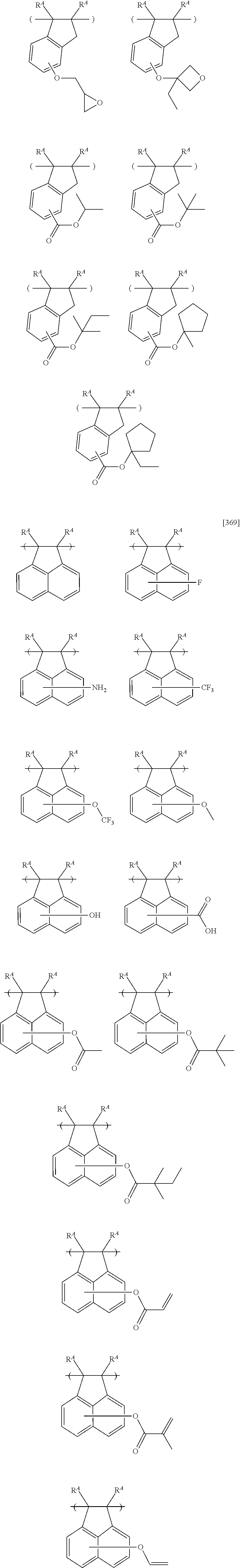

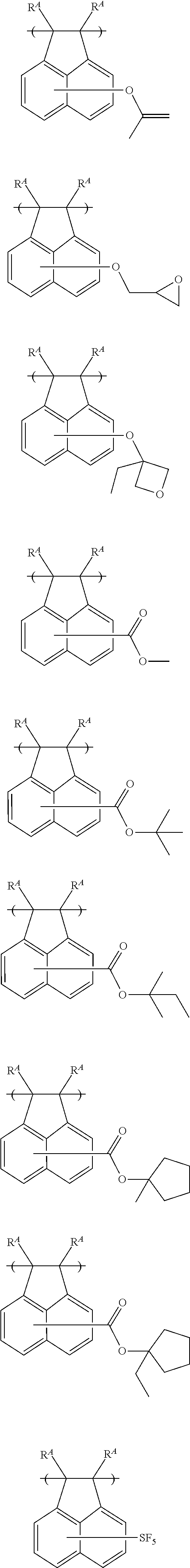

In the inventive resist composition, the content ratio of the hypervalent iodine compound relative to the carboxy group-containing compound (the polymer having the repeating unit represented by the formula (4) and/or the compound represented by the formula (5)) (when the carboxy group-containing compound is a carboxy group-containing polymer, the content ratio of the hypervalent iodine compound (mol) relative to the carboxylic acid-containing repeating unit (mol) in the polymer, and when the carboxy group-containing compound is a low-molecular compound represented by the formula (5), the content ratio of the hypervalent iodine compound (mol) relative to the low-molecular compound (mol)) is preferably hypervalent iodine compound:carboxy group-containing compound=10:90 to 90:10, more preferably 20:80 to 80:20, and further preferably 30:70 to 70:30 in a mole ratio.

One kind of the hypervalent iodine compound may be used, or two or more kinds thereof may be used in combination. One kind of the carboxy group-containing polymer may be used, or two or more kinds thereof having different composition ratios, weight-average molecular weights (Mw), and/or molecular weight distributions (Mw/Mn) may be used in combination. One kind of the low-molecular compound may be used, or two or more kinds thereof may be used in combination. One of the carboxy group-containing polymer and the low-molecular compound may be used, or both may be used in combination.

As described later, the hypervalent iodine compound is the main component of the inventive resist composition. Therefore, relative to 100 parts by mass of the total solids content of the inventive resist composition, the total amount of the hypervalent iodine compound and the carboxy group-containing compound is preferably 30 to 100 parts by mass, more preferably 50 to 100 parts by mass, further preferably 80 to 100 parts by mass, extremely preferably 90 to 100 parts by mass, particularly preferably 95 to 100 parts by mass, and even more preferably 98 to 100 parts by mass.

In the carboxy group-containing polymer, the content ratio (mole ratio) of the carboxy group-containing repeating unit (the repeating unit presented by the formula (4)) and the other repeating units is preferably carboxy group-containing repeating unit: other repeating unit=10:90 to 90:10, more preferably 15:85 to 85:15, and further preferably 20:80 to 80:20.

The carboxy group-containing polymer preferably has a weight-average molecular weight (Mw) of 1000 to 500000, more preferably 3000 to 100000, further preferably 3000 to 20000. It should be understood that, in the present invention, the weight-average molecular weight Mw and the number-average molecular weight Mn are values measured in terms of standard polystyrene according to gel permeation chromatography (GPC) using tetrahydrofuran (THF) as an eluent, and the dispersity Mw/Mn is a value determined based on these values. When GPC is performed, it is typically performed at room temperature around 23° C., but it may be performed at a higher or lower temperature.

Furthermore, when the carboxy group-containing polymer has a broad molecular weight distribution (Mw/Mn), polymers having a lower molecular weight or a higher molecular weight are present. Therefore, there is a risk that a foreign substance may be found on a pattern after exposure and that the pattern shape may be degraded. Accordingly, as a pattern rule becomes smaller, the influence of Mw and Mw/Mn is likely to be greater. Accordingly, in order to obtain a resist composition that can be used suitably for a fine pattern dimension, the carboxy group-containing polymer preferably has a narrow dispersity Mw/Mn of 1.0 to 2.0.

Examples of methods for synthesizing the carboxy group-containing polymer include a method of polymerizing a monomer to give the repeating unit described above by heating in an organic solvent in the presence of a radical polymerization initiator.

Specific examples of the organic solvent to be used for the polymerization reaction include toluene, benzene, THF, diethyl ether, dioxane, cyclohexane, cyclopentane, cyclopentanone, cyclohexanone, methyl ethyl ketone (MEK), propylene glycol monomethyl ether (PGME), propylene glycol monomethyl ether acetate (PGMEA), and y-butyrolactone (GBL). Specific examples of the polymerization initiator include 2,2′-azobisisobutyronitrile (AIBN), 2,2′-azobis(2,4-dimethylvaleronitrile), dimethyl-2,2-azobis(2-methylpropionate), 1,1′-azobis(1-acetoxy-1-phenylethane), benzoyl peroxide, and lauroyl peroxide. The amount of the polymerization initiator to be added is preferably 0.01 to 25 mol % of the total amount of the monomers to be polymerized. The reaction temperature is preferably 50 to 150° C., more preferably 60 to 100° C. The reaction time is preferably 2 to 24 hours, and from the viewpoint of production efficiency, more preferably 2 to 12 hours.

The polymerization initiator may be added to the solution of the monomer and provided as the solution to the reaction vessel, or a solution of the initiator may be prepared separately from the solution of the monomer, and each solution may be provided to the reaction vessel independently. There is a possibility that the polymerization reaction may progress during waiting time due to radicals generated from the initiator, and that an ultra-high molecular weight polymer may be generated. Therefore, from the viewpoint of quality control, it is preferable to prepare each of the monomer solution and the initiator solution independently and add the solutions dropwise. Furthermore, in order to adjust the molecular weight, a publicly known chain transfer agent, such as dodecyl mercaptan or 2-mercaptoethanol, may also be used. In this case, the amount of the chain transfer agent to be added is preferably 0.01 to 20 mol % of the total amount of the monomers to be polymerized.

It should be understood that the amount of each monomer in the monomer solution may be, for example, set appropriately to achieve the preferable content ratios of the above-described repeating units.

### [Solvent]

The inventive resist composition contains a solvent. The solvent is not particularly limited as long as the solvent dissolves the hypervalent iodine compound, the carboxy group-containing compound, and other components described later, if contained, and allows film formation.

For such a solvent, organic solvents are preferable. Specific examples thereof include: ketones, such as cyclohexanone, methyl-2-n-pentyl ketone, and methyl isoamyl ketone; alcohols, such as 3-methoxybutanol, 3-methyl-3-methoxybutanol, 1-methoxy-2-propanol, 1-ethoxy-2-propanol, diacetone alcohol, 4-methyl-2-pentanol, and methyl 2-hydroxyisobutyrate; ethers, such as propylene glycol monomethyl ether, ethylene glycol monomethyl ether, propylene glycol monoethyl ether, ethylene glycol monoethyl ether, propylene glycol dimethyl ether, and diethylene glycol dimethyl ether; esters, such as propylene glycol monomethyl ether acetate, propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; carboxylic acids, such as formic acid, acetic acid, and propionic acid; lactones, such as y-butyrolactone; and mixed solvents thereof.

In the inventive resist composition, the amount of the solvent contained is preferably such an amount that the concentration of the solid contents in the resist composition is 0.1 to 20 mass %, more preferably 0.1 to 15 mass %, and further preferably 0.1 to 10 mass %. It is understood that, in the present invention, “solid content” is a general term for the components other than the solvent out of all the components of the resist composition. One kind of the solvent may be used, or two or more kinds thereof may be used in mixture.

When two or more solvents are used in combination, the combination is not particularly limited, but an aprotic solvent and a protic solvent can be combined. The aprotic solvent may be selected from the ketones, the ethers, the esters, and the lactones, each of which is described above, and the protic solvent may be selected from the alcohols and the carboxylic acids, each of which is described above. A combination of an ester as the aprotic solvent and a carboxylic acid as the protic solvent is preferable. When an aprotic solvent and a protic solvent are combined, the mixing ratio is not particularly limited, but the amount of the aprotic solvent relative to the total mass of both solvents is preferably 50 to 90 mass %, more preferably 60 to 80 mass %. When the aprotic solvent is within the above range, the balance between the aprotic solvent and the protic solvent is favorable, the solubility of the solid components in the resist composition is good, the resist composition becomes homogeneous, and the protic solvent cooperates with the hypervalent iodine compound to improve the resolvability of the resist composition.

### [Other Components]

The resist composition may further contain a surfactant. As the surfactant, a fluorine-based and/or silicone-based surfactant is preferable. Examples of such a surfactant include surfactants disclosed in paragraph [0276] of US2008/0248425A1. Furthermore, it is also possible to use a surfactant other than the fluorine-containing and/or silicone-containing surfactant, disclosed in paragraph [0280] of US2008/0248425A1.

When the resist composition contains the surfactant, the contained amount is preferably 0.0001 to 2 mass % of the total solid content. One kind of the surfactant may be used, or two or more kinds thereof may be used in combination.

The resist composition may further contain a radical scavenger. By adding a radical scavenger, photoreaction during photolithography can be controlled, and sensitivity can be adjusted.

Examples of the radical scavenger include hindered phenols, quinones, hindered amines, and thiol compounds. Specifically, examples of the hindered phenols include dibutylhydroxytoluene (BHT) and 2,2′-methylenebis (4-methyl-6-tert-butylphenol). Examples of the quinones include 4-methoxyphenol (methoquinone) and hydroquinone. Examples of the hindered amines include 2,2,6,6-tetramethylpiperidine and 2,2,6,6-tetramethylpiperidine-N-oxy radical. Examples of the thiols include dodecanethiol and hexadecanethiol.

When the resist composition contains the radical scavenger, the contained amount is preferably 0.01 to 10 mass % of the total solid content. One kind of the radical scavenger may be used, or two or more kinds thereof may be used in combination.

The resist composition may further contain a crosslinking agent. By adding a crosslinking agent, the crosslinking reaction during photolithography can be promoted, glass transition temperature of the pattern can be increased, and a pattern having excellent resolution of a thin line can be obtained.

Examples of the crosslinking agent include compounds having a carbon-carbon unsaturated bond as a functional group, such as a vinyl group, a (meth)acrylate group, an allyl group, an alkynyl group, or an aromatic ring. Specifically, examples of the compounds having a vinyl group include chain alkenes, branched alkenes, and cyclic alkenes, each optionally having a substituent. Examples of the compounds having a (meth)acrylate group include acrylic acid, methacrylic acid, acrylic acid ester, and methacrylic acid ester, each optionally having a substituent. Examples of the compounds having an allyl group include allyl alcohol, allyl ether, allyl ester, allyl amide, allylamine, and allyl-group-containing isocyanurates, each optionally having a substituent. Examples of the compounds having an alkynyl group include chain alkynes, branched alkynes, cyclic alkynes, alkynyl alcohols, alkynyl ethers, alkynyl esters, alkynyl amides, alkynyl amines, and alkynyl-group-containing isocyanurates, each optionally having a substituent. Examples of the compounds having an aromatic ring include arenes, heteroarenes, styrene, stilbene, phenylacetylene, acenaphthylene, and chalcone, each optionally having a substituent. The crosslinking agent may have only one of the above functional groups, or may have a plurality thereof. The number of the above functional groups contained in the crosslinking agent is preferably 1 or more and 10 or fewer, more preferably 2 or more and 8 or fewer.

When the resist composition contains the crosslinking agent, the contained amount is preferably 0.01 to 50 mass % of the total solid content. One kind of the crosslinking agent may be used, or two or more kinds thereof may be used in combination.

When the resist composition contains the crosslinking agent, a photo-polymerization initiator (photo-radical generator) may further be contained. The photo-polymerization initiator generates radicals upon irradiation with a high-energy beam, and can promote the crosslinking of the crosslinking agent.

Specific examples of the photo-polymerization initiator include: benzophenone derivatives, such as benzophenone, methyl O-benzoylbenzoate, 4-benzoyl-4′-methyldiphenyl ketone, dibenzyl ketone, and fluorenone; acetophenone derivatives, such as 2,2′-diethoxyacetophenone, 2-hydroxy-2-methylpropiophenone, 2,2-dimethoxy-1,2-diphenylethan-1-one, 1-hydroxycyclohexyl phenyl ketone, 2-methyl-1-[4-(methylthio)phenyl]-2-morpholinopropan-1-one, 2-hydroxy-1-{4-[4-(2-hydroxy-2-methylpropionyl)-benzyl]-phenyl}-2-methylpropan-1-one, and methyl phenylglyoxylate; thioxanthone derivatives, such as thioxanthone, 2-methylthioxanthone, 2-isopropylthioxanthone, 4-isopropylthioxanthone, 2-chlorothioxanthone, and diethylthioxanthone; benzyl derivatives, such as benzyl, benzyldimethyl ketal, and benzyl-B-methoxyethyl acetal; benzoin derivatives, such as benzoin, benzoin methyl ether, and 2-hydroxy-2-methyl-1-phenylpropan-1-one; oxime compounds, such as 1-phenyl-1,2-butanedione-2-(O-methoxycarbonyl)oxime, 1-phenyl-1,2-propanedione-2-(O-methoxycarbonyl)oxime, 1-phenyl-1,2-propanedione-2-(O-ethoxycarbonyl)oxime, 1-phenyl-1,2-propanedione-2-(O-benzoyl)oxime, 1,3-diphenylpropanetrione-2-(O-ethoxycarbonyl)oxime, 1-phenyl-3-ethoxypropantrione-2-(O-benzoyl)oxime 1,2-octanedione, 1-[4-(phenylthio)-2-(O-benzoyloxime)]ethanone, and 1-[9-ethyl-6-(2-methylbenzoyl)-9H-carbazol-3-yl]-1-(O-acetyloxime); α-hydroxyketone compounds, such as 2-hydroxy-2-methyl-1-phenylpropan-1-one, 1-[4-(2-hydroxyethoxy)phenyl]-2-hydroxy-2-methyl-1-propan-1-one and 2-hydroxy-1-{4-[4-(2-hydroxy-2-methylpropionyl)-benzyl]phenyl}-2-methylpropane; α-aminoalkylphenone compounds, such as 2-benzyl-2-dimethylamino-1-(4-morpholinophenyl)-butanone-1 and 2-dimethylamino-2-(4-methylbenzyl)-1-(4-morpholin-4-yl-phenyl)butan-1-one; phosphine oxide compounds, such as bis(2,4,6-trimethylbenzoyl)-phenylphosphine oxide, bis(2,6-dimethoxybenzoyl)-2,4,4-trimethyl-pentylphosphine oxide, and 2,4,6-trimethylbenzoyldiphenylphosphine oxide; and titanocene compounds, such as bis(η5-2,4-cyclopentadien-1-yl)-bis(2,6-difluoro-3-(1H-pyrrol-1-yl) phenyl)titanium.

When the resist composition contains the photo-polymerization initiator, the contained amount is preferably 0.1 to 10 mass %, more preferably 0.1 to 5 mass %, and most preferably 0.1 to 1 mass % of the total solid contents. When the amount is 0.1 mass % or more, a sufficient blending effect can be achieved.

The resist composition mainly contains the hypervalent iodine compound and the carboxy group-containing compound as described above, and does not require polymers having an acid-labile group and a photoacid generator, each of which is contained in conventional chemically amplified resist compositions.

However, the inventive resist composition makes it possible to form a positive pattern, where exposed areas become soluble in a developer, or a negative pattern, where exposed areas become insoluble in a developer, especially by exposure to EB or EUV. The mechanism is not completely clear, but the following conjecture can be made, for example.

The hypervalent iodine compound having a carboxylate coordinated to the hypervalent iodine and represented by the formula (1), (2), or (3) is a compound having a tricoordinate hypervalent iodine having a carboxylate ligand. It can be assumed that when such a tricoordinate iodine compound is mixed with a carboxy group-containing compound, carboxylate ligand exchange occurs as an equilibrium reaction. In this event, if the original carboxylate ligand can be removed by some method, a hypervalent iodine compound having a new ligand is generated. For example, when the hypervalent iodine compound represented by the following formula (1′) and a carboxy group-containing compound are mixed, and the generated acetic acid having a low boiling point is removed, ligand exchange is completed. Here, obtained is a polymer in which the carboxy group-containing compound is bonded to the hypervalent iodine compound.

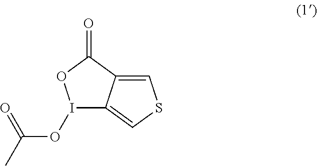

The polymer with the hypervalent iodine compound bonded is produced at the time of film formation. This is because such a polymer is insoluble in most organic solvents, and therefore, a solution cannot be prepared even if the polymer is synthesized beforehand. This is conjectured to be because the hypervalent iodine compound, originally having low solvent solubility due to high polarization, has the carboxy group-containing compound as a ligand, which further reduces its solubility. Accordingly, it is desirable to remove the original low-molecular-weight carboxylic acid component during film formation and subsequent baking process, thereby completing the ligand-exchange reaction and also forming a resist film.

The resist film obtained from the inventive resist composition changes in polarity by decomposition of the hypervalent iodine compound by light, which is the main component of the resist film, and a pattern is formed by a development process. The mechanism is not completely clear, but the following conjecture can be made, for example. It should be understood that, by selecting an appropriate developer, a positive-type or negative-type pattern can be formed.

The inventive resist composition can be either positive or negative, depending on the choice of components. In the case of a positive type, a polymer to which a hypervalent iodine compound is bonded is contained during film formation. By the polymer being decomposed by light, a monovalent iodine compound is formed. At the same time, the bond between the carboxy group-containing compound and the hypervalent iodine compound is released, and the molecular weight is reduced. It is conjectured that, as a result, a positive pattern, where exposed areas are removed with an organic solvent, is formed.

On the other hand, in the case of a negative type, a polymer generated during film formation and crosslinked with a hypervalent iodine compound is contained. By the polymer being decomposed by light, exchange in crosslinking or bonding occurs, thereby increasing molecular weight and converting polarity. It is conjectured that, as a result, a negative pattern, where unexposed areas are removed with an alkaline aqueous solution, is formed.

In the hypervalent iodine compounds represented by formula (1), (2), or (3), a thiophene skeleton, a pyrazole skeleton, or a pyridine skeleton bonds to an iodine atom. Therefore, different from tricoordinate hypervalent iodine compounds having an aryl group and a carboxylate ligand, they have a small molecular weight, excellent solubility in a developer, and are presumed to have significantly different photoreactivity.

Therefore, they can form a pattern with high sensitivity and excellent resolution having small roughness.

The hypervalent iodine compounds represented by formula (1), (2), or (3) can undergo ligand exchange with a carboxyl group-containing compound at one of the two ligands at the apical position of the hypervalent iodine. Therefore, the crosslink density of the crosslinked structure after ligand exchange with a carboxyl group-containing compound is lower than that of a compound such as diacetoxyiodothiophene, which can undergo ligand exchange with a carboxyl group-containing compound at two positions of repeating units. Accordingly, it is possible to induce crosslink cleavage and bond-recombination reactions upon exposure to light by using low energy. Therefore, resists using the hypervalent iodine compound used in the present invention can form a pattern with higher sensitivity than resist compositions using hypervalent iodine, such as diacetoxyiodothiophene, which can undergo ligand exchange with carboxyl group-containing compounds at two positions of repeating units.

Based on the above-described conjecture, it can be said that the inventive resist composition is a non-chemically amplified resist composition. The inventive resist composition does not require an acid-labile group-containing polymer or a photoacid generator, unlike conventional chemically amplified resist compositions. Therefore, adverse effects (e.g., image blur) due to acid diffusion do not occur, and a fine pattern can be resolved.

The inventive resist composition is extremely effective, especially in EUV lithography. This result is attributed to having an iodine atom, which is capable of greatly absorbing EUV light. That is, shot noise can be reduced, and higher resolution and lower LWR can be achieved.

As a resist composition for EUV lithography with which a fine pattern can be formed, a metal resist that mainly contains a metal tin compound, which has a high absorbance of EUV light as iodine atoms (e.g., Patent Document 2), is reported. However, as described above, such a metal resist has many problems, such as insufficient solubility in a solvent, storage stability, and defects due to residues after etching due to containing a metal element. On the other hand, the inventive resist composition has an advantage over metal resists in terms of defects, since a metal element is not used, and there is no problem in terms of solubility in a solvent either. Moreover, the inventive resist composition is applicable to either a positive-type or a negative-type and therefore has a wide range of uses. For example, in a contact-hole formation step, a reversal process is necessary after forming a pillar pattern in the case of a metal resist to be subjected to negative development, but such a process is unnecessary in the case of a positive resist. Therefore, it can be said that the inventive resist composition is more useful than metal resists from the viewpoint of the simplicity and convenience of the process as well.

JP2015-180928A and JP2018-95853A disclose a resist composition containing a hypervalent iodine compound as an additive and a resist composition in which a hypervalent iodine compound is incorporated in the polymer skeleton of a base polymer. However, the characteristic of the resist compositions disclosed in these Patent Documents is only improvement of line edge roughness, and there is no mention whatsoever regarding the possibility that the hypervalent iodine compound may be photolysed, or the possibility that the resist composition may function as material for a non-chemically amplified resist composition. Furthermore, according to the description and specific examples regarding the contained amount, the hypervalent iodine compound is not the main component. Accordingly, it is considered that a material that makes it possible to reduce shot noise in EUV lithography and also form a fine pattern as a non-chemically amplified resist composition material, like the material of the present invention, cannot be conceived from these Patent Documents. That is, it can be said that the present invention provides a clearly novel resist composition and a clearly novel patterning process.

### [Laminate]

The present invention provides a laminate including: a substrate; and a resist film, which is a film of the above-described resist composition, on the substrate. In such a laminate, including a resist film obtained from the non-chemically amplified resist composition of the present invention, the resist film, which is a film of the above-described resist composition, has extremely high sensitivity, also exhibits excellent resolution, is extremely effective for precise fine processing, and in addition, is applicable to either positive or negative patterning. Therefore, the laminate has a wide range of uses, and is extremely highly useful in resist process technology.

In this case, a resist underlayer film may be further provided as necessary between the substrate and the resist film.

Furthermore, in the inventive laminate, the resist film preferably contains a product of a ligand-exchange reaction between the hypervalent iodine compound and the carboxy group-containing compound. That is, the laminate is obtained by forming a substrate and a resist film, obtained from the inventive resist composition, on the substrate, and the resist film is preferably one formed by ligand exchange between the hypervalent iodine compound and the carboxy group-containing compound.

As described above, by removing low-molecular-weight carboxylic acid by-produced during film formation and in the subsequent baking process, the ligand-exchange reaction between the hypervalent iodine compound and the carboxy group-containing compound progresses, and a resist film containing a product of the ligand-exchange reaction is formed (that is, a film is produced). By the ligand exchange being completed, the carboxy group-containing compound is crosslinked with the hypervalent iodine compound to form a polymer. It is preferable to form the resist film by completing the ligand-exchange reaction in this manner.

### [Patterning Process]

When the inventive resist composition is used for manufacturing various integrated circuits, a publicly known lithography technique can be applied. Examples of patterning processes include a method including the steps of: forming a resist film on a substrate or on a resist underlayer film of a substrate having the resist underlayer film laminated thereon, using the above-described resist composition; exposing the resist film by a high-energy beam; and developing the exposed resist film by using a developer. Hereinafter, the resist underlayer film is also simply referred to as “underlayer film”.

Firstly, the inventive resist composition is applied onto a substrate for manufacturing an integrated circuit, onto an underlayer film of a substrate (Si, SiO₂, SiN, SiON, TiN, WSi, BPSG, SOG, organic antireflective film, etc.) on which the underlayer film is laminated, onto a substrate for manufacturing a mask circuit, or onto an underlayer film of a substrate (Cr, CrO, CrON, MoSi₂, SiO₂, etc.) on which the underlayer film is laminated, by an appropriate coating method, such as spin coating, roll coating, flow coating, dip coating, spray coating, or doctor coating, so that the thickness of the coating film is 0.01 to 2 μm. The resultant is prebaked on a hot plate preferably at 60 to 200° C. for 10 seconds to 30 minutes, more preferably 80 to 180° C. for 30 seconds to 20 minutes. Thus, a resist film is formed. It should be understood that an underlayer film is a film formed between a substrate and a resist film in a multilayer resist process. The underlayer film is not particularly limited, and a conventionally publicly known film can be used.

Subsequently, the resist film is exposed by using a high-energy beam. Examples of the high-energy beam include ultraviolet ray (g-line (436 nm), h-line (405 nm), i-line (365 nm), etc.), deep ultraviolet ray, EB, EUV, X-ray, soft X-ray, excimer laser beam (KrF excimer laser beam, ArF excimer laser beam, etc.), y-ray, and synchrotron radiation. As the high-energy beam, it is preferable to use an i-line, a KrF excimer laser beam, an ArF excimer laser beam, an electron beam, or an extreme ultraviolet ray. When ultraviolet ray, deep ultraviolet ray, EUV, X-ray, soft X-ray, an excimer laser beam, γ-ray, synchrotron radiation, or the like is employed as the high-energy beam, the irradiation is performed directly or while using a mask for forming a target pattern at an exposure dose of preferably about 1 to 300 mJ/cm², more preferably about 10 to 200 mJ/cm². When an EB is employed as the high-energy beam, the drawing is performed directly or while using a mask for forming a target pattern at an exposure dose of preferably about 0.1 to 8000 μC/cm², more preferably about 0.5 to 5000 μC/cm². It should be understood that the inventive resist composition is particularly suitable for fine patterning with EB or EUV, among the high-energy beams.

After the exposure, PEB is performed as necessary. In this event, after the exposure, the PEB is preferably performed on a hot plate or in an oven under the conditions of 30 to 200° C. for 10 seconds to 30 minutes, more preferably 60 to 180° C. for 30 seconds to 20 minutes.

After the exposure or after the PEB, development is performed by using a developer to perform patterning. Examples of the developer used in this event include: aqueous alkaline solutions, such as an aqueous solution of tetramethylammonium hydroxide and an aqueous solution of tetrabutylammonium hydroxide; and organic solvents, such as 2-octanone, 2-nonanone, 2-heptanone, 3-heptanone, 4-heptanone, 2-hexanone, 3-hexanone, diisobutyl ketone, 5-methyl-2-hexanone, methylcyclohexanone, acetophenone, methylacetophenone, isopropyl alcohol, isoamyl alcohol, n-butanol, tert-butyl alcohol, tert-pentyl alcohol, n-pentanol, cyclohexanol, formic acid, acetic acid, propionic acid, propyl acetate, butyl acetate, isobutyl acetate, pentyl acetate, butenyl acetate, isopentyl acetate, cyclohexyl acetate, 4-tert-butylcyclohexyl acetate, octyl acetate, isobornyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, phenylmethyl acetate, phenylethyl acetate, benzyl formate, phenylethyl formate, methyl 3-phenylpropionate, benzyl propionate, 2-phenylethyl acetate, 1-propanol, 3-methoxybutanol, 3-methyl-3-methoxybutanol, 1-methoxy-2-propanol, 1-ethoxy-2-propanol, 3-methyl-1-butanol, diacetone alcohol, 4-methyl-2-pentanol, 3-methylcyclohexanol, 3,5,5-trimethylhexyl alcohol, 2,6-dimethyl-4-heptanol, toluene, anisole, and s-caprolactone. One kind of these developers may be used, or two or more kinds thereof may be used in mixture.

After the development, rinsing is performed as necessary. The rinsing liquid is preferably a solvent that is miscible with the developer but does not dissolve the resist film. As such a solvent, it is preferable to use: an alcohol having 3 to 10 carbon atoms; an ether compound having 8 to 12 carbon atoms; and an alkane, alkene, alkyne, and aromatic solvent, each having 6 to 12 carbon atoms.

The rinsing can reduce collapse of a resist pattern and defect occurrence. Meanwhile, the rinsing is not necessarily essential, and the amount of solvent used can be reduced by not performing the rinsing.

In the inventive resist composition, a difference in the solubility occurs between exposed areas and unexposed areas by the exposure as described above, and a positive-type or negative-type pattern can be formed. Therefore, it is possible to use a developer that dissolves exposed areas and does not dissolve unexposed areas, or a developer that dissolves unexposed areas and does not dissolve exposed areas. Thus, the inventive patterning process makes it possible to form a positive-type or negative-type pattern by selecting an appropriate developer, and therefore is widely applicable to various kinds of fine patterning.

As described above, the present invention relates to a non-chemically amplified resist composition and is characterized in that a heterocyclic ring-containing hypervalent iodine compound is used as a resist material containing a hypervalent iodine compound. This improves solvent solubility, thereby improving sensitivity, enhancing uniformity, and reducing roughness, making it effective for forming fine patterns.

### EXAMPLE

Hereinafter, the present invention will be specifically described with reference to Synthesis Examples, Examples, and Comparative Example. However, the present invention is not limited to the following Examples.

### [1] Synthesis of Hypervalent Iodine Compound

### [Synthesis Example 1-1] Synthesis of Hypervalent Iodine Compound I-1

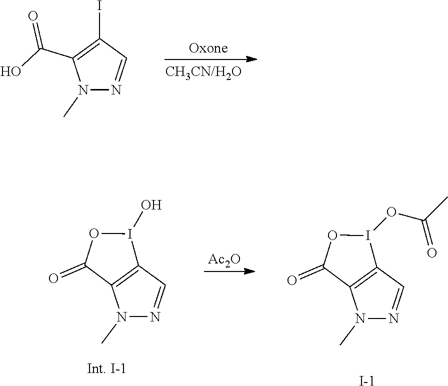

To a solution prepared by dissolving 40 g of OXONE (registered trademark) (potassium peroxymonosulfate) in 40 g of water, a solution prepared by dissolving 28.0 g of 4-iodo-1-methyl pyrazole-5-carboxylic acid as a raw material compound in 40 g of acetonitrile was added dropwise at room temperature. After the dropwise addition, the mixture was stirred for 2 hours, and an oily mixture was obtained. The obtained reaction solution was added with diisopropylether, and the supernatant was removed to obtain an intermediate Int.I-1 in an oily form (yield: 23.8 g, yield rate: 80%). Next, 17 g of Int.I-1 and 27 mL of acetic anhydride were mixed, stirred at 140° C. for 2 hours, and then the reaction solution was cooled to room temperature. The resultant reaction solution was added with diisopropylether, and the supernatant was removed to obtain the target hypervalent iodine compound I-1 in the solid state (yield: 8.5 g, yield rate: 43%).

### [Synthesis Example 1-2 to 1-3] Synthesis of Hypervalent Iodine Compounds I-2 and I-3

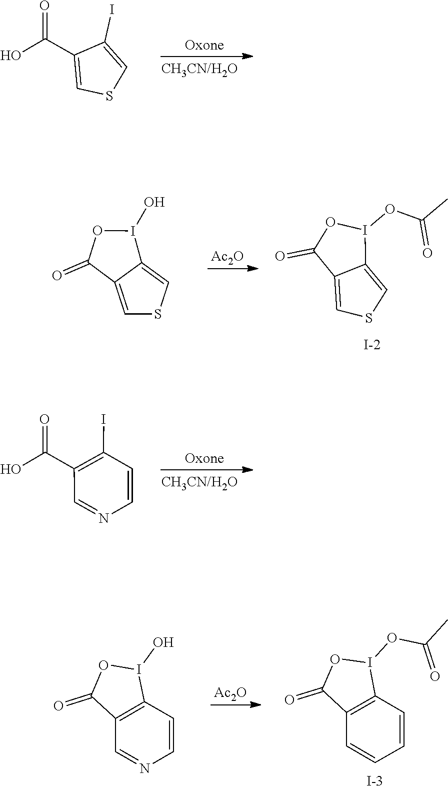

I-2 and I-3 were synthesized in the same manner as I-1, except that the raw materials were changed as shown in the above formulae. (I-2: Yield rate 36%, I-3: Yield rate 22%)

### [2] Synthesis of Carboxy Group-Containing Polymer

The monomers a-1 to a-3, b-1 to b-3, and c-1 to c-3 used for the synthesis of the carboxy group-containing polymers are as follows.

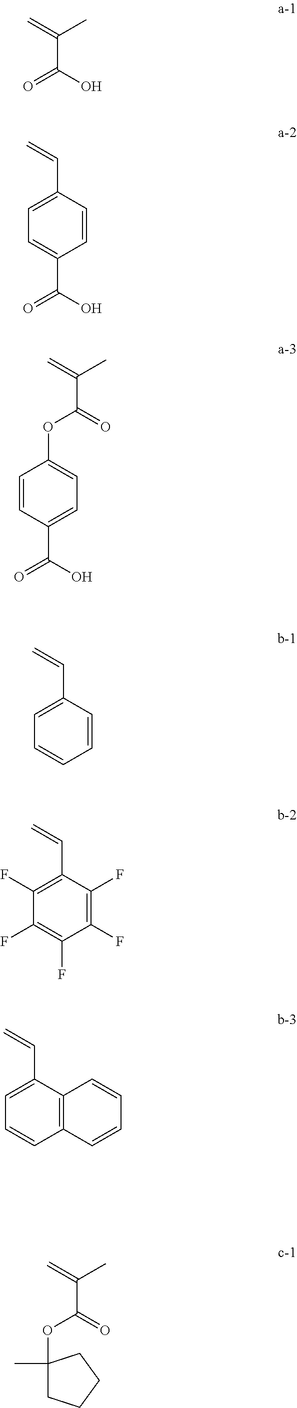

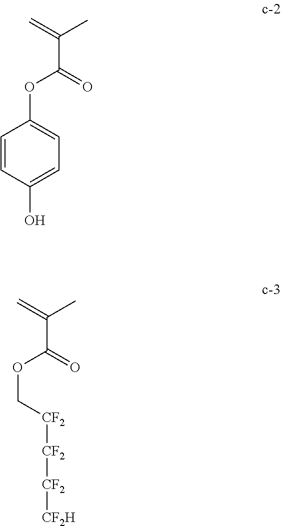 [SMILES: C=C(C)C(=O)Oc1ccc(O)cc1 ; C=C(C)C(=O)OCC(F)(F)C(F)(F)C(F)(F)C(F)F]

### [Synthesis Example 2-1] Synthesis of Polymer P-1

Monomer a-1 (56 g), monomer b-1 (36 g), 5.4 g of V-601 (FUJIFILM Wako Pure Chemical Industries, Ltd.), and 180 g of MEK were placed in a flask under a nitrogen atmosphere to prepare a monomer-polymerization initiator solution. 55 g of MEK was placed in another flask under a nitrogen atmosphere and heated to 80° C. while stirring, and then the monomer-polymerization initiator solution was added dropwise thereto over 4 hours. After completion of the dropwise addition, the stirring was continued for 2 hours while maintaining the temperature of the polymerization solution at 80° C., and then the polymerization solution was cooled to room temperature. The obtained polymerization solution was added dropwise to 4000 g of vigorously stirred hexane, and the precipitated polymer was separated by filtration. The obtained polymer was washed twice with 1200 g of hexane and then dried under vacuum at 50° C. for 20 hours to obtain a polymer P-1 in a form of a white powder (yield: 90 g, yield rate 98%). The Mw of the polymer P-1 was 8000, and Mw/Mn was 1.42. It should be understood that Mw and Mn were values in terms of standard polystyrene, measured by GPC using THF as an eluent. Specifically, the measurements were taken under the following conditions (The same shall apply hereafter.).
Apparatus: HLC-8320GPC
Column: TSK guardcolumn
   +TSKgel G4000HXL
   +TSKgel G2000HXL
   +TSKgel superH5000
Pump, Column temperature: 40° C.
Eluent: THF
Detector: RI (differential refractive index) detector
Injection amount: 100 μL

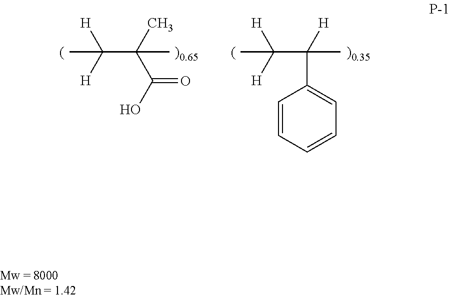

### [Synthesis Examples 2-2 to 2-13] Synthesis of Polymers P-2 to P-13

Each polymer shown in Table 1 was synthesized in the same manner as in Synthesis Example 2-1 except that the types of monomers and the composition ratios thereof were changed. Herein, the polymer P-13 is a polymer having no carboxy group (—COOH) and does not correspond to the carboxy group-containing compound of the present invention.

TABLE 1

| | | Introduction | | Introduction | | |
|---|---|---|---|---|---|---|
| | | ratio | | ratio | | |
| Polymer | Unit 1 | (mol %) | Unit 2 | (mol %) | Mw | Mw/Mn |
| P-1 | a-1 | 65 | b-1 | 35 | 8000 | 1.42 |
| P-2 | a-1 | 50 | b-2 | 50 | 8400 | 1.51 |
| P-3 | a-1 | 60 | b-3 | 40 | 8100 | 1.42 |
| P-4 | a-1 | 60 | c-3 | 40 | 8100 | 1.42 |
| P-5 | a-2 | 65 | b-1 | 35 | 8000 | 1.44 |
| P-6 | a-2 | 50 | b-2 | 50 | 8600 | 1.41 |
| P-7 | a-2 | 60 | b-3 | 40 | 7900 | 1.49 |
| P-8 | a-2 | 65 | c-3 | 35 | 9800 | 1.45 |
| P-9 | a-3 | 65 | b-1 | 35 | 9400 | 1.40 |
| P-10 | a-3 | 50 | b-2 | 50 | 9000 | 1.45 |
| P-11 | a-3 | 60 | b-3 | 40 | 7500 | 1.46 |
| P-12 | a-3 | 65 | c-3 | 35 | 7000 | 1.48 |
| P-13 | c-1 | 60 | c-2 | 40 | 8500 | 1.45 |

### [3] Preparation of Resist Compositions

### Examples 1-1 to 1-21 and Comparative Examples 1-1 to 1-6

By dissolving the hypervalent iodine compound and the carboxy group-containing compound in a solvent containing 0.01 mass % of a surfactant (PF-636, manufactured by OMNOVA Solutions Inc.) at the composition ratios shown in Table 2 below, and filtrating the resulting solution with a 0.2-μm Teflon (registered trademark) filter, the resist compositions (R-01 to R-21) of Examples 1-1 to 1-21 and the resist compositions (CR-01 to CR-04) of Comparative Examples 1-1 to 1-4 were prepared. Further, by dissolving the polymer, the photoacid generator, and the sensitivity modifier in a solvent containing 0.01 mass % of a surfactant (PF-636, manufactured by OMNOVA Solutions Inc.) at the composition ratios shown in Table 3, and filtrating the resulting solution with a 0.2-μm Teflon (registered trademark) filter, the resist compositions (CR-05 to CR-06) of Comparative Examples 1-5 to 1-6 were prepared. The resist composition R-04 of Example 1-4 was a combination of a pyrazole-type hypervalent iodine compound represented by the formula (1) and a thiophene-type hypervalent iodine compound represented by formula (2).

TABLE 2

| | | | Other | | | |
|---|---|---|---|---|---|---|
| | | Hypervalent | hypervalent | Carboxylic | | |
| | | iodine | Iodine | Acid | | |
| | | compound | Compound | compound | Solvent 1 | Solvent 2 |
| | | (parts by | (parts by | (parts by | (parts by | (parts by |
| | Resist | mass) | mass | mass) | mass) | mass) |
| Example 1-1 | R-01 | I-1 (20) | | P-1 (9) | PGMEA | AcOH |
| | | | | | (800) | (200) |
| Example 1-2 | R-02 | I-2 (26) | | P-1 (9) | PGMEA | AcOH |
| | | | | | (800) | (200) |
| Example 1-3 | R-03 | I-3 (20) | | P-1 (9) | PGMEA | AcOH |
| | | | | | (800) | (200) |
| Example 1-4 | R-04 | I-1 (10) | I-2 (13) | P-1 (9) | PGMEA | AcOH |
| | | | | | (800) | (200) |
| Example 1-5 | R-05 | I-2 (26) | | P-2 (17) | PGMEA | AcOH |
| | | | | | (800) | (200) |
| Example 1-6 | R-06 | I-2 (26) | | P-3 (11) | PGMEA | AcOH |
| | | | | | (800) | (200) |
| Example 1-7 | R-07 | I-2 (26) | | P-4 (17) | PGMEA | AcOH |
| | | | | | (800) | (200) |
| Example 1-8 | R-08 | I-2 (26) | | P-5 (12) | PGMEA | AcOH |
| | | | | | (800) | (200) |
| Example 1-9 | R-09 | I-2 (26) | | P-6 (21) | PGMEA | AcOH |
| | | | | | (800) | (200) |
| Example 1-10 | R-10 | I-2 (26) | | P-7 (15) | PGMEA | AcOH |
| | | | | | (800) | (200) |
| Example 1-11 | R-11 | I-2 (26) | | P-8 (19) | PGMEA | AcOH |
| | | | | | (800) | (200) |
| Example 1-12 | R-12 | I-2 (26) | | P-9 (21) | PGMEA | AcOH |
| | | | | | (800) | (200) |
| Example 1-13 | R-13 | I-2 (26) | | P-10 (29) | PGMEA | AcOH |
| | | | | | (800) | (200) |
| Example 1-14 | R-14 | I-2 (26) | | P-11 (24) | PGMEA | AcOH |
| | | | | | (800) | (200) |
| Example 1-15 | R-15 | I-2 (26) | | P-12 (27) | PGMEA | AcOH |
| | | | | | (800) | (200) |
| Example 1-16 | R-16 | I-2 (26) | | m-1 (7) | PGMEA | AcOH |
| | | | | | (800) | (200) |
| Example 1-17 | R-17 | I-2 (26) | | m-2 (4) | PGMEA | AcOH |
| | | | | | (800) | (200) |
| Example 1-18 | R-18 | I-2 (26) | | m-3 (7) | PGMEA | AcOH |
| | | | | | (800) | (200) |
| Example 1-19 | R-19 | I-2 (26) | | m-4 (7) | PGMEA | AcOH |
| | | | | | (800) | (200) |
| Example 1-20 | R-20 | I-2 (26) | | m-5 (10) | PGMEA | AcOH |
| | | | | | (800) | (200) |
| Example 1-21 | R-21 | I-2 (26) | | m-6 (8) | PGMEA | AcOH |
| | | | | | (800) | (200) |
| Comparative | CR-01 | I-4 (10) | | P-1(9) | PGMEA | AcOH |
| Example 1-1 | | | | | (800) | (200) |
| Comparative | CR-02 | I-4 (10) | | P-9(12) | PGMEA | AcOH |
| Example 1-2 | | | | | (800) | (200) |
| Comparative | CR-03 | I-5 (20) | | P-1(9) | PGMEA | AcOH |
| Example 1-3 | | | | | (800) | (200) |
| Comparative | CR-04 | I-5 (20) | | P-9(12) | PGMEA | AcOH |
| Example 1-4 | | | | | (800) | (200) |

TABLE 3

| | | | Photoacid | Sensitivity | | |
|---|---|---|---|---|---|---|
| | | Polymer | generator | Modifier | Solvent 1 | Solvent 2 |
| | Resist | (parts by | (parts by | (parts by | (parts by | (parts by |
| | composition | mass) | mass) | mass) | mass) | mass) |
| Comparative | CR-05 | P-13 (80) | PAG-1 (19) | Q-1 (6) | PGMEA | GBL |
| Example 1-5 | | | | | (1890) | (210) |
| Comparative | CR-06 | P-13 (80) | PAG-1 (19) | I-6 (5) | PGMEA | GBL |
| Example 1-6 | | | | | (1890) | (210) |

In Tables 2 and 3, the hypervalent iodine compounds I-4 and I-5, the carboxy group-containing compounds m-1 to m-6, the photoacid generator PAG-1, the sensitivity modifier Q-1, I-6, and solvents are as follows.

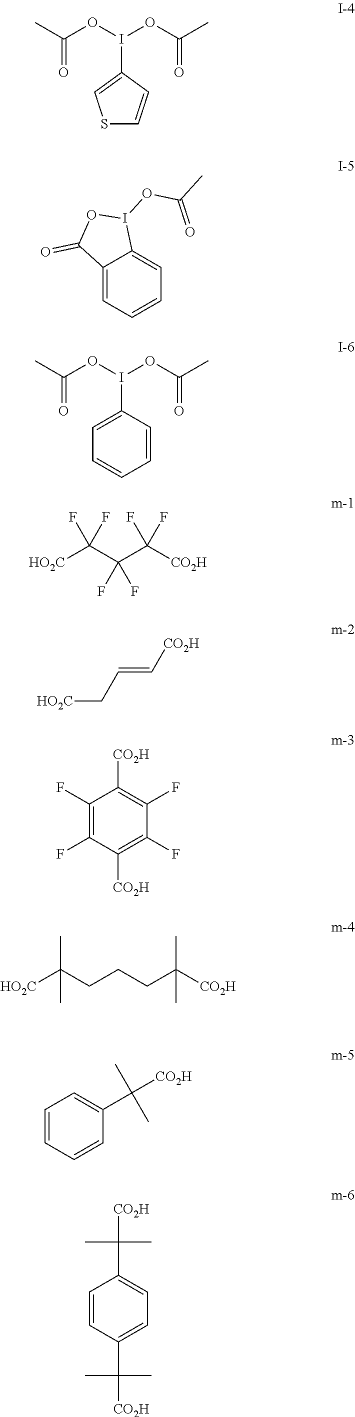

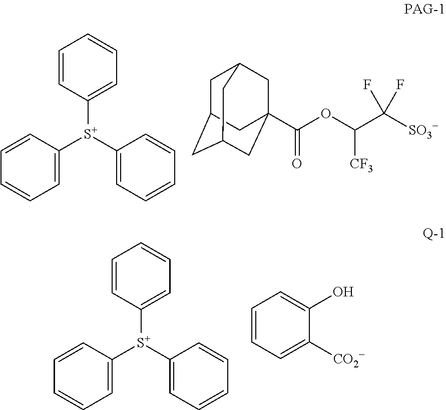

### Solvents:

PGMEA (propylene glycol monomethyl ether acetate)
AcOH (acetic acid)
GBL (y-butyrolactone)

### [4] EUV Lithography Evaluation (Line-and-Space Pattern)

### Examples 2-1 to 2-21 and Comparative Examples 2-1 to 2-6

Each of the resist compositions (R-01 to R-21 and CR-01 to CR-06) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness, and subjected to a post-apply bake (PAB) on a hot plate at the temperature shown in Table 4 for 60 seconds to form a resist film having a film thickness of 40 nm. The resist film was exposed using an EUV scanner NXE3400 (NA 0.33, σ 0.9, 90° dipole illumination), manufactured by ASML Holding N.V., to form a 36-nm 1:1 line-and-space (LS) pattern. Then, PEB was performed on a hot plate at the temperature shown in Table 4 for 60 seconds, and then development was performed with the developer shown in Table 4 for 30 seconds to form an LS pattern with a space width of 18 nm and a pitch of 36 nm.

Regarding the obtained resist pattern, the following evaluations were performed. The results are shown in Table 4.

### [Sensitivity Evaluation]

The LS pattern was observed using a CD-SEM (CG-6300), manufactured by Hitachi High-Tech Corporation, and an optimum exposure dose EoP (mJ/cm²) to yield the LS pattern with a space width of 18 nm and a pitch of 36 nm was determined and specified as sensitivity.

### [LWR Evaluation]

In the LS pattern obtained by irradiation at the optimum exposure dose, space widths at 10 positions in the longitudinal direction were measured with a CD-SEM (CG-6300), manufactured by Hitachi High-Tech Corporation. Using the results, a tripled value (3σ) of the standard variation (σ) was calculated and specified as LWR. A smaller LWR value can yield a pattern with smaller roughness and a more uniform space width.

### [Resolution Limit Evaluation]

A limit line width (nm) that can be resolved when forming a pattern while gradually increasing the exposure dose from the optimum exposure dose at which the LS pattern is formed was determined with a CD-SEM (CG-6300), manufactured by Hitachi High-Tech Corporation, and specified as the resolution limit (nm) A smaller value indicates that the resist composition has an excellent resolution limit and can form a finer pattern.

TABLE 4

| | Resist | PAB/PEB | | Type of | EoP | LWR | Resolution |
|---|---|---|---|---|---|---|---|
| | composition | (° C.) | Developer | pattern | (mJ/cm²) | (nm) | limit (nm) |
| Evaluation | R-01 | 110/90 | nBA | Positive | 28 | 3.0 | 13 |
| Example 2-1 | | | | | | | |
| Evaluation | R-02 | 110/90 | nBA | Positive | 29 | 3.2 | 13 |
| Example 2-2 | | | | | | | |
| Evaluation | R-03 | 110/90 | nBA | Positive | 28 | 3.2 | 13 |
| Example 2-3 | | | | | | | |
| Evaluation | R-04 | 110/90 | nBA | Positive | 28 | 3.0 | 13 |
| Example 2-4 | | | | | | | |
| Evaluation | R-05 | 110/90 | nBA | Positive | 28 | 3.0 | 13 |
| Example 2-5 | | | | | | | |
| Evaluation | R-06 | 110/90 | nBA | Positive | 27 | 3.1 | 13 |
| Example 2-6 | | | | | | | |
| Evaluation | R-07 | 110/90 | nBA | Positive | 28 | 3.3 | 14 |
| Example 2-7 | | | | | | | |
| Evaluation | R-08 | 110/90 | TMAH | Negative | 30 | 3.4 | 14 |
| Example 2-8 | | | | | | | |
| Evaluation | R-09 | 110/90 | TMAH | Negative | 30 | 3.3 | 14 |
| Example 2-9 | | | | | | | |
| Evaluation | R-10 | 110/90 | TMAH | Negative | 28 | 3.4 | 14 |
| Example 2-10 | | | | | | | |
| Evaluation | R-11 | 110/90 | TMAH | Negative | 28 | 3.3 | 14 |
| Example 2-11 | | | | | | | |
| Evaluation | R-12 | 110/90 | TMAH | Negative | 28 | 3.4 | 13 |
| Example 2-12 | | | | | | | |
| Evaluation | R-13 | 110/90 | TMAH | Negative | 26 | 3.2 | 13 |
| Example 2-13 | | | | | | | |
| Evaluation | R-14 | 110/90 | TMAH | Negative | 26 | 3.4 | 14 |
| Example 2-14 | | | | | | | |
| Evaluation | R-15 | 110/90 | TMAH | Negative | 25 | 3.4 | 14 |
| Example 2-15 | | | | | | | |
| Evaluation | R-16 | 110/90 | TMAH | Negative | 26 | 3.4 | 14 |
| Example 2-16 | | | | | | | |
| Evaluation | R-17 | 110/90 | TMAH | Negative | 27 | 3.4 | 14 |
| Example 2-17 | | | | | | | |
| Evaluation | R-18 | 110/90 | TMAH | Negative | 28 | 3.1 | 13 |
| Example 2-18 | | | | | | | |
| Evaluation | R-19 | 110/90 | nBA | Positive | 29 | 3.4 | 14 |
| Example 2-19 | | | | | | | |
| Evaluation | R-20 | 110/90 | nBA | Positive | 26 | 3.4 | 14 |
| Example 2-20 | | | | | | | |
| Evaluation | R-21 | 110/90 | nBA | Positive | 28 | 3.2 | 13 |
| Example 2-21 | | | | | | | |
| Comparative | CR-01 | 110/90 | nBA | Positive | 30 | 3.0 | 13 |
| Evaluation | | | | | | | |
| Example 2-1 | | | | | | | |
| Comparative | CR-02 | 110/90 | TMAH | Negative | 32 | 3.2 | 14 |
| Evaluation | | | | | | | |
| Example 2-2 | | | | | | | |
| Comparative | CR-03 | 110/90 | nBA | Positive | 32 | 3.5 | 15 |
| Evaluation | | | | | | | |
| Example 2-3 | | | | | | | |
| Comparative | CR-04 | 110/90 | TMAH | Negative | 32 | 3.6 | 15 |
| Evaluation | | | | | | | |
| Example 2-4 | | | | | | | |
| Comparative | CR-05 | 105/90 | TMAH | Positive | 85 | 4.4 | 18 |
| Evaluation | | | | | | | |
| Example 2-5 | | | | | | | |
| Comparative | CR-06 | 105/90 | TMAH | Positive | 85 | 5.0 | 18 |
| Evaluation | | | | | | | |
| Example 2-6 | | | | | | | |

Developers:
nBA (butyl acetate)
TMAH (2.38 mass % aqueous solution of tetramethylammonium hydroxide)

From the results shown in Table 4, it was found, in Example 2-1 to 2-21 where the resist compositions containing the inventive heterocyclic ring-containing hypervalent iodine compound was used, that it was possible to form both positive-type and negative-type patterns depending on the developer used. In addition, when comparing the resist compositions of Comparative Examples 2-1 and 2-2 with the inventive resist compositions while focusing only on the positive-type resist compositions and only on the negative-type resist compositions, it was found that the inventive resist composition had excellent resolution. By comparing the inventive resist compositions with the resist compositions of Comparative Examples 2-3 and 2-4, it was found that the inventive resist composition had excellent sensitivity, excellent resolution, and excellent LWR. Further, it was found that, even when compared with the resist compositions of Comparative Examples 2-5 and 2-6, which were chemically amplified resist compositions using an acid catalytic reaction, the inventive resist composition had excellent sensitivity, excellent resolution, and excellent LWR. The results of Example 2-4 using the resist composition R-04 demonstrate that the combination of the inventive heterocycle-containing hypervalent iodine compounds is also effective. Thus, it was found that the inventive resist composition was excellent in resolution etc., in LS pattern formation by EUV exposure.

### [5] EUV Lithography Evaluation (Contact Hole Pattern)

### Examples 3-1 to 3-21 and Comparative Examples 3-1 to 3-6

Each of the resist compositions (R-01 to R-21 and CR-01 to CR-06) was applied by spin-coating on a Si substrate on which a silicon-containing spin-on hard mask SHB-A940, manufactured by Shin-Etsu Chemical Co., Ltd. (silicon content of 43 mass %), was formed with 20 nm in film thickness. Then, PAB was performed at the temperature shown in Table 5 for 60 seconds using a hot plate to form a resist film with 50 nm in film thickness. Subsequently, the resist film was exposed using an EUV scanner NXE3400 (NA 0.33, σ 0.9/0.6, quadrupole illumination, 64 nm in pitch on wafer size, hole pattern mask with +20% bias), manufactured by ASML Holding N.V. Then, PEB was performed at the temperature shown in Table 5 for 60 seconds on a hot plate. Thereafter, development was performed with the developer shown in Table 5 for 30 seconds to obtain a hole pattern with a size of 32 nm.

Regarding the obtained resist pattern, the following evaluations were performed. The results are shown in Table 5.

### [Sensitivity Evaluation]

The contact hole patterns were observed using a CD-SEM (CG-6300), manufactured by Hitachi High-Tech Corporation, and an optimum exposure dose EoP (mJ/cm²) to yield the hole pattern with a size of 22 nm was determined and specified as sensitivity.

### [CDU Evaluation]

Sizes of 50 hole patterns obtained by irradiation at the optimum exposure dose were measured, and using the results, a tripled value (3σ) of the standard variation (σ) was calculated and specified as CDU. The smaller this value is, the more uniform the hole diameters in a pattern can be.

### [Resolution Limit Evaluation]

A limit hole diameter (nm) that can be resolved when forming a hole pattern while gradually decreasing the exposure dose from the optimum exposure dose at which the hole pattern is formed was determined with a CD-SEM (CG-6300), manufactured by Hitachi High-Tech Corporation, and specified as the resolution limit (nm). A smaller value indicates that the resist composition has an excellent resolution limit and can form a pattern with a finer hole diameter.

TABLE 5

| | Resist | PAB/PEB | | Type of | EoP | CDU | Resolution |
|---|---|---|---|---|---|---|---|
| | composition | (° C.) | Developer | pattern | (mJ/cm²) | (nm) | limit (nm) |
| Evaluation | R-01 | 110/90 | nBA | Positive | 24 | 2.7 | 25 |
| Example 3-1 | | | | | | | |
| Evaluation | R-02 | 110/90 | nBA | Positive | 28 | 2.1 | 24 |
| Example 3-2 | | | | | | | |
| Evaluation | R-03 | 110/90 | nBA | Positive | 29 | 2.1 | 24 |
| Example 3-3 | | | | | | | |
| Evaluation | R-04 | 110/90 | nBA | Positive | 26 | 2.1 | 24 |
| Example 3-4 | | | | | | | |
| Evaluation | R-05 | 110/90 | nBA | Positive | 28 | 2.2 | 25 |
| Example 3-5 | | | | | | | |
| Evaluation | R-06 | 110/90 | nBA | Positive | 29 | 2.3 | 25 |
| Example 3-6 | | | | | | | |
| Evaluation | R-07 | 110/90 | nBA | Positive | 26 | 2.4 | 26 |
| Example 3-7 | | | | | | | |
| Evaluation | R-08 | 110/90 | TMAH | Negative | 27 | 2.5 | 26 |
| Example 3-8 | | | | | | | |
| Evaluation | R-09 | 110/90 | TMAH | Negative | 27 | 2.4 | 26 |
| Example 3-9 | | | | | | | |
| Evaluation | R-10 | 110/90 | TMAH | Negative | 27 | 2.9 | 28 |
| Example 3-10 | | | | | | | |
| Evaluation | R-11 | 110/90 | TMAH | Negative | 27 | 2.4 | 26 |
| Example 3-11 | | | | | | | |
| Evaluation | R-12 | 110/90 | TMAH | Negative | 26 | 2.4 | 26 |
| Example 3-12 | | | | | | | |
| Evaluation | R-13 | 110/90 | TMAH | Negative | 26 | 2.2 | 26 |
| Example 3-13 | | | | | | | |
| Evaluation | R-14 | 110/90 | TMAH | Negative | 26 | 2.4 | 29 |
| Example 3-14 | | | | | | | |
| Evaluation | R-15 | 110/90 | TMAH | Negative | 26 | 2.9 | 29 |
| Example 3-15 | | | | | | | |
| Evaluation | R-16 | 110/90 | TMAH | Negative | 24 | 2.5 | 27 |
| Example 3-16 | | | | | | | |
| Evaluation | R-17 | 110/90 | TMAH | Negative | 24 | 2.5 | 25 |
| Example 3-17 | | | | | | | |
| Evaluation | R-18 | 110/90 | TMAH | Negative | 24 | 2.1 | 24 |
| Example 3-18 | | | | | | | |
| Evaluation | R-19 | 110/90 | nBA | Positive | 30 | 2.7 | 24 |
| Example 3-19 | | | | | | | |
| Evaluation | R-20 | 110/90 | nBA | Positive | 28 | 2.4 | 27 |
| Example 3-20 | | | | | | | |
| Evaluation | R-21 | 110/90 | nBA | Positive | 25 | 2.2 | 27 |
| Example 3-21 | | | | | | | |
| Comparative | CR-01 | 110/90 | nBA | Positive | 30 | 2.7 | 25 |
| Evaluation | | | | | | | |
| Example 3-1 | | | | | | | |
| Comparative | CR-02 | 110/90 | TMAH | Negative | 32 | 2.9 | 25 |
| Evaluation | | | | | | | |
| Example 3-2 | | | | | | | |
| Comparative | CR-03 | 110/90 | nBA | Positive | 34 | 3.0 | 26 |
| Evaluation | | | | | | | |
| Example 3-3 | | | | | | | |
| Comparative | CR-04 | 110/90 | TMAH | Negative | 34 | 3.1 | 26 |
| Evaluation | | | | | | | |
| Example 3-4 | | | | | | | |
| Comparative | CR-05 | 105/90 | TMAH | Positive | 50 | 4.2 | 32 |
| Evaluation | | | | | | | |
| Example 3-5 | | | | | | | |
| Comparative | CR-06 | 105/90 | TMAH | Positive | 50 | 4.2 | 32 |
| Evaluation | | | | | | | |
| Example 3-6 | | | | | | | |

From the results shown in Table 5, it was found that, in Examples 3-1 to 3-21 where the used resist compositions contain the inventive heterocyclic ring-containing hypervalent iodine compound, it was possible to form both positive-type and negative-type patterns depending on the choice of the developer used. In addition, when comparing the inventive resist compositions with the resist compositions of Comparative Evaluation Examples 3-1 and 3-2, it was found that excellent sensitivity was achieved. Furthermore, when comparing the inventive resist compositions with the resist compositions of Comparative Evaluation Examples 3-3 and 3-4, it was found that excellent sensitivity and excellent CDU were achieved. Even when compared with Comparative Evaluation Examples 3-5 and 3-6, which were chemically amplified resist compositions using an acid catalytic reaction, it was found that excellent sensitivity, excellent solution, and excellent CDU were achieved. The results of Example 3-4 using the resist composition R-04 demonstrate that the combination of the inventive heterocycle-containing hypervalent iodine compounds is also effective. Thus, it was found that the inventive resist composition was excellent in resolution, etc., in contact hole pattern formation by EUV exposure.

The present description includes the following invention.

[1]: A resist composition comprising at least one kind of hypervalent iodine compounds selected from the following formulae (1), (2), and (3), a carboxy group-containing compound, and a solvent,

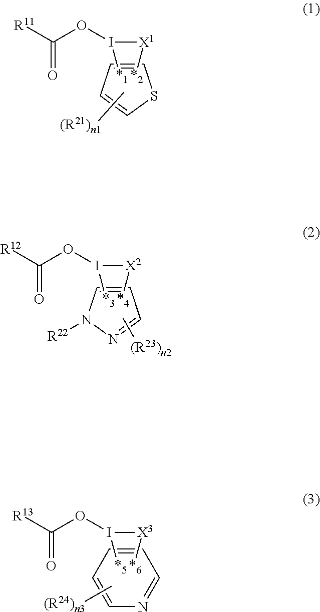

wherein n1 represents 0, 1, or 2; n2 represents 0, 1, or 2; n3 represents 0, 1, 2, or 3; R¹¹ to R¹³ each independently represent a halogen atom or a hydrocarbyl group having 1 to 10 carbon atoms and optionally having a heteroatom; X¹ to X³ each independently represent *—O—X^{A1}— or *—X^{A2} (—R¹⁴)—C(═O)—; X³ may be *—S—X^{A1}—; X^{A1} represents a carbonyl group or a hydrocarbylene group having 1 to 10 carbon atoms and optionally having a heteroatom; X^{A2} represents nitrogen or sulfur, and when X^{A2} is nitrogen, R¹⁴ may be included; R¹⁴ represents a hydrogen atom, a halogen atom, or a hydrocarbyl group or ester having 1 to 20 carbon atoms and optionally having a heteroatom; “*” represents a bond to an iodine atom in the formula; *1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle; R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN; R²² may be a hydrogen atom; When n1 is 2 or more, R²¹s each may be identical to or different from each other, and a plurality of R²¹s may be bonded to each other to form a ring together with the carbon atoms of the heteroaromatic ring to which they are bonded; and when n3 is 2 or more, R²⁴s each may be identical to or different from each other, and a plurality of R²⁴s may be bonded to each other to form a ring together with the carbon atoms of the heteroaromatic ring to which they are bonded, provided that R²² is X² when X² is bonded to a nitrogen atom to which R²² is bonded.
   [2]: The resist composition of the above [1], wherein the carboxy group-containing compound is either or both of a polymer having a repeating unit represented by the following formula (4) and a compound represented by the following formula (5),

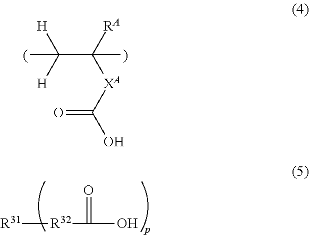

wherein R^{A} represents a hydrogen atom, a halogen atom, a methyl group, or a trifluoromethyl group; X^{A} represents a single bond, a phenylene group, a naphthylene group, or *—C(═O)—O—X^{A3}—; X^{A3} represents a saturated hydrocarbylene group having 1 to 20 carbon atoms, a phenylene group, or a naphthylene group, and the saturated hydrocarbylene group may have a hydroxy group, an ether bond, an ester bond, or a lactone ring; “*” represents a bond to a carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, and when “p” is 2, R³¹ may be an ether bond, a carbonyl group, an azo group, a thioether bond, a carbonate bond, a carbamate bond, a sulfinyl group, or a sulfonyl group; In addition, part or all of the hydrogen atoms of the p-valent hydrocarbon group or the p-valent heterocyclic group may be substituted with a group having a heteroatom, and part of the —CH₂— groups of the p-valent hydrocarbon group may be substituted with a group having a heteroatom; R³² represents a single bond or a hydrocarbylene group having 1 to 20 carbon atoms, part or all of the hydrogen atoms of the hydrocarbylene group may be substituted with a group having a heteroatom, and part of the —CH₂— groups of the hydrocarbylene group may be substituted with a group having a heteroatom; and when “p” is 2, 3, or 4, R³²S each may be identical to or different from each other.
   [3]: A laminate comprising a substrate and a resist film, which is a film of the resist composition of the above [1] or [2].
   [4]: The laminate of the above [3], further comprising a resist underlayer film between the substrate and the resist film.
   [5]: The laminate of the above [3] or [4], wherein the resist film contains a product of a ligand-exchange reaction between the hypervalent iodine compound and the carboxy group-containing compound.
   [6]: A patterning process comprising the steps of: forming a resist film on a substrate or on a resist underlayer film of a substrate having the resist underlayer film laminated thereon, using the resist composition of the above [1] or [2]; exposing the resist film by a high-energy beam; and developing the exposed resist film by using a developer.
   [7]: The patterning process of the above [6], wherein an i-line, a KrF excimer laser beam, an ArF excimer laser beam, an electron beam, or an extreme ultraviolet ray is used as the high-energy beam.
   [8]: The patterning process of the above [6] or [7], wherein the developer used is one that dissolves exposed areas and does not dissolve unexposed areas.
   [9]: The patterning process of the above [6] or [7], wherein the developer used is one that dissolves unexposed areas and does not dissolve exposed areas.

It should be noted that the present invention is not limited to the above-described embodiments. The embodiments are just examples, and any examples that have substantially the same feature and demonstrate the same functions and effects as those in the technical concept disclosed in claims of the present invention are included in the technical scope of the present invention.

1. A resist composition comprising at least one kind of hypervalent iodine compounds selected from the following formulae (1), (2), and (3), a carboxy group-containing compound, and a solvent, 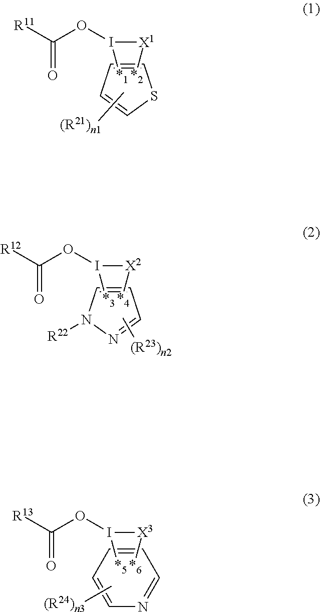
wherein n1 represents 0, 1, or 2; n2 represents 0, 1, or 2; n3 represents 0, 1, 2, or 3; R¹¹ to R¹³ each independently represent a halogen atom or a hydrocarbyl group having 1 to 10 carbon atoms and optionally having a heteroatom; X¹ to X³ each independently represent *—O—X^{A1}— or *—X^{A2}(—R¹⁴)—C(═O)—; X³ may be *—S—X^{A1}—; X^{A1} represents a carbonyl group or a hydrocarbylene group having 1 to 10 carbon atoms and optionally having a heteroatom; X^{A2} represents nitrogen or sulfur, and when X^{A2} is nitrogen, R¹⁴ may be included; R¹⁴ represents a hydrogen atom, a halogen atom, or a hydrocarbyl group or ester having 1 to 20 carbon atoms and optionally having a heteroatom; “*” represents a bond to an iodine atom in the formula; *1, *2, *3, *4, *5, and *6 represent a bond to a carbon atom or a heteroatom of the heterocycle in the formula, and *1, *2, *3, *4, *5, and *6 are bonded to adjacent carbon atoms or heteroatoms of the heterocycle; R²¹ to R²⁴ each independently represent a halogen atom, a hydrocarbyl group having 1 to 40 carbon atoms and optionally having a heteroatom, —NH₂, —NO₂, —SF₅, —N(CH₃)₂, —N(C₂H₅)₂, —OH, or —CN; R²² may be a hydrogen atom; When n1 is 2 or more, R²¹s each may be identical to or different from each other, and a plurality of R²¹s may be bonded to each other to form a ring together with the carbon atoms of the heteroaromatic ring to which they are bonded; and when n3 is 2 or more, R²⁴s each may be identical to or different from each other, and a plurality of R²⁴s may be bonded to each other to form a ring together with the carbon atoms of the heteroaromatic ring to which they are bonded, provided that R²² is X² when X² is bonded to a nitrogen atom to which R²² is bonded.

2. The resist composition according to claim 1, wherein the carboxy group-containing compound is either or both of a polymer having a repeating unit represented by the following formula (4) and a compound represented by the following formula (5), 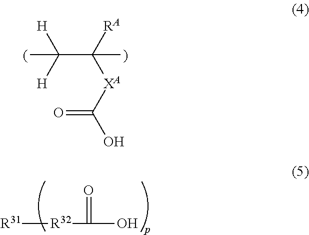
wherein R^{A} represents a hydrogen atom, a halogen atom, a methyl group, or a trifluoromethyl group; X^{A} represents a single bond, a phenylene group, a naphthylene group, or *—C(═O)—O—X^{A3}—; X^{A3} represents a saturated hydrocarbylene group having 1 to 20 carbon atoms, a phenylene group, or a naphthylene group, and the saturated hydrocarbylene group may have a hydroxy group, an ether bond, an ester bond, or a lactone ring; “*” represents a bond to a carbon atom of the main chain; “p” represents 1, 2, 3, or 4; R³¹ represents a p-valent hydrocarbon group having 1 to 40 carbon atoms or a p-valent heterocyclic group having 2 to 40 carbon atoms, and when “p” is 2, R³¹ may be an ether bond, a carbonyl group, an azo group, a thioether bond, a carbonate bond, a carbamate bond, a sulfinyl group, or a sulfonyl group; In addition, part or all of the hydrogen atoms of the p-valent hydrocarbon group or the p-valent heterocyclic group may be substituted with a group having a heteroatom, and part of the —CH₂— groups of the p-valent hydrocarbon group may be substituted with a group having a heteroatom; R³² represents a single bond or a hydrocarbylene group having 1 to 20 carbon atoms, part or all of the hydrogen atoms of the hydrocarbylene group may be substituted with a group having a heteroatom, and part of the —CH₂— groups of the hydrocarbylene group may be substituted with a group having a heteroatom; and when “p” is 2, 3, or 4, R³²S each may be identical to or different from each other.

3. A laminate comprising a substrate and a resist film, which is a film of the resist composition according to claim 1, on the substrate.

4. A laminate comprising a substrate and a resist film, which is a film of the resist composition according to claim 2, on the substrate.

5. The laminate according to claim 3, further comprising a resist underlayer film between the substrate and the resist film.

6. The laminate according to claim 4, further comprising a resist underlayer film between the substrate and the resist film.

7. The laminate according to claim 3, wherein the resist film contains a product of a ligand-exchange reaction between the hypervalent iodine compound and the carboxy group-containing compound.

8. The laminate according to claim 4, wherein the resist film contains a product of a ligand-exchange reaction between the hypervalent iodine compound and the carboxy group-containing compound.

9. A patterning process comprising the steps of:
forming a resist film on a substrate or on a resist underlayer film of a substrate having the resist underlayer film laminated thereon, using the resist composition according to claim 1;
exposing the resist film by a high-energy beam; and
developing the exposed resist film by using a developer.

10. A patterning process comprising the steps of:
forming a resist film on a substrate or on a resist underlayer film of a substrate having the resist underlayer film laminated thereon, using the resist composition according to claim 2;
exposing the resist film by a high-energy beam; and
developing the exposed resist film by using a developer.

11. The patterning process according to claim 9, wherein an i-line, a KrF excimer laser beam, an ArF excimer laser beam, an electron beam, or an extreme ultraviolet ray is used as the high-energy beam.

12. The patterning process according to claim 10, wherein an i-line, a KrF excimer laser beam, an ArF excimer laser beam, an electron beam, or an extreme ultraviolet ray is used as the high-energy beam.

13. The patterning process according to claim 9, wherein the developer used is one that dissolves exposed areas and does not dissolve unexposed areas.

14. The patterning process according to claim 10, wherein the developer used is one that dissolves exposed areas and does not dissolve unexposed areas.

15. The patterning process according to claim 11, wherein the developer used is one that dissolves exposed areas and does not dissolve unexposed areas.

16. The patterning process according to claim 12, wherein the developer used is one that dissolves exposed areas and does not dissolve unexposed areas.

17. The patterning process according to claim 9, wherein the developer used is one that dissolves unexposed areas and does not dissolve exposed areas.

18. The patterning process according to claim 10, wherein the developer used is one that dissolves unexposed areas and does not dissolve exposed areas.

19. The patterning process according to claim 11, wherein the developer used is one that dissolves unexposed areas and does not dissolve exposed areas.

20. The patterning process according to claim 12, wherein the developer used is one that dissolves unexposed areas and does not dissolve exposed areas.
